SEQUENCE LISTING (151 sequences)

SEQ ID NO: 1 - protein, 5 aa, Artificial Sequence
  source: /note=“Description of Artificial Sequence Synthetic peptide”
SYAIS

SEQ ID NO: 2 - protein, 17 aa, Artificial Sequence
  source: /note=“Description of Artificial Sequence Synthetic peptide”
VIFPAEGAPGYAQKFQG

SEQ ID NO: 3 - protein, 9 aa, Artificial Sequence
  source: /note=“Description of Artificial Sequence Synthetic peptide”
GGYISDFDV

SEQ ID NO: 4 - protein, 7 aa, Artificial Sequence
  source: /note=“Description of Artificial Sequence Synthetic peptide”
GGTFSSY

SEQ ID NO: 5 - protein, 6 aa, Artificial Sequence
  source: /note=“Description of Artificial Sequence Synthetic peptide”
FPAEGA

SEQ ID NO: 6 - protein, 10 aa, Artificial Sequence
  source: /note=“Description of Artificial Sequence Synthetic peptide”
GGTFSSYAIS

SEQ ID NO: 7 - protein, 8 aa, Artificial Sequence
  source: /note=“Description of Artificial Sequence Synthetic peptide”
GGTFSSYA

SEQ ID NO: 8 - protein, 8 aa, Artificial Sequence
  source: /note=“Description of Artificial Sequence Synthetic peptide”
IFPAEGAP

SEQ ID NO: 9 - protein, 11 aa, Artificial Sequence
  source: /note=“Description of Artificial Sequence Synthetic peptide”
ARGGYISDFDV

SEQ ID NO: 10 - protein, 118 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGVIFPAEGAPGYAQKFQGRVTITADESTSTAYMELSSLRSEDTAVYYCARGGYISDFDVWGQGTLVTVSS

SEQ ID NO: 11 - DNA, 354 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttagcagctatgcgattagctgggtgcgccaggccccgggccagggcctcgagtggatgggcgttatcttcccggctgaaggcgctccgggttacgcccagaaatttcagggccgggtgaccattaccgccgatgaaagcaccagcaccgcctatatggaactgagcagcctgcgcagcgaagatacggccgtgtattattgcgcgcgtggtggttacatctctgacttcgatgtttggggccaaggcaccctggtgactgttagctca

SEQ ID NO: 12 - protein, 448 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGVIFPAEGAPGYAQKFQGRVTITADESTSTAYMELSSLRSEDTAVYYCARGGYISDFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 1344 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttagcagctatgcgattagctgggtgcgccaggccccgggccagggcctcgagtggatgggcgttatcttcccggctgaaggcgctccgggttacgcccagaaatttcagggccgggtgaccattaccgccgatgaaagcaccagcaccgcctatatggaactgagcagcctgcgcagcgaagatacggccgtgtattattgcgcgcgtggtggttacatctctgacttcgatgtttggggccaaggcaccctggtgactgttagctcagctagcaccaagggccccagcgtgttccccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 14 - protein, 237 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGVIFPAEGAPGYAQKFQGRVTITADESTSTAYMELSSLRSEDTAVYYCARGGYISDFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLG

SEQ ID NO: 15 - DNA, 711 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttagcagctatgcgattagctgggtgcgccaggccccgggccagggcctcgagtggatgggcgttatcttcccggctgaaggcgctccgggttacgcccagaaatttcagggccgggtgaccattaccgccgatgaaagcaccagcaccgcctatatggaactgagcagcctgcgcagcgaagatacggccgtgtattattgcgcgcgtggtggttacatctctgacttcgatgtttggggccaaggcaccctggtgactgttagctcagctagcaccaagggccccagcgtgttccccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctggga

SEQ ID NO: 16 - protein, 11 aa, Artificial Sequence
  source: /note=“Description of Artificial Sequence Synthetic peptide”
RASQSISNYLA

SEQ ID NO: 17 - protein, 7 aa, Artificial Sequence
  source: /note=“Description of Artificial Sequence Synthetic peptide”
DASSLQS

SEQ ID NO: 18 - protein, 9 aa, Artificial Sequence
  source: /note=“Description of Artificial Sequence Synthetic peptide”
QQYYYESIT

SEQ ID NO: 19 - protein, 7 aa, Artificial Sequence
  source: /note=“Description of Artificial Sequence Synthetic peptide”
SQSISNY

SEQ ID NO: 20 - protein, 3 aa, Artificial Sequence
  source: /note=“Description of Artificial Sequence Synthetic peptide”
DAS

SEQ ID NO: 21 - protein, 6 aa, Artificial Sequence
  source: /note=“Description of Artificial Sequence Synthetic peptide”
YYYESI

SEQ ID NO: 22 - protein, 6 aa, Artificial Sequence
  source: /note=“Description of Artificial Sequence Synthetic peptide”
QSISNY

SEQ ID NO: 23 - protein, 107 aa, Artificial Sequence
  source: /note=“Description of Artificial Sequence Synthetic polypeptide”
DIQMTQSPSSLSASVGDRVTITCRASQSISNYLAWYQQKPGKAPKLLIYDASSLQSGVPSRFSGSGSGTDFTLTISSLQPEDFATYYCQQYYYESITFGQGTKVEIK

SEQ ID NO: 24 - DNA, 321 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagtctatttctaactacctggcttggtaccagcagaaaccgggcaaagcgccgaaactattaatctacgacgcttcttctctgcaaagcggcgtgccgagccgctttagcggcagcggatccggcaccgatttcaccctgaccattagctctctgcaaccggaagactttgcgacctattattgccagcagtactactacgaatctatcacctttggccagggcacgaaagttgaaattaaa

SEQ ID NO: 25 - protein, 214 aa, Artificial Sequence
  source: /note=“Description of Artificial Sequence Synthetic polypeptide”
DIQMTQSPSSLSASVGDRVTITCRASQSISNYLAWYQQKPGKAPKLLIYDASSLQSGVPSRFSGSGSGTDFTLTISSLQPEDFATYYCQQYYYESITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - DNA, 642 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccagtctatttctaactacctggcttggtaccagcagaaaccgggcaaagcgccgaaactattaatctacgacgcttcttctctgcaaagcggcgtgccgagccgctttagcggcagcggatccggcaccgatttcaccctgaccattagctctctgcaaccggaagactttgcgacctattattgccagcagtactactacgaatctatcacctttggccagggcacgaaagttgaaattaaacgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 27 - protein, 5 aa, Artificial Sequence
  source: /note=“Description of Artificial Sequence Synthetic peptide”
SHALS

SEQ ID NO: 28 - protein, 17 aa, Artificial Sequence
  source: /note=“Description of Artificial Sequence Synthetic peptide”
GIIPSFGTADYAQKFQG

SEQ ID NO: 29 - protein, 7 aa, Artificial Sequence
  source: /note=“Description of Artificial Sequence Synthetic peptide”
GLYDFDY

SEQ ID NO: 30 - protein, 7 aa, Artificial Sequence
  source: /note=“Description of Artificial Sequence Synthetic peptide”
GGTFSSH

SEQ ID NO: 31 - protein, 6 aa, Artificial Sequence
  source: /note=“Description of Artificial Sequence Synthetic peptide”
IPSFGT

SEQ ID NO: 32 - protein, 10 aa, Artificial Sequence
  source: /note=“Description of Artificial Sequence Synthetic peptide”
GGTFSSHALS

SEQ ID NO: 33 - protein, 8 aa, Artificial Sequence
  source: /note=“Description of Artificial Sequence Synthetic peptide”
GGTFSSHA

SEQ ID NO: 34 - protein, 8 aa, Artificial Sequence
  source: /note=“Description of Artificial Sequence Synthetic peptide”
IIPSFGTA

SEQ ID NO: 35 - protein, 9 aa, Artificial Sequence
  source: /note=“Description of Artificial Sequence Synthetic peptide”
ARGLYDFDY

SEQ ID NO: 36 - protein, 116 aa, Artificial Sequence
  source: /note=“Description of Artificial Sequence Synthetic polypeptide”
QVQLVQSGAEVKKPGSSVKVSCKASGGTFSSHALSWVRQAPGQGLEWMGGIIPSFGTADYAQKFQGRVTITADESTSTAYMELSSLRSEDTAVYYCARGLYDFDYWGQGTLVTVSS

SEQ ID NO: 37 - DNA, 348 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgttttcttctcatgctctgtcttgggtgcgccaggccccgggccagggcctcgagtggatgggcggtatcatcccgtctttcggcactgcggactacgcccagaaatttcagggccgggtgaccattaccgccgatgaaagcaccagcaccgcctatatggaactgagcagcctgcgcagcgaagatacggccgtgtattattgcgcgcgtggtctgtacgacttcgactactggggccaaggcaccctggtgactgttagctca

SEQ ID NO: 38 - protein, 446 aa, Artificial Sequence
  source: /note=“Description of Artificial Sequence Synthetic polypeptide”
QVQLVQSGAEVKKPGSSVKVSCKASGGTFSSHALSWVRQAPGQGLEWMGGIIPSFGTADYAQKFQGRVTITADESTSTAYMELSSLRSEDTAVYYCARGLYDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - DNA, 1338 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgttttcttctcatgctctgtcttgggtgcgccaggccccgggccagggcctcgagtggatgggcggtatcatcccgtctttcggcactgcggactacgcccagaaatttcagggccgggtgaccattaccgccgatgaaagcaccagcaccgcctatatggaactgagcagcctgcgcagcgaagatacggccgtgtattattgcgcgcgtggtctgtacgacttcgactactggggccaaggcaccctggtgactgttagctcagctagcaccaagggccccagcgtgttccccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 40 - protein, 235 aa, Artificial Sequence
  source: /note=“Description of Artificial Sequence Synthetic polypeptide”
QVQLVQSGAEVKKPGSSVKVSCKASGGTFSSHALSWVRQAPGQGLEWMGGIIPSFGTADYAQKFQGRVTITADESTSTAYMELSSLRSEDTAVYYCARGLYDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLG

SEQ ID NO: 41 - DNA, 705 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgttttcttctcatgctctgtcttgggtgcgccaggccccgggccagggcctcgagtggatgggcggtatcatcccgtctttcggcactgcggactacgcccagaaatttcagggccgggtgaccattaccgccgatgaaagcaccagcaccgcctatatggaactgagcagcctgcgcagcgaagatacggccgtgtattattgcgcgcgtggtctgtacgacttcgactactggggccaaggcaccctggtgactgttagctcagctagcaccaagggccccagcgtgttccccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctggga

SEQ ID NO: 42 - protein, 11 aa, Artificial Sequence
  source: /note=“Description of Artificial Sequence Synthetic peptide”
RASQDISQDLA

SEQ ID NO: 43 - protein, 9 aa, Artificial Sequence
  source: /note=“Description of Artificial Sequence Synthetic peptide”
QQYYYLPST

SEQ ID NO: 44 - protein, 7 aa, Artificial Sequence
  source: /note=“Description of Artificial Sequence Synthetic peptide”
SQDISQD

SEQ ID NO: 45 - protein, 6 aa, Artificial Sequence
  source: /note=“Description of Artificial Sequence Synthetic peptide”
YYYLPS

SEQ ID NO: 46 - protein, 6 aa, Artificial Sequence
  source: /note=“Description of Artificial Sequence Synthetic peptide”
QDISQD

SEQ ID NO: 47 - protein, 107 aa, Artificial Sequence
  source: /note=“Description of Artificial Sequence Synthetic polypeptide”
DIQMTQSPSSLSASVGDRVTITCRASQDISQDLAWYQQKPGKAPKLLIYDASSLQSGVPSRFSGSGSGTDFTLTISSLQPEDFAVYYCQQYYYLPSTFGQGTKVEIK

SEQ ID NO: 48 - DNA, 321 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccaggacatttctcaggacctggcttggtaccagcagaaaccgggcaaagcgccgaaactattaatctacgacgcttcttctctgcaaagcggcgtgccgagccgctttagcggcagcggatccggcaccgatttcaccctgaccattagctctctgcaaccggaagactttgcggtgtattattgccagcagtactactacctgccgtctacctttggccagggcacgaaagttgaaattaaa

SEQ ID NO: 49 - protein, 214 aa, Artificial Sequence
  source: /note=“Description of Artificial Sequence Synthetic polypeptide”
DIQMTQSPSSLSASVGDRVTITCRASQDISQDLAWYQQKPGKAPKLLIYDASSLQSGVPSRFSGSGSGTDFTLTISSLQPEDFAVYYCQQYYYLPSTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - DNA, 642 bp, Artificial Sequence
  source: /note=“Description of Artificial Sequence Synthetic polynucleotide”
gatatccagatgacccagagcccgagcagcctgagcgccagcgtgggcgatcgcgtgaccattacctgcagagccagccaggacatttctcaggacctggcttggtaccagcagaaaccgggcaaagcgccgaaactattaatctacgacgcttcttctctgcaaagcggcgtgccgagccgctttagcggcagcggatccggcaccgatttcaccctgaccattagctctctgcaaccggaagactttgcggtgtattattgccagcagtactactacctgccgtctacctttggccagggcacgaaagttgaaattaaacgtacggtggccgctcccagcgtgttcatcttcccccccagcgacgagcagctgaagagtggcaccgccagcgtggtgtgcctgctgaacaacttctacccccgggaggccaaggtgcagtggaaggtggacaacgccctgcagagcggcaacagccaggagagcgtcaccgagcaggacagcaaggactccacctacagcctgagcagcaccctgaccctgagcaaggccgactacgagaagcataaggtgtacgcctgcgaggtgacccaccagggcctgtccagccccgtgaccaagagcttcaacaggggcgagtgc

SEQ ID NO: 51 - protein, 17 aa, Artificial Sequence
  source: /note=“Description of Artificial Sequence Synthetic peptide”
TIGPFEGQPRYAQKFQG

SEQ ID NO: 52 - protein, 6 aa, Artificial Sequence
  source: /note=“Description of Artificial Sequence Synthetic peptide”
GPFEGQ

SEQ ID NO: 53 - protein, 8 aa, Artificial Sequence
  source: /note=“Description of Artificial Sequence Synthetic peptide”
IGPFEGQP

SEQ ID NO: 54 - protein, 118 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGTIGPFEGQPRYAQKFQGRVTITADESTSTAYMELSSLRSEDTAVYYCARGGYISDFDVWGQGTLVTVSS

SEQ ID NO: 55 - DNA, 354 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttagcagctatgcgattagctgggtgcgccaggccccgggccagggcctcgagtggatgggcactatcggtccgttcgaaggccagccgcgttacgcccagaaatttcagggccgggtgaccattaccgccgatgaaagcaccagcaccgcctatatggaactgagcagcctgcgcagcgaagatacggccgtgtattattgcgcgcgtggtggttacatctctgacttcgatgtttggggccaaggcaccctggtgactgttagctca

SEQ ID NO: 56 - protein, 448 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGTIGPFEGQPRYAQKFQGRVTITADESTSTAYMELSSLRSEDTAVYYCARGGYISDFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 57 - DNA, 1344 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttagcagctatgcgattagctgggtgcgccaggccccgggccagggcctcgagtggatgggcactatcggtccgttcgaaggccagccgcgttacgcccagaaatttcagggccgggtgaccattaccgccgatgaaagcaccagcaccgcctatatggaactgagcagcctgcgcagcgaagatacggccgtgtattattgcgcgcgtggtggttacatctctgacttcgatgtttggggccaaggcaccctggtgactgttagctcagctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 58 - protein, 237 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGTIGPFEGQPRYAQKFQGRVTITADESTSTAYMELSSLRSEDTAVYYCARGGYISDFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLG

SEQ ID NO: 59 - DNA, 711 bp, Artificial Sequence
  source: /note=“Description of Artificial Sequence Synthetic polynucleotide”
caggtgcaattggtgcagagcggtgccgaagtgaaaaaaccgggcagcagcgtgaaagttagctgcaaagcatccggagggacgtttagcagctatgcgattagctgggtgcgccaggccccgggccagggcctcgagtggatgggcactatcggtccgttcgaaggccagccgcgttacgcccagaaatttcagggccgggtgaccattaccgccgatgaaagcaccagcaccgcctatatggaactgagcagcctgcgcagcgaagatacggccgtgtattattgcgcgcgtggtggttacatctctgacttcgatgtttggggccaaggcaccctggtgactgttagctcagctagcaccaagggcccaagtgtgtttcccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctggga

SEQ ID NO: 60 - protein, 7 aa, Artificial Sequence
  source: /note=“Description of Artificial Sequence Synthetic peptide”
TNSAAWN

SEQ ID NO: 61 - protein, 18 aa, Artificial Sequence
  source: /note=“Description of Artificial Sequence Synthetic peptide”
RIYYRSQWLNDYAVSVKS

SEQ ID NO: 62 - protein, 15 aa, Artificial Sequence
  source: /note=“Description of Artificial Sequence Synthetic peptide”
QLTYPYTVYHKALDV

SEQ ID NO: 63 - protein, 9 aa, Artificial Sequence
  source: /note=“Description of Artificial Sequence Synthetic peptide”
GDSVSTNSA

SEQ ID NO: 64 - protein, 7 aa, Artificial Sequence
  source: /note=“Description of Artificial Sequence Synthetic peptide”
YYRSQWL

SEQ ID NO: 65 - protein, 12 aa, Artificial Sequence
  source: /note=“Description of Artificial Sequence Synthetic peptide”
GDSVSTNSAAWN

SEQ ID NO: 66 - protein, 10 aa, Artificial Sequence
  source: /note=“Description of Artificial Sequence Synthetic peptide”
GDSVSTNSAA

SEQ ID NO: 67 - protein, 9 aa, Artificial Sequence
  source: /note=“Description of Artificial Sequence Synthetic peptide”
IYYRSQWLN

SEQ ID NO: 68 - protein, 17 aa, Artificial Sequence
  source: /note=“Description of Artificial Sequence Synthetic peptide”
ARQLTYPYTVYHKALDV

SEQ ID NO: 69 - protein, 127 aa, Artificial Sequence
  source: /note=“Description of Artificial Sequence Synthetic polypeptide”
QVQLQQSGPGLVKPSQTLSLTCAISGDSVSTNSAAWNWIRQSPSRGLEWLGRIYYRSQWLNDYAVSVKSRITINPDTSKNQFSLQLNSVTPEDTAVYYCARQLTYPYTVYHKALDVWGQGTLVTVSS

SEQ ID NO: 70 - DNA, 381 bp, Artificial Sequence
  source: /note=“Description of Artificial Sequence Synthetic polynucleotide”
caggtgcaattgcagcagagcggtccgggcctggtgaaaccgagccagaccctgagcctgacctgcgcgatttccggagatagcgtgagcactaactctgctgcttggaactggattcgtcagagcccgagccgtggcctcgagtggctgggccgtatctactaccgtagccagtggctgaacgactatgccgtgagcgtgaaaagccgcattaccattaacccggatacttcgaaaaaccagtttagcctgcaactgaacagcgtgaccccggaagatacggccgtgtattattgcgcgcgtcagctgacttacccgtacactgtttaccataaagctctggatgtttggggtcaaggaaccctggtcaccgtctcctcg

SEQ ID NO: 71 - protein, 457 aa, Artificial Sequence
  source: /note=“Description of Artificial Sequence Synthetic polypeptide”
QVQLQQSGPGLVKPSQTLSLTCAISGDSVSTNSAAWNWIRQSPSRGLEWLGRIYYRSQWLNDYAVSVKSRITINPDTSKNQFSLQLNSVTPEDTAVYYCARQLTYPYTVYHKALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - DNA, 1371 bp, Artificial Sequence
  source: /note=“Description of Artificial Sequence Synthetic polynucleotide”
caggtgcaattgcagcagagcggtccgggcctggtgaaaccgagccagaccctgagcctgacctgcgcgatttccggagatagcgtgagcactaactctgctgcttggaactggattcgtcagagcccgagccgtggcctcgagtggctgggccgtatctactaccgtagccagtggctgaacgactatgccgtgagcgtgaaaagccgcattaccattaacccggatacttcgaaaaaccagtttagcctgcaactgaacagcgtgaccccggaagatacggccgtgtattattgcgcgcgtcagctgacttacccgtacactgtttaccataaagctctggatgtttggggtcaaggaaccctggtcaccgtctcctcggctagcaccaagggccccagcgtgttccccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 73 - protein, 246 aa, Artificial Sequence
  source: /note=“Description of Artificial Sequence Synthetic polypeptide”
QVQLQQSGPGLVKPSQTLSLTCAISGDSVSTNSAAWNWIRQSPSRGLEWLGRIYYRSQWLNDYAVSVKSRITINPDTSKNQFSLQLNSVTPEDTAVYYCARQLTYPYTVYHKALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLG

SEQ ID NO: 74 - DNA, 738 bp, Artificial Sequence
  source: /note=“Description of Artificial Sequence Synthetic polynucleotide”
caggtgcaattgcagcagagcggtccgggcctggtgaaaccgagccagaccctgagcctgacctgcgcgatttccggagatagcgtgagcactaactctgctgcttggaactggattcgtcagagcccgagccgtggcctcgagtggctgggccgtatctactaccgtagccagtggctgaacgactatgccgtgagcgtgaaaagccgcattaccattaacccggatacttcgaaaaaccagtttagcctgcaactgaacagcgtgaccccggaagatacggccgtgtattattgcgcgcgtcagctgacttacccgtacactgtttaccataaagctctggatgtttggggtcaaggaaccctggtcaccgtctcctcggctagcaccaagggccccagcgtgttccccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctggga

SEQ ID NO: 75 - protein, 11 aa, Artificial Sequence
  source: /note=“Description of Artificial Sequence Synthetic peptide”
SGDNLGDQYVS

SEQ ID NO: 76 - protein, 7 aa, Artificial Sequence
  source: /note=“Description of Artificial Sequence Synthetic peptide”
DDTDRPS

SEQ ID NO: 77 - protein, 9 aa, Artificial Sequence
  source: /note=“Description of Artificial Sequence Synthetic peptide”
QSTDSKSVV

SEQ ID NO: 78 - protein, 7 aa, Artificial Sequence
  source: /note=“Description of Artificial Sequence Synthetic peptide”
DNLGDQY

SEQ ID NO: 79 - protein, 3 aa, Artificial Sequence
  source: /note=“Description of Artificial Sequence Synthetic peptide”
DDT

SEQ ID NO: 80 - protein, 6 aa, Artificial Sequence
  source: /note=“Description of Artificial Sequence Synthetic peptide”
TDSKSV

SEQ ID NO: 81 - protein, 6 aa, Artificial Sequence
  source: /note=“Description of Artificial Sequence Synthetic peptide”
NLGDQY

SEQ ID NO: 82 - protein, 106 aa, Artificial Sequence
  source: /note=“Description of Artificial Sequence Synthetic polypeptide”
DIELTQPPSVSVSPGQTASITCSGDNLGDQYVSWYQQKPGQAPVLVIYDDTDRPSGIPERFSGSNSGNTATLTISGTQAEDEADYYCQSTDSKSVVFGGGTKLTVL

SEQ ID NO: 83 - DNA, 318 bp, Artificial Sequence
  source: /note=“Description of Artificial Sequence Synthetic polynucleotide”
gatatcgaactgacccagccgccgagcgtgagcgtgagcccgggccagaccgcgagcattacctgtagcggcgataacctgggtgaccaatacgtttcttggtaccagcagaaaccgggccaggcgccggtgctggtgatctacgacgacactgaccgtccgagcggcatcccggaacgttttagcggatccaacagcggcaacaccgcgaccctgaccattagcggcacccaggcggaagacgaagcggattattactgccagtctactgactctaaatctgttgtgtttggcggcggcacgaagttaaccgtccta

SEQ ID NO: 84 - protein, 212 aa, Artificial Sequence
  source: /note=“Description of Artificial Sequence Synthetic polypeptide”
DIELTQPPSVSVSPGQTASITCSGDNLGDQYVSWYQQKPGQAPVLVIYDDTDRPSGIPERFSGSNSGNTATLTISGTQAEDEADYYCQSTDSKSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 85 - DNA, 636 bp, Artificial Sequence
  source: /note=“Description of Artificial Sequence Synthetic polynucleotide”
gatatcgaactgacccagccgccgagcgtgagcgtgagcccgggccagaccgcgagcattacctgtagcggcgataacctgggtgaccaatacgtttcttggtaccagcagaaaccgggccaggcgccggtgctggtgatctacgacgacactgaccgtccgagcggcatcccggaacgttttagcggatccaacagcggcaacaccgcgaccctgaccattagcggcacccaggcggaagacgaagcggattattactgccagtctactgactctaaatctgttgtgtttggcggcggcacgaagttaaccgtcctaggccagcctaaggccgctccctccgtgaccctgttcccccccagctccgaggaactgcaggccaacaaggccaccctggtgtgcctgatcagcgacttctaccctggcgccgtgaccgtggcctggaaggccgacagcagccccgtgaaggccggcgtggagacaaccacccccagcaagcagagcaacaacaagtacgccgccagcagctacctgagcctgacccccgagcagtggaagagccacagaagctacagctgccaggtcacccacgagggcagcaccgtggagaaaaccgtggcccccaccgagtgcagc

SEQ ID NO: 86 - protein, 5 aa, Artificial Sequence
  source: /note=“Description of Artificial Sequence Synthetic peptide”
NYWIA

SEQ ID NO: 87 - protein, 17 aa, Artificial Sequence
  source: /note=“Description of Artificial Sequence Synthetic peptide”
IIYPSNSYTLYSPSFQG

SEQ ID NO: 88 - protein, 14 aa, Artificial Sequence
  source: /note=“Description of Artificial Sequence Synthetic peptide”
VPPGGSISYPAFDH

SEQ ID NO: 89 - protein, 7 aa, Artificial Sequence
  source: /note=“Description of Artificial Sequence Synthetic peptide”
GYSFTNY

SEQ ID NO: 90 - protein, 6 aa, Artificial Sequence
  source: /note=“Description of Artificial Sequence Synthetic peptide”
YPSNSY

SEQ ID NO: 91 - protein, 10 aa, Artificial Sequence
  source: /note=“Description of Artificial Sequence Synthetic peptide”
GYSFTNYWIA

SEQ ID NO: 92 - protein, 8 aa, Artificial Sequence
  source: /note=“Description of Artificial Sequence Synthetic peptide”
GYSFTNYW

SEQ ID NO: 93 - protein, 8 aa, Artificial Sequence
  source: /note=“Description of Artificial Sequence Synthetic peptide”
IYPSNSYT

SEQ ID NO: 94 - protein, 16 aa, Artificial Sequence
  source: /note=“Description of Artificial Sequence Synthetic peptide”
ARVPPGGSISYPAFDH

SEQ ID NO: 95 - protein, 123 aa, Artificial Sequence
  source: /note=“Description of Artificial Sequence Synthetic polypeptide”
QVQLVQSGAEVKKPGESLKISCKGSGYSFTNYWIAWVRQMPGKGLEWMGIIYPSNSYTLYSPSFQGQVTISADKSISTAYLQWSSLKASDTAMYYCARVPPGGSISYPAFDHWGQGTLVTVSS

SEQ ID NO: 96 - DNA, 369 bp, Artificial Sequence
  source: /note=“Description of Artificial Sequence Synthetic polynucleotide”
caggtgcaattggtgcagagcggtgcggaagtgaaaaaaccgggcgaaagcctgaaaattagctgcaaaggctccggatatagcttcactaactactggatcgcttgggtgcgccagatgccgggcaaaggtctcgagtggatgggcatcatctacccgtctaacagctacaccctgtatagcccgagctttcagggccaggtgaccattagcgcggataaaagcatcagcaccgcgtatctgcaatggagcagcctgaaagcgagcgataccgcgatgtattattgcgcgcgtgttccgccgggtggttctatctcttacccggctttcgatcattggggccaaggcaccctggtgactgttagctca

SEQ ID NO: 97 - protein, 453 aa, Artificial Sequence
  source: /note=“Description of Artificial Sequence Synthetic polypeptide”
QVQLVQSGAEVKKPGESLKISCKGSGYSFTNYWIAWVRQMPGKGLEWMGIIYPSNSYTLYSPSFQGQVTISADKSISTAYLQWSSLKASDTAMYYCARVPPGGSISYPAFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - DNA, 1359 bp, Artificial Sequence
  source: /note=“Description of Artificial Sequence Synthetic polynucleotide”
caggtgcaattggtgcagagcggtgcggaagtgaaaaaaccgggcgaaagcctgaaaattagctgcaaaggctccggatatagcttcactaactactggatcgcttgggtgcgccagatgccgggcaaaggtctcgagtggatgggcatcatctacccgtctaacagctacaccctgtatagcccgagctttcagggccaggtgaccattagcgcggataaaagcatcagcaccgcgtatctgcaatggagcagcctgaaagcgagcgataccgcgatgtattattgcgcgcgtgttccgccgggtggttctatctcttacccggctttcgatcattggggccaaggcaccctggtgactgttagctcagctagcaccaagggccccagcgtgttccccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctgggagggccttccgtgttcctgttcccccccaagcccaaggacaccctgatgatcagcaggacccccgaggtgacctgcgtggtggtggacgtgtcccacgaggacccagaggtgaagttcaactggtacgtggacggcgtggaggtgcacaacgccaagaccaagcccagagaggagcagtacaacagcacctacagggtggtgtccgtgctgaccgtgctgcaccaggactggctgaacggcaaagaatacaagtgcaaagtctccaacaaggccctgccagccccaatcgaaaagacaatcagcaaggccaagggccagccacgggagccccaggtgtacaccctgccccccagccgggaggagatgaccaagaaccaggtgtccctgacctgtctggtgaagggcttctaccccagcgatatcgccgtggagtgggagagcaacggccagcccgagaacaactacaagaccacccccccagtgctggacagcgacggcagcttcttcctgtacagcaagctgaccgtggacaagtccaggtggcagcagggcaacgtgttcagctgcagcgtgatgcacgaggccctgcacaaccactacacccagaagtccctgagcctgagccccggcaag

SEQ ID NO: 99 - protein, 242 aa, Artificial Sequence
  source: /note=“Description of Artificial Sequence Synthetic polypeptide”
QVQLVQSGAEVKKPGESLKISCKGSGYSFTNYWIAWVRQMPGKGLEWMGIIYPSNSYTLYSPSFQGQVTISADKSISTAYLQWSSLKASDTAMYYCARVPPGGSISYPAFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLG

SEQ ID NO: 100 - DNA, 726 bp, Artificial Sequence
  source: /note=“Description of Artificial Sequence Synthetic polynucleotide”
caggtgcaattggtgcagagcggtgcggaagtgaaaaaaccgggcgaaagcctgaaaattagctgcaaaggctccggatatagcttcactaactactggatcgcttgggtgcgccagatgccgggcaaaggtctcgagtggatgggcatcatctacccgtctaacagctacaccctgtatagcccgagctttcagggccaggtgaccattagcgcggataaaagcatcagcaccgcgtatctgcaatggagcagcctgaaagcgagcgataccgcgatgtattattgcgcgcgtgttccgccgggtggttctatctcttacccggctttcgatcattggggccaaggcaccctggtgactgttagctcagctagcaccaagggccccagcgtgttccccctggcccccagcagcaagtctacttccggcggaactgctgccctgggttgcctggtgaaggactacttccccgagcccgtgacagtgtcctggaactctggggctctgacttccggcgtgcacaccttccccgccgtgctgcagagcagcggcctgtacagcctgagcagcgtggtgacagtgccctccagctctctgggaacccagacctatatctgcaacgtgaaccacaagcccagcaacaccaaggtggacaagagagtggagcccaagagctgcgacaagacccacacctgccccccctgcccagctccagaactgctggga

SEQ ID NO: 101 - protein, 11 aa, Artificial Sequence
  source: /note=“Description of Artificial Sequence Synthetic peptide”
SGDNIGSIYAS

SEQ ID NO: 102 - protein, 7 aa, Artificial Sequence
  source: /note=“Description of Artificial Sequence Synthetic peptide”
RDNKRPS

SEQ ID NO: 103 - protein, 10 aa, Artificial Sequence
  source: /note=“Description of Artificial Sequence Synthetic peptide”
SVTDMEQHSV

SEQ ID NO: 104 - protein, 7 aa, Artificial Sequence
  source: /note=“Description of Artificial Sequence Synthetic peptide”
DNIGSIY

SEQ ID NO: 105 - protein, 3 aa, Artificial Sequence
  source: /note=“Description of Artificial Sequence Synthetic peptide”
RDN

SEQ ID NO: 106 - protein, 7 aa, Artificial Sequence
  source: /note=“Description of Artificial Sequence Synthetic peptide”
TDMEQHS

SEQ ID NO: 107 - protein, 6 aa, Artificial Sequence
  source: /note=“Description of Artificial Sequence Synthetic peptide”
NIGSIY

SEQ ID NO: 108 - protein, 107 aa, Artificial Sequence
  source: /note=“Description of Artificial Sequence Synthetic polypeptide”
DIELTQPPSVSVSPGQTASITCSGDNIGSIYASWYQQKPGQAPVLVIYRDNKRPSGIPERFSGSNSGNTATLTISGTQAEDEADYYCSVTDMEQHSVFGGGTKLTVL

SEQ ID NO: 109 - DNA, 321 bp, Artificial Sequence
  source: /note=“Description of Artificial Sequence Synthetic polynucleotide”
gatatcgaactgacccagccgccgagcgtgagcgtgagcccgggccagaccgcgagcattacctgtagcggcgataacatcggttctatctacgcttcttggtaccagcagaaaccgggccaggcgccggtgctggtgatctaccgtgacaacaaacgtccgagcggcatcccggaacgttttagcggatccaacagcggcaacaccgcgaccctgaccattagcggcacccaggcggaagacgaagcggattattactgctccgttactgacatggaacagcattctgtgtttggcggcggcacgaagttaaccgtccta

SEQ ID NO: 110 - protein, 213 aa, Artificial Sequence
  source: /note=“Description of Artificial Sequence Synthetic polypeptide”
DIELTQPPSVSVSPGQTASITCSGDNIGSIYASWYQQKPGQAPVLVIYRDNKRPSGIPERFSGSNSGNTATLTISGTQAEDEADYYCSVTDMEQHSVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 111 - DNA, 639 bp, Artificial Sequence
  source: /note=“Description of Artificial Sequence Synthetic polynucleotide”
gatatcgaactgacccagccgccgagcgtgagcgtgagcccgggccagaccgcgagcattacctgtagcggcgataacatcggttctatctacgcttcttggtaccagcagaaaccgggccaggcgccggtgctggtgatctaccgtgacaacaaacgtccgagcggcatcccggaacgttttagcggatccaacagcggcaacaccgcgaccctgaccattagcggcacccaggcggaagacgaagcggattattactgctccgttactgacatggaacagcattctgtgtttggcggcggcacgaagttaaccgtcctaggccagcctaaggccgctccctccgtgaccctgttcccccccagctccgaggaactgcaggccaacaaggccaccctggtgtgcctgatcagcgacttctaccctggcgccgtgaccgtggcctggaaggccgacagcagccccgtgaaggccggcgtggagacaaccacccccagcaagcagagcaacaacaagtacgccgccagcagctacctgagcctgacccccgagcagtggaagagccacagaagctacagctgccaggtcacccacgagggcagcaccgtggagaaaaccgtggcccccaccgagtgcagc

SEQ ID NO: 112 - protein, 503 aa, Artificial Sequence
  source: /note=“Description of Artificial Sequence Synthetic polypeptide”
QPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHTLFTPRSHHHHHH

SEQ ID NO: 113 - protein, 294 aa, Artificial Sequence
  source: /note=“Description of Artificial Sequence Synthetic polypeptide”
QPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGRSHHHHHH

SEQ ID NO: 114 - protein, 222 aa, Artificial Sequence
  source: /note=“Description of Artificial Sequence Synthetic polypeptide”
GFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTPRSHHHHHH

SEQ ID NO: 115 - protein, 741 aa, Artificial Sequence
  source: /note=“Description of Artificial Sequence Synthetic polypeptide”
SQPSASPGEPSPPSIHPAQSELIVEAGDTLSLTCIDPDFVRWTFKTYFNEMVENKKNEWIQEKAEATRTGTYTCSNSNGLTSSIYVFVRDPAKLFLVGLPLFGKEDSDALVRCPLTDPQVSNYSLIECDGKSLPTDLTFVPNPKAGITIKNVKRAYHRLCVRCAAQRDGTWLHSDKFTLKVRAAIKAIPVVSVPETSHLLKKGDTFTVVCTIKDVSTSVNSMWLKMNPQPQHIAQVKHNSWHRGDFNYERQETLTISSARVDDSGVFMCYANNTFGSANVTTTLKVVEKGFINISPVKNTTVFVTDGENVDLVVEYEAYPKPEHQQWIYMNRTSANKGKDYVKSDNKSNIRYVNQLRLTRLKGTEGGTYTFLVSNSDASASVTFNVYVNTKPEILTYDRLINGMLQCVAEGFPEPTIDWYFCTGAEQRCTTPVSPVDVQVQNVSVSPFGKLVVQSSIDSSVFRHNGTVECKASNDVGKSSAFFNFAFKEQIQAHTLFTPLEVLFQGPRSPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 116 - protein, 741 aa, Artificial Sequence
  source: /note=“Description of Artificial Sequence Synthetic polypeptide”
SQPSASPGEPSPPSIQPAQSELIVEAGDTIRLTCTDPAFVKWTFEILDVRIENKQSEWIREKAEATHTGKYTCVSGSGLRSSIYVFVRDPAVLFLVGLPLFGKEDNDALVRCPLTDPQVSNYSLIECDGKSLPTDLKFVPNPKAGITIKNVKRAYHRLCIRCAAQREGKWMRSDKFTLKVRAAIKAIPVVSVPETSHLLKEGDTFTVICTIKDVSTSVDSMWIKLNPQPQSKAQVKRNSWHQGDFNYERQETLTISSARVNDSGVFMCYANNTFGSANVTTTLKVVEKGFINIFPVKNTTVFVTDGENVDLVVEFEAYPKPEHQQWIYMNRTPTNRGEDYVKSDNQSNIRYVNELRLTRLKGTEGGTYTFLVSNSDVSASVTFDVYVNTKPEILTYDRLMNGRLQCVAAGFPEPTIDWYFCTGAEQRCTVPVPPVDVQIQNASVSPFGKLVVQSSIDSSVFRHNGTVECKASNAVGKSSAFFNFAFKGNSKEQIQPHTLFTPRSLEVLFQGPGSPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 117 - protein, 527 aa, Artificial Sequence
  source: /note=“Description of Artificial Sequence Synthetic polypeptide”
MYRMQLLSCIALSLALVTNSQPSVSPGEPSPPSIHPAKSELIVRVGNEIRLLCIDPGFVKWTFEILDETNENKQNEWITEKAEATNTGKYTCTNKHGLSSSIYVFVRDPAKLFLVDRSLYGKEDNDTLVRCPLTDPEVTSYSLKGCQGKPLPKDLRFVPDPKAGITIKSVKRAYHRLCLHCSADQEGKSVLSDKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNASIAFNVYVNTKPEILTYDRLVNGMLQCVAAGFPEPTIDWYFCPGTEQRCSASVLPVDVQTLNASGPPFGKLVVQSSIDSSAFKHNGTVECKAYNDVGKTSAYFNFAFKGNNKEQIHPHTLFTPRSHHHHHH

SEQ ID NO: 118 - protein, 222 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGVIFPAEGAPGYAQKFQGRVTITADESTSTAYMELSSLRSEDTAVYYCARGGYISDFDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCD

SEQ ID NO: 119 - protein, 231 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGVIFPAEGAPGYAQKFQGRVTITADESTSTAYMELSSLRSEDTAVYYCARGGYISDFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCP

SEQ ID NO: 120 - protein, 233 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGVIFPAEGAPGYAQKFQGRVTITADESTSTAYMELSSLRSEDTAVYYCARGGYISDFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAP

SEQ ID NO: 121 - protein, 238 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGVIFPAEGAPGYAQKFQGRVTITADESTSTAYMELSSLRSEDTAVYYCARGGYISDFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGP

SEQ ID NO: 122 - protein, 214 aa, Artificial Sequence
  source: /note=“Description of Artificial Sequence Synthetic polypeptide”
DIQMTQSPSSLSASVGDRVTITCRASQSISNYLAWYQQKPGKAPKLLIYDASSLQSGVPSRFSGSGSGTDFTLTISSLQPEDFATYYCQQYYYESITFGQGTKVEIKRTVAAPSVFIFPPSDEQLKSGTASVVCLLNNFYPREAKVQWKVDNALQSGNSQESVTCQDSKDSTYSLSSTLTLSKADYEKHKVYACEVTHQGLSSPVTKSFNRGEC

SEQ ID NO: 123 - protein, 214 aa, Artificial Sequence
  source: /note=“Description of Artificial Sequence Synthetic polypeptide”
DIQMTQSPSSLSASVGDRVTITCRASQSISNYLAWYQQKPGKAPKLLIYDASSLQSGVPSRFSGSGSGTDFTLTISSLQPEDFATYYCQQYYYESITFGQGTKVEIKRTVAAPCVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 220 aa, Artificial Sequence
  source: /note=“Description of Artificial Sequence Synthetic polypeptide”
QVQLVQSGAEVKKPGSSVKVSCKASGGTFSSHALSWVRQAPGQGLEWMGGIIPSFGTADYAQKFQGRVTITADESTSTAYMELSSLRSEDTAVYYCARGLYDFDYWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCD

SEQ ID NO: 125 - protein, 229 aa, Artificial Sequence
  source: /note=“Description of Artificial Sequence Synthetic polypeptide”
QVQLVQSGAEVKKPGSSVKVSCKASGGTFSSHALSWVRQAPGQGLEWMGGIIPSFGTADYAQKFQGRVTITADESTSTAYMELSSLRSEDTAVYYCARGLYDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCP

SEQ ID NO: 126 - protein, 231 aa, Artificial Sequence
  source: /note=“Description of Artificial Sequence Synthetic polypeptide”
QVQLVQSGAEVKKPGSSVKVSCKASGGTFSSHALSWVRQAPGQGLEWMGGIIPSFGTADYAQKFQGRVTITADESTSTAYMELSSLRSEDTAVYYCARGLYDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAP

SEQ ID NO: 127 - protein, 236 aa, Artificial Sequence
  source: /note=“Description of Artificial Sequence Synthetic polypeptide”
QVQLVQSGAEVKKPGSSVKVSCKASGGTFSSHALSWVRQAPGQGLEWMGGIIPSFGTADYAQKFQGRVTITADESTSTAYMELSSLRSEDTAVYYCARGLYDFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGP

SEQ ID NO: 128 - protein, 214 aa, Artificial Sequence
  source: /note=“Description of Artificial Sequence Synthetic polypeptide”
DIQMTQSPSSLSASVGDRVTITCRASQDISQDLAWYQQKPGKAPKLLIYDASSLQSGVPSRFSGSGSGTDFTLTISSLQPEDFAVYYCQQYYYLPSTFGQGTKVEIKRTVAAPSVFIFPPSDEQLKSGTASVVCLLNNFYPREAKVQWKVDNALQSGNSQESVTCQDSKDSTYSLSSTLTLSKADYEKHKVYACEVTHQGLSSPVTKSFNRGEC

SEQ ID NO: 129 - protein, 214 aa, Artificial Sequence
  source: /note=“Description of Artificial Sequence Synthetic polypeptide”
DIQMTQSPSSLSASVGDRVTITCRASQDISQDLAWYQQKPGKAPKLLIYDASSLQSGVPSRFSGSGSGTDFTLTISSLQPEDFAVYYCQQYYYLPSTFGQGTKVEIKRTVAAPCVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 222 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGTIGPFEGQPRYAQKFQGRVTITADESTSTAYMELSSLRSEDTAVYYCARGGYISDFDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKKVEPKSCD

SEQ ID NO: 131 - protein, 231 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGTIGPFEGQPRYAQKFQGRVTITADESTSTAYMELSSLRSEDTAVYYCARGGYISDFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCP

SEQ ID NO: 132 - protein, 233 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGTIGPFEGQPRYAQKFQGRVTITADESTSTAYMELSSLRSEDTAVYYCARGGYISDFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAP

SEQ ID NO: 133 - protein, 238 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGTIGPFEGQPRYAQKFQGRVTITADESTSTAYMELSSLRSEDTAVYYCARGGYISDFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGP

SEQ ID NO: 134 - protein, 214 aa, Artificial Sequence; identical to SEQ ID NO: 122
  source: /note=“Description of Artificial Sequence Synthetic polypeptide”

SEQ ID NO: 135 - protein, 214 aa, Artificial Sequence; identical to SEQ ID NO: 123
  source: /note=“Description of Artificial Sequence Synthetic polypeptide”

SEQ ID NO: 136 - protein, 231 aa, Artificial Sequence
  source: /note=“Description of Artificial Sequence Synthetic polypeptide”
QVQLQQSGPGLVKPSQTLSLTCAISGDSVSTNSAAWNWIRQSPSRGLEWLGRIYYRSQWLNDYAVSVKSRITINPDTSKNQFSLQLNSVTPEDTAVYYCARQLTYPYTVYHKALDVWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCD

SEQ ID NO: 137 - protein, 240 aa, Artificial Sequence
  source: /note=“Description of Artificial Sequence Synthetic polypeptide”
QVQLQQSGPGLVKPSQTLSLTCAISGDSVSTNSAAWNWIRQSPSRGLEWLGRIYYRSQWLNDYAVSVKSRITINPDTSKNQFSLQLNSVTPEDTAVYYCARQLTYPYTVYHKALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCP

SEQ ID NO: 138 - protein, 242 aa, Artificial Sequence
  source: /note=“Description of Artificial Sequence Synthetic polypeptide”
QVQLQQSGPGLVKPSQTLSLTCAISGDSVSTNSAAWNWIRQSPSRGLEWLGRIYYRSQWLNDYAVSVKSRITINPDTSKNQFSLQLNSVTPEDTAVYYCARQLTYPYTVYHKALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAP

SEQ ID NO: 139 - protein, 247 aa, Artificial Sequence
  source: /note=“Description of Artificial Sequence Synthetic polypeptide”
QVQLQQSGPGLVKPSQTLSLTCAISGDSVSTNSAAWNWIRQSPSRGLEWLGRIYYRSQWLNDYAVSVKSRITINPDTSKNQFSLQLNSVTPEDTAVYYCARQLTYPYTVYHKALDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGP

SEQ ID NO: 140 - protein, 212 aa, Artificial Sequence
  source: /note=“Description of Artificial Sequence Synthetic polypeptide”
DIELTQPPSVSVSPGQTASITCSGDNLGDQYVSWYQQKPGQAPVLVIYDDTDRPSGIPERFSGSNSGNTATLTISGTQAEDEADYYCQSTDSKSVVFGGGTKLTVLGQPKAAPSVTLFPPSSEELQANKATLVCLISDFYPGCVTVAWKADSSPVKAGVETTTPSKQSNNKYAASSYLSLTPEQWKSHRSYSCQVTHEGSTVEKTVAPTECS

SEQ ID NO: 141 - protein, 227 aa, Artificial Sequence
  source: /note=“Description of Artificial Sequence Synthetic polypeptide”
QVQLVQSGAEVKKPGESLKISCKGSGYSFTNYWIAWVRQMPGKGLEWMGIIYPSNSYTLYSPSFQGQVTISADKSISTAYLQWSSLKASDTAMYYCARVPPGGSISYPAFDHWGQGTLVTVSSASTKGPSVFPLAPSSKSTSGGTAALGCLVKDYFPCPVTVSWNSGALTSGVHTFPAVLQSSGLYSLSSVVTVPSSSLGTQTYICNVNHKPSNTKVDKRVEPKSCD

SEQ ID NO: 142 - protein, 236 aa, Artificial Sequence
  source: /note=“Description of Artificial Sequence Synthetic polypeptide”
QVQLVQSGAEVKKPGESLKISCKGSGYSFTNYWIAWVRQMPGKGLEWMGIIYPSNSYTLYSPSFQGQVTISADKSISTAYLQWSSLKASDTAMYYCARVPPGGSISYPAFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCP

SEQ ID NO: 143 - protein, 238 aa, Artificial Sequence
  source: /note=“Description of Artificial Sequence Synthetic polypeptide”
QVQLVQSGAEVKKPGESLKISCKGSGYSFTNYWIAWVRQMPGKGLEWMGIIYPSNSYTLYSPSFQGQVTISADKSISTAYLQWSSLKASDTAMYYCARVPPGGSISYPAFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAP

SEQ ID NO: 144 - protein, 243 aa, Artificial Sequence
  source: /note=“Description of Artificial Sequence Synthetic polypeptide”
QVQLVQSGAEVKKPGESLKISCKGSGYSFTNYWIAWVRQMPGKGLEWMGIIYPSNSYTLYSPSFQGQVTISADKSISTAYLQWSSLKASDTAMYYCARVPPGGSISYPAFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGP

SEQ ID NO: 145 - protein, 213 aa, Artificial Sequence
  source: /note=“Description of Artificial Sequence Synthetic polypeptide”
DIELTQPPSVSVSPGQTASITCSGDNIGSIYASWYQQKPGQAPVLVIYRDNKRPSGIPERFSGSNSGNTATLTISGTQAEDEADYYCSVTDMEQHSVFGGGTKLTVLGQPKAAPSVTLFPPSSEELQANKATLVCLISDFYPGCVTVAWKADSSPVKAGVETTTPSKQSNNKYAASSYLSLTPEQWKSHRSYSCQVTHEGSTVEKTVAPTECS

SEQ ID NO: 146 - protein, 239 aa, Artificial Sequence
  source: /note=“Description of Artificial Sequence Synthetic polypeptide”
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLG

SEQ ID NO: 147 - protein, 214 aa, Artificial Sequence
  source: /note=“Description of Artificial Sequence Synthetic polypeptide”
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 148 - protein, 450 aa, Artificial Sequence
  source: /note=“Description of Artificial Sequence Synthetic polypeptide”
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 16 aa, Human cytomegalovirus
MPLLLLLPLLWAGALA

SEQ ID NO: 150 - protein, 20 aa, Artificial Sequence
  source: /note=“Description of Artificial Sequence Synthetic peptide”
MSVLTQVLALLLLWLTGTRC

SEQ ID NO: 151 - protein, 6 aa, Artificial Sequence
  source: /note=“Description of Artificial Sequence Synthetic 6xHis tag”
HHHHHH

(19) United States
(12) Patent Application Publication
(10) Pub. No.: US 2021/0228731 A1
(43) Pub. Date: Jul. 29, 2021

(54) ANTIBODY DRUG CONJUGATES FOR ABLATING HEMATOPOIETIC STEM CELLS

(71) Applicant: NOVARTIS AG, Basel (CH)

(72) Inventors: Anthony Edward BOITANO, Waban, MA (US); Matthew T. BURGER, Belmont, MA (US); Susan E. CELLITTI, San Diego, CA (US); Michael Paul COOKE, Brookline, MA (US); Catrin FINNER, Neuried (DE); Bernhard Hubert GEIERSTANGER, Solana Beach, CA (US); Si-Tuen LEE-HOEFLICH, Lexington, MA (US); HongNgoc Thi PHAM, San Diego, CA (US); Siew Ho SCHLEYER, El Cerrito, CA (US); Kathrin TISSOT, Neuried (DE); Tetsuo UNO, San Diego, CA (US); Yongqin WAN, San Diego, CA (US); Ben WEN, Encinitas, CA (US); Qiang ZHANG, East Brunswick, NJ (US)

(21) Appl. No.: 17/253,584

(22) PCT Filed: Jun. 19, 2019
(86) PCT No.: PCT/IB2019/055178

§ 371 Date: Dec. 17, 2020

Related U.S. Application Data
(60) Provisional application No. 62/687,382, filed on Jun. 20, 2018.

(51) Int. Cl. A61K 47/68; C07K 16/28; A61K 38/07

(52) U.S. Cl. CPC A61K 47/6849; A61K 47/6803; C07K 16/2803; A61K 38/07; C07K 2317/55; A61K 2039/505; C07K 2317/21; C07K 2317/24; C07K 2317/92; C07K 2317/33; C07K 2317/73; C07K 2317/34

(57) ABSTRACT

The present invention provides antibody drug conjugates, wherein an antibody or antibody fragment that specifically binds to human cKIT is linked to a drug moiety, optionally through a linker. The present invention further provides pharmaceutical compositions comprising the antibody drug conjugates; and methods of making and using such pharmaceutical compositions for ablating hematopoietic stem cells in a patient in need thereof.

### CROSS-REFERENCE TO RELATED APPLICATIONS

This application claims the benefit of U.S. Provisional Application No. 62/687,382 filed Jun. 20, 2018, the content of which is hereby incorporated by reference in its entirety.

### FIELD OF THE INVENTION

The present disclosure is directed to anti-cKIT antibody drug conjugates, and their uses for ablating hematopoietic stem cells in a patient in need thereof, e.g., a hematopoietic stem cell transplantation recipient.

### SEQUENCE LISTING

The instant application contains a Sequence Listing which has been submitted electronically in ASCII format and is hereby incorporated by reference in its entirety. Said ASCII copy, created on May 2, 2019, is named PAT058157-WO-PCT_SL.txt and is 186,540 bytes in size.

### BACKGROUND OF THE INVENTION

cKIT (CD117) is a single transmembrane receptor tyrosine kinase that binds the ligand Stem Cell Factor (SCF). SCF induces homodimerization of cKIT which activates its tyrosine kinase activity and signals through both the PI3-AKT and MAPK pathways (Kindblom et al., Am J. Path. 1998 152(5):1259). cKIT was initially discovered as an oncogene as a truncated form expressed by a feline retrovirus (Besmer et al., Nature 1986 320:415-421). Cloning of the corresponding human gene demonstrated that cKIT is a member of the type III class of receptor tyrosine kinases, which count among the family members, FLT3, CSF-1 receptor and PDGF receptor. cKIT is required for the development of hematopoietic cells, germ cells, mast cells and melanocytes. Hematopoietic progenitor cells, e.g., hematopoietic stem cells (HSC), in the bone marrow, express high level of cKIT on cell surface. In addition, mast cells, melanocytes in the skin, and interstitial cells of Cajal in the digestive tract express cKIT.

Hematopoietic stem cells (HSCs) are capable of regenerating all blood and immune cells in a transplant recipient and therefore have great therapeutic potential. Hematopoietic stem cell transplantation is widely used as therapies for leukemia, lymphoma, and other life-threatening diseases. Many risks, however, are associated with such transplantation, including poor engraftment, immunological rejection, graft-versus-host disease (GVHD), or infection. Allogeneic hematopoietic stem cell transplantation generally requires conditioning of the recipient through cyto-reductive treatments to prevent immunological rejection of the graft. Current conditioning regimens are often so toxic to the host that they are contra-indicated for large groups of transplantation patients and/or cannot be provided in sufficient amounts to prevent graft-versus-host disease. Thus, there is a need for improving the conditioning and transplantation methods and decreasing the risks associated with hematopoietic stem cell transplantation and increasing its effectiveness for various disorders.

### SUMMARY OF THE INVENTION

The present disclosure provides antibody drug conjugates, wherein an antibody or antibody fragment (e.g., Fab or Fab′) that specifically binds to human cKIT is linked to a drug moiety (e.g., a cytotoxic agent), optionally through a linker. Those antibody drug conjugates can selectively deliver a cytotoxic agent to cells expressing cKIT, e.g., hematopoietic stem cells, thereby selectively ablate those cells in a patient, e.g., a hematopoietic stem cell transplantation recipient. Preferably, the cKIT antibody drug conjugates have pharmacokinetic properties such that it will not be present and/or active in a patient's circulation for an extended time, so they can be used for conditioning hematopoietic stem cell transplant recipients prior to hematopoietic stem cell transplantation. In some embodiments, provided herein are conjugates comprising an antibody fragment (e.g., Fab or Fab′) that specifically binds to cKIT, linked to a drug moiety (e.g., a cytotoxic agent), optionally through a linker. Surprisingly, the present inventors found that the full length anti-cKIT antibodies (e.g., full-length IgGs), F(ab′)₂ fragments, and toxin conjugates thereof cause mast cell degranulation, but the anti-cKIT Fab′ or Fab-toxin conjugates do not cause mast cell degranulation, even when crosslinked and/or multimerized into larger complexes as could be observed if a patient developed or had pre-existing anti-drug antibodies recognizing Fab fragments. The present disclosure further provides pharmaceutical compositions comprising the antibody drug conjugates, and methods of making and using such pharmaceutical compositions for ablating hematopoietic stem cells in a patient in need thereof, e.g., a hematopoietic stem cell transplantation recipient.

In one aspect, the present disclosure is directed to a conjugate of Formula (I):

A-(L_{B}-(D)ₙ)_{y} Formula (I);

wherein:
A is an antibody fragment that specifically binds to human cKIT;
L_{B} is a linker;
D is a cytotoxic agent;
n is an integer from 1 to 10, and y is an integer from 1 to 10.

In one aspect, the present disclosure is directed to a conjugate of having the structure of Formula (E):

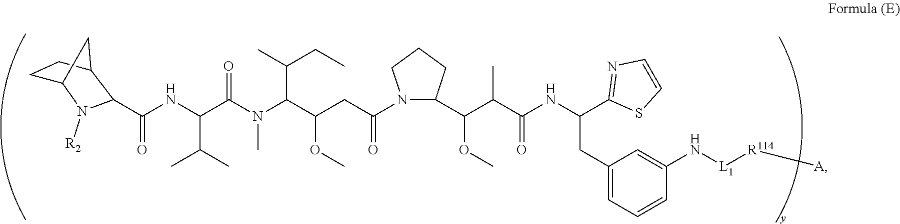

wherein R², A, L₁, y and R¹¹⁴, are as defined herein.

In one aspect, the present disclosure is directed to a conjugate of having the structure of Formula (G):

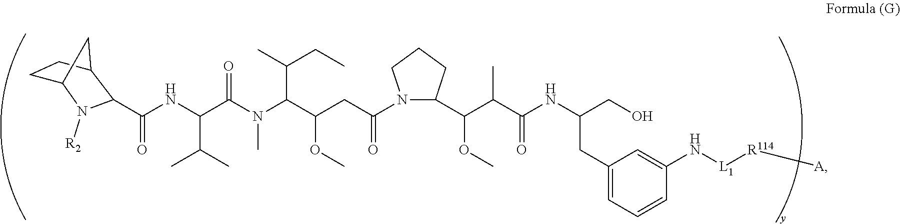

wherein R², A, L₁, y and R¹¹⁴, are as defined herein.

In another aspect, provided herein are antibodies and antibody fragments (e.g., Fab or Fab′) that specifically bind to human cKIT. Such anti-cKIT antibodies and antibody fragments (e.g., Fab or Fab′) can be used in any of the conjugates described herein.

In some embodiments, the antibody or antibody fragment (e.g., Fab or Fab′) that specifically binds to human cKIT is an antibody or antibody fragment (e.g., Fab or Fab′) that specifically binds to the extracellular domain of human cKIT (SEQ ID NO: 112).

In some embodiments, the antibody or antibody fragment (e.g., Fab or Fab′) that specifically binds to human cKIT is an antibody or antibody fragment (e.g., Fab or Fab′) that specifically binds to an epitope in domains 1-3 of human cKIT (SEQ ID NO: 113).

In some embodiments, the antibody or antibody fragment (e.g., Fab or Fab′) that specifically binds to human cKIT is an antibody or antibody fragment (e.g., Fab or Fab′) described in Table 1.

In some embodiments, the antibody or antibody fragment (e.g., Fab or Fab′) that specifically binds to human cKIT comprises a HCDR1 of SEQ ID NO: 1, a HCDR2 of SEQ ID NO: 2; a HCDR3 of SEQ ID NO: 3; a LCDR1 of SEQ ID NO: 16; a LCDR2 of SEQ ID NO: 17; and a LCDR3 of SEQ ID NO: 18.

In some embodiments, the antibody or antibody fragment (e.g., Fab or Fab′) that specifically binds to human cKIT comprises a HCDR1 of SEQ ID NO: 4, a HCDR2 of SEQ ID NO: 5; a HCDR3 of SEQ ID NO: 3; a LCDR1 of SEQ ID NO:19; a LCDR2 of SEQ ID NO: 20; and a LCDR3 of SEQ ID NO: 21.

In some embodiments, the antibody or antibody fragment (e.g., Fab or Fab′) that specifically binds to human cKIT comprises a HCDR1 of SEQ ID NO: 6, a HCDR2 of SEQ ID NO: 2; a HCDR3 of SEQ ID NO: 3; a LCDR1 of SEQ ID NO:16; a LCDR2 of SEQ ID NO: 17; and a LCDR3 of SEQ ID NO: 18.

In some embodiments, the antibody or antibody fragment (e.g., Fab or Fab′) that specifically binds to human cKIT comprises a HCDR1 of SEQ ID NO: 7, a HCDR2 of SEQ ID NO: 8; a HCDR3 of SEQ ID NO: 9; a LCDR1 of SEQ ID NO: 22; a LCDR2 of SEQ ID NO: 20; and a LCDR3 of SEQ ID NO: 18.

In some embodiments, the antibody or antibody fragment (e.g., Fab or Fab′) that specifically binds to human cKIT comprises a HCDR1 of SEQ ID NO: 27, a HCDR2 of SEQ ID NO: 28; a HCDR3 of SEQ ID NO: 29; a LCDR1 of SEQ ID NO: 42; a LCDR2 of SEQ ID NO: 17; and a LCDR3 of SEQ ID NO: 43.

In some embodiments, the antibody or antibody fragment (e.g., Fab or Fab′) that specifically binds to human cKIT comprises a HCDR1 of SEQ ID NO: 30, a HCDR2 of SEQ ID NO: 31; a HCDR3 of SEQ ID NO: 29; a LCDR1 of SEQ ID NO: 44; a LCDR2 of SEQ ID NO: 20; and a LCDR3 of SEQ ID NO: 45.

In some embodiments, the antibody or antibody fragment (e.g., Fab or Fab′) that specifically binds to human cKIT comprises a HCDR1 of SEQ ID NO: 32, a HCDR2 of SEQ ID NO: 28; a HCDR3 of SEQ ID NO: 29; a LCDR1 of SEQ ID NO: 42; a LCDR2 of SEQ ID NO: 17; and a LCDR3 of SEQ ID NO: 43.

In some embodiments, the antibody or antibody fragment (e.g., Fab or Fab′) that specifically binds to human cKIT comprises a HCDR1 of SEQ ID NO: 33, a HCDR2 of SEQ ID NO: 34; a HCDR3 of SEQ ID NO: 35; a LCDR1 of SEQ ID NO: 46; a LCDR2 of SEQ ID NO: 20; and a LCDR3 of SEQ ID NO: 43.

In some embodiments, the antibody or antibody fragment (e.g., Fab or Fab′) that specifically binds to human cKIT comprises a HCDR1 of SEQ ID NO: 1, a HCDR2 of SEQ ID NO: 51; a HCDR3 of SEQ ID NO: 3; a LCDR1 of SEQ ID NO:16; a LCDR2 of SEQ ID NO: 17; and a LCDR3 of SEQ ID NO: 18.

In some embodiments, the antibody or antibody fragment (e.g., Fab or Fab′) that specifically binds to human cKIT comprises a HCDR1 of SEQ ID NO: 4, a HCDR2 of SEQ ID NO: 52; a HCDR3 of SEQ ID NO: 3; a LCDR1 of SEQ ID NO:19; a LCDR2 of SEQ ID NO: 20; and a LCDR3 of SEQ ID NO: 21.

In some embodiments, the antibody or antibody fragment (e.g., Fab or Fab′) that specifically binds to human cKIT comprises a HCDR1 of SEQ ID NO: 6, a HCDR2 of SEQ ID NO: 51; a HCDR3 of SEQ ID NO: 3; a LCDR1 of SEQ ID NO:16; a LCDR2 of SEQ ID NO: 17; and a LCDR3 of SEQ ID NO: 18.

In some embodiments, the antibody or antibody fragment (e.g., Fab or Fab′) that specifically binds to human cKIT comprises a HCDR1 of SEQ ID NO: 7, a HCDR2 of SEQ ID NO: 53; a HCDR3 of SEQ ID NO: 9; a LCDR1 of SEQ ID NO: 22; a LCDR2 of SEQ ID NO: 20; and a LCDR3 of SEQ ID NO: 18.

In some embodiments, the antibody or antibody fragment (e.g., Fab or Fab′) that specifically binds to human cKIT comprises a HCDR1 of SEQ ID NO: 60, a HCDR2 of SEQ ID NO: 61; a HCDR3 of SEQ ID NO: 62; a LCDR1 of SEQ ID NO: 75; a LCDR2 of SEQ ID NO: 76; and a LCDR3 of SEQ ID NO: 77.

In some embodiments, the antibody or antibody fragment (e.g., Fab or Fab′) that specifically binds to human cKIT comprises a HCDR1 of SEQ ID NO: 63, a HCDR2 of SEQ ID NO: 64; a HCDR3 of SEQ ID NO: 62; a LCDR1 of SEQ ID NO: 78; a LCDR2 of SEQ ID NO: 79; and a LCDR3 of SEQ ID NO: 80.

In some embodiments, the antibody or antibody fragment (e.g., Fab or Fab′) that specifically binds to human cKIT comprises a HCDR1 of SEQ ID NO: 65, a HCDR2 of SEQ ID NO: 61; a HCDR3 of SEQ ID NO: 62; a LCDR1 of SEQ ID NO:75; a LCDR2 of SEQ ID NO: 76; and a LCDR3 of SEQ ID NO: 77.

In some embodiments, the antibody or antibody fragment (e.g., Fab or Fab′) that specifically binds to human cKIT comprises a HCDR1 of SEQ ID NO: 66, a HCDR2 of SEQ ID NO: 67; a HCDR3 of SEQ ID NO: 68; a LCDR1 of SEQ ID NO: 81; a LCDR2 of SEQ ID NO: 79; and a LCDR3 of SEQ ID NO: 77.

In some embodiments, the antibody or antibody fragment (e.g., Fab or Fab′) that specifically binds to human cKIT comprises a HCDR1 of SEQ ID NO: 86, a HCDR2 of SEQ ID NO: 87; a HCDR3 of SEQ ID NO: 88; a LCDR1 of SEQ ID NO: 101; a LCDR2 of SEQ ID NO: 102; and a LCDR3 of SEQ ID NO: 103.

In some embodiments, the antibody or antibody fragment (e.g., Fab or Fab′) that specifically binds to human cKIT comprises a HCDR1 of SEQ ID NO: 89, a HCDR2 of SEQ ID NO: 90; a HCDR3 of SEQ ID NO: 88; a LCDR1 of SEQ ID NO: 104; a LCDR2 of SEQ ID NO: 105; and a LCDR3 of SEQ ID NO: 106.

In some embodiments, the antibody or antibody fragment (e.g., Fab or Fab′) that specifically binds to human cKIT comprises a HCDR1 of SEQ ID NO: 91, a HCDR2 of SEQ ID NO: 87; a HCDR3 of SEQ ID NO: 88; a LCDR1 of SEQ ID NO: 101; a LCDR2 of SEQ ID NO: 102; and a LCDR3 of SEQ ID NO: 103.

In some embodiments, the antibody or antibody fragment (e.g., Fab or Fab′) that specifically binds to human cKIT comprises a HCDR1 of SEQ ID NO: 92, a HCDR2 of SEQ ID NO: 93; a HCDR3 of SEQ ID NO: 94; a LCDR1 of SEQ ID NO: 107; a LCDR2 of SEQ ID NO: 105; and a LCDR3 of SEQ ID NO: 103.

In some embodiments, the antibody or antibody fragment (e.g., Fab or Fab′) that specifically binds to human cKIT comprises a heavy chain variable region (VH) comprising the amino acid sequence of SEQ ID NO: 10, and a light chain variable region (VL) comprising the amino acid sequence of SEQ ID NO: 23.

In some embodiments, the antibody or antibody fragment (e.g., Fab or Fab′) that specifically binds to human cKIT comprises a VH comprising the amino acid sequence of SEQ ID NO: 36, and a VL comprising the amino acid sequence of SEQ ID NO: 47.

In some embodiments, the antibody or antibody fragment (e.g., Fab or Fab′) that specifically binds to human cKIT comprises a VH comprising the amino acid sequence of SEQ ID NO: 54, and a VL comprising the amino acid sequence of SEQ ID NO: 23.

In some embodiments, the antibody or antibody fragment (e.g., Fab or Fab′) that specifically binds to human cKIT comprises a VH comprising the amino acid sequence of SEQ ID NO: 69, and a VL comprising the amino acid sequence of SEQ ID NO: 82.

In some embodiments, the antibody or antibody fragment (e.g., Fab or Fab′) that specifically binds to human cKIT comprises a VH comprising the amino acid sequence of SEQ ID NO: 95, and a VL comprising the amino acid sequence of SEQ ID NO: 108.

In some embodiments, the antibody fragment (e.g., Fab′) that specifically binds to human cKIT comprises a heavy chain comprising the amino acid sequence of SEQ ID NO: 14, and a light chain comprising the amino acid sequence of SEQ ID NO: 25.

In some embodiments, the antibody fragment (e.g., Fab′) that specifically binds to human cKIT comprises a heavy chain comprising the amino acid sequence of SEQ ID NO: 40, and a light chain comprising the amino acid sequence of SEQ ID NO: 49.

In some embodiments, the antibody fragment (e.g., Fab′) that specifically binds to human cKIT comprises a heavy chain comprising the amino acid sequence of SEQ ID NO: 58, and a light chain comprising the amino acid sequence of SEQ ID NO: 25.

In some embodiments, the antibody fragment (e.g., Fab′) that specifically binds to human cKIT comprises a heavy chain comprising the amino acid sequence of SEQ ID NO: 73, and a light chain comprising the amino acid sequence of SEQ ID NO: 84.

In some embodiments, the antibody fragment (e.g., Fab′) that specifically binds to human cKIT comprises a heavy chain comprising the amino acid sequence of SEQ ID NO: 99, and a light chain comprising the amino acid sequence of SEQ ID NO: 110.

In some embodiments, the antibody fragment (e.g., Fab) that specifically binds to human cKIT comprises a heavy chain comprising the amino acid sequence of SEQ ID NO: 118, and a light chain comprising the amino acid sequence of SEQ ID NO: 122.

In some embodiments, the antibody fragment (e.g., Fab) that specifically binds to human cKIT comprises a heavy chain comprising the amino acid sequence of SEQ ID NO: 118, and a light chain comprising the amino acid sequence of SEQ ID NO: 123.

In some embodiments, the antibody fragment (e.g., Fab) that specifically binds to human cKIT comprises a heavy chain comprising the amino acid sequence of SEQ ID NO: 124, and a light chain comprising the amino acid sequence of SEQ ID NO: 128.

In some embodiments, the antibody fragment (e.g., Fab) that specifically binds to human cKIT comprises a heavy chain comprising the amino acid sequence of SEQ ID NO: 124, and a light chain comprising the amino acid sequence of SEQ ID NO: 129.

In some embodiments, the antibody fragment (e.g., Fab) that specifically binds to human cKIT comprises a heavy chain comprising the amino acid sequence of SEQ ID NO: 130, and a light chain comprising the amino acid sequence of SEQ ID NO: 134.

In some embodiments, the antibody fragment (e.g., Fab) that specifically binds to human cKIT comprises a heavy chain comprising the amino acid sequence of SEQ ID NO: 130, and a light chain comprising the amino acid sequence of SEQ ID NO: 135.

In some embodiments, the antibody fragment (e.g., Fab) that specifically binds to human cKIT comprises a heavy chain comprising the amino acid sequence of SEQ ID NO: 136, and a light chain comprising the amino acid sequence of SEQ ID NO: 140.

In some embodiments, the antibody fragment (e.g., Fab) that specifically binds to human cKIT comprises a heavy chain comprising the amino acid sequence of SEQ ID NO: 141, and a light chain comprising the amino acid sequence of SEQ ID NO: 145.

In some embodiments, the antibody fragment (e.g., Fab′) that specifically binds to human cKIT comprises a heavy chain comprising an amino acid sequence selected from SEQ ID NO: 119, 120 or 121, and a light chain comprising the amino acid sequence of SEQ ID NO: 25.

In some embodiments, the antibody fragment (e.g., Fab′) that specifically binds to human cKIT comprises a heavy chain comprising an amino acid sequence selected from SEQ ID NO: 125, 126, or 127, and a light chain comprising the amino acid sequence of SEQ ID NO: 49.

In some embodiments, the antibody fragment (e.g., Fab′) that specifically binds to human cKIT comprises a heavy chain comprising an amino acid sequence selected from SEQ ID NO: 131, 132, or 133, and a light chain comprising the amino acid sequence of SEQ ID NO: 25.

In some embodiments, the antibody fragment (e.g., Fab′) that specifically binds to human cKIT comprises a heavy chain comprising an amino acid sequence selected from SEQ ID NO: 137, 138, or 139, and a light chain comprising the amino acid sequence of SEQ ID NO: 84.

In some embodiments, the antibody fragment (e.g., Fab′) that specifically binds to human cKIT comprises a heavy chain comprising an amino acid sequence selected from SEQ ID NO: 142, 143, or 144, and a light chain comprising the amino acid sequence of SEQ ID NO: 110.

In some embodiments, the antibody that specifically binds to human cKIT comprises a heavy chain comprising the amino acid sequence of SEQ ID NO: 12, and a light chain comprising the amino acid sequence of SEQ ID NO: 25.

In some embodiments, the antibody that specifically binds to human cKIT comprises a heavy chain comprising the amino acid sequence of SEQ ID NO: 38, and a light chain comprising the amino acid sequence of SEQ ID NO: 49.

In some embodiments, the antibody that specifically binds to human cKIT comprises a heavy chain comprising the amino acid sequence of SEQ ID NO: 56, and a light chain comprising the amino acid sequence of SEQ ID NO: 25.

In some embodiments, the antibody that specifically binds to human cKIT comprises a heavy chain comprising the amino acid sequence of SEQ ID NO: 71, and a light chain comprising the amino acid sequence of SEQ ID NO: 84.

In some embodiments, the antibody that specifically binds to human cKIT comprises a heavy chain comprising the amino acid sequence of SEQ ID NO: 97, and a light chain comprising the amino acid sequence of SEQ ID NO: 110.

In some embodiments, provided herein are conjugates comprising an antibody fragment (e.g., Fab or Fab′) that specifically binds to cKIT (anti-cKIT Fab or Fab′), linked to a drug moiety (e.g., a cytotoxic agent), optionally through a linker. The anti-cKIT Fab or Fab′ can be any of the Fab or Fab′ described herein, e.g., any of the Fab or Fab′ in Table 1. As described herein, such anti-cKIT Fab′ or Fab-toxin conjugates are able to ablate human HSC cells in vitro and in vivo, but do not cause mast cell degranulation even when crosslinked and/or multimerized into larger complexes.

### BRIEF DESCRIPTION OF THE DRAWINGS

FIGS. 1A-1C show line graphs of a subset of anti-cKIT Fab′-DAR4 conjugate samples (see Table 2 for conjugate details), killing of human stem and progenitor cells (cKIT⁺/CD90⁺ cells) in vitro with approximately equal potency. FIG. 1A shows data from J4 (diamonds), J5 (open circles, dashed line), J8 (squares), and J9 (open triangles, dotted line). FIG. 1B shows data from J10 (open squares) and J11 (circles). FIG. 1C shows anti-cKit Fab′1-DAR4 and anti-cKit Fab′2-DAR4 conjugates relative to isotype control anti-HER2 Fab′-DAR4 conjugates and untreated cells (squares, dotted line): J10 (diamonds), J15 (circles), J16 (open squares), J19 (open circles, dashed line), and J20 (open triangles).

FIGS. 2A-2O are line graphs showing representative results of in vitro human mast cell degranulation assays, which used human peripheral blood HSC-derived mast cells and beta-hexosaminidase release as the readout (assessed by absorbance at 405 nm with baseline subtraction based on reference absorbance at 620 nm). Data shown here were collected in the absence of SCF. The line graphs show mast cell degranulation level triggered by antibodies or antibody fragments at various concentrations: 0.006 nM (triangles); 0.098 nM (diamonds); 1.6 nM (circles); and 25 nM (squares), when the test agents were cross-linked using an antibody specific for the Fab portion on the antibody test agents (titrated on x-axis). For reference, the cross-linker antibody alone is plotted on each graph (open diamonds, dashed line). FIGS. 2A-2C show that full length anti-cKIT Ab4 (FIG. 2A) and anti-cKIT F(ab′4)₂ fragment (FIG. 2B) caused mast cell degranulation when cross-linked, while no mast cell degranulation was triggered by anti-cKIT Fab4 (HC-E152C) fragment at all tested concentrations (FIG. 2C). FIGS. 2D-2F show that full length anti-cKIT Ab (FIG. 2D) and anti-cKIT F(ab′1)₂ fragment (FIG. 2E) caused mast cell degranulation when cross-linked, while no mast cell degranulation was triggered by anti-cKIT Fab1 (HC-E152C) fragment at all tested concentrations (FIG. 2F). FIGS. 2G-2I show that full length anti-cKIT Ab2 (FIG. 2G) and anti-cKIT F(ab′2)₂ fragment (FIG. 2H) caused mast cell degranulation when cross-linked, while no mast cell degranulation was triggered by anti-cKIT Fab2 (HC-E152C) fragment at all tested concentrations (FIG. 2I). FIGS. 2J-2L show that full length anti-cKIT Ab3 (FIG. 2J) and anti-cKIT F(ab′3)₂ fragment (FIG. 2K) caused mast cell degranulation when cross-linked, while no mast cell degranulation was triggered by anti-cKIT Fab3 (HC-E152C) fragment at all tested concentrations (FIG. 2L). FIGS. 2M-20 are line graphs showing no mast cell degranulation caused by anti-Her2 antibody (FIG. 2M), anti-Her2-F(ab′)₂ fragment (FIG. 2N), or anti-Her2-Fab (HC-E152C) fragment (FIG. 2O) when cross-linked.

FIG. 3 is a dot plot showing in vivo ablation of human HSCs from mouse host using anti-cKit conjugates.

### DETAILED DESCRIPTION

The present disclosure provides antibody drug conjugates, wherein an antibody or antibody fragment (e.g., Fab or Fab′) that specifically binds to human cKIT is linked to a drug moiety (e.g., a cytotoxic agent), optionally through a linker. Those antibody drug conjugates can selectively deliver a cytotoxic agent to cells expressing cKIT, e.g., hematopoietic stem cells, thereby selectively ablate those cells in a patient, e.g., a hematopoietic stem cell transplantation recipient. Preferably, the cKIT antibody drug conjugates have pharmacokinetic properties such that it will not be present and/or active in a patient's circulation for an extended time (e.g., half-life is less than 24-48 hours), so they can be used for conditioning hematopoietic stem cell transplant recipients prior to hematopoietic stem cell transplantation. In some embodiments, provided herein are conjugates comprising an antibody fragment (e.g., Fab or Fab′) that specifically binds to cKIT, linked to a drug moiety (e.g., a cytotoxic agent), optionally through a linker. Surprisingly, the present inventors found that the full length anti-cKIT antibodies (e.g., full-length IgGs), F(ab′)₂ fragments, and toxin conjugates thereof cause mast cell degranulation, but the anti-cKIT Fab′ or Fab-toxin conjugates do not cause mast cell degranulation, even when crosslinked and/or multimerized into larger complexes as could be observed if a patient developed or had pre-existing anti-drug antibodies recognizing Fab fragments. The present disclosure further provides pharmaceutical compositions comprising the antibody drug conjugates, and methods of making and using such pharmaceutical compositions for ablating hematopoietic stem cells in a patient in need thereof, e.g., a hematopoietic stem cell transplantation recipient.

### Definitions

Unless stated otherwise, the following terms and phrases as used herein are intended to have the following meanings:

The term “alkyl” refers to a monovalent saturated hydrocarbon chain having the specified number of carbon atoms. For example, C₁₋₆Calkyl refers to an alkyl group having from 1 to 6 carbon atoms. Alkyl groups may be straight or branched. Representative branched alkyl groups have one, two, or three branches. Examples of alkyl groups include, but are not limited to, methyl, ethyl, propyl (n-propyl and isopropyl), butyl (n-butyl, isobutyl, sec-butyl, and t-butyl), pentyl (n-pentyl, isopentyl, and neopentyl), and hexyl.

The term “antibody,” as used herein, refers to a protein, or polypeptide sequence derived from an immunoglobulin molecule that specifically binds to an antigen. Antibodies can be polyclonal or monoclonal, multiple or single chain, or intact immunoglobulins, and may be derived from natural sources or from recombinant sources. A naturally occurring “antibody” is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxyl-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. An antibody can be a monoclonal antibody, human antibody, humanized antibody, camelid antibody, or chimeric antibody. The antibodies can be of any isotype (e.g., IgG, IgE, IgM, IgD, IgA and IgY), class (e.g., IgG1, IgG2, IgG3, IgG4, IgA1 and IgA2) or subclass.

“Complementarity-determining domains” or “complementary-determining regions” (“CDRs”) interchangeably refer to the hypervariable regions of VL and VH. The CDRs are the target protein-binding site of the antibody chains that harbors specificity for such target protein. There are three CDRs (CDR1-3, numbered sequentially from the N-terminus) in each human VL or VH, constituting about 15-20% of the variable domains. CDRs can be referred to by their region and order. For example, “VHCDR1” or “HCDR1” both refer to the first CDR of the heavy chain variable region. The CDRs are structurally complementary to the epitope of the target protein and are thus directly responsible for the binding specificity. The remaining stretches of the VL or VH, the so-called framework regions, exhibit less variation in amino acid sequence (Kuby, Immunology, 4th ed., Chapter 4. W.H. Freeman & Co., New York, 2000).

The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), A1-Lazikani et al., (1997) JMB 273, 927-948 (“Chothia” numbering scheme) and ImMunoGenTics (IMGT) numbering (Lefranc, M.-P., The Immunologist, 7, 132-136 (1999); Lefranc, M.-P. et al., Dev. Comp. Immunol., 27, 55-77 (2003) (“IMGT” numbering scheme). For example, for classic formats, under Kabat, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under Chothia the CDR amino acids in the VH are numbered 26-32 (HCDR1), 52-56 (HCDR2), and 95-102 (HCDR3); and the amino acid residues in VL are numbered 26-32 (LCDR1), 50-52 (LCDR2), and 91-96 (LCDR3). By combining the CDR definitions of both Kabat and Chothia, the CDRs consist of amino acid residues 26-35 (HCDR1), 50-65 (HCDR2), and 95-102 (HCDR3) in human VH and amino acid residues 24-34 (LCDR1), 50-56 (LCDR2), and 89-97 (LCDR3) in human VL. Under IMGT the CDR amino acid residues in the VH are numbered approximately 26-35 (CDR1), 51-57 (CDR2) and 93-102 (CDR3), and the CDR amino acid residues in the VL are numbered approximately 27-32 (CDR1), 50-52 (CDR2), and 89-97 (CDR3) (numbering according to “Kabat”). Under IMGT, the CDR regions of an antibody can be determined using the program IMGT/DomainGap Align.

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3, and in some cases, CH4) confer important biological properties such as secretion, transplacental mobility, Fc receptor binding, complement binding, FcRn receptor binding, half-life, pharmacokinetics and the like. By convention, the numbering of the constant region domains increases as they become more distal from the antigen binding site or amino-terminus of the antibody. The N-terminus is a variable region and at the C-terminus is a constant region; the CH3 and CL domains actually comprise the carboxy-terminal domains of the heavy and light chain, respectively.

The term “antibody fragment” or “antigen binding fragment”, as used herein, refers to one or more portions of an antibody that retain the ability to specifically interact with (e.g., by binding, steric hindrance, stabilizing/destabilizing, spatial distribution) an epitope of an antigen (e.g., cKIT). Examples of antibody fragments include, but are not limited to, a Fab fragment, which is a monovalent fragment consisting of the VL, VH, CL and CH1 domains; a Fab′ fragment, which is a monovalent fragment consisting of the VL, VH, CL, CH1 domains, and the hinge region; a F(ab′)2 fragment, which is a bivalent fragment comprising two Fab fragments linked by a disulfide bridge at the hinge region; a half antibody, which includes a single heavy chain and a single light chain linked by a disulfide bridge; an one-arm antibody, which includes a Fab fragment linked to an Fc region; a CH2 domain-deleted antibody, which includes two Fab fragments linked to the CH3 domain dimers (see Glaser, J Biol Chem. 2005; 280(50):41494-503); a single-chain Fv (scFv); a disulfide-linked Fv (sdFv); a Fd fragment consisting of the VH and CH1 domains; a Fv fragment consisting of the VL and VH domains of a single arm of an antibody; a dAb fragment (Ward et al., Nature 341:544-546, 1989), which consists of a VH domain; and an isolated complementarity determining region (CDR), or other epitope-binding fragments of an antibody. For example, a Fab fragment can include amino acid residues 1-222 (EU numbering) of the heavy chain of an antibody; whereas a Fab′ fragment can include amino acid residues 1-236 (EU numbering) of the heavy chain of an antibody. The Fab or Fab′ fragment of an antibody can be generated recombinantly or by enzymatic digestion of a parent antibody. Recombinantly generated Fab or Fab′ may be engineered to introduce amino acids for site-specific conjugation such as cysteines (Junutula, J. R.; et al., Nature biotechnology 2008, 26, 925), pyrroline-carboxy-lysines (Ou, W. et al., Proc Natl Acad Sci USA 2011; 108(26):10437-42) or unnatural amino acids (for example Tian, F. et al., Proc Natl Acad Sci USA 2014, 111, 1766, Axup, J. Y. et al., Proc Natl Acad Sci USA. 2012, 109, 16101. Similarly, mutations or peptide tags can be added to facilitate conjugation through phosphopantetheine transferases (Grunewald, J. et al., Bioconjugate chemistry 2015, 26, 2554), formyl glycine forming enzyme (Drake, P. M. et al., Bioconjugate chemistry 2014, 25, 1331), transglutaminase (Strop, P. et al., Chemistry & biology 2013, 20, 161), sortase (Beerli, R. R.; Hell, T.; Merkel, A. S.; Grawunder, U. PloS one 2015, 10, e0131177) or other enzymatic conjugation strategies. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (“scFv”); see, e.g., Bird et al., Science 242:423-426, 1988; and Huston et al., Proc. Natl. Acad. Sci. 85:5879-5883, 1988). Such single chain antibodies are also intended to be encompassed within the term “antigen binding fragment.” These antigen binding fragments are obtained using conventional techniques known to those of skill in the art, and the fragments are screened for utility in the same manner as are intact antibodies.

Antibody fragments or antigen binding fragments can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can be grafted into scaffolds based on polypeptides such as fibronectin type III (Fn3) (see U.S. Pat. No. 6,703,199, which describes fibronectin polypeptide monobodies).

Antibody fragments or antigen binding fragments can be incorporated into single chain molecules comprising a pair of tandem Fv segments (VH-CH1-VH-CH1) which, together with complementary light chain polypeptides, form a pair of antigen binding regions (Zapata et al., Protein Eng. 8:1057-1062, 1995; and U.S. Pat. No. 5,641,870).

The term “monoclonal antibody” or “monoclonal antibody composition” as used herein refers to polypeptides, including antibodies and antigen binding fragments that have substantially identical amino acid sequence or are derived from the same genetic source. This term also includes preparations of antibody molecules of single molecular composition. A monoclonal antibody composition displays a single binding specificity and affinity for a particular epitope.

The term “human antibody”, as used herein, includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000.

The human antibodies of the present disclosure can include amino acid residues not encoded by human sequences (e.g., mutations introduced by random or site-specific mutagenesis in vitro or by somatic mutation in vivo, or a conservative substitution to promote stability or manufacturing).

The term “recognize” as used herein refers to an antibody or antigen binding fragment thereof that finds and interacts (e.g., binds) with its epitope, whether that epitope is linear or conformational. The term “epitope” refers to a site on an antigen to which an antibody or antigen binding fragment of the disclosure specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art, for example, x-ray crystallography and 2-dimensional nuclear magnetic resonance (see, e.g., Epitope Mapping Protocols in Methods in Molecular Biology, Vol. 66, G. E. Morris, Ed. (1996)). A “paratope” is the part of the antibody which recognizes the epitope of the antigen.

The phrase “specifically binds” or “selectively binds,” when used in the context of describing the interaction between an antigen (e.g., a protein) and an antibody, antibody fragment, or antibody-derived binding agent, refers to a binding reaction that is determinative of the presence of the antigen in a heterogeneous population of proteins and other biologics, e.g., in a biological sample, e.g., a blood, serum, plasma or tissue sample. Thus, under certain designated immunoassay conditions, the antibodies or binding agents with a particular binding specificity bind to a particular antigen at least two times the background and do not substantially bind in a significant amount to other antigens present in the sample. In one aspect, under designated immunoassay conditions, the antibody or binding agent with a particular binding specificity binds to a particular antigen at least ten (10) times the background and does not substantially bind in a significant amount to other antigens present in the sample. Specific binding to an antibody or binding agent under such conditions may require the antibody or agent to have been selected for its specificity for a particular protein. As desired or appropriate, this selection may be achieved by subtracting out antibodies that cross-react with molecules from other species (e.g., mouse or rat) or other subtypes. Alternatively, in some aspects, antibodies or antibody fragments are selected that cross-react with certain desired molecules.

The term “affinity” as used herein refers to the strength of interaction between antibody and antigen at single antigenic sites. Within each antigenic site, the variable region of the antibody “arm” interacts through weak non-covalent forces with antigen at numerous sites; the more interactions, the stronger the affinity.

The term “isolated antibody” refers to an antibody that is substantially free of other antibodies having different antigenic specificities. An isolated antibody that specifically binds to one antigen may, however, have cross-reactivity to other antigens. Moreover, an isolated antibody may be substantially free of other cellular material and/or chemicals.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other all other known variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91%, 92%, 93%, 94%, 95%, 96%, 97%, 98%, 99%, or 100% sequence identity with the reference variable region nucleic acid or amino acid sequence.

A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least 10 to 100 times over the background.

The term “equilibrium dissociation constant (KD [M])” refers to the dissociation rate constant (kd [s⁻¹]) divided by the association rate constant (ka [s⁻¹, M⁻¹]). Equilibrium dissociation constants can be measured using any known method in the art. The antibodies of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

The term “bioavailability” refers to the systemic availability (i.e., blood/plasma levels) of a given amount of drug administered to a patient. Bioavailability is an absolute term that indicates measurement of both the time (rate) and total amount (extent) of drug that reaches the general circulation from an administered dosage form.

As used herein, the phrase “consisting essentially of” refers to the genera or species of active pharmaceutical agents included in a method or composition, as well as any excipients inactive for the intended purpose of the methods or compositions. In some aspects, the phrase “consisting essentially of” expressly excludes the inclusion of one or more additional active agents other than an antibody drug conjugate of the present disclosure. In some aspects, the phrase “consisting essentially of” expressly excludes the inclusion of one or more additional active agents other than an antibody drug conjugate of the present disclosure and a second co-administered agent.

The term “amino acid” refers to naturally occurring, synthetic, and unnatural amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some aspects, the term “conservative sequence modifications” are used to refer to amino acid modifications that do not significantly affect or alter the binding characteristics of the antibody containing the amino acid sequence.

The term “optimized” as used herein refers to a nucleotide sequence that has been altered to encode an amino acid sequence using codons that are preferred in the production cell or organism, generally a eukaryotic cell, for example, a yeast cell, a Pichia cell, a fungal cell, a Trichoderma cell, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence.

The terms “percent identical” or “percent identity,” in the context of two or more nucleic acids or polypeptide sequences, refers to the extent to which two or more sequences or subsequences that are the same. Two sequences are “identical” if they have the same sequence of amino acids or nucleotides over the region being compared. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 30 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, Adv. Appl. Math. 2:482c (1970), by the homology alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, 2003).

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff, (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, Comput. Appl. Biosci. 4:11-17, (1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, J. Mol. Biol. 48:444-453, (1970) algorithm which has been incorporated into the GAP program in the GCG software package (available at www.gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., (1991) Nucleic Acid Res. 19:5081; Ohtsuka et al., (1985) J. Biol. Chem. 260:2605-2608; and Rossolini et al., (1994) Mol. Cell. Probes 8:91-98).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “conjugate” or “antibody drug conjugate” as used herein refers to the linkage of an antibody or an antigen binding fragment thereof with another agent, such as a chemotherapeutic agent, a toxin, an immunotherapeutic agent, an imaging probe, and the like. The linkage can be covalent bonds, or non-covalent interactions such as through electrostatic forces. Various linkers, known in the art, can be employed in order to form the conjugate. Additionally, the conjugate can be provided in the form of a fusion protein that may be expressed from a polynucleotide encoding the conjugate. As used herein, “fusion protein” refers to proteins created through the joining of two or more genes or gene fragments which originally coded for separate proteins (including peptides and polypeptides). Translation of the fusion gene results in a single protein with functional properties derived from each of the original proteins.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The term “toxin”, “cytotoxin” or “cytotoxic agent” as used herein, refers to any agent that is detrimental to the growth and proliferation of cells and may act to reduce, inhibit, or destroy a cell or malignancy.

The term “anti-cancer agent” as used herein refers to any agent that can be used to treat a cell proliferative disorder such as cancer, including but not limited to, cytotoxic agents, chemotherapeutic agents, radiotherapy and radiotherapeutic agents, targeted anti-cancer agents, and immunotherapeutic agents.

The term “drug moiety” or “payload” as used herein refers to a chemical moiety that is conjugated or is suitable for conjugation to an antibody or antigen binding fragment, and can include any therapeutic or diagnostic agent and a metabolite of the antibody drug conjugate disclosed herein that has the desired therapeutic or diagnostic properties, for example, an anti-cancer, anti-inflammatory, anti-infective (e.g., anti-fungal, antibacterial, anti-parasitic, anti-viral), or an anesthetic agent. In certain aspects, a drug moiety is selected from an Eg5 inhibitor, a V-ATPase inhibitor, a HSP90 inhibitor, an IAP inhibitor, an mTor inhibitor, a microtubule stabilizer, a microtubule destabilizer, an auristatin, a dolastatin, a maytansinoid, a MetAP (methionine aminopeptidase), an inhibitor of nuclear export of proteins CRM1, a DPPIV inhibitor, an inhibitor of phosphoryl transfer reactions in mitochondria, a protein synthesis inhibitor, a kinase inhibitor, a CDK2 inhibitor, a CDK9 inhibitor, a proteasome inhibitor, a kinesin inhibitor, an HDAC inhibitor, a DNA damaging agent, a DNA alkylating agent, a DNA intercalator, a DNA minor groove binder, an RNA polymerase inhibitor, an amanitin, a spliceosome inhibitor, a topoisomerase inhibitor and a DHFR inhibitor. Methods for attaching each of these to a linker compatible with the antibodies and method of the present disclosure are known in the art. See, e.g., Singh et al., (2009) Therapeutic Antibodies: Methods and Protocols, vol. 525, 445-457. In addition, a payload can be a biophysical probe, a fluorophore, a spin label, an infrared probe, an affinity probe, a chelator, a spectroscopic probe, a radioactive probe, a lipid molecule, a polyethylene glycol, a polymer, a spin label, DNA, RNA, a protein, a peptide, a surface, an antibody, an antibody fragment, a nanoparticle, a quantum dot, a liposome, a PLGA particle, a saccharide or a polysaccharide.

The term “cancer” includes primary malignant tumors (e.g., those whose cells have not migrated to sites in the subject's body other than the site of the original tumor) and secondary malignant tumors (e.g., those arising from metastasis, the migration of tumor cells to secondary sites that are different from the site of the original tumor).

The term “cKIT” (also known as KIT, PBT, SCFR, C-Kit, CD117) refers to a tyrosine kinase receptor that is a member of the receptor tyrosine kinase III family. The nucleic acid and amino acid sequences of human cKIT isoforms are known, and have been published in GenBank with the following Accession Nos:
NM_000222.2→NP_000213.1 Mast/stem cell growth factor receptor Kit isoform 1 precursor;
NM_001093772.1→NP_001087241.1 Mast/stem cell growth factor receptor Kit isoform 2 precursor.
   Structurally, cKIT receptor is a type I transmembrane protein and contains a signal peptide, 5 Ig-like C2 domains in the extracellular domain and has a protein kinase domain in its intracellular domain. As used herein, the term “cKIT” is used to refer collectively to all naturally occurring isoforms of cKIT protein, or a variant thereof.

The term “variant” refers to a polypeptide that has a substantially identical amino acid sequence to a reference polypeptide, or is encoded by a substantially identical nucleotide sequence, and is capable of having one or more activities of the reference polypeptide. For example, a variant can have about 85%, 90%, 91%, 92%, 93%, 94%, 95%, 96%, 97%, 98%, 99% or higher sequence identity to a reference polypeptide, while retain one or more activities of the reference polypeptide.

As used herein, the terms “treat”, “treating,” or “treatment” of any disease or disorder refer in one aspect, to ameliorating the disease or disorder (i.e., slowing or arresting or reducing the development of the disease or at least one of the clinical symptoms thereof). In another aspect, “treat”, “treating,” or “treatment” refers to alleviating or ameliorating at least one physical parameter including those which may not be discernible by the patient. In yet another aspect, “treat”, “treating,” or “treatment” refers to modulating the disease or disorder, either physically, (e.g., stabilization of a discernible symptom), physiologically, (e.g., stabilization of a physical parameter), or both. In yet another aspect, “treat”, “treating,” or “treatment” refers to preventing or delaying the onset or development or progression of the disease or disorder.

The term “therapeutically acceptable amount” or “therapeutically effective dose” interchangeably refers to an amount sufficient to effect the desired result (i.e., a reduction in tumor size, inhibition of tumor growth, prevention of metastasis, inhibition or prevention of viral, bacterial, fungal or parasitic infection). In some aspects, a therapeutically acceptable amount does not induce or cause undesirable side effects. A therapeutically acceptable amount can be determined by first administering a low dose, and then incrementally increasing that dose until the desired effect is achieved. A “therapeutically effective dosage,” of the molecules of the present disclosure can prevent the onset of, or result in a decrease in severity of, respectively, disease symptoms, including symptoms associated with cancer.

The term “co-administer” refers to the simultaneous presence of two active agents in the blood of an individual. Active agents that are co-administered can be concurrently or sequentially delivered.

The term ‘thiol-maleimide’ as used herein refers to a group formed by reaction of a thiol with maleimide, having this general formula:

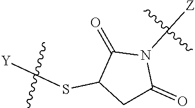

where Y and Z are groups to be connected via the thiol-maleimide linkage and can comprise linker components, antibodies or payloads. The thiol-maleimide may form the following ring opened structures

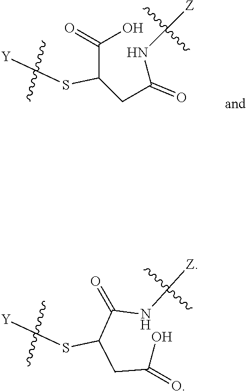

“Cleavable” as used herein refers to a linking group or linker component that connects two moieties by covalent connections, but breaks down to sever the covalent connection between the moieties under physiologically relevant conditions, typically a cleavable linking group is severed in vivo more rapidly in an intracellular environment than when outside a cell, causing release of the payload to preferentially occur inside a targeted cell. Cleavage may be enzymatic or non-enzymatic, but generally releases a payload from an antibody without degrading the antibody. Cleavage may leave some portion of a linking group or linker component attached to the payload, or it may release the payload without any residue of the linking group.

“Non-cleavable” as used herein refers to a linking group or linker component that is not especially susceptible to breaking down under physiological conditions, e.g., it is at least as stable as the antibody or antigen binding fragment portion of the conjugate. Such linking groups are sometimes referred to as ‘stable’, meaning they are sufficiently resistant to degradation to keep the payload connected to antibody or antigen binding fragment until the antibody or antigen binding fragment is itself at least partially degraded, i.e., the degradation of the antibody or antigen binding fragment precedes cleavage of the linking group in vivo. Degradation of the antibody portion of an ADC having a stable or non-cleavable linking group may leave some or all of the linking group, e.g., one or more amino acid groups from an antibody, attached to the payload or drug moiety that is delivered in vivo.

### Drug Moiety (D)

In one aspect, the Drug moiety of the invention is a compound of Formula (A):

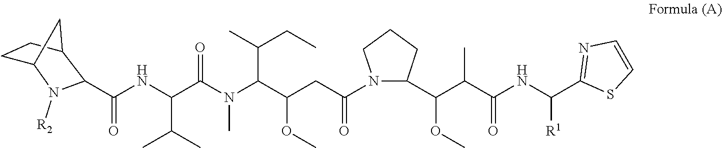

wherein:
R¹ is

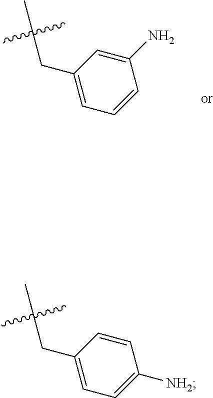 [SMILES: *Cc1cccc(N)c1 ; "or" ; *Cc1ccc(N)cc1]

R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)R⁴ or C₁-C₆alkyl which is optionally substituted with —CN, —C(═O)NH₂ or 1 to 5 hydroxyl;
each R³ is independently selected from C₁-C₆alkyl and C₁-C₆alkyl which is optionally substituted with 1 to 5 hydroxyl;
and
each R⁴ is independently selected from H and C₁-C₆alkyl.

In one aspect, the Drug moiety of the invention is a compound of Formula (B):

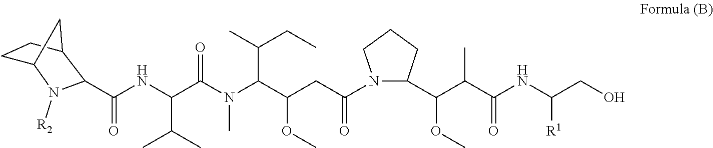

wherein:
R¹ is

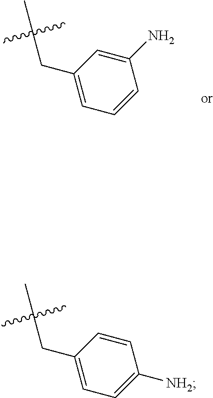 [SMILES: *Cc1cccc(N)c1 ; "or" ; *Cc1ccc(N)cc1]

R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)R⁴ or C₁-C₆alkyl which is optionally substituted with —CN, —C(═O)NH₂ or 1 to 5 hydroxyl;
each R³ is independently selected from C₁-C₆alkyl and C₁-C₆alkyl which is optionally substituted with 1 to 5 hydroxyl;
and
each R⁴ is independently selected from H and C₁-C₆alkyl.

Certain aspects and examples of the Drug moiety of the invention are provided in the following listing of additional, enumerated embodiments. It will be recognized that features specified in each embodiment may be combined with other specified features to provide further embodiments of the present invention.

Embodiment 1. The compound of Formula (A), or a pharmaceutically acceptable salt thereof, having the structure of Formula (A-1), or a pharmaceutically acceptable salt thereof:

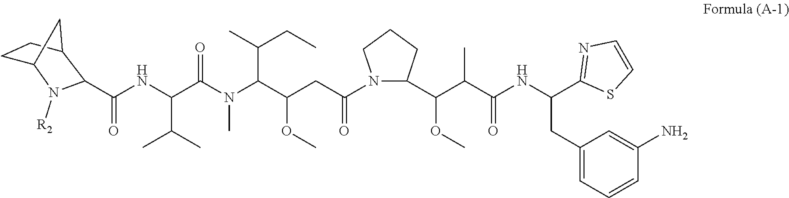

wherein:
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)ₙR⁴ or C₁-C₆alkyl which is optionally substituted with —CN, —C(═O)NH₂ or 1 to 5 hydroxyl;
each R³ is independently selected from C₁-C₆alkyl and C₁-C₆alkyl which is optionally substituted with 1 to 5 hydroxyl;
and
each R⁴ is independently selected from H and C₁-C₆alkyl.

Embodiment 2. The compound of Formula (A), or a pharmaceutically acceptable salt thereof, having the structure of Formula (A-2), or a pharmaceutically acceptable salt thereof:

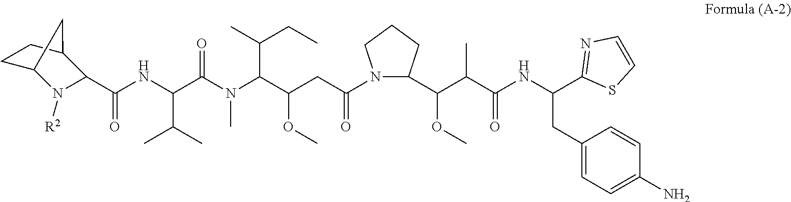

wherein:
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)ₙR⁴ or C₁-C₆alkyl which is optionally substituted with —CN, —C(═O)NH₂ or 1 to 5 hydroxyl;
each R³ is independently selected from C₁-C₆alkyl and C₁-C₆alkyl which is optionally substituted with 1 to 5 hydroxyl;
and
each R⁴ is independently selected from H and C₁-C₆alkyl.

Embodiment 3. The compound of Formula (A), or a pharmaceutically acceptable salt thereof, having the structure of Formula (A-3), or a pharmaceutically acceptable salt thereof:

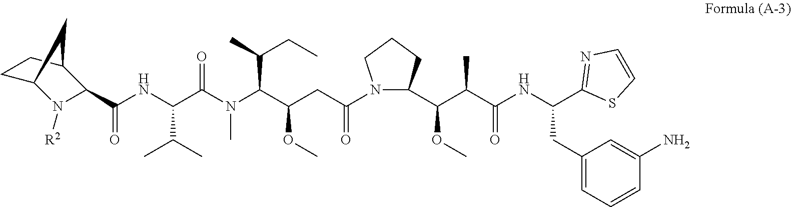

wherein:
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)ₙR⁴ or C₁-C₆alkyl which is optionally substituted with —CN, —C(═O)NH₂ or 1 to 5 hydroxyl;
each R³ is independently selected from C₁-C₆alkyl and C₁-C₆alkyl which is optionally substituted with 1 to 5 hydroxyl;
and
each R⁴ is independently selected from H and C₁-C₆alkyl.

Embodiment 2. The compound of Formula (A), or a pharmaceutically acceptable salt thereof, having the structure of Formula (A-4), or a pharmaceutically acceptable salt thereof:

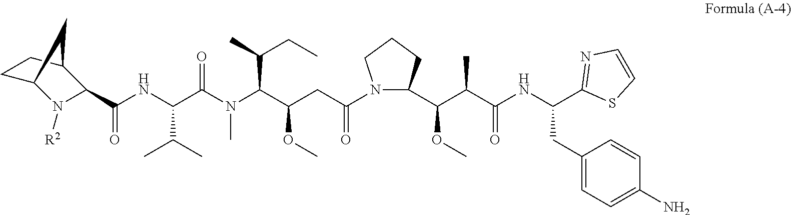

wherein:
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)ₙR⁴ or C₁-C₆alkyl which is optionally substituted with —CN, —C(═O)NH₂ or 1 to 5 hydroxyl;
each R³ is independently selected from C₁-C₆alkyl and C₁-C₆alkyl which is optionally substituted with 1 to 5 hydroxyl;
and
each R⁴ is independently selected from H and C₁-C₆alkyl.

Embodiment 3. The compound of Formula (A), Formula (A-1), Formula (A-2), Formula (A-3) or Formula (A-4), or a pharmaceutically acceptable salt thereof, wherein: R² is H or C₁-C₆alkyl.

Embodiment 4. The compound of Formula (A), Formula (A-1), Formula (A-2), Formula (A-3) or Formula (A-4), or a pharmaceutically acceptable salt thereof, wherein: R² is H or methyl.

Embodiment 5. The compound of Formula (A), Formula (A-1), Formula (A-2), Formula (A-3) or Formula (A-4), or a pharmaceutically acceptable salt thereof, wherein: R² is H.

Embodiment 6. The compound of Formula (A), Formula (A-1), Formula (A-2), Formula (A-3) or Formula (A-4), or a pharmaceutically acceptable salt thereof, wherein: R² is methyl.

Embodiment 7. The compound of Formula (A), Formula (A-1) or Formula (A-3), or a pharmaceutically acceptable salt thereof, wherein the compound is

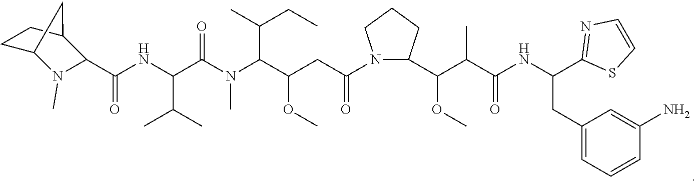 [SMILES: CCC(C)C(C(CC(=O)N1CCCC1C(OC)C(C)C(=O)NC(Cc1cccc(N)c1)c1nccs1)OC)N(C)C(=O)C(NC(=O)C1C2CCC(C2)N1C)C(C)C]

Embodiment 8. The compound of Formula (A), Formula (A-1) or Formula (A-3), or a pharmaceutically acceptable salt thereof, wherein the compound is

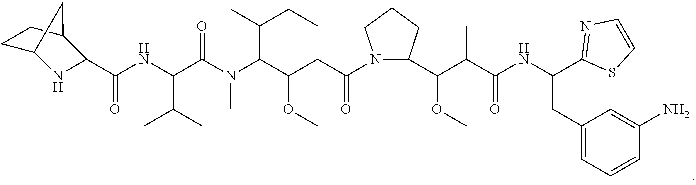 [SMILES: CCC(C)C(C(CC(=O)N1CCCC1C(OC)C(C)C(=O)NC(Cc1cccc(N)c1)c1nccs1)OC)N(C)C(=O)C(NC(=O)C1NC2CCC1C2)C(C)C]

Embodiment 9. The compound of Formula (A), Formula (A-1) or Formula (A-3), or a pharmaceutically acceptable salt thereof, wherein the compound is

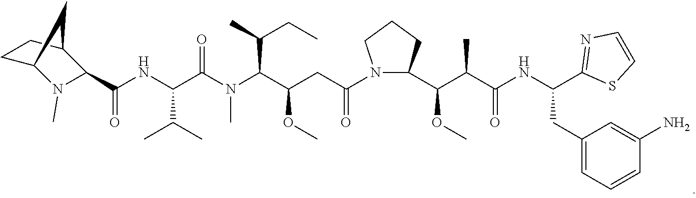 [SMILES: CC[C@H](C)[C@@H]([C@@H](CC(=O)N1CCC[C@H]1[C@H](OC)[C@@H](C)C(=O)N[C@@H](Cc1cccc(N)c1)c1nccs1)OC)N(C)C(=O)[C@@H](NC(=O)[C@@H]1[C@H]2CC[C@@H](C2)N1C)C(C)C]

Embodiment 10. The compound of Formula (A), Formula (A-1) or Formula (A-3), or a pharmaceutically acceptable salt thereof, wherein the compound is

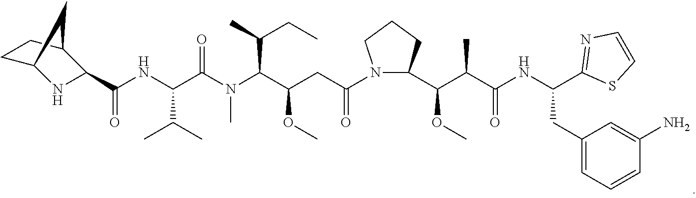 [SMILES: CC[C@H](C)[C@@H]([C@@H](CC(=O)N1CCC[C@H]1[C@H](OC)[C@@H](C)C(=O)N[C@@H](Cc1cccc(N)c1)c1nccs1)OC)N(C)C(=O)[C@@H](NC(=O)[C@H]1N[C@H]2CC[C@H]1C2)C(C)C]

Embodiment 11. The compound of Formula (A), Formula (A-2) or Formula (A-4), or a pharmaceutically acceptable salt thereof, wherein the compound is

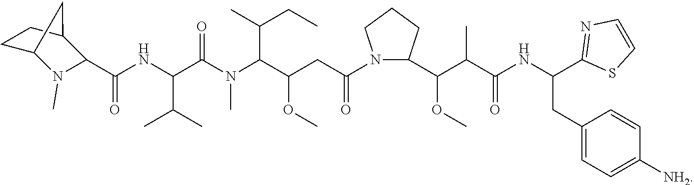 [SMILES: CCC(C)C(C(CC(=O)N1CCCC1C(OC)C(C)C(=O)NC(Cc1ccc(N)cc1)c1nccs1)OC)N(C)C(=O)C(NC(=O)C1C2CCC(C2)N1C)C(C)C]

Embodiment 12. The compound of Formula (A), Formula (A-2) or Formula (A-4), or a pharmaceutically acceptable salt thereof, wherein the compound is

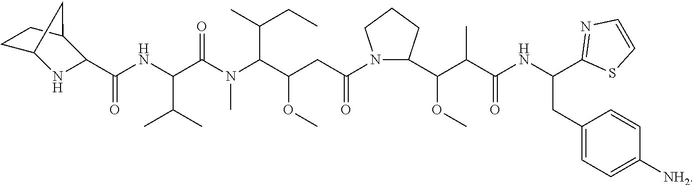 [SMILES: CCC(C)C(C(CC(=O)N1CCCC1C(OC)C(C)C(=O)NC(Cc1ccc(N)cc1)c1nccs1)OC)N(C)C(=O)C(NC(=O)C1NC2CCC1C2)C(C)C]

Embodiment 13. The compound of Formula (A), Formula (A-2) or Formula (A-4), or a pharmaceutically acceptable salt thereof, wherein the compound is

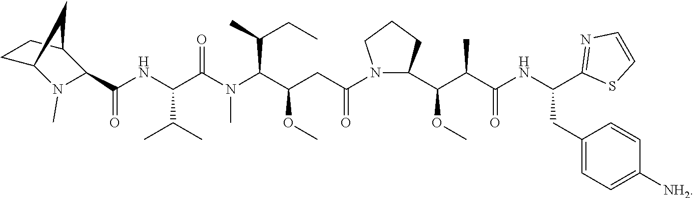 [SMILES: CC[C@H](C)[C@@H]([C@@H](CC(=O)N1CCC[C@H]1[C@H](OC)[C@@H](C)C(=O)N[C@@H](Cc1ccc(N)cc1)c1nccs1)OC)N(C)C(=O)[C@@H](NC(=O)[C@@H]1[C@H]2CC[C@@H](C2)N1C)C(C)C]

Embodiment 14. The compound of Formula (A), Formula (A-2) or Formula (A-4), or a pharmaceutically acceptable salt thereof, wherein the compound is

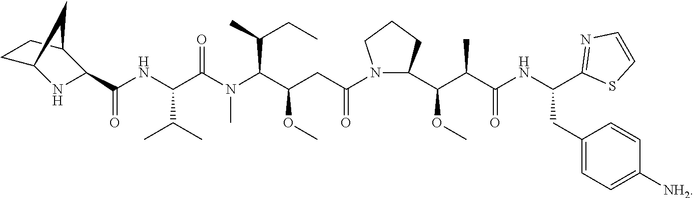 [SMILES: CC[C@H](C)[C@@H]([C@@H](CC(=O)N1CCC[C@H]1[C@H](OC)[C@@H](C)C(=O)N[C@@H](Cc1ccc(N)cc1)c1nccs1)OC)N(C)C(=O)[C@@H](NC(=O)[C@H]1N[C@H]2CC[C@H]1C2)C(C)C]

Embodiment 15. The compound of Formula (B), or a pharmaceutically acceptable salt thereof, having the structure of Formula (B-1), or a pharmaceutically acceptable salt thereof:

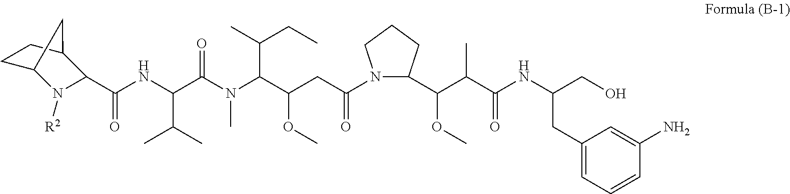

wherein:
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)ₙR⁴ or C₁-C₆alkyl which is optionally substituted with —CN, —C(═O)NH₂ or 1 to 5 hydroxyl;
each R³ is independently selected from C₁-C₆alkyl and C₁-C₆alkyl which is optionally substituted with 1 to 5 hydroxyl;
and
each R⁴ is independently selected from H and C₁-C₆alkyl.

Embodiment 16. The compound of Formula (B), or a pharmaceutically acceptable salt thereof, having the structure of Formula (B-2), or a pharmaceutically acceptable salt thereof:

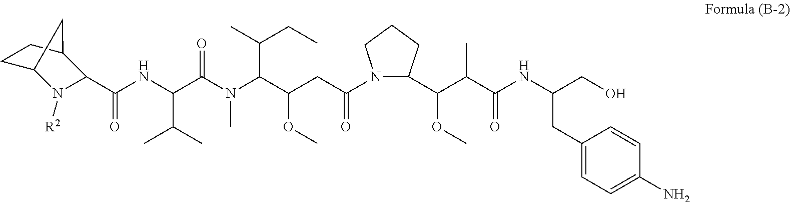

wherein:
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)ₙR⁴ or C₁-C₆alkyl which is optionally substituted with —CN, —C(═O)NH₂ or 1 to 5 hydroxyl;
each R³ is independently selected from C₁-C₆alkyl and C₁-C₆alkyl which is optionally substituted with 1 to 5 hydroxyl;
and
each R⁴ is independently selected from H and C₁-C₆alkyl.

Embodiment 17. The compound of Formula (B), or a pharmaceutically acceptable salt thereof, having the structure of Formula (B-3), or a pharmaceutically acceptable salt thereof:

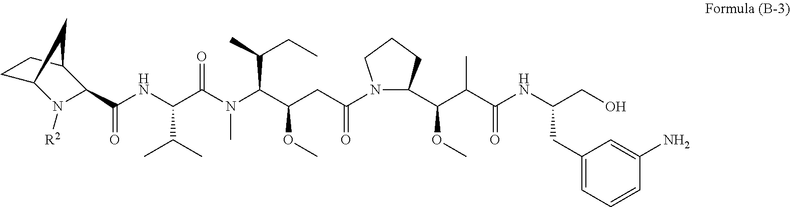

wherein:
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)ₙR⁴ or C₁-C₆alkyl which is optionally substituted with —CN, —C(═O)NH₂ or 1 to 5 hydroxyl;
each R³ is independently selected from C₁-C₆alkyl and C₁-C₆alkyl which is optionally substituted with 1 to 5 hydroxyl;
and
each R⁴ is independently selected from H and C₁-C₆alkyl.

Embodiment 18. The compound of Formula (B), or a pharmaceutically acceptable salt thereof, having the structure of Formula (B-4), or a pharmaceutically acceptable salt thereof:

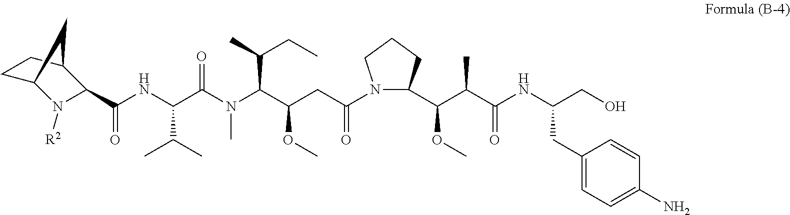

wherein:
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)ₙR⁴ or C₁-C₆alkyl which is optionally substituted with —CN, —C(═O)NH₂ or 1 to 5 hydroxyl;
each R³ is independently selected from C₁-C₆alkyl and C₁-C₆alkyl which is optionally substituted with 1 to 5 hydroxyl;
and
each R⁴ is independently selected from H and C₁-C₆alkyl.

Embodiment 19. The compound of Formula (B), Formula (B-1), Formula (B-2), Formula (B-3) or Formula (B-4), or a pharmaceutically acceptable salt thereof, wherein: R² is H or C₁-C₆alkyl.

Embodiment 20. The compound Formula (B), Formula (B-1), Formula (B-2), Formula (B-3) or Formula (B-4), or a pharmaceutically acceptable salt thereof, wherein: R² is H or methyl.

Embodiment 21. The compound of Formula (B), Formula (B-1), Formula (B-2), Formula (B-3) or Formula (B-4), or a pharmaceutically acceptable salt thereof, wherein: R² is H.

Embodiment 22. The compound of Formula (B), Formula (B-1), Formula (B-2), Formula (B-3) or Formula (B-4), or a pharmaceutically acceptable salt thereof, wherein: R² is methyl.

Embodiment 23. The compound of Formula (B), Formula (B-1) or Formula (B-3), or a pharmaceutically acceptable salt thereof, wherein the compound is

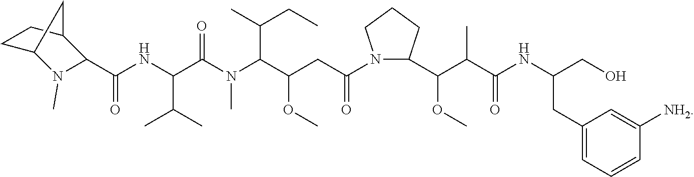 [SMILES: CCC(C)C(C(CC(=O)N1CCCC1C(OC)C(C)C(=O)NC(CO)Cc1cccc(N)c1)OC)N(C)C(=O)C(NC(=O)C1C2CCC(C2)N1C)C(C)C]

Embodiment 24 The compound of Formula (B), Formula (B-1) or Formula (B-3), or a pharmaceutically acceptable salt thereof, wherein the compound is

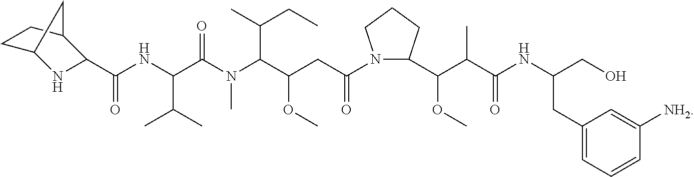 [SMILES: CCC(C)C(C(CC(=O)N1CCCC1C(OC)C(C)C(=O)NC(CO)Cc1cccc(N)c1)OC)N(C)C(=O)C(NC(=O)C1NC2CCC1C2)C(C)C]

Embodiment 25. The compound of Formula (B), Formula (B-1) or Formula (B-3), or a pharmaceutically acceptable salt thereof, wherein the compound is

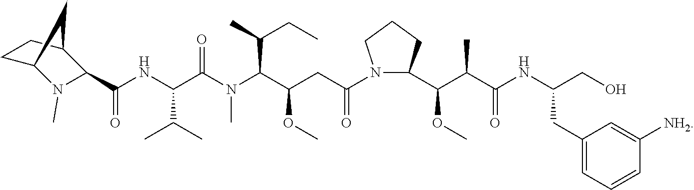 [SMILES: CC[C@H](C)[C@@H]([C@@H](CC(=O)N1CCC[C@H]1[C@H](OC)[C@@H](C)C(=O)N[C@H](CO)Cc1cccc(N)c1)OC)N(C)C(=O)[C@@H](NC(=O)[C@@H]1[C@H]2CC[C@@H](C2)N1C)C(C)C]

Embodiment 26. The compound of Formula (B), Formula (B-1) or Formula (B-3), or a pharmaceutically acceptable salt thereof, wherein the compound is

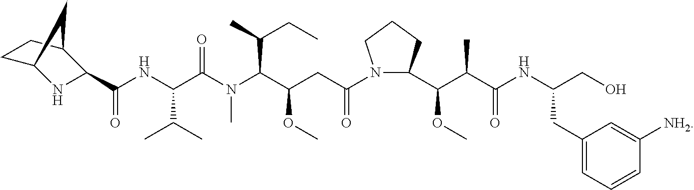 [SMILES: CC[C@H](C)[C@@H]([C@@H](CC(=O)N1CCC[C@H]1[C@H](OC)[C@@H](C)C(=O)N[C@H](CO)Cc1cccc(N)c1)OC)N(C)C(=O)[C@@H](NC(=O)[C@H]1N[C@H]2CC[C@H]1C2)C(C)C]

Embodiment 27. The compound of Formula (B), Formula (B-2) or Formula (B-4), or a pharmaceutically acceptable salt thereof, wherein the compound is

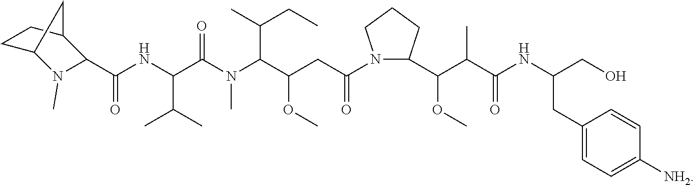 [SMILES: CCC(C)C(C(CC(=O)N1CCCC1C(OC)C(C)C(=O)NC(CO)Cc1ccc(N)cc1)OC)N(C)C(=O)C(NC(=O)C1C2CCC(C2)N1C)C(C)C]

Embodiment 28. The compound of Formula (B), Formula (B-2) or Formula (B-4), or a pharmaceutically acceptable salt thereof, wherein the compound is

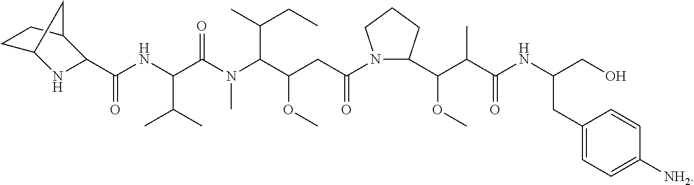 [SMILES: CCC(C)C(C(CC(=O)N1CCCC1C(OC)C(C)C(=O)NC(CO)Cc1ccc(N)cc1)OC)N(C)C(=O)C(NC(=O)C1NC2CCC1C2)C(C)C]

Embodiment 29. The compound of Formula (B), Formula (B-2) or Formula (B-4), or a pharmaceutically acceptable salt thereof, wherein the compound is

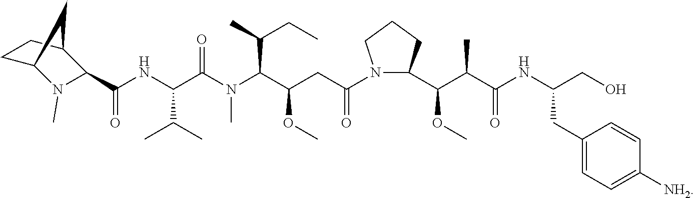 [SMILES: CC[C@H](C)[C@@H]([C@@H](CC(=O)N1CCC[C@H]1[C@H](OC)[C@@H](C)C(=O)N[C@H](CO)Cc1ccc(N)cc1)OC)N(C)C(=O)[C@@H](NC(=O)[C@@H]1[C@H]2CC[C@@H](C2)N1C)C(C)C]

Embodiment 30. The compound of Formula (A), Formula (A-2) or Formula (A-4), or a pharmaceutically acceptable salt thereof, wherein the compound is

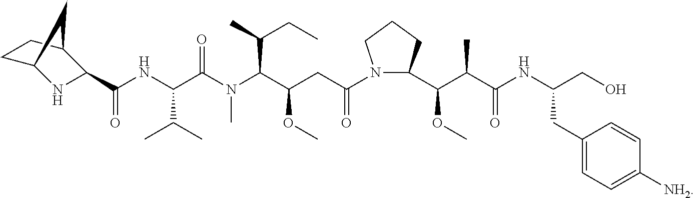 [SMILES: CC[C@H](C)[C@@H]([C@@H](CC(=O)N1CCC[C@H]1[C@H](OC)[C@@H](C)C(=O)N[C@H](CO)Cc1ccc(N)cc1)OC)N(C)C(=O)[C@@H](NC(=O)[C@H]1N[C@H]2CC[C@H]1C2)C(C)C]

Linker-Drug Moiety (L_{B}-(D)ₙ)

In one aspect, the Linker-Drug moiety of the invention comprises one or more cytotoxins covalently attached to a linker (L_{B}), wherein the one or more cytotoxins are independently selected from compound of Formula (A), Formula (A-1), Formula (A-2), Formula (A-3), Formula (A-4), Formula (B), Formula (B-1), Formula (B-2), Formula (B-3) or Formula (B-4) or a compound of any one of Embodiments 7 to 14 or any one of Embodiments 23 to 30

In one aspect, the Linker-Drug moiety of the invention comprises one or more cytotoxins covalently attached to a linker (L_{B}), wherein the one or more cytotoxins are independently selected from compound of Formula (A), Formula (A-1), Formula (A-2), Formula (A-3) or Formula (A-4), or a compound of any one of Embodiments 7 to 14.

In one aspect, the Linker-Drug moiety of the invention comprises one or more cytotoxins covalently attached to a linker (L_{B}), wherein the one or more cytotoxins are independently selected from compound of Formula (B), Formula (B-1), Formula (B-2), Formula (B-3) or Formula (B-4) or a compound of any one of Embodiments 23 to 30.

In one aspect the Linker-Drug moiety of the invention is a compound having the structure of Formula (C), or stereoisomers or pharmaceutically acceptable salts thereof,

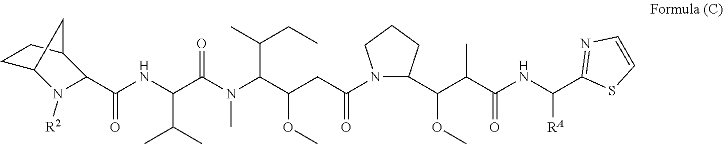

wherein:
R^{A} is

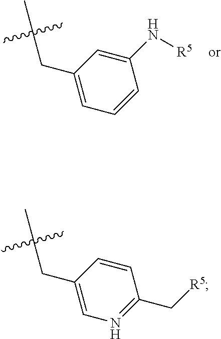

R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)ₙR⁴ or C₁-C₆alkyl which is optionally substituted with —CN, —C(═O)NH₂ or 1 to 5 hydroxyl;
each R³ is independently selected from C₁-C₆alkyl and C₁-C₆alkyl which is optionally substituted with 1 to 5 hydroxyl;
each R⁴ is independently selected from H and C₁-C₆alkyl; R⁵ is -L₁R¹⁴, -L₁R²⁴, -L₁R³⁴ or -L₁R⁴⁴;
L₁ is —X₁C(═O)((CH₂)ₘO)ₚ(CH₂)ₘ—**, —X₁C(═O)(CH₂)ₘ—**, —X₂X₁C(═O)((CH₂)ₘO)ₚ(CH₂)ₘ—**, —X₂X₁C(═O)(CH₂)ₘ—**, —X₃C(═O)((CH₂)ₘO)ₚ(CH₂)ₘ—**, —X₃C(═O)(CH₂)ₘ—**, —X₃C(═O)(CH₂)ₘNHC(═O)((CH₂)ₘO)ₚ(CH₂)ₘ—**, —X₃C(═O)(CH₂)ₘNHC(═O)(CH₂)ₘ—**, —X₃C(═O)(CH₂)ₘ—**, —X₁C(═O)((CH₂)ₘO)ₚ(CH₂)ₘX₄(CH₂)ₘ—**, —X₁C(═O)(CH₂)ₘX₄(CH₂)ₘ—**, —X₂X₁C(═O)((CH₂)ₘO)ₚ(CH₂)ₘX₄(CH₂)ₘ—** or —X₂X₁C(═O)(CH₂)ₘX₄(CH₂)ₘ**, where ** indicates the point of attachment to R¹¹⁴;
X₁ is

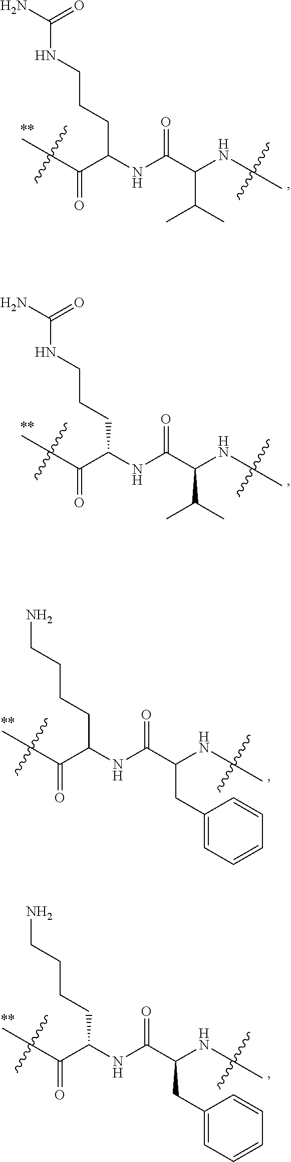 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O]

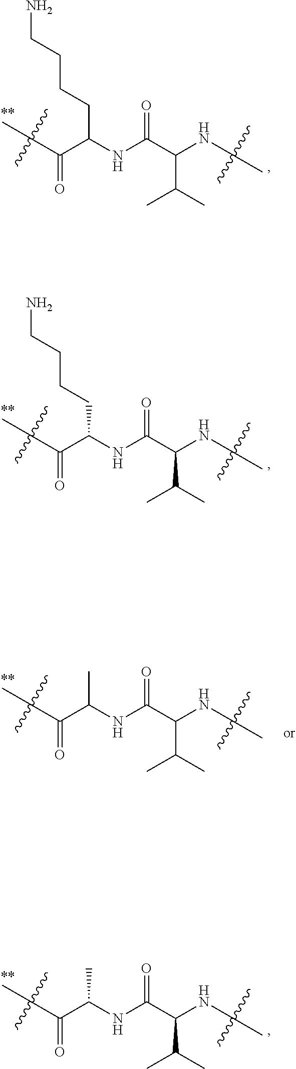 [SMILES: *NC(C(=O)NC(CCCCN)C(*)=O)C(C)C ; *N[C@H](C(=O)N[C@@H](CCCCN)C(*)=O)C(C)C ; *NC(C(=O)NC(C)C(*)=O)C(C)C ; *N[C@H](C(=O)N[C@@H](C)C(*)=O)C(C)C]

where ** indicates the point of attachment to the —NH— or to X₂;
X₂ is

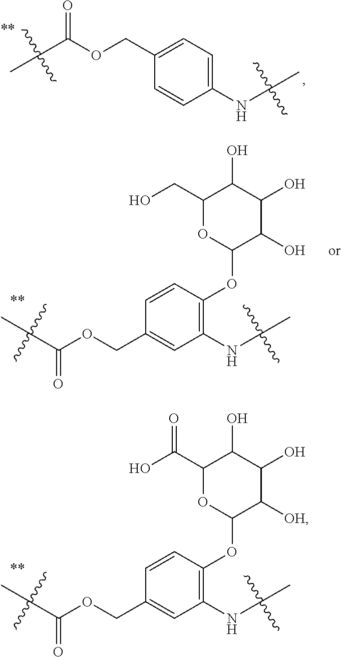

where ** indicates the point of attachment to the —NH—;
X₃ is

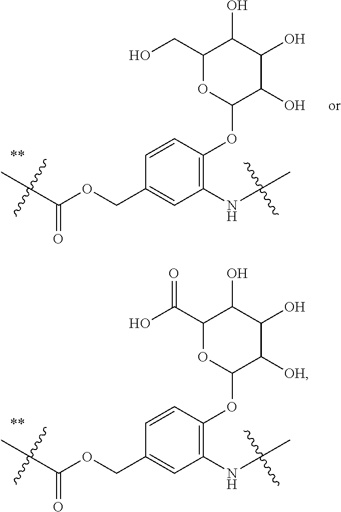 [SMILES: *Nc1cc(COC(*)=O)ccc1OC1OC(CO)C(O)C(O)C1O ; *Nc1cc(COC(*)=O)ccc1OC1OC(C(=O)O)C(O)C(O)C1O]

where ** indicates the point of attachment to to the —NH—;
X₄ is

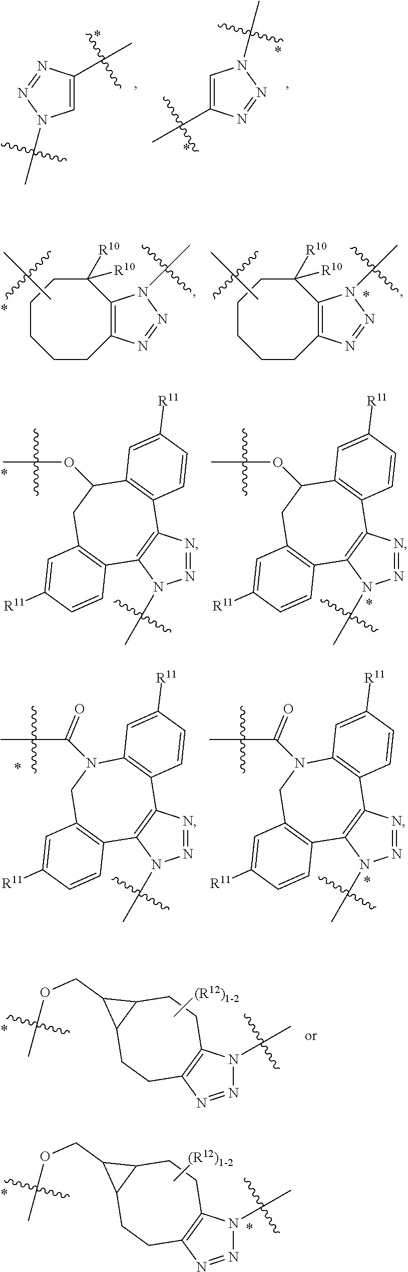

where the * indicates the point of attachment is toward R¹⁴, R²⁴, R³⁴ or R⁴⁴;
R¹⁴ is

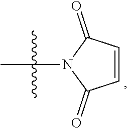 [SMILES: *N1C(=O)C=CC1=O]

—N₃, —ONH₂, —NR⁶C(═O)CH═CH₂, SH, —SSR⁷, —S(═O)₂(CH═CH₂), —NR⁶S(═O)₂(CH═CH₂), —NR⁶C(═O)CH₂Br, —NR⁶C(═O)CH₂I, —NHC(═O)CH₂Br, —NHC(═O)CH₂I, —C(═O)NHNH₂,

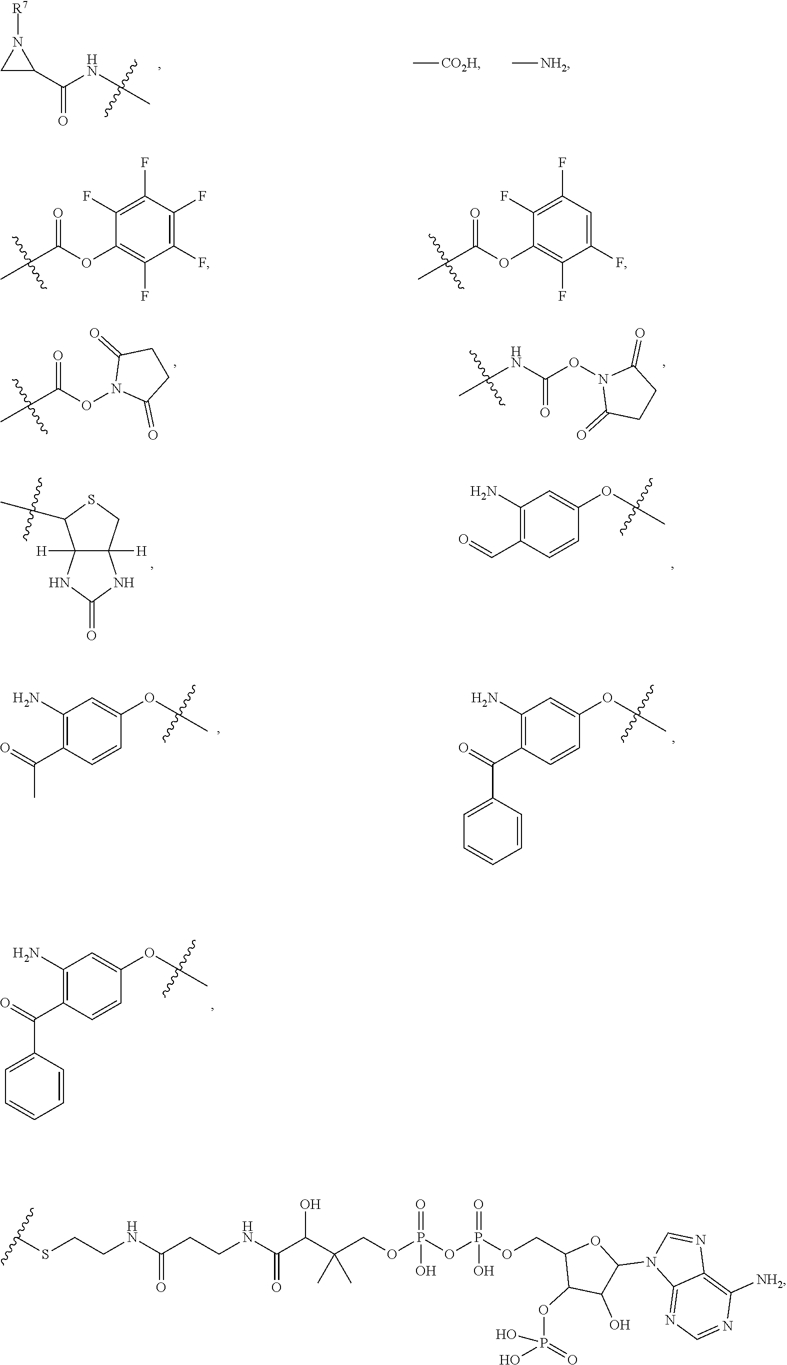

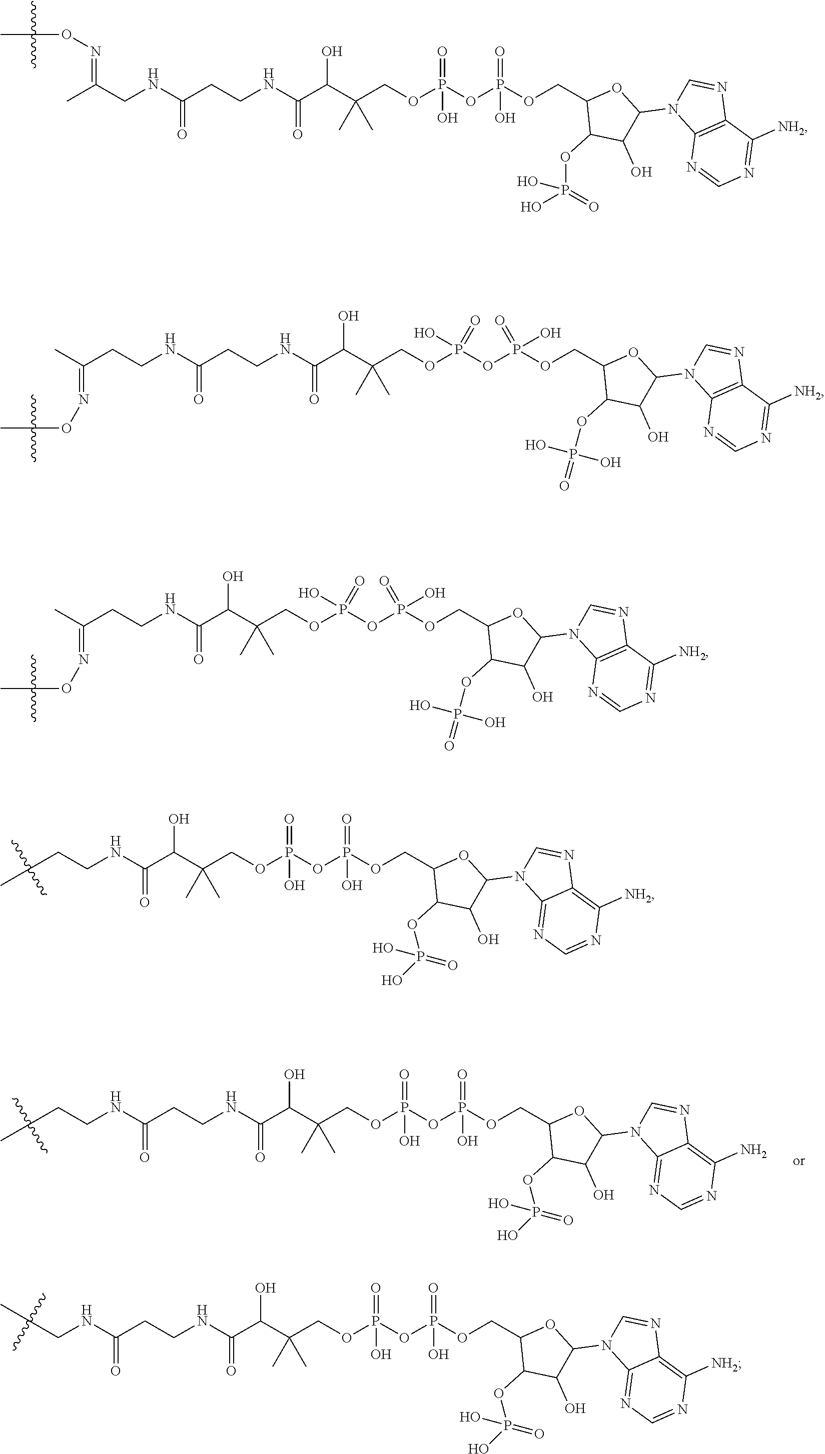

R²⁴ is

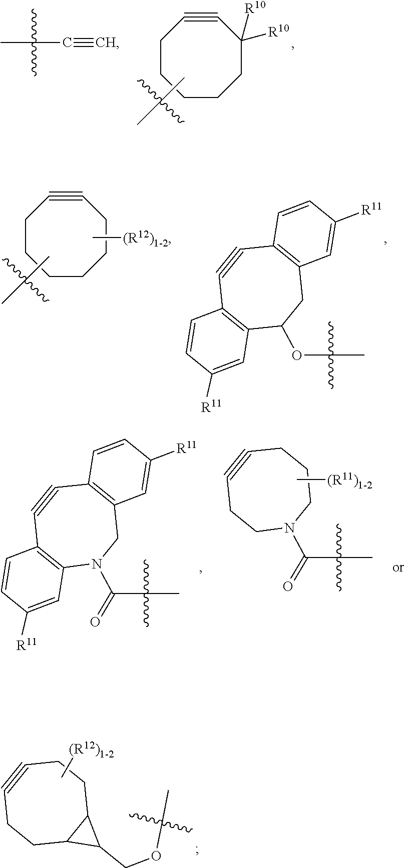

R³⁴ is, —N₃, —ONH₂, —NR⁷C(═O)CH═CH₂, —C(═O)NHNH₂, —CO₂H, —NH₂,

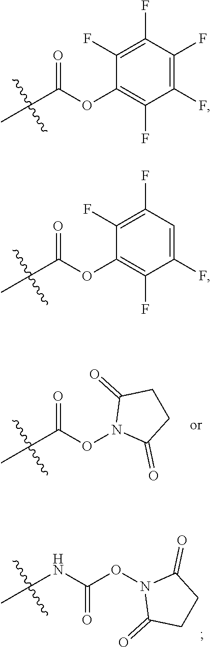

R⁴⁴ is

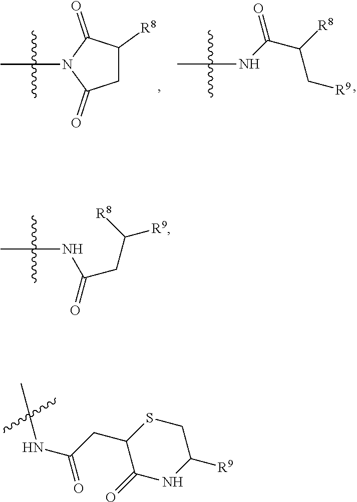

or —NR⁷C(═O)CH₂R;
each R⁶ is independently selected from H and C₁-C₆alkyl;
R⁷ is 2-pyridyl or 4-pyridyl;
R⁸ is —S(CH₂)ₙCHR⁹NH₂;
R⁹ is —C(═O)OR⁷;
each R¹⁰ is independently selected from H, C₁-C₆alkyl, F, Cl, and —OH;
each R¹¹ is independently selected from H, C₁-C₆alkyl, F, Cl, —NH₂, —OCH₃, —OCH₂CH₃, —N(CH₃)₂, —CN, —NO₂ and —OH;
each R¹² is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each m is independently selected from 1, 2, 3, 4, 5, 6, 7, 8, 9 and 10, and each p is independently selected from 1, 2, 3, 4, 5, 6, 7, 8, 9, 10, 11, 12, 13 and 14.

Certain aspects and examples of the Linker-Drug moiety of the invention are provided in the following listing of additional, enumerated embodiments. It will be recognized that features specified in each embodiment may be combined with other specified features to provide further embodiments of the present invention.

Embodiment 31. The compound of Formula (C), or a pharmaceutically acceptable salt thereof, having the structure of Formula (C-1), or a pharmaceutically acceptable salt thereof:

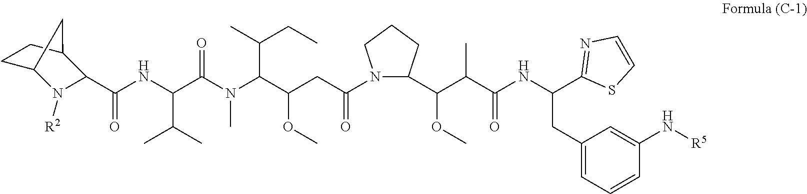

wherein: R² and R⁵ are as defined above for compounds of Formula (C).

Embodiment 32. The compound of Formula (C), or a pharmaceutically acceptable salt thereof, having the structure of Formula (C-2), or a pharmaceutically acceptable salt thereof:

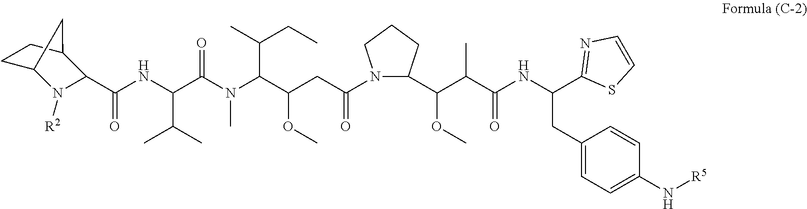

wherein: R² and R⁵ are as defined above for compounds of Formula (C).

Embodiment 33. The compound of Formula (C), or a pharmaceutically acceptable salt thereof, having the structure of Formula (C-3), or a pharmaceutically acceptable salt thereof:

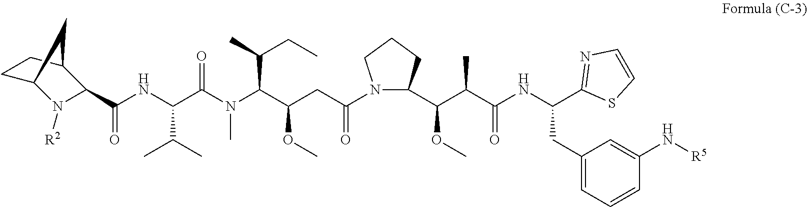

wherein: R² and R⁵ are as defined above for compounds of Formula (C).

Embodiment 32. The compound of Formula (C), or a pharmaceutically acceptable salt thereof, having the structure of Formula (C-4), or a pharmaceutically acceptable salt thereof:

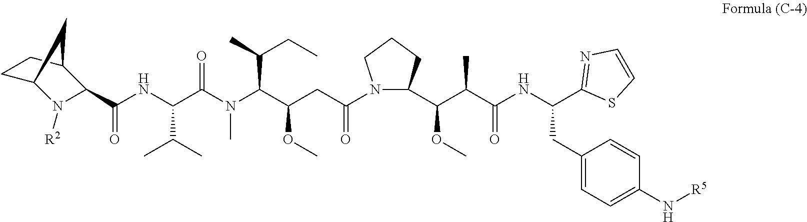

wherein: R² and R⁵ are as defined above for compounds of Formula (C).

Embodiment 33. The compound of Formula (C), Formula (C-1), Formula (C-2), Formula (C-3) or Formula (C-4), or a pharmaceutically acceptable salt thereof, wherein: R² is H or C₁-C₆alkyl.

Embodiment 34. The compound of Formula (C), Formula (C-1), Formula (C-2), Formula (C-3) or Formula (C-4), or a pharmaceutically acceptable salt thereof, wherein: R² is H or methyl.

Embodiment 35. The compound of Formula (C), Formula (C-1), Formula (C-2), Formula (C-3) or Formula (C-4), or a pharmaceutically acceptable salt thereof, wherein: R² is H.

Embodiment 36. The compound of Formula (C), Formula (C-1), Formula (C-2), Formula (C-3) or Formula (C-4), or a pharmaceutically acceptable salt thereof, wherein: R² is methyl.

Embodiment 37. The compound of Formula (C), Formula (C-1) or Formula (C-3), or a pharmaceutically acceptable salt thereof, having the structure of Formula (C-5), or a pharmaceutically acceptable salt thereof:

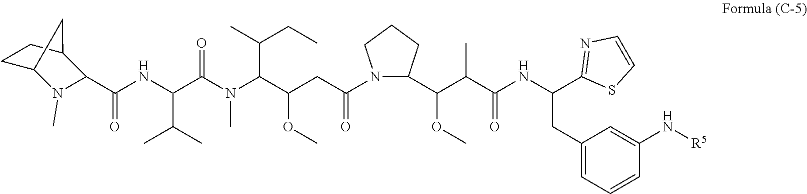

wherein: R⁵ are as defined above for compounds of Formula (C).

Embodiment 38. The compound of Formula (C), Formula (C-1) or Formula (C-3), or a pharmaceutically acceptable salt thereof, having the structure of Formula (C-6), or a pharmaceutically acceptable salt thereof:

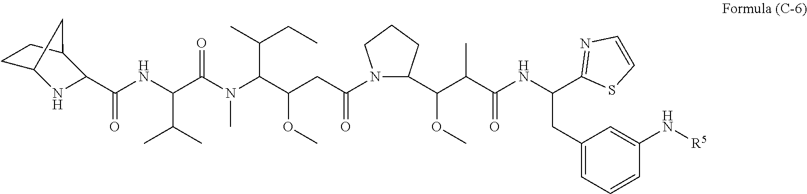

wherein: R⁵ are as defined above for compounds of Formula (C).

Embodiment 39. The compound of Formula (C), Formula (C-1) or Formula (C-3), or a pharmaceutically acceptable salt thereof, having the structure of Formula (C-7), or a pharmaceutically acceptable salt thereof:

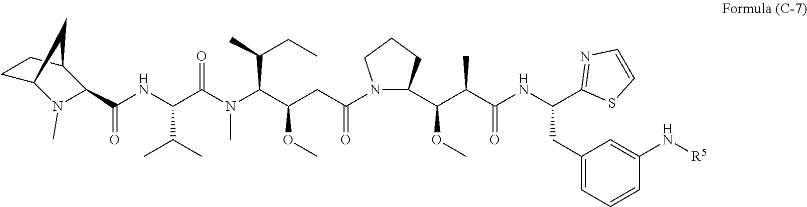

wherein: R⁵ are as defined above for compounds of Formula (C).

Embodiment 40. The compound of Formula (C), Formula (C-1) or Formula (C-3), or a pharmaceutically acceptable salt thereof, having the structure of Formula (C-8), or a pharmaceutically acceptable salt thereof:

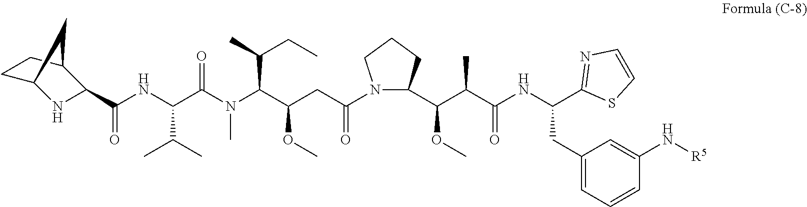

wherein: R⁵ are as defined above for compounds of Formula (C).

Embodiment 41. The compound of Formula (C), Formula (C-1) or Formula (C-3), or a pharmaceutically acceptable salt thereof, having the structure of Formula (C-9), or a pharmaceutically acceptable salt thereof:

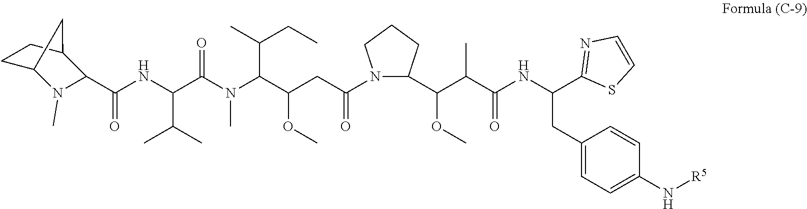

wherein: R⁵ are as defined above for compounds of Formula (C).

Embodiment 42. The compound of Formula (C), Formula (C-1) or Formula (C-3), or a pharmaceutically acceptable salt thereof, having the structure of Formula (C-10), or a pharmaceutically acceptable salt thereof:

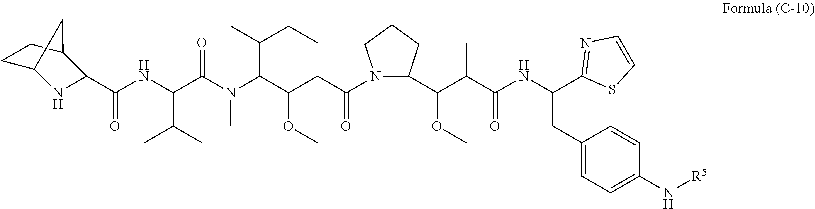

wherein: R⁵ are as defined above for compounds of Formula (C).

Embodiment 43. The compound of Formula (C), Formula (C-1) or Formula (C-3), or a pharmaceutically acceptable salt thereof, having the structure of Formula (C-11), or a pharmaceutically acceptable salt thereof:

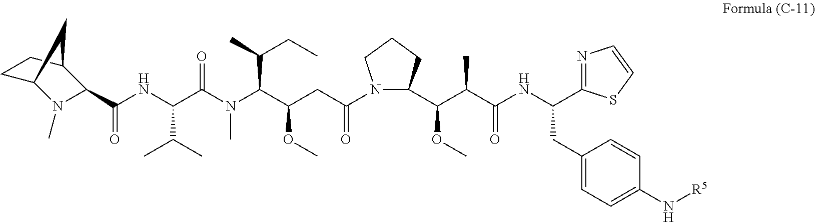

wherein: R⁵ are as defined above for compounds of Formula (C).

Embodiment 44. The compound of Formula (C), Formula (C-1) or Formula (C-3), or a pharmaceutically acceptable salt thereof, having the structure of Formula (C-12), or a pharmaceutically acceptable salt thereof:

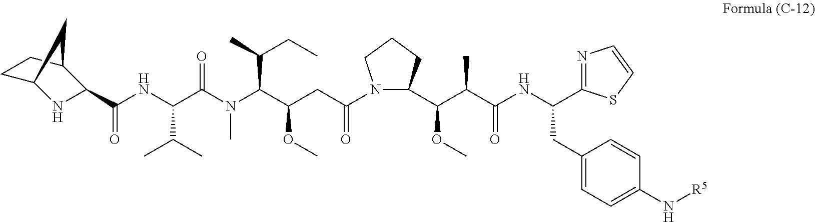

wherein: R⁵ are as defined above for compounds of Formula (C).

Embodiment 45. The compound of Formula (C), Formula (C-1) or Formula (C-3), or Embodiment 37, wherein the compound is

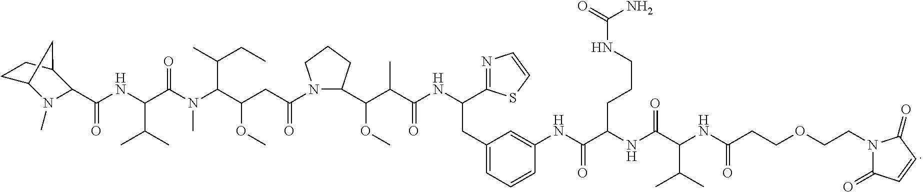 [SMILES: CCC(C)C(C(CC(=O)N1CCCC1C(OC)C(C)C(=O)NC(Cc1cccc(NC(=O)C(CCCNC(N)=O)NC(=O)C(NC(=O)CCOCCN2C(=O)C=CC2=O)C(C)C)c1)c1nccs1)OC)N(C)C(=O)C(NC(=O)C1C2CCC(C2)N1C)C(C)C]

Embodiment 46. The compound of Formula (C), Formula (C-1) or Formula (C-3), or Embodiment 37, wherein the compound is

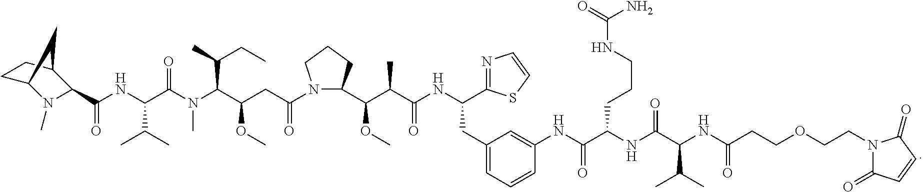 [SMILES: CC[C@H](C)[C@@H]([C@@H](CC(=O)N1CCC[C@H]1[C@H](OC)[C@@H](C)C(=O)N[C@@H](Cc1cccc(NC(=O)[C@H](CCCNC(N)=O)NC(=O)[C@@H](NC(=O)CCOCCN2C(=O)C=CC2=O)C(C)C)c1)c1nccs1)OC)N(C)C(=O)[C@@H](NC(=O)[C@@H]1[C@H]2CC[C@@H](C2)N1C)C(C)C]

Embodiment 47. The compound of Formula (C), Formula (C-1) or Formula (C-3), or Embodiment 38, wherein the compound is

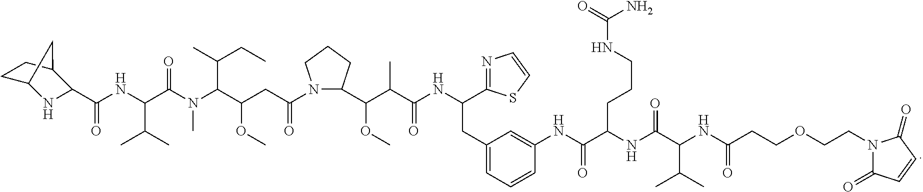 [SMILES: CCC(C)C(C(CC(=O)N1CCCC1C(OC)C(C)C(=O)NC(Cc1cccc(NC(=O)C(CCCNC(N)=O)NC(=O)C(NC(=O)CCOCCN2C(=O)C=CC2=O)C(C)C)c1)c1nccs1)OC)N(C)C(=O)C(NC(=O)C1NC2CCC1C2)C(C)C]

Embodiment 48. The compound of Formula (C), Formula (C-1) or Formula (C-3), or Embodiment 38, wherein the compound is

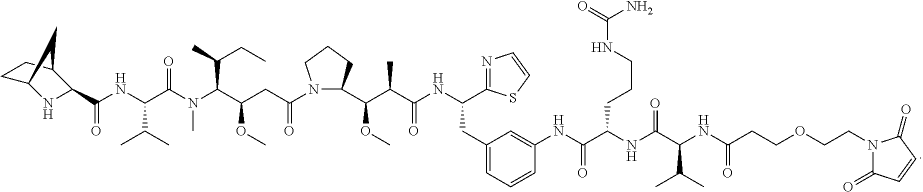 [SMILES: CC[C@H](C)[C@@H]([C@@H](CC(=O)N1CCC[C@H]1[C@H](OC)[C@@H](C)C(=O)N[C@@H](Cc1cccc(NC(=O)[C@H](CCCNC(N)=O)NC(=O)[C@@H](NC(=O)CCOCCN2C(=O)C=CC2=O)C(C)C)c1)c1nccs1)OC)N(C)C(=O)[C@@H](NC(=O)[C@H]1N[C@H]2CC[C@H]1C2)C(C)C]

In one aspect the Linker-Drug moiety of the invention is a compound having the structure of Formula (C), or stereoisomers or pharmaceutically acceptable salts thereof,

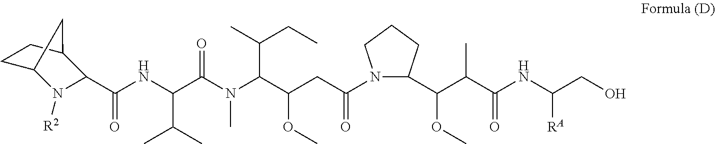

wherein:
R^{A} is

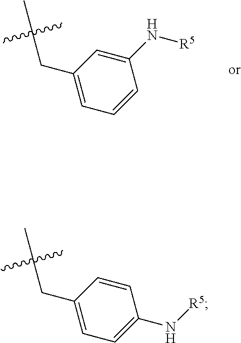

R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)ₙR⁴ or C₁-C₆alkyl which is optionally substituted with —CN, —C(═O)NH₂ or 1 to 5 hydroxyl;
each R³ is independently selected from C₁-C₆alkyl and C₁-C₆alkyl which is optionally substituted with 1 to 5 hydroxyl;
each R⁴ is independently selected from H and C₁-C₆alkyl;
R⁵ is -L₁R¹⁴, -L₁R²⁴, -L₁R³⁴ or -L₁R⁴⁴;
L₁ is —X₁C(═O)((CH₂)ₘO)ₚ(CH₂)ₘ—**, —X₁C(═O)(CH₂)ₘ—**, —X₂X₁C(═O)((CH₂)ₘO)ₚ(CH₂)ₘ—**, —X₂X₁C(═O)(CH₂)ₘ—**, —X₃C(═O)((CH₂)ₘO)ₚ(CH₂)ₘ—**, —X₃C(═O)(CH₂)ₘ—**, —X₃C(═O)(CH₂)ₘNHC(═O)((CH₂)ₘO)ₚ(CH₂)ₘ—**, —X₃C(═O)(CH₂)ₘNHC(═O)(CH₂)ₘ—**, —X₃C(═O)(CH₂)ₘ—**, —X₁C(═O)((CH₂)ₘO)ₚ(CH₂)ₘX₄(CH₂)ₘ—**, —X₁C(═O)(CH₂)ₘX₄(CH₂)ₘ—**, —X₂X₁C(═O)((CH₂)ₘO)ₚ(CH₂)ₘX₄(CH₂)ₘ—** or —X₂X₁C(═O)(CH₂)ₘX₄(CH₂)ₘ**, where ** indicates the point of attachment to R¹¹⁴;
X₁ is

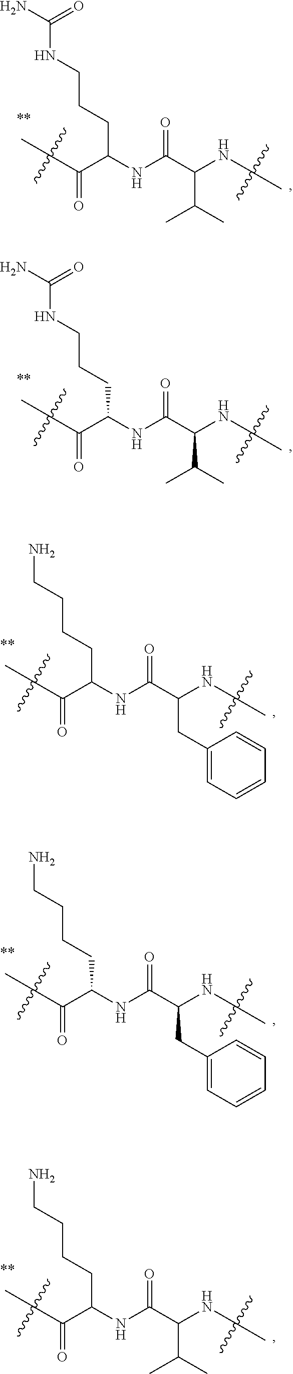 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O ; *NC(C(=O)NC(CCCCN)C(*)=O)C(C)C]

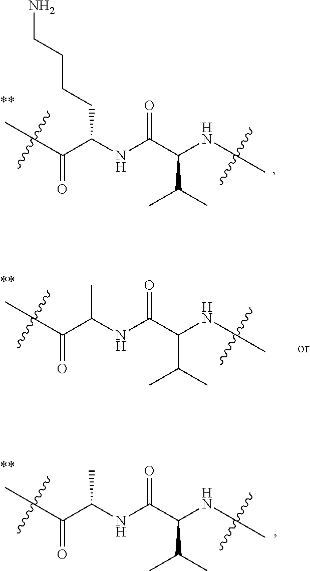 [SMILES: *N[C@H](C(=O)N[C@@H](CCCCN)C(*)=O)C(C)C ; *NC(C(=O)NC(C)C(*)=O)C(C)C ; *N[C@H](C(=O)N[C@@H](C)C(*)=O)C(C)C]

where ** indicates the point of attachment to the —NH— or to X₂;
X₂ is

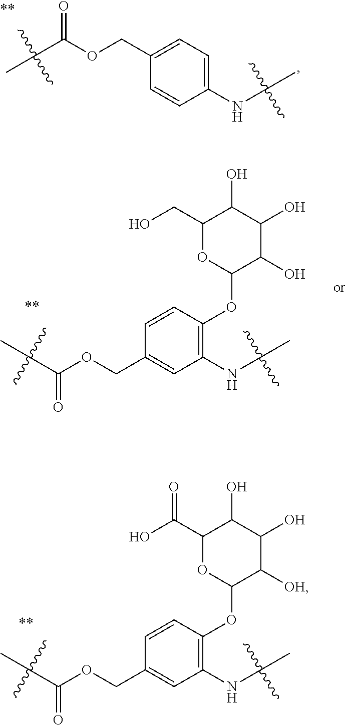 [SMILES: *Nc1ccc(COC(*)=O)cc1 ; *Nc1cc(COC(*)=O)ccc1OC1OC(CO)C(O)C(O)C1O ; *Nc1cc(COC(*)=O)ccc1OC1OC(C(=O)O)C(O)C(O)C1O]

where ** indicates the point of attachment to the —NH—;
X₃ is

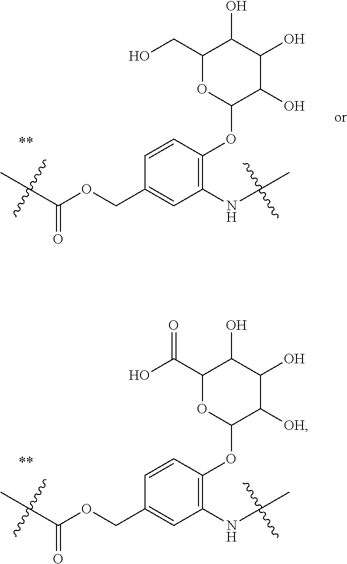 [SMILES: *Nc1cc(COC(*)=O)ccc1OC1OC(CO)C(O)C(O)C1O ; *Nc1cc(COC(*)=O)ccc1OC1OC(C(=O)O)C(O)C(O)C1O]

where ** indicates the point of attachment to the —NH—;
X₄ is

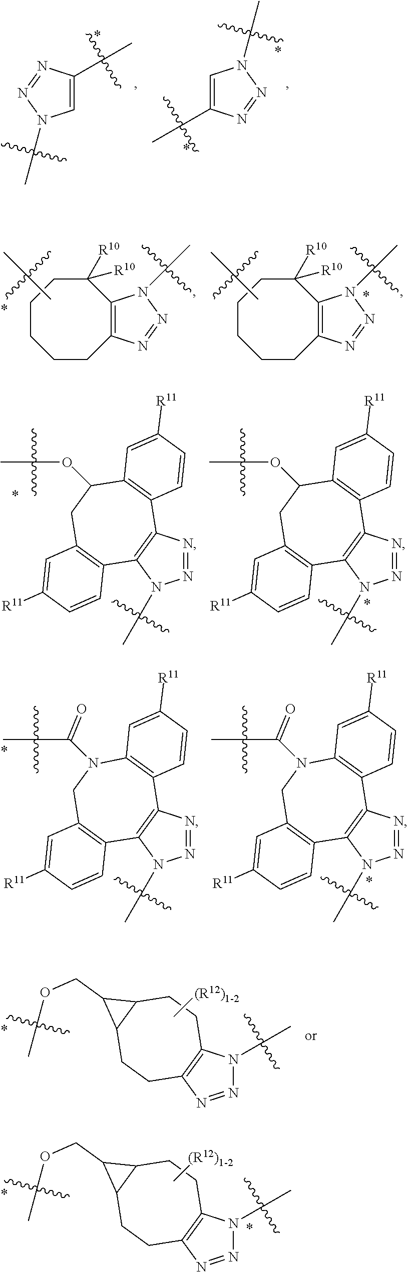

where the * indicates the point of attachment is toward R¹⁴, R²⁴, R³⁴ or R⁴⁴;
R¹⁴ is

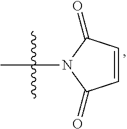 [SMILES: *N1C(=O)C=CC1=O]

—N₃, —ONH₂, —NR⁶C(═O)CH═CH₂, SH, —SSR⁷, —S(═O)₂(CH═CH₂), —NR⁶S(═O)₂(CH═CH₂), —NR⁶C(═O)CH₂Br, —NR⁶C(═O)CH₂I, —NHC(═O)CH₂Br, —NHC(═O)CH₂I, —C(═O)NHNH₂,

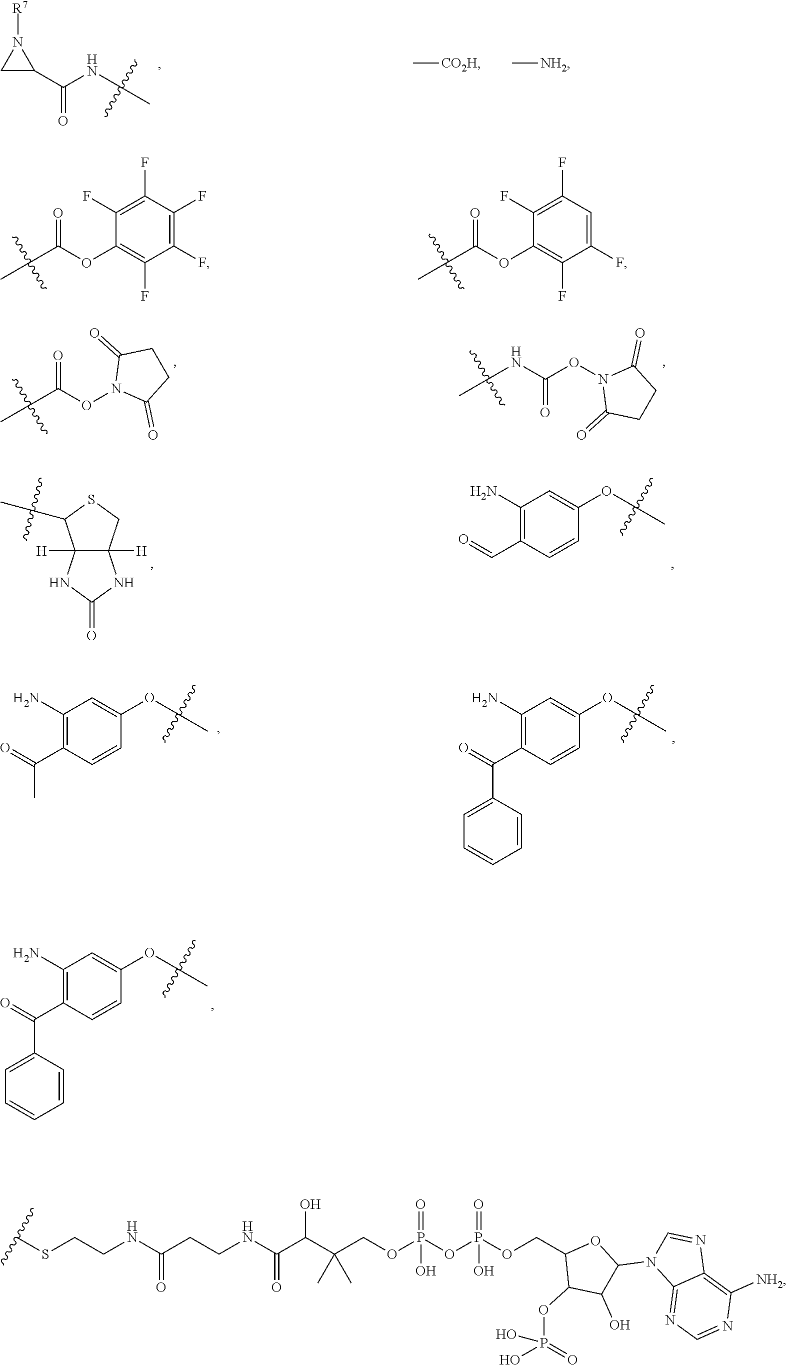

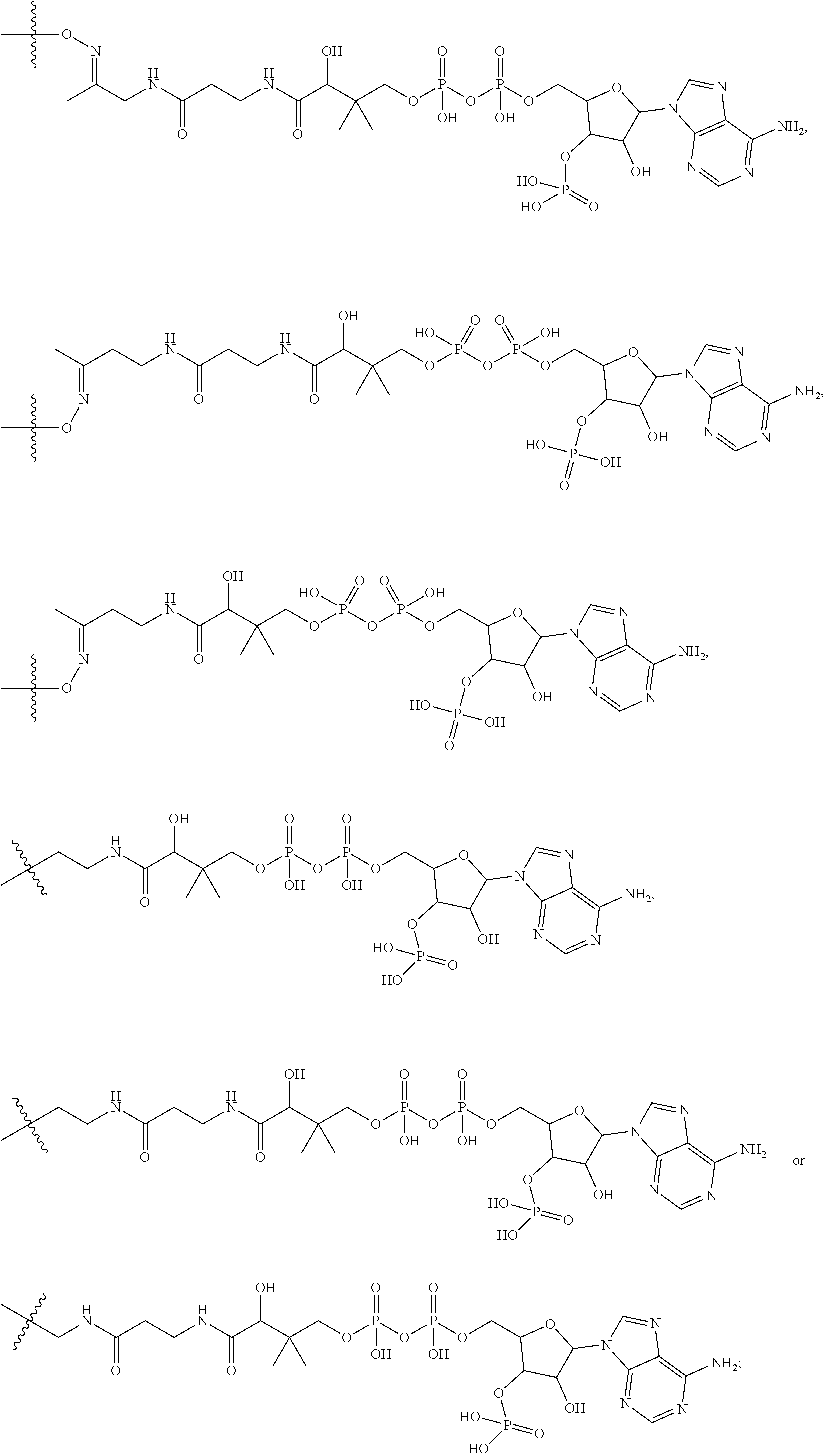

R²⁴ is

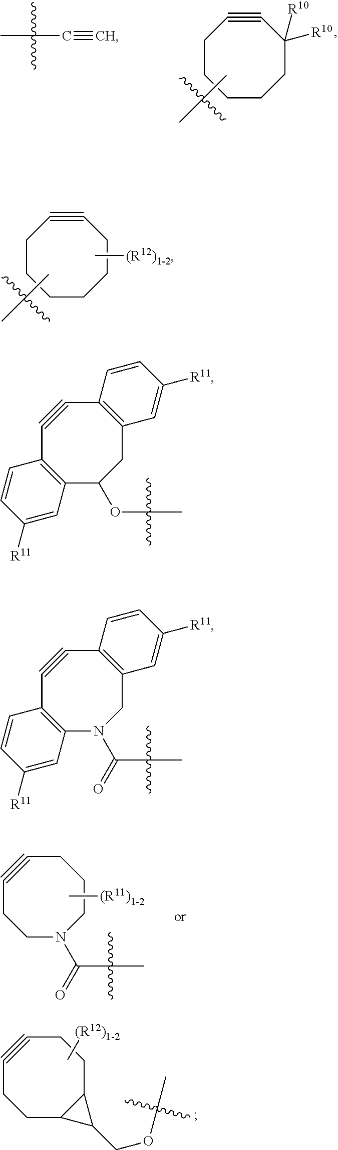

R³⁴ is, —N₃, —ONH₂, —NR⁷C(═O)CH═CH₂, —C(═O)NHNH₂, —CO₂H, —NH₂,

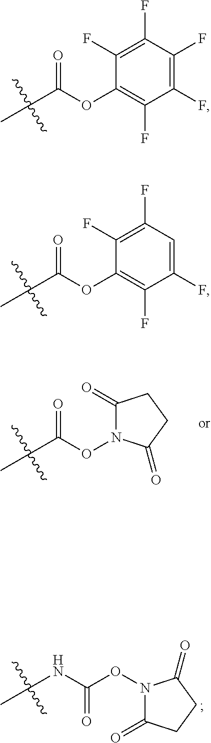 [SMILES: *C(=O)Oc1c(F)c(F)c(F)c(F)c1F ; *C(=O)Oc1c(F)c(F)cc(F)c1F ; *C(=O)ON1C(=O)CCC1=O ; *NC(=O)ON1C(=O)CCC1=O]

R⁴⁴ is

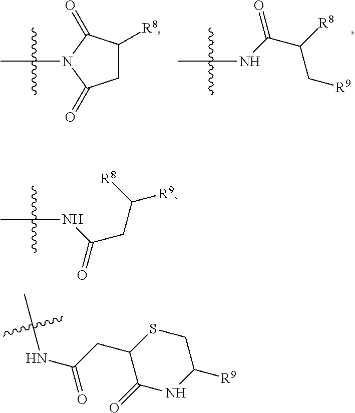

or —NR⁷C(═O)CH₂R⁸;
each R⁶ is independently selected from H and C₁-C₆alkyl;
R⁷ is 2-pyridyl or 4-pyridyl;
R⁸ is —S(CH₂)ₙCHR⁹NH₂;
R⁹ is —C(═O)OR⁷;
each R¹⁰ is independently selected from H, C₁-C₆alkyl, F, Cl, and —OH;
each R¹¹ is independently selected from H, C₁-C₆alkyl, F, Cl, —NH₂, —OCH₃, —OCH₂CH₃, —N(CH₃)₂, —CN, —NO₂ and —OH;
each R¹² is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each m is independently selected from 1, 2, 3, 4, 5, 6, 7, 8, 9 and 10, and
each p is independently selected from 1, 2, 3, 4, 5, 6, 7, 8, 9, 10, 11, 12, 13 and 14.

Certain aspects and examples of the Linker-Drug moiety of the invention are provided in the following listing of additional, enumerated embodiments. It will be recognized that features specified in each embodiment may be combined with other specified features to provide further embodiments of the present invention.

Embodiment 49. The compound of Formula (D), or a pharmaceutically acceptable salt thereof, having the structure of Formula (D-1), or a pharmaceutically acceptable salt thereof:

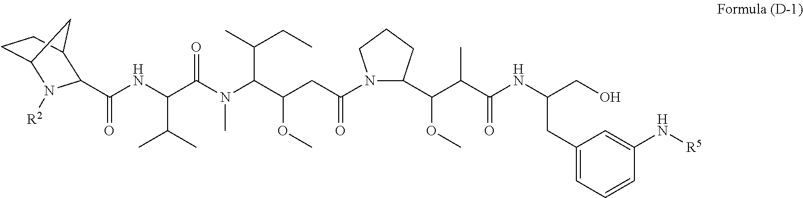

wherein: R² and R⁵ are as defined above for compounds of Formula (D).

Embodiment 50. The compound of Formula (D), or a pharmaceutically acceptable salt thereof, having the structure of Formula (D-2), or a pharmaceutically acceptable salt thereof:

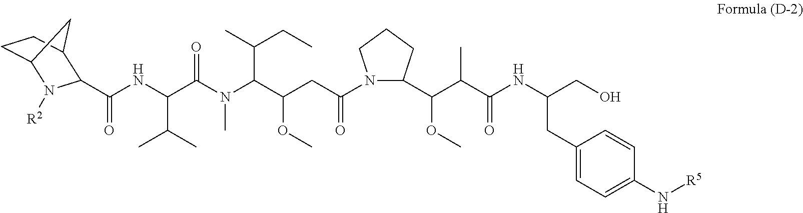

wherein: R² and R⁵ are as defined above for compounds of Formula (D).

Embodiment 51. The compound of Formula (D), or a pharmaceutically acceptable salt thereof, having the structure of Formula (D-3), or a pharmaceutically acceptable salt thereof:

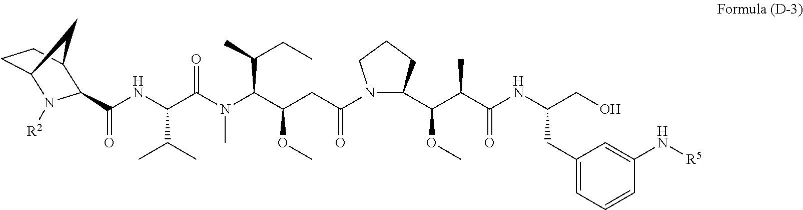

wherein: R² and R⁵ are as defined above for compounds of Formula (D).

Embodiment 52. The compound of Formula (D), or a pharmaceutically acceptable salt thereof, having the structure of Formula (D-4), or a pharmaceutically acceptable salt thereof:

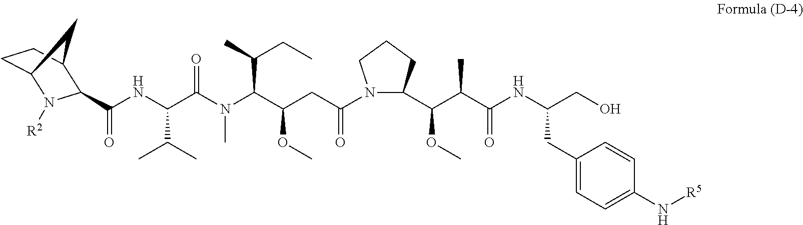

wherein: R² and R⁵ are as defined above for compounds of Formula (D).

Embodiment 53. The compound of Formula (D), Formula (D-1), Formula (D-2), Formula (D-3) or Formula (D-4), or a pharmaceutically acceptable salt thereof, wherein: R² is H or C₁-C₆alkyl.

Embodiment 54. The compound of Formula (D), Formula (D-1), Formula (D-2), Formula (D-3) or Formula (D-4), or a pharmaceutically acceptable salt thereof, wherein: R² is H or methyl.

Embodiment 55. The compound of Formula (D), Formula (D-1), Formula (D-2), Formula (D-3) or Formula (D-4), or a pharmaceutically acceptable salt thereof, wherein: R² is H.

Embodiment 56. The compound of Formula (D), Formula (D-1), Formula (D-2), Formula (D-3) or Formula (D-4), or a pharmaceutically acceptable salt thereof, wherein: R² is methyl.

Embodiment 57. The compound of Formula (D), Formula (D-1) or Formula (D-3), or a pharmaceutically acceptable salt thereof, having the structure of Formula (D-5), or a pharmaceutically acceptable salt thereof:

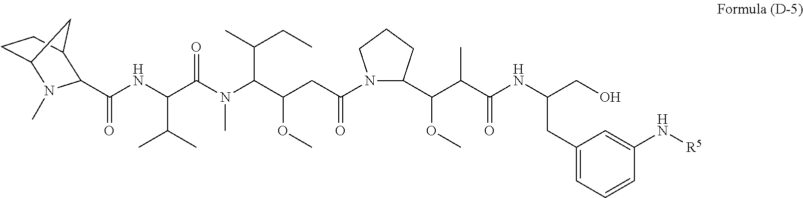

wherein: R⁵ are as defined above for compounds of Formula (D).

Embodiment 58. The compound of Formula (D), Formula (D-1) or Formula (D-3), or a pharmaceutically acceptable salt thereof, having the structure of Formula (D-6), or a pharmaceutically acceptable salt thereof:

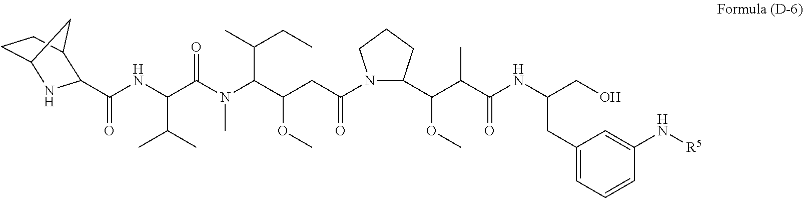

wherein: R⁵ are as defined above for compounds of Formula (D).

Embodiment 59. The compound of Formula (D), Formula (D-1) or Formula (D-3), or a pharmaceutically acceptable salt thereof, having the structure of Formula (D-7), or a pharmaceutically acceptable salt thereof:

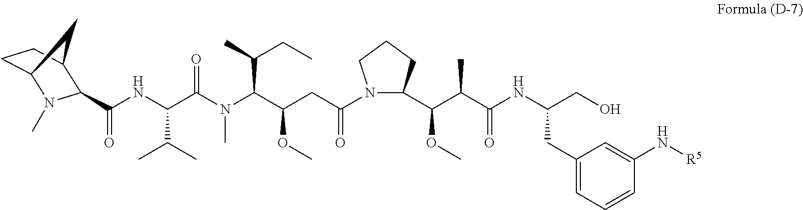

wherein: R⁵ are as defined above for compounds of Formula (D).

Embodiment 60. The compound of Formula (D), Formula (D-1) or Formula (D-3), or a pharmaceutically acceptable salt thereof, having the structure of Formula (D-8), or a pharmaceutically acceptable salt thereof:

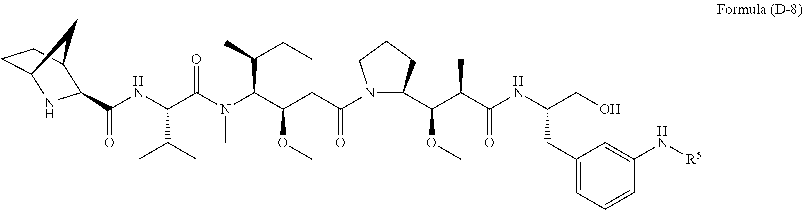

wherein: R⁵ are as defined above for compounds of Formula (D).

Embodiment 61. The compound of Formula (D), Formula (D-1) or Formula (D-3), or a pharmaceutically acceptable salt thereof, having the structure of Formula (D-9), or a pharmaceutically acceptable salt thereof:

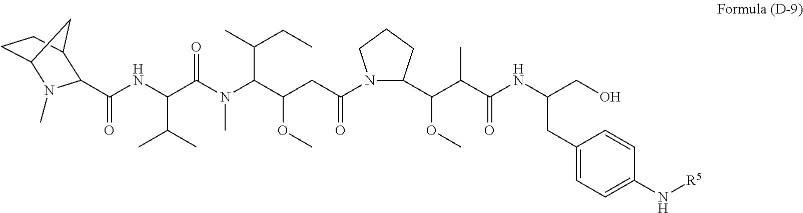

wherein: R⁵ are as defined above for compounds of Formula (D).

Embodiment 62. The compound of Formula (D), Formula (D-1) or Formula (D-3), or a pharmaceutically acceptable salt thereof, having the structure of Formula (D-10), or a pharmaceutically acceptable salt thereof:

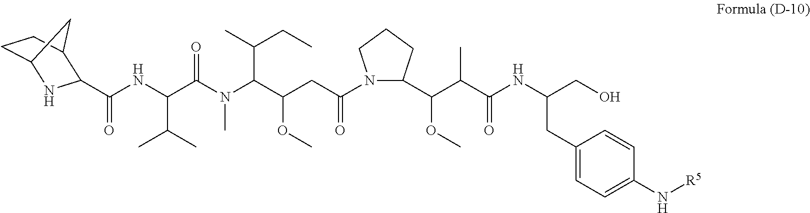

wherein: R⁵ are as defined above for compounds of Formula (D).

Embodiment 63. The compound of Formula (D), Formula (D-1) or Formula (D-3), or a pharmaceutically acceptable salt thereof, having the structure of Formula (D-11), or a pharmaceutically acceptable salt thereof:

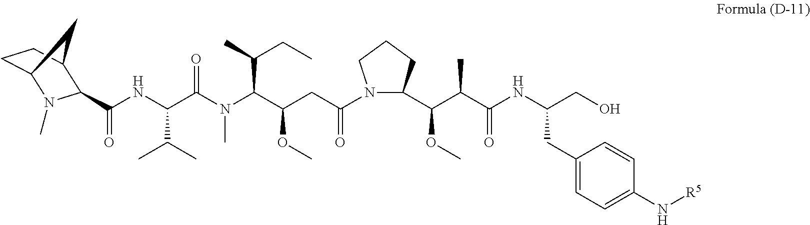

wherein: R⁵ are as defined above for compounds of Formula (D).

Embodiment 64. The compound of Formula (D), Formula (D-1) or Formula (D-3), or a pharmaceutically acceptable salt thereof, having the structure of Formula (D-12), or a pharmaceutically acceptable salt thereof:

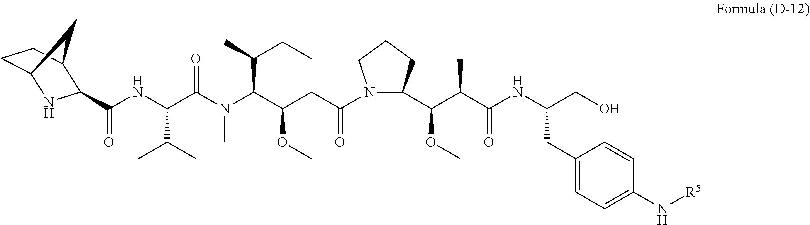

wherein: R⁵ are as defined above for compounds of Formula (D).

Embodiment 65. The compound of Formula (D), Formula (D-1) or Formula (D-3), or Embodiment 57, wherein the compound is

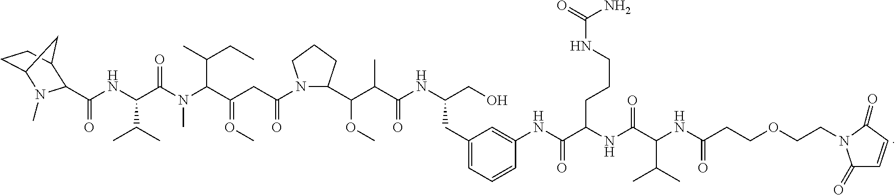

Embodiment 66. The compound of Formula (D), Formula (D-1) or Formula (D-3), or Embodiment 57, wherein the compound is

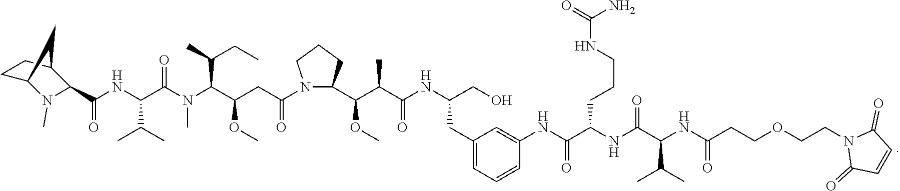 [SMILES: CC[C@H](C)[C@@H]([C@@H](CC(=O)N1CCC[C@H]1[C@H](OC)[C@@H](C)C(=O)N[C@H](CO)Cc1cccc(NC(=O)[C@H](CCCNC(N)=O)NC(=O)[C@@H](NC(=O)CCOCCN2C(=O)C=CC2=O)C(C)C)c1)OC)N(C)C(=O)[C@@H](NC(=O)[C@@H]1[C@H]2CC[C@@H](C2)N1C)C(C)C]

Embodiment 67. The compound of Formula (D), Formula (D-1) or Formula (D-3), or Embodiment 58, wherein the compound is

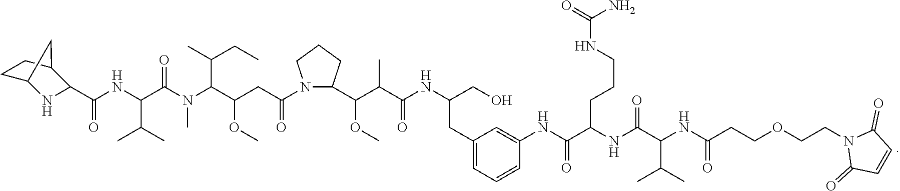 [SMILES: CCC(C)C(C(CC(=O)N1CCCC1C(OC)C(C)C(=O)NC(CO)Cc1cccc(NC(=O)C(CCCNC(N)=O)NC(=O)C(NC(=O)CCOCCN2C(=O)C=CC2=O)C(C)C)c1)OC)N(C)C(=O)C(NC(=O)C1NC2CCC1C2)C(C)C]

Embodiment 68. The compound of Formula (D), Formula (D-1) or Formula (D-3), or Embodiment 58, wherein the compound is

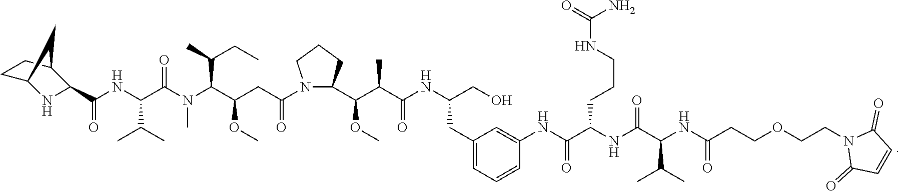 [SMILES: CC[C@H](C)[C@@H]([C@@H](CC(=O)N1CCC[C@H]1[C@H](OC)[C@@H](C)C(=O)N[C@H](CO)Cc1cccc(NC(=O)[C@H](CCCNC(N)=O)NC(=O)[C@@H](NC(=O)CCOCCN2C(=O)C=CC2=O)C(C)C)c1)OC)N(C)C(=O)[C@@H](NC(=O)[C@H]1N[C@H]2CC[C@H]1C2)C(C)C]

### Antibody Drug Conjugates

The present disclosure provides antibody drug conjugates, wherein an antibody or antibody fragment (e.g., Fab or Fab′) that specifically binds to cKIT is linked to a drug moiety (e.g., a cytotoxic agent), optionally through a linker. In one aspect, the antibody or antibody fragment (e.g., Fab or Fab′) is linked, via covalent attachment by a linker, to a drug moiety that is a cytotoxic agent.

The antibody drug conjugates can selectively deliver a cytotoxic agent to cells expressing cKIT, e.g., hematopoietic stem cells, thereby selectively ablate those cells in a patient, e.g., a hematopoietic stem cell transplantation recipient. Preferably, the cKIT antibody drug conjugates have short half-life and will be cleared from a patient's circulation so they can be used for conditioning hematopoietic stem cell transplant recipients prior to hematopoietic stem cell transplantation.

In some embodiments, the cKIT antibody drug conjugates disclosed herein are modified to have reduced ability to induce mast cell degranulation, even when cross-linked and/or multimerized into larger complexes. For example, the cKIT antibody drug conjugates disclosed herein are modified to have a reduced ability to induce mast cell degranulation that is, is about, or is at least 10%, 20%, 30%, 40%, 50%, 60%, 70%, 80%, 90%, 95%, 99% reduced in comparison to a full-length cKIT antibody, an F(ab′)₂ or an F(ab)₂ fragment, or conjugate thereof, even when cross-linked and/or multimerized into larger complexes. In some embodiments, the cKIT antibody drug conjugates disclosed herein may comprise an anti-cKIT Fab or Fab′ fragment. In some embodiments, the anti-cKIT antibody drug conjugates disclosed herein may have minimal activity to induce mast cell degranulation, e.g., a baseline corrected O.D. readout of less than 0.25, e.g., less than 0.2, less than 0.15, or less than 0.1, in a beta-hexosaminidase release assay, even when cross-linked and/or multimerized into larger complexes.

In some embodiments, provided herein are conjugates comprising an antibody fragment (e.g., Fab or Fab′) that specifically binds to cKIT (anti-cKIT Fab or Fab′), linked to a drug moiety (e.g., a cytotoxic agent), optionally through a linker. As described herein, such anti-cKIT Fab′ or Fab-toxin conjugates are able to ablate human HSC cells in vitro and in vivo, but do not cause mast cell degranulation even when crosslinked and/or multimerized into larger complexes.

In one aspect, the disclosure provides for an conjugate of Formula (I):

A-(L_{B}-(D)ₙ)_{y} Formula (I);

wherein:
A is an antibody fragment (e.g., Fab or Fab′) that specifically binds to human cKIT;
L_{B} is a linker;
D is a cytotoxic agent;
n is an integer from 1 to 10, and
y is an integer from 1 to 10,
   where the Linker-Drug moiety (L_{B}-(D)ₙ) is covalently attached to the antibody fragment (A).

In one aspect, the present disclosure is directed to a conjugate of Formula (II):

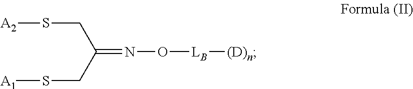

A₁ is an antibody fragment (e.g., Fab or Fab′) or chain (e.g. HC or LC) that specifically binds to human cKIT;
A₂ is an antibody fragment (e.g., Fab or Fab′) or chain (e.g. HC or LC) that specifically binds to human cKIT;
L_{B} is a linker;
D is a cytotoxic agent, and
n is an integer from 1 to 10,
where the Linker-Drug moiety (L_{B}-(D)ₙ) covalently couples the antibody fragments A₁ and A₂.

In one aspect, the conjugates of the invention comprises one or more cytotoxins covalently attached to a linker (L_{B}), wherein the one or more cytotoxins are independently selected from compound of Formula (A), Formula (A-1), Formula (A-2), Formula (A-3), Formula (A-4), Formula (B), Formula (B-1), Formula (B-2), Formula (B-3) or Formula (B-4) or a compound of any one of Embodiments 7 to 14 or any one of Embodiments 23 to 30.

In one aspect, the conjugates of the invention comprises one or more cytotoxins covalently attached to a linker (L_{B}), wherein the one or more cytotoxins are independently selected from compound of Formula (A), Formula (A-1), Formula (A-2), Formula (A-3) or Formula (A-4), or a compound of any one of Embodiments 7 to 14.

In one aspect, the conjugates of the invention comprises one or more cytotoxins covalently attached to a linker (L_{B}), wherein the one or more cytotoxins are independently selected from compound of Formula (B), Formula (B-1), Formula (B-2), Formula (B-3) or Formula (B-4) or a compound of any one of Embodiments 23 to 30.

In the conjugates of Formula (I), one or more Linker-Drug moiety (L_{B}-(D)ₙ) can be covalently attached to the antibody fragment, A (e.g. Fab or Fab′), thereby covalently attaching one or more drug moieties, D, to the antibody fragment, A (e.g. Fab or Fab′), through linker, L_{B}. L_{B} is any chemical moiety that is capable of linking the antibody fragment, A (e.g. Fab or Fab′) to one or more drug moieties, D. The conjugates of Formula (I), wherein one or more drug moieties, D, are covalently linked to an antibody fragment, A (e.g. Fab or Fab′), can be formed using a bifunctional or multifunctional linker reagent having one or more reactive functional groups that are the same or different. One of the reactive functional groups of the bifunctional or multifunctional linker reagent is used to react with a group on the antibody fragment, A, by way of example, a thiol or an amine (e.g. a cysteine, an N-terminus or amino acid side chain such as lysine) to form a covalent linkage with one end of the linker L_{B}. Such reactive functional groups of the bifunctional or multifunctional linker reagent include, but are not limited to, a maleimide, a thiol and an NHS ester. The other reactive functional group or groups of the bifunctional or multifunctional linker reagent are used to covalently attached one or more drug moieties, D, to linker L_{B}.

In the conjugates of Formula (II), a ketone bridge is formed by reaction of pendent thiols on antibody fragments A₁ and A₂ and a 1,3-dihaloacetone, such as 1,3-dichloroacetone, 1,3-dibromoacetone, 1,3-diiodoacetone, and bissulfonate esters of 1,3-dihydroxyacetone, which thereby covalently couples the antibody fragments A₁ and A₂. This ketone bridge moiety is used to covalently attach one or more drug moieties, D, to the antibody fragments A₁ and A₂ through a linker L_{B}. L_{B} is any chemical moiety that is capable of linking the antibody fragment, A₁ and A₂ to one or more drug moieties, D. The conjugates of Formula (II), wherein one or more drug moieties, D, are covalently linked to antibody fragments A₁ and A₂, can be formed using a bifunctional or multifunctional linker reagent having one or more reactive functional groups that are the same or different. In an embodiment, one the reactive functional groups of the bifunctional or multifunctional linker reagent is an alkoxyamine which is used to react with the ketone bridge to form an oxime linkage with one end of the linker L_{B}, and the other reactive functional group or groups of the bifunctional or multifunctional linker reagent are used to covalently attached one or more drug moieties, D, to linker L_{B}. In another embodiment, one the reactive functional groups of the bifunctional or multifunctional linker reagent is an hydrazine which is used to react with the ketone bridge to form a hydrazone linkage with one end of the linker L_{B}, and the other reactive functional group or groups of the bifunctional or multifunctional linker reagent are used to covalently attached one or more drug moieties, D, to linker L_{B}.

In one aspect, L_{B} is a cleavable linker. In another aspect, L_{B} is a non-cleavable linker. In some aspects, L_{B} is an acid-labile linker, photo-labile linker, peptidase cleavable linker, esterase cleavable linker, glycosidase cleavable linker, phosphodiesterase cleavable linker, a disulfide bond reducible linker, a hydrophilic linker, or a dicarboxylic acid based linker.

While the drug to antibody ratio has an exact integer value for a specific conjugate molecule (e.g., the product of n and y in Formula (I) and “n” in Formula (II)), it is understood that the value will often be an average value when used to describe a sample containing many molecules, due to some degree of inhomogeneity, typically associated with the conjugation step. The average loading for a sample of a conjugate is referred to herein as the drug to antibody (or Fab′) ratio, or “DAR.” In some aspects, the DAR is between about 1 and about 5, and typically is about 1, 2, 3, or 4. In some aspects, at least 50% of a sample by weight is compound having the average DAR plus or minus 2, and preferably at least 50% of the sample is a conjugate that contains the average DAR plus or minus 1. Other aspects include conjugates wherein the DAR is about 2. In some aspects, a DAR of ‘about y’ means the measured value for DAR is within 20% of the product of n and y in Formula (I). In some aspects, a DAR of ‘about n’ means the measured value for DAR is within 20% of n in Formula (II).

In one aspect, the average molar ratio of the drug to the antibody fragment (Fab or Fab′) in the conjugates of Formula (I) (i.e., average value of the product of n and y, also known as drug to antibody ratio (DAR)) is about 1 to about 10, about 1 to about 6 (e.g., 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0), about 1 to about 5, about 1.5 to about 4.5, or about 2 to about 4.

In one aspect, the average molar ratio of the drug to the antibody fragments A₁ and A₂ in the conjugates of Formula (II) (i.e., average value of n, also known as drug to antibody ratio (DAR)) is about 1 to about 10, about 1 to about 6 (e.g., 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0), about 1 to about 5, about 1.5 to about 4.5, or about 2 to about 4.

In one aspect provided by the disclosure, the conjugate has substantially high purity and has one or more of the following features: (a) greater than about 90% (e.g., greater than or equal to about 91%, 92%, 93%, 94%, 95%, 96%, 97%, 98%, 99%, or 100%), preferably greater than about 95%, of conjugate species are monomeric, (b) unconjugated linker level in the conjugate preparation is less than about 10% (e.g., less than or equal to about 9%, 8%, 7%, 6%, 5%, 4%, 3%, 2%, 1%, or 0%) (relative to total linker), (c) less than 10% of conjugate species are crosslinked (e.g., less than or equal to about 9%, 8%, 7%, 6%, 5%, 4%, 3%, 2%, 1%, or 0%), (d) free drug (e.g., auristatin, amanitin, maytansinoid or saporin) level in the conjugate preparation is less than about 2% (e.g., less than or equal to about 1.5%, 1.4%, 1.3%, 1.2%, 1.1%, 1.0%, 0.9%, 0.8%, 0.7%, 0.6%, 0.5%, 0.4%, 0.3%, 0.2%, 0.1%, or 0%) (mol/mol relative to total cytotoxic agent).

In one aspect the conjugates of the invention have the structure of Formula (E):

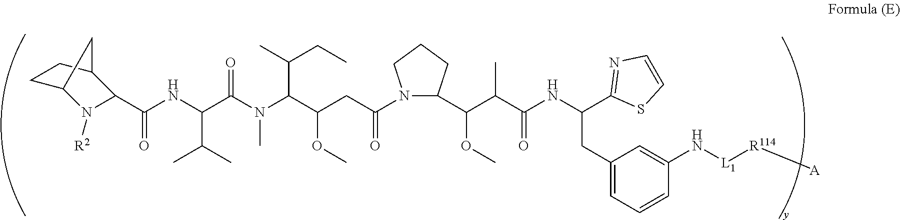

wherein:
A represents an antibody fragment (e.g., Fab or Fab′) that specifically binds to human cKIT;
y is an integer from 1 to 10;
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)R⁴ or C₁-C₆alkyl which is optionally substituted with —CN, —C(═O)NH₂ or 1 to 5 hydroxy;
each R³ is independently selected from C₁-C₆alkyl and C₁-C₆alkyl which is optionally substituted with 1 to 5 hydroxyl;
each R⁴ is independently selected from H and C₁-C₆alkyl;
L₁ is —X₁C(═O)((CH₂)ₘO)ₚ(CH₂)ₘ—**, —X₁C(═O)(CH₂)ₘ—**, —X₂X₁C(═O)((CH₂)ₘO)ₚ(CH₂)ₘ—**, —X₂X₁C(═O)(CH₂)ₘ—**, —X₃C(═O)((CH₂)ₘO)ₚ(CH₂)ₘ—**, —X₃C(═O)(CH₂)ₘ—**, —X₃C(═O)(CH₂)ₘNHC(═O)((CH₂)ₘO)ₚ(CH₂)ₘ—**, —X₃C(═O)(CH₂)ₘNHC(═O)(CH₂)ₘ—**, —X₃C(═O)(CH₂)ₘ—**, —X₁C(═O)((CH₂)ₘO)ₚ(CH₂)ₘX₄(CH₂)ₘ—**, —X₁C(═O)(CH₂)ₘX₄(CH₂)ₘ—**, —X₂X₁C(═O)((CH₂)ₘO)ₚ(CH₂)ₘX₄(CH₂)ₘ—** or —X₂X₁C(═O)(CH₂)ₘX₄(CH₂)ₘ—**, where ** indicates the point of attachment to R¹¹⁴;
X₁ is

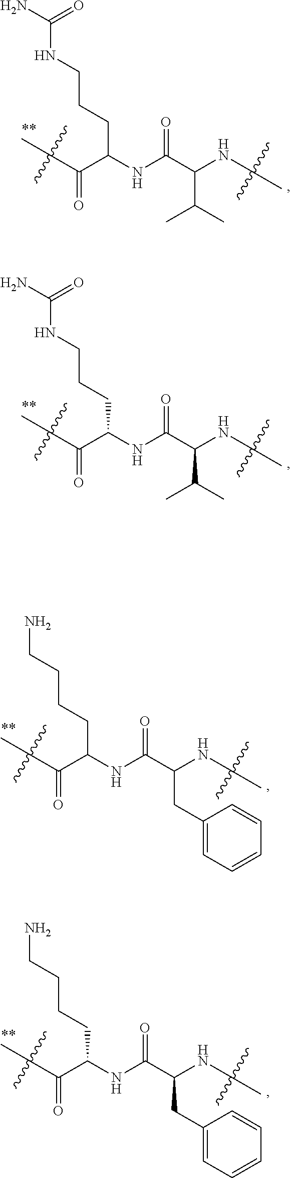 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O]

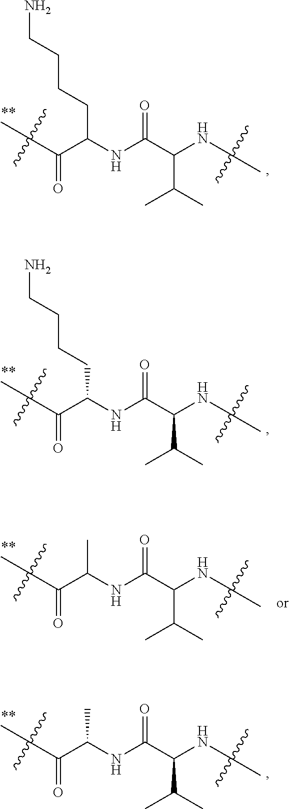 [SMILES: *NC(C(=O)NC(CCCCN)C(*)=O)C(C)C ; *N[C@H](C(=O)N[C@@H](CCCCN)C(*)=O)C(C)C ; *NC(C(=O)NC(C)C(*)=O)C(C)C ; *N[C@H](C(=O)N[C@@H](C)C(*)=O)C(C)C]

where ** indicates the point of attachment to the —NH— or to X₂;
X₂ is

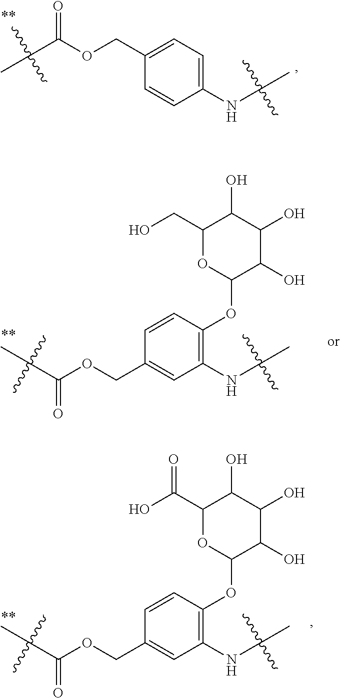 [SMILES: *Nc1ccc(COC(*)=O)cc1 ; *Nc1cc(COC(*)=O)ccc1OC1OC(CO)C(O)C(O)C1O ; *Nc1cc(COC(*)=O)ccc1OC1OC(C(=O)O)C(O)C(O)C1O]

where ** indicates the point of attachment to the —NH—;
X₃ is

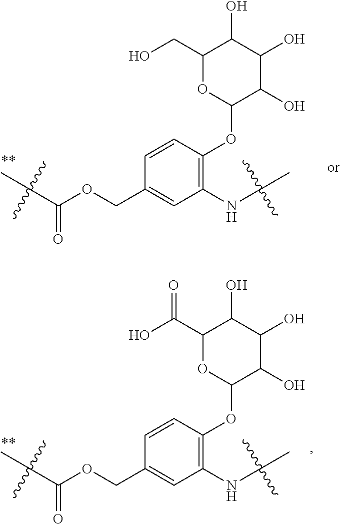 [SMILES: *Nc1cc(COC(*)=O)ccc1OC1OC(CO)C(O)C(O)C1O ; *Nc1cc(COC(*)=O)ccc1OC1OC(C(=O)O)C(O)C(O)C1O]

where ** indicates the point of attachment to the —NH—;
X₄ is

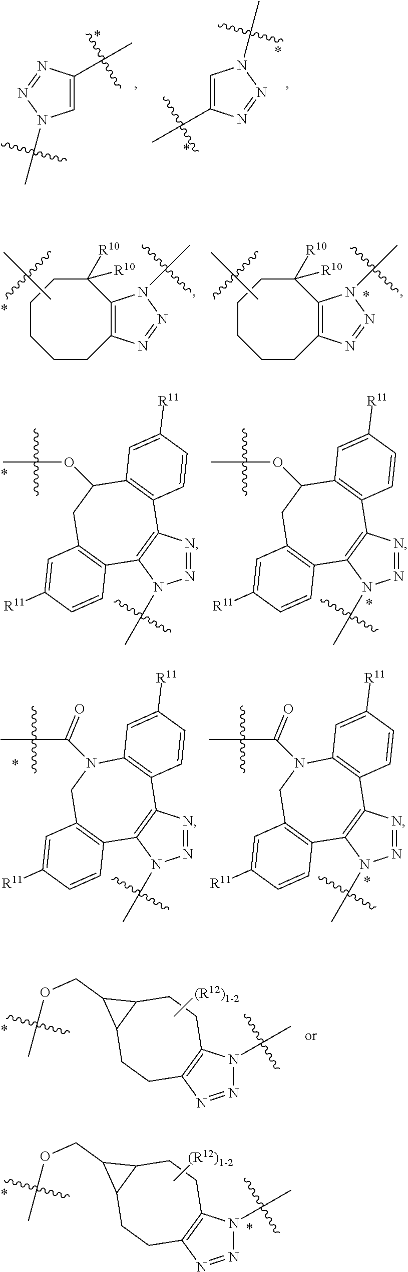

where the * indicates the point of attachment is toward R¹¹⁴;
R¹¹⁴ is

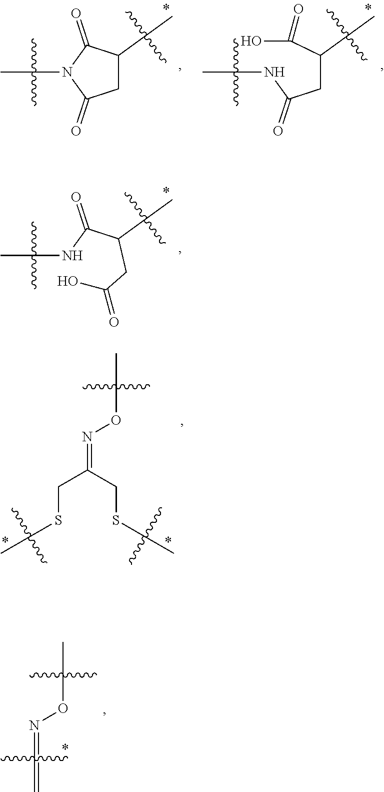

—NR⁶C(═O)CH₂—*, —NHC(═O)CH₂—*, —S(═O)₂CH₂CH₂—*, —(CH₂)₂S(═O)₂CH₂CH₂—*, —NR'S(═O)₂CH₂CH₂—*, —NR⁶C(═O)CH₂CH₂—*, —NH—, —C(═O)—, —NHC(═O)—*, —CH₂NHCH₂CH₂—*, —NHCH₂CH₂—*, —S—,

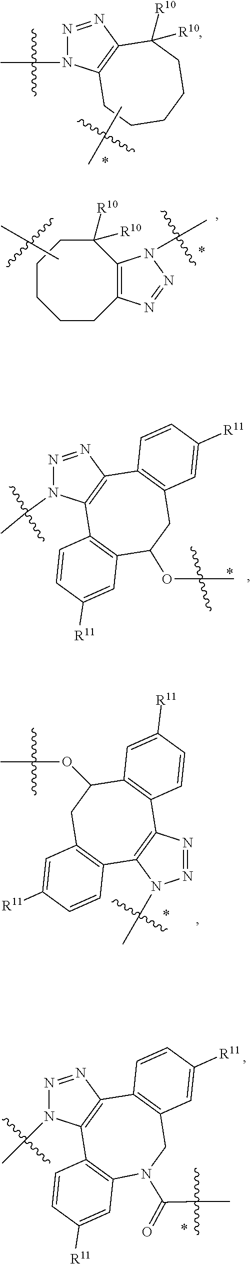

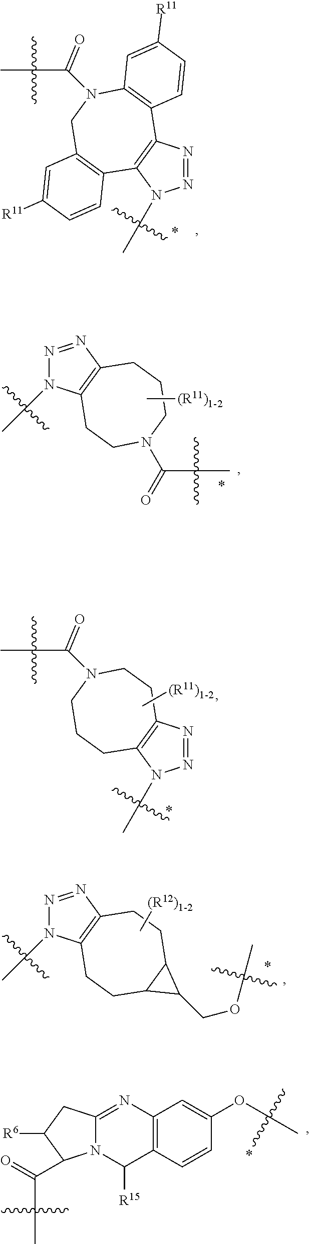

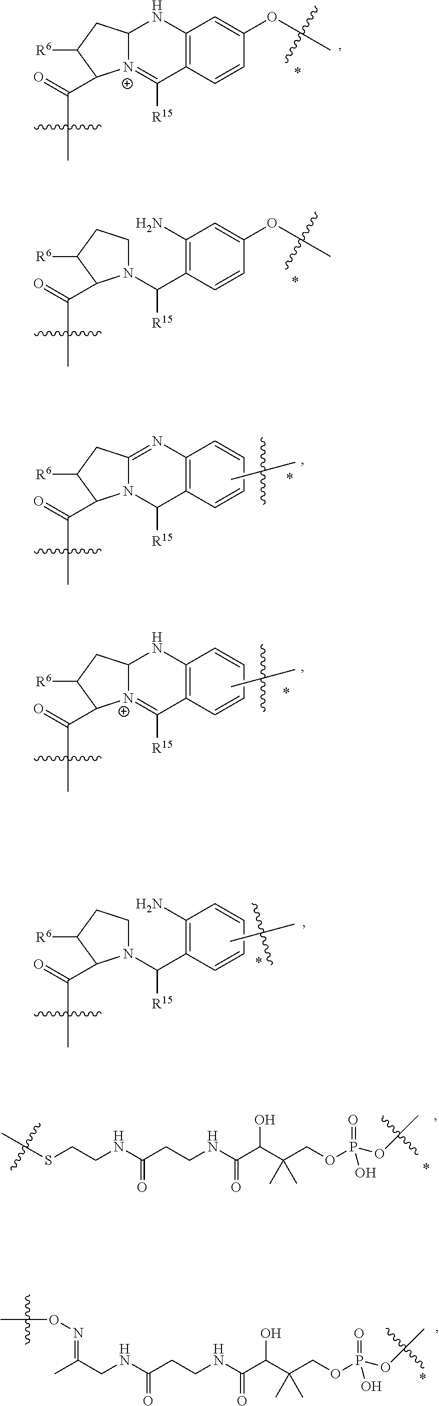

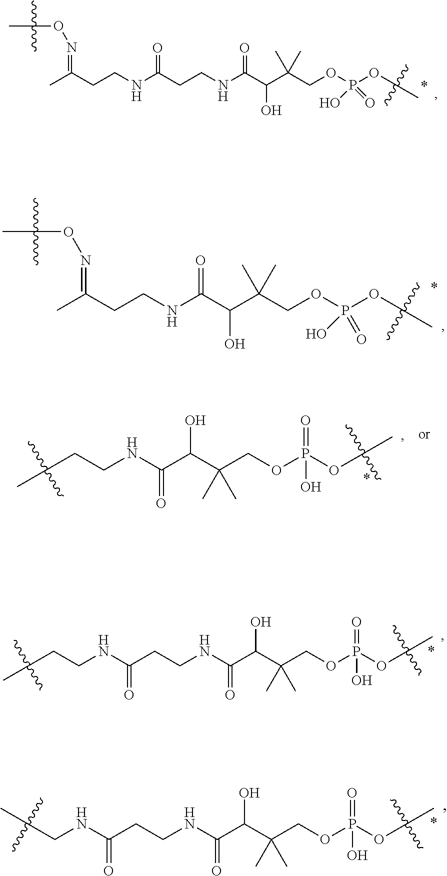 [SMILES: *O/N=C(\C)CCNC(=O)CCNC(=O)C(O)C(C)(C)COP(=O)(O)O* ; *O/N=C(\C)CCNC(=O)C(O)C(C)(C)COP(=O)(O)O* ; *CCNC(=O)C(O)C(C)(C)COP(=O)(O)O* ; *CCNC(=O)CCNC(=O)C(O)C(C)(C)COP(=O)(O)O* ; *CNC(=O)CCNC(=O)C(O)C(C)(C)COP(=O)(O)O*]

where the * indicates the point of attachment to A;
each R⁶ is independently selected from H and C₁-C₆alkyl;
each R¹⁰ is independently selected from H, C₁-C₆alkyl, F, Cl, and —OH;
each R¹¹ is independently selected from H, C₁-C₆alkyl, F, Cl, —NH₂, —OCH₃, —OCH₂CH₃, —N(CH₃)₂, —CN, —NO₂ and —OH;
each R¹² is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each R¹⁵ is independently selected from H, —CH₃ and phenyl;
each m is independently selected from 1, 2, 3, 4, 5, 6, 7, 8, 9 and 10, and
each p is independently selected from 1, 2, 3, 4, 5, 6, 7, 8, 9, 10, 11, 12, 13 and 14.

In one aspect the conjugates of the invention have the structure of Formula (F):

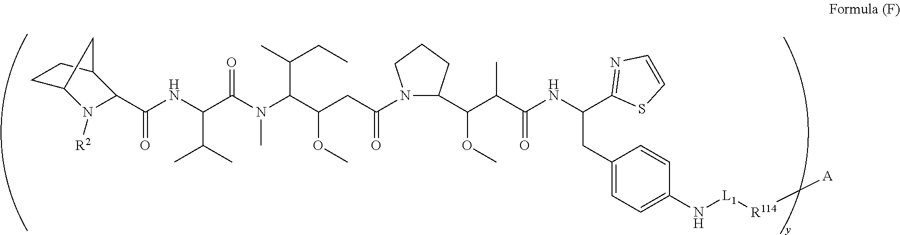

wherein:
A represents an antibody fragment (e.g., Fab or Fab′) that specifically binds to human cKIT;
y is an integer from 1 to 10;
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)R⁴ or C₁-C₆alkyl which is optionally substituted with —CN, —C(═O)NH₂ or 1 to 5 hydroxy;
each R³ is independently selected from C₁-C₆alkyl and C₁-C₆alkyl which is optionally substituted with 1 to 5 hydroxyl;
each R⁴ is independently selected from H and C₁-C₆alkyl;
L₁ is —X₁C(═O)((CH₂)ₘO)ₚ(CH₂)ₘ—**, —X₁C(═O)(CH₂)ₘ—**, —X₂X₁C(═O)((CH₂)ₘO)ₚ(CH₂)ₘ—**, —X₂X₁C(═O)(CH₂)ₘ—**, —X₃C(═O)((CH₂)ₘO)ₚ(CH₂)ₘ—**, —X₃C(═O)(CH₂)ₘ—**, —X₃C(═O)(CH₂)ₘNHC(═O)((CH₂)ₘO)ₚ(CH₂)ₘ—**, —X₃C(═O)(CH₂)ₘNHC(═O)(CH₂)ₘ—**, —X₃C(═O)(CH₂)ₘ—**, —X₁C(═O)((CH₂)ₘO)ₚ(CH₂)ₘX₄(CH₂)ₘ—**, —X₁C(═O)(CH₂)ₘX₄(CH₂)ₘ—**, —X₂X₁C(═O)((CH₂)ₘO)ₚ(CH₂)ₘX₄(CH₂)ₘ—** or —X₂X₁C(═O)(CH₂)ₘX₄(CH₂)ₘ—**, where ** indicates the point of attachment to R¹¹⁴;
X₁ is

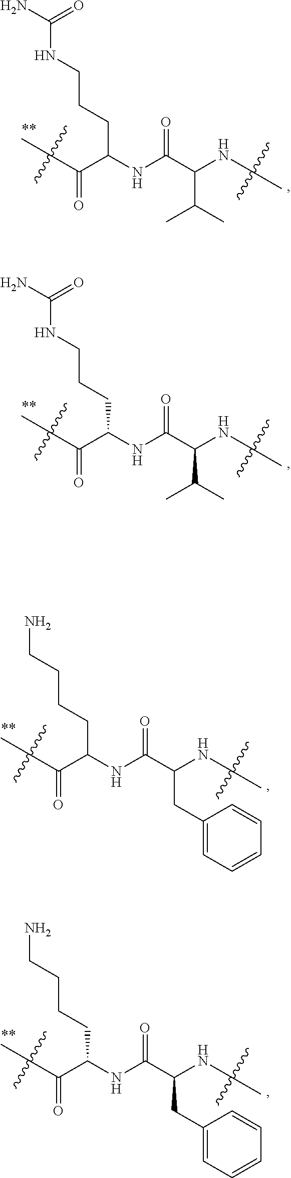 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O]

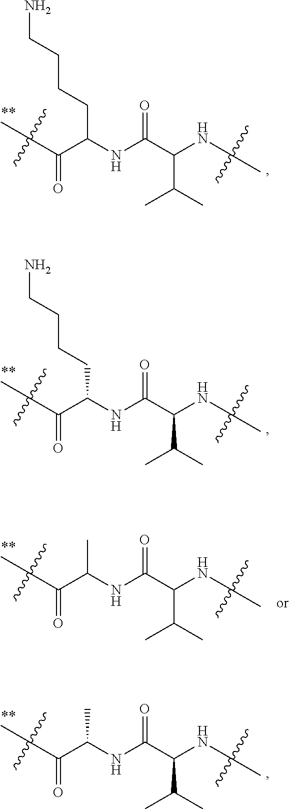 [SMILES: *NC(C(=O)NC(CCCCN)C(*)=O)C(C)C ; *N[C@H](C(=O)N[C@@H](CCCCN)C(*)=O)C(C)C ; *NC(C(=O)NC(C)C(*)=O)C(C)C ; *N[C@H](C(=O)N[C@@H](C)C(*)=O)C(C)C]

where ** indicates the point of attachment to the —NH— or to X₂
X₂ is

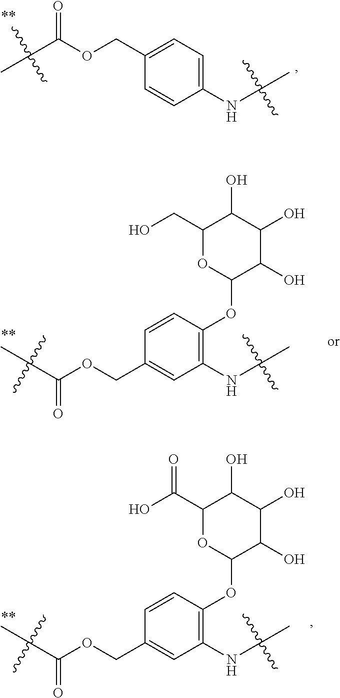 [SMILES: *Nc1ccc(COC(*)=O)cc1 ; *Nc1cc(COC(*)=O)ccc1OC1OC(CO)C(O)C(O)C1O ; *Nc1cc(COC(*)=O)ccc1OC1OC(C(=O)O)C(O)C(O)C1O]

where ** indicates the point of attachment to the —NH—;
X₃ is

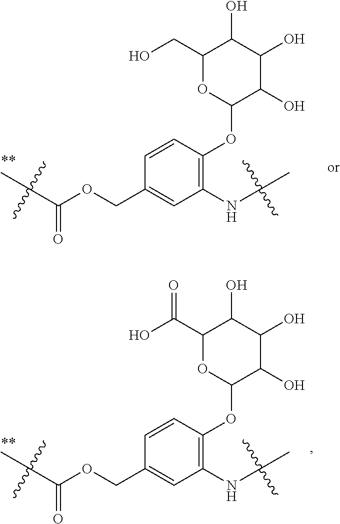 [SMILES: *Nc1cc(COC(*)=O)ccc1OC1OC(CO)C(O)C(O)C1O ; *Nc1cc(COC(*)=O)ccc1OC1OC(C(=O)O)C(O)C(O)C1O]

where ** indicates the point of attachment to the —NH—;
X₄ is

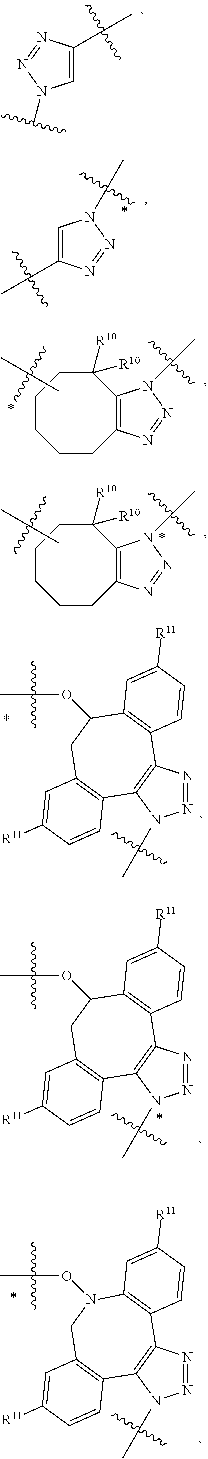

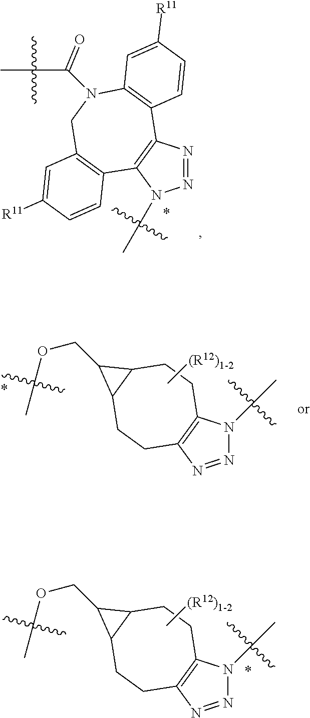

where the * indicates the point of attachment is toward R¹¹⁴;
R¹¹⁴ is

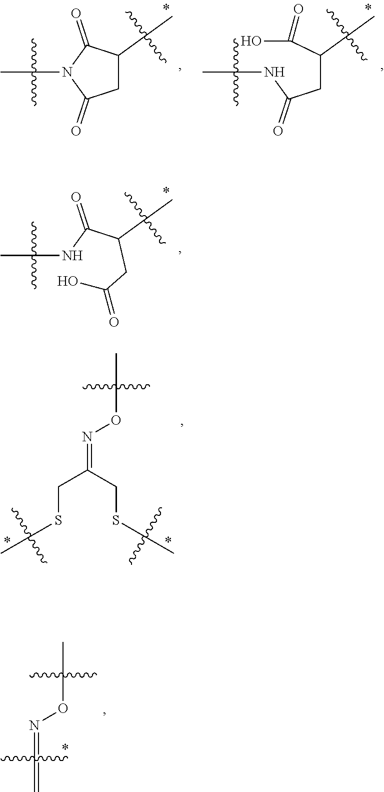

NR⁶C(═O)CH₂—*, —NHC(═O)CH₂—*, —S(═O)₂CH₂CH₂—*, —(CH₂)₂S(═O)₂CH₂CH₂—*, —NR⁶S(═O)₂CH₂CH₂—*, —NR⁶C(═O)CH₂CH₂—*, —NH—, —C(═O)—, —NHC(═O)*, —CH₂NHCH₂CH₂—*, —NHCH₂CH₂—*, —S—,

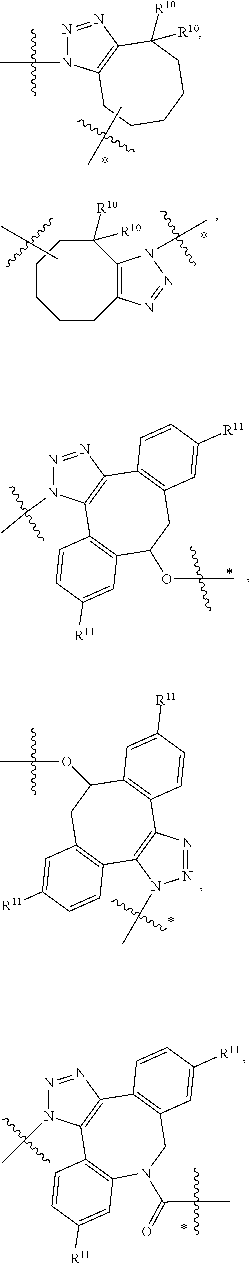

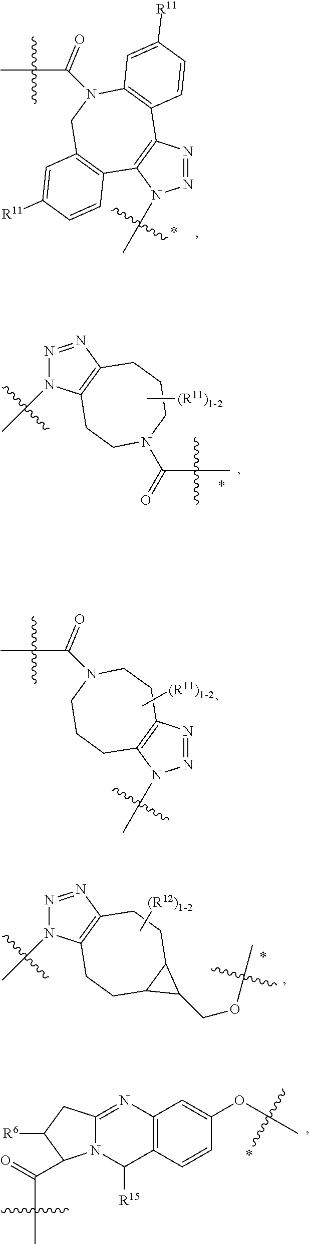

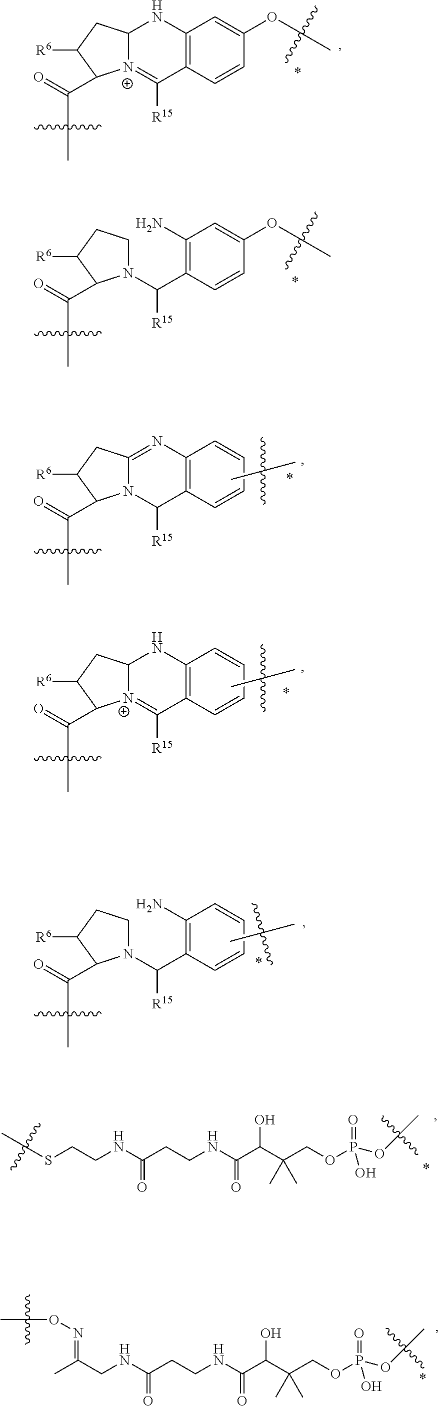

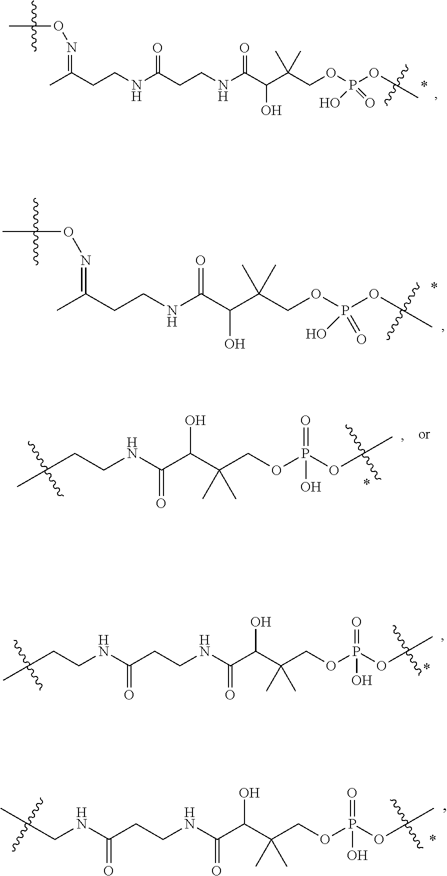 [SMILES: *O/N=C(\C)CCNC(=O)CCNC(=O)C(O)C(C)(C)COP(=O)(O)O* ; *O/N=C(\C)CCNC(=O)C(O)C(C)(C)COP(=O)(O)O* ; *CCNC(=O)C(O)C(C)(C)COP(=O)(O)O* ; *CCNC(=O)CCNC(=O)C(O)C(C)(C)COP(=O)(O)O* ; *CNC(=O)CCNC(=O)C(O)C(C)(C)COP(=O)(O)O*]

where the * indicates the point of attachment to A;
each R⁶ is independently selected from H and C₁-C₆alkyl;
each R¹⁰ is independently selected from H, C₁-C₆alkyl, F, Cl, and —OH;
each R¹¹ is independently selected from H, C₁-C₆alkyl, F, Cl, —NH₂, —OCH₃, —OCH₂CH₃, —N(CH₃)₂, —CN, —NO₂ and —OH;
each R¹² is independently selected from H, C₁₋₆alkyl, fluoro, benzyloxy substituted with —C(═O)OH, benzyl substituted with —C(═O)OH, C1₄alkoxy substituted with —C(═O)OH and C1₄alkyl substituted with —C(═O)OH;
each R¹⁵ is independently selected from H, —CH₃ and phenyl;
each m is independently selected from 1, 2, 3, 4, 5, 6, 7, 8, 9 and 10, and
each p is independently selected from 1, 2, 3, 4, 5, 6, 7, 8, 9, 10, 11, 12, 13 and 14.

In one aspect the conjugates of the invention have the structure of Formula (G):

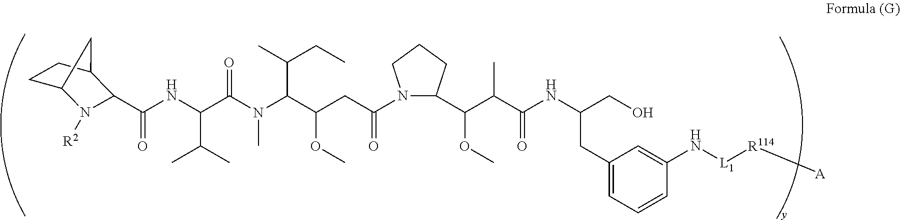

wherein:
A represents an antibody fragment (e.g., Fab or Fab′) that specifically binds to human cKIT;
y is an integer from 1 to 10;
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)R⁴ or C₁-C₆alkyl which is optionally substituted with —CN, —C(═O)NH₂ or 1 to 5 hydroxy;
each R³ is independently selected from C₁-C₆alkyl and C₁-C₆alkyl which is optionally substituted with 1 to 5 hydroxyl;
each R⁴ is independently selected from H and C₁-C₆alkyl;
L₁ is —X₁C(═O)((CH₂)ₘO)ₚ(CH₂)ₘ—**, —X₁C(═O)(CH₂)ₘ—**, —X₂X₁C(═O)((CH₂)ₘO)ₚ(CH₂)ₘ—**, —X₂X₁C(═O)(CH₂)ₘ—**, —X₃C(═O)((CH₂)ₘO)ₚ(CH₂)ₘ—**, —X₃C(═O)(CH₂)ₘ—**, —X₃C(═O)(CH₂)ₘNHC(═O)((CH₂)ₘO)ₚ(CH₂)ₘ—**, —X₃C(═O)(CH₂)ₘNHC(═O)(CH₂)ₘ—**, —X₃C(═O)(CH₂)ₘ—**, —X₁C(═O)((CH₂)ₘO)ₚ(CH₂)ₘX₄(CH₂)ₘ—**, —X₁C(═O)(CH₂)ₘX₄(CH₂)ₘ—**, —X₂X₁C(═O)((CH₂)ₘO)ₚ(CH₂)ₘX₄(CH₂)ₘ—** or —X₂X₁C(═O)(CH₂)ₘX₄(CH₂)ₘ—**, where ** indicates the point of attachment to R¹¹⁴;
X₁ is

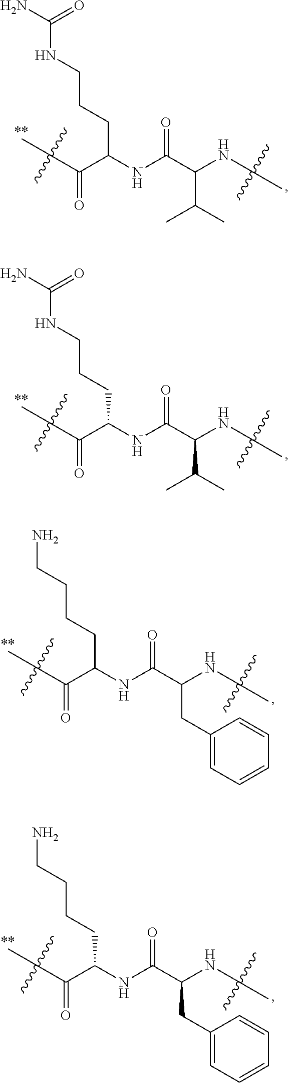 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O]

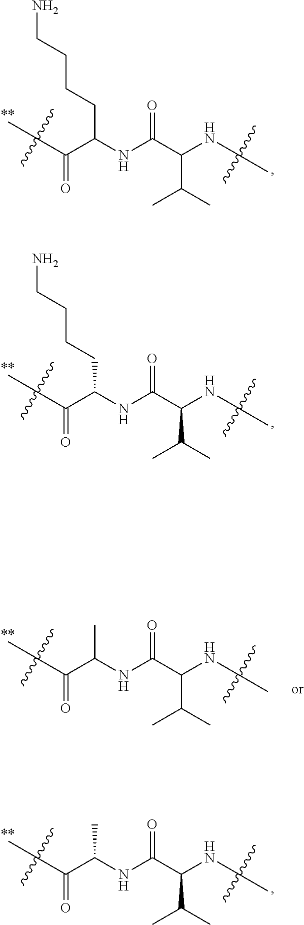 [SMILES: *NC(C(=O)NC(CCCCN)C(*)=O)C(C)C ; *N[C@H](C(=O)N[C@@H](CCCCN)C(*)=O)C(C)C ; *NC(C(=O)NC(C)C(*)=O)C(C)C ; *N[C@H](C(=O)N[C@@H](C)C(*)=O)C(C)C]

where ** indicates the point of attachment to the —NH— or to X₂;
X₂ is

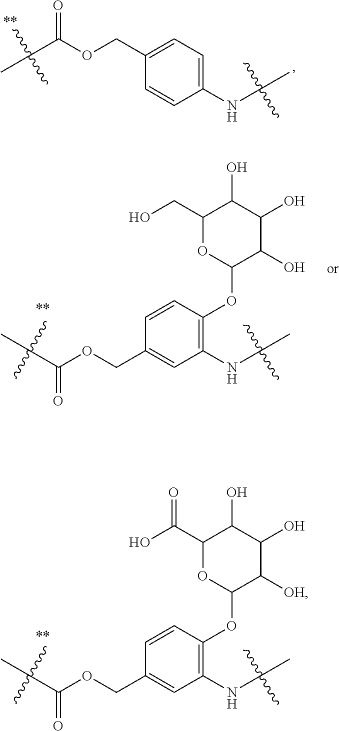 [SMILES: *Nc1ccc(COC(*)=O)cc1 ; *Nc1cc(COC(*)=O)ccc1OC1OC(CO)C(O)C(O)C1O ; *Nc1cc(COC(*)=O)ccc1OC1OC(C(=O)O)C(O)C(O)C1O]

where ** indicates the point of attachment to the —NH—;
X₃ is

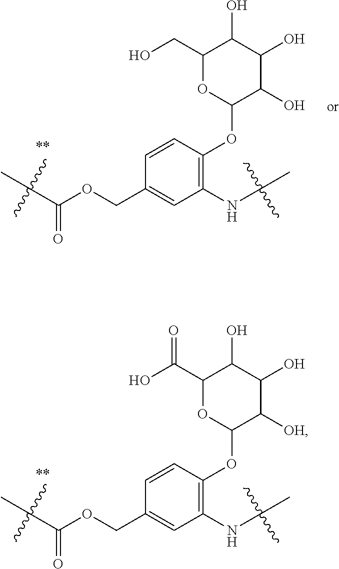 [SMILES: *Nc1cc(COC(*)=O)ccc1OC1OC(CO)C(O)C(O)C1O ; *Nc1cc(COC(*)=O)ccc1OC1OC(C(=O)O)C(O)C(O)C1O]

where ** indicates the point of attachment to the —NH—;
X₄ is

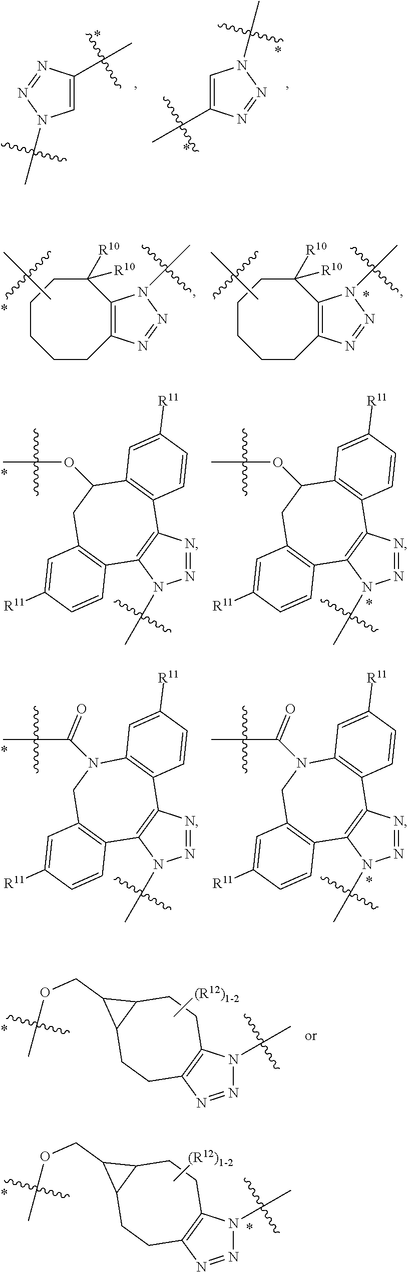

where the * indicates the point of attachment is toward R¹¹⁴;
R¹¹⁴ is

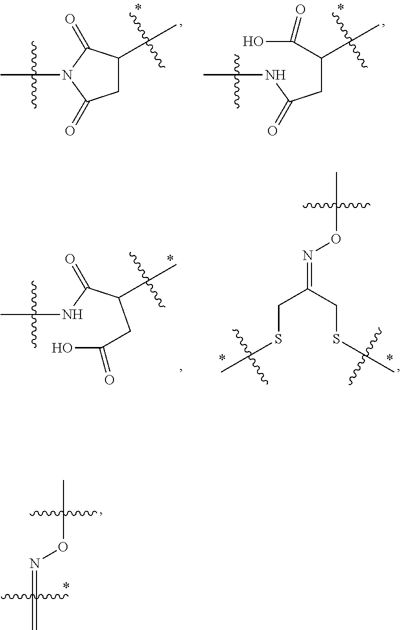

—NR⁶C(═O)CH₂—*, —NHC(═O)CH₂—*, —S(═O)₂CH₂CH₂—*, —(CH₂)₂S(═O)₂CH₂CH₂—*, —NR⁶S(═O)₂CH₂CH₂—*, —NR⁶C(═O)CH₂CH₂—*, —NH—, —C(═O)—, —NHC(═O)—*, —CH₂NHCH₂CH₂—*, —NHCH₂CH₂—*, —S—,

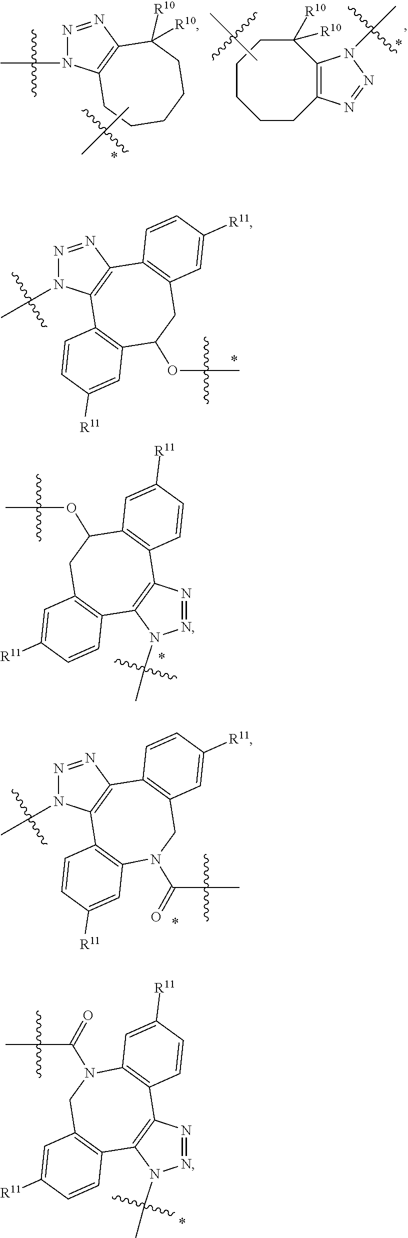

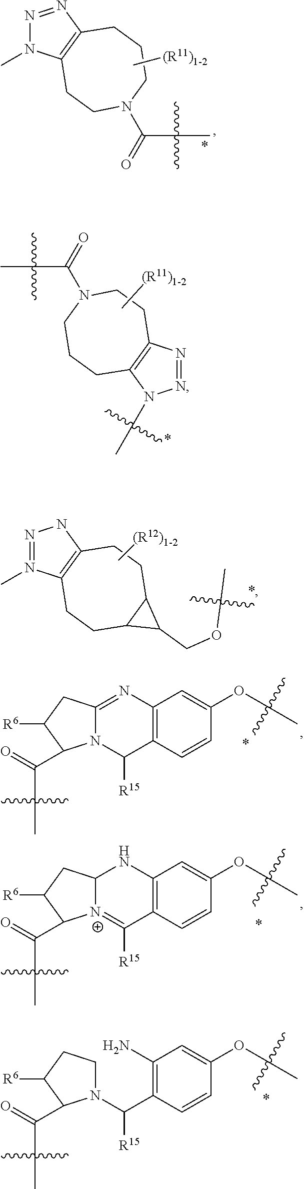

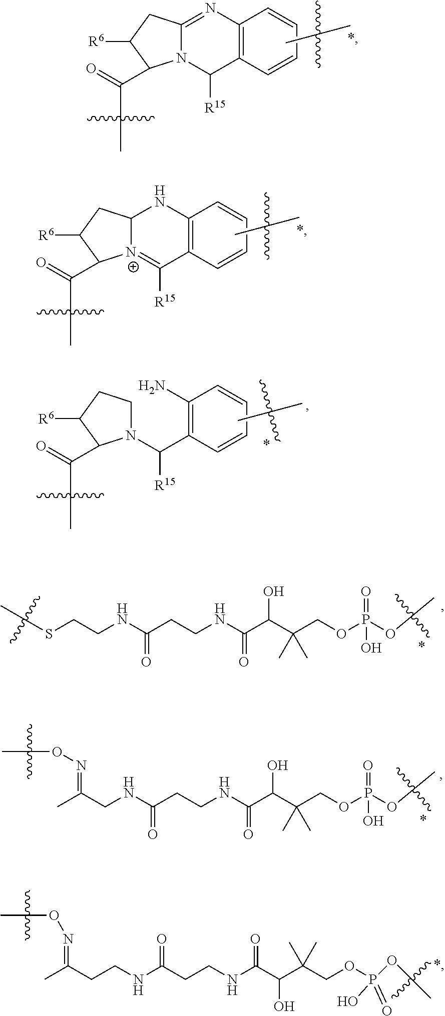

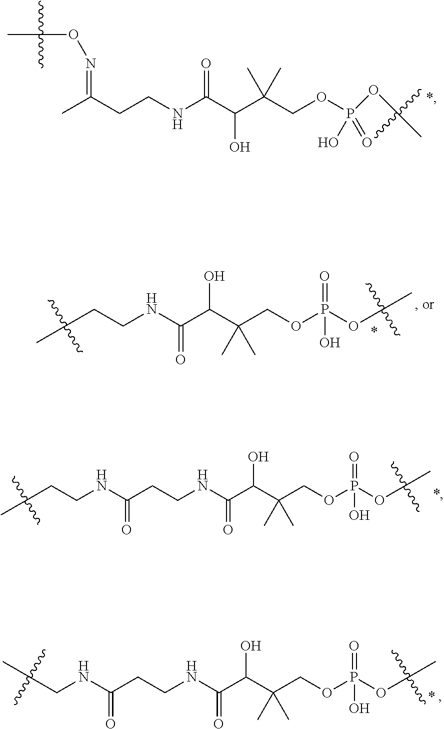 [SMILES: *O/N=C(\C)CCNC(=O)C(O)C(C)(C)COP(=O)(O)O* ; *CCNC(=O)C(O)C(C)(C)COP(=O)(O)O* ; *CCNC(=O)CCNC(=O)C(O)C(C)(C)COP(=O)(O)O* ; *CNC(=O)CCNC(=O)C(O)C(C)(C)COP(=O)(O)O*]

where the * indicates the point of attachment to A;
each R⁶ is independently selected from H and C₁-C₆alkyl;
each R¹⁰ is independently selected from H, C₁-C₆alkyl, F, Cl, and —OH;
each R¹¹ is independently selected from H, C₁-C₆alkyl, F, Cl, —NH₂, —OCH₃, —OCH₂CH₃, —N(CH₃)₂, —CN, —NO₂ and —OH;
each R¹² is independently selected from H, C₁₋₆alkyl, fluoro, benzyloxy substituted with —C(═O)OH, benzyl substituted with —C(═O)OH, C₁₋₄alkoxy substituted with —C(═O)OH and C₁₋₄alkyl substituted with —C(═O)OH;
each R¹⁵ is independently selected from H, —CH₃ and phenyl;
each m is independently selected from 1, 2, 3, 4, 5, 6, 7, 8, 9 and 10, and
each p is independently selected from 1, 2, 3, 4, 5, 6, 7, 8, 9, 10, 11, 12, 13 and 14.

In one aspect the conjugates of the invention have the structure of Formula (H):

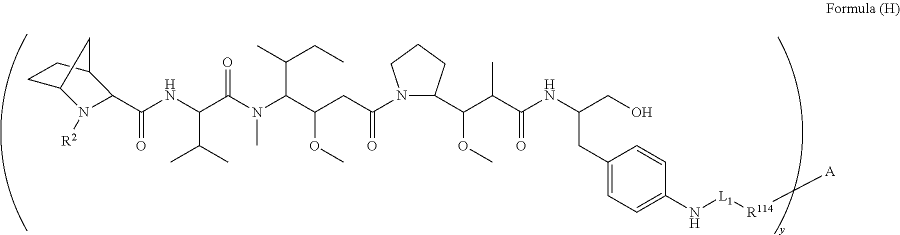

wherein:
A represents an antibody fragment (e.g., Fab or Fab′) that specifically binds to human cKIT;
y is an integer from 1 to 10;
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)R⁴ or C₁-C₆alkyl which is optionally substituted with —CN, —C(═O)NH₂ or 1 to 5 hydroxy;
each R³ is independently selected from C₁-C₆alkyl and C₁-C₆alkyl which is optionally substituted with 1 to 5 hydroxyl;
each R⁴ is independently selected from H and C₁-C₆alkyl;
L₁ is —X₁C(═O)((CH₂)ₘO)ₚ(CH₂)ₘ—**, —X₁C(═O)(CH₂)ₘ—**, —X₂X₁C(═O)((CH₂)ₘO)ₚ(CH₂)ₘ—**, —X₂X₁C(═O)(CH₂)ₘ—**, —X₃C(═O)((CH₂)ₘO)ₚ(CH₂)ₘ—**, —X₃C(═O)(CH₂)ₘ—**, —X₃C(═O)(CH₂)ₘNHC(═O)((CH₂)ₘO)ₚ(CH₂)ₘ—**, —X₃C(═O)(CH₂)ₘNHC(═O)(CH₂)ₘ—**, —X₃C(═O)(CH₂)ₘ—**, —X₁C(═O)((CH₂)ₘO)ₚ(CH₂)ₘX₄(CH₂)ₘ—**, —X₁C(═O)(CH₂)ₘX₄(CH₂)ₘ—**, —X₂X₁C(═O)((CH₂)ₘO)ₚ(CH₂)ₘX₄(CH₂)ₘ—** or —X₂X₁C(═O)(CH₂)ₘX₄(CH₂)ₘ—**, where ** indicates the point of attachment to R⁴;
X₁ is

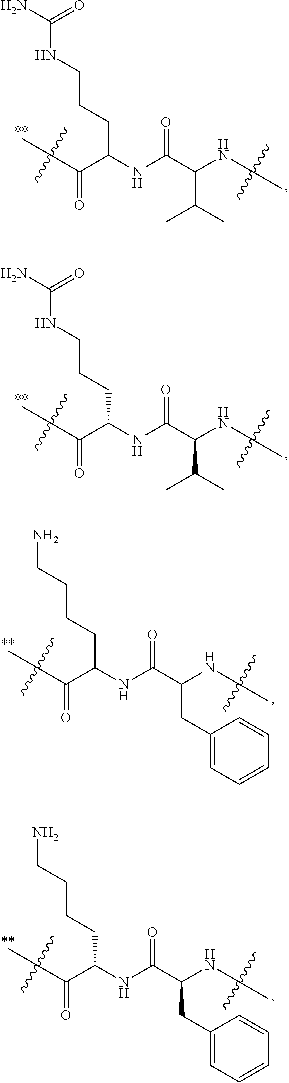 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O]

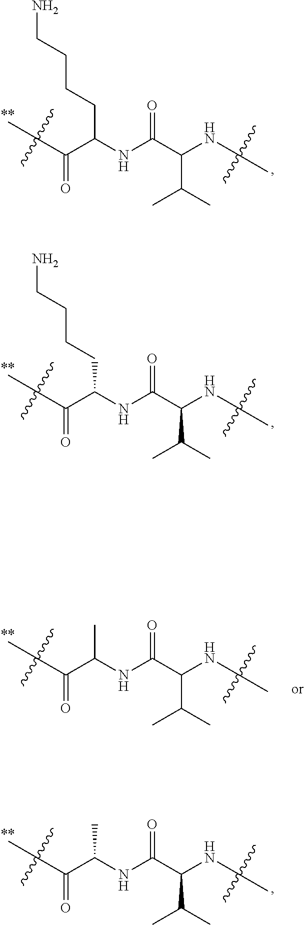 [SMILES: *NC(C(=O)NC(CCCCN)C(*)=O)C(C)C ; *N[C@H](C(=O)N[C@@H](CCCCN)C(*)=O)C(C)C ; *NC(C(=O)NC(C)C(*)=O)C(C)C ; *N[C@H](C(=O)N[C@@H](C)C(*)=O)C(C)C]

where ** indicates the point of attachment to the —NH— or to X₂;
X₂ is

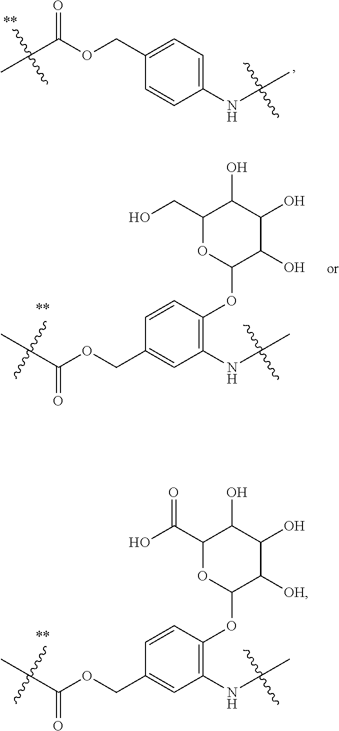 [SMILES: *Nc1ccc(COC(*)=O)cc1 ; *Nc1cc(COC(*)=O)ccc1OC1OC(CO)C(O)C(O)C1O ; *Nc1cc(COC(*)=O)ccc1OC1OC(C(=O)O)C(O)C(O)C1O]

where ** indicates the point of attachment to the —NH—;
X₃ is

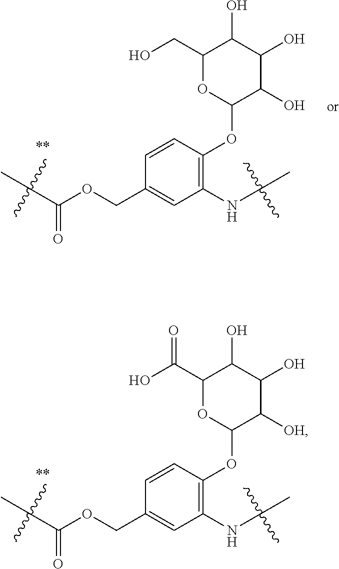 [SMILES: *Nc1cc(COC(*)=O)ccc1OC1OC(CO)C(O)C(O)C1O ; *Nc1cc(COC(*)=O)ccc1OC1OC(C(=O)O)C(O)C(O)C1O]

where ** indicates the point of attachment to the —NH—;
X₄ is

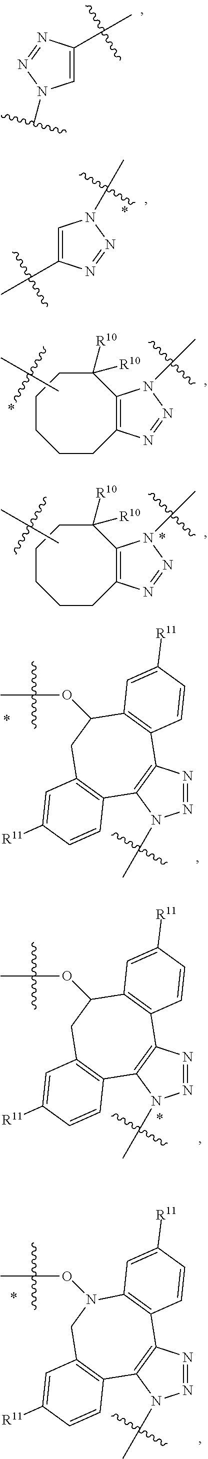

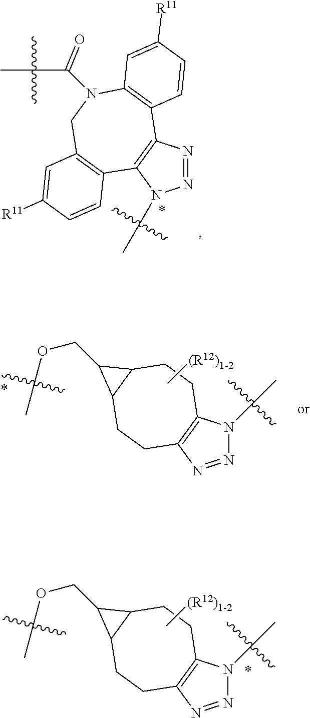

where the * indicates the point of attachment is toward R¹¹⁴;
R¹¹⁴ is

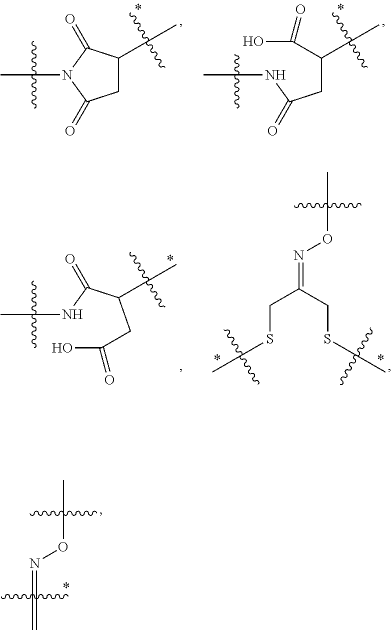

NR⁶C(═O)CH₂—*, —NHC(═O)CH₂—*, —S(═O)₂CH₂CH₂—*, —(CH₂)₂S(═O)₂CH₂CH₂—*, —NR⁶S(═O)₂CH₂CH₂—*, —NR⁶C(═O)CH₂CH₂—*, —NH—, —C(═O)—, —NHC(═O)*, CH₂NHCH₂CH₂—*, —NHCH₂CH₂—*, —S—,

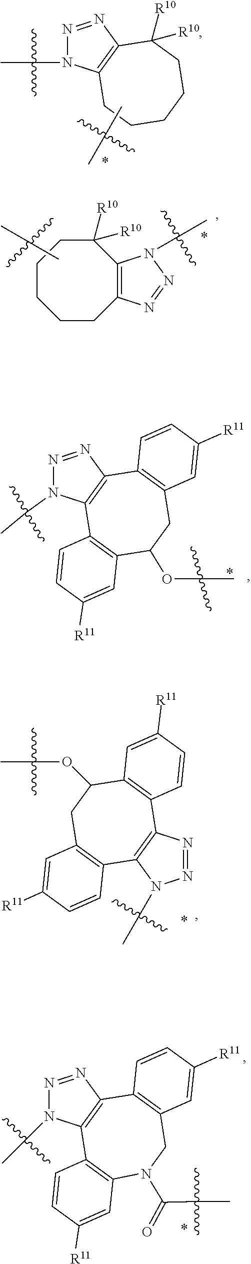

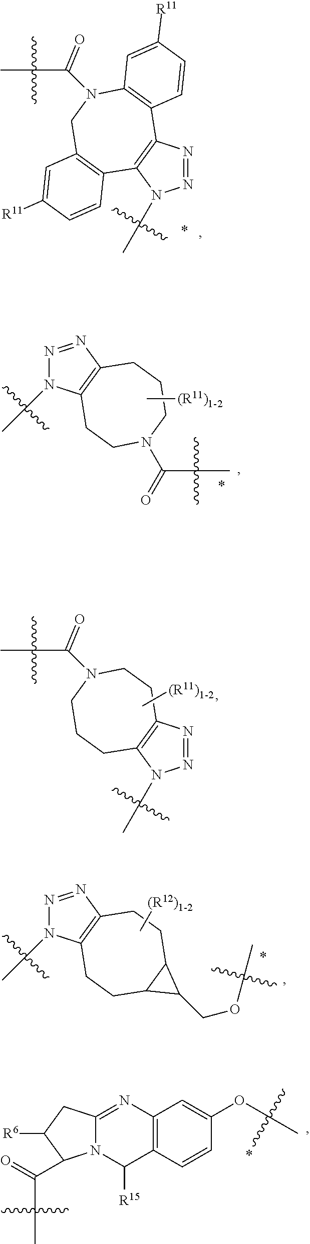

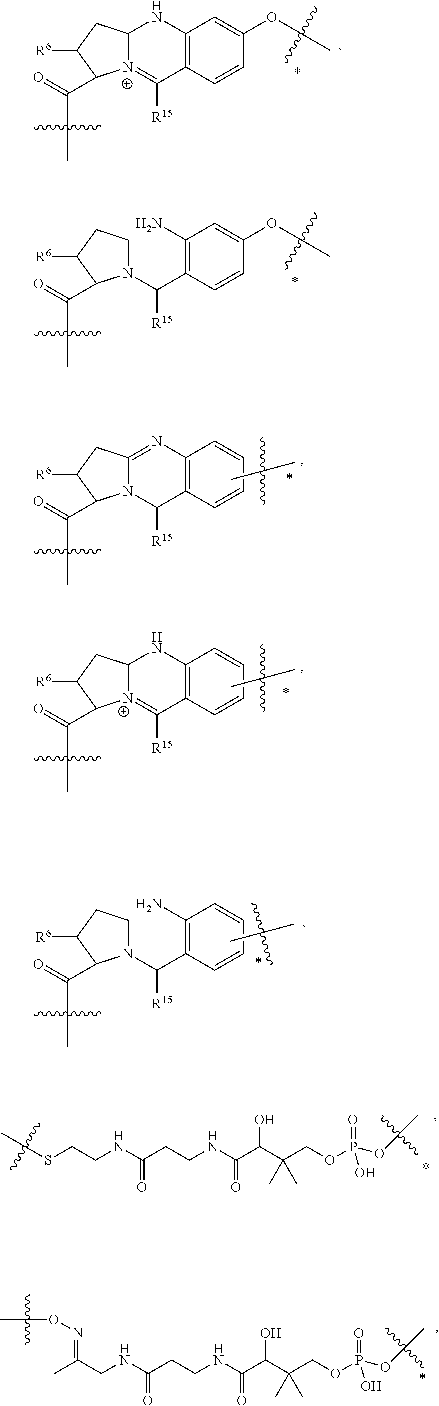

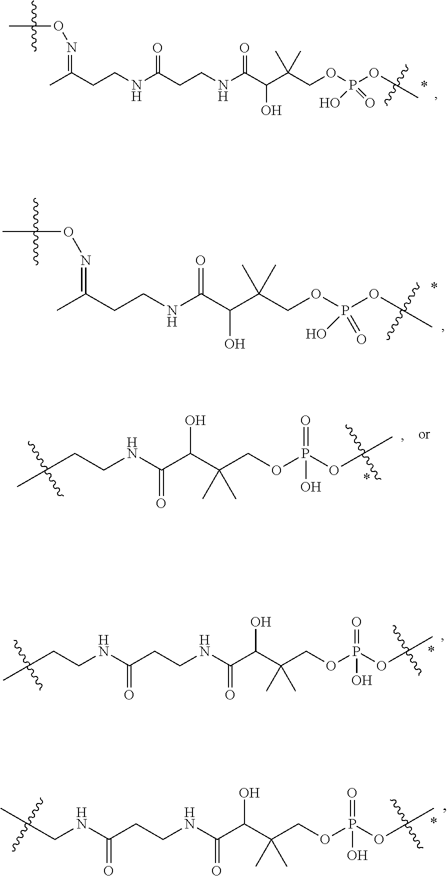 [SMILES: *O/N=C(\C)CCNC(=O)CCNC(=O)C(O)C(C)(C)COP(=O)(O)O* ; *O/N=C(\C)CCNC(=O)C(O)C(C)(C)COP(=O)(O)O* ; *CCNC(=O)C(O)C(C)(C)COP(=O)(O)O* ; *CCNC(=O)CCNC(=O)C(O)C(C)(C)COP(=O)(O)O* ; *CNC(=O)CCNC(=O)C(O)C(C)(C)COP(=O)(O)O*]

where the * indicates the point of attachment to A;
each R⁶ is independently selected from H and C₁-C₆alkyl;
each R¹⁰ is independently selected from H, C₁-C₆alkyl, F, Cl, and —OH;
each R¹¹ is independently selected from H, C₁-C₆alkyl, F, Cl, —NH₂, —OCH₃, —OCH₂CH₃, —N(CH₃)₂, —CN, —NO₂ and —OH;
each R¹² is independently selected from H, C₁₋₆alkyl, fluoro, benzyloxy substituted with —C(═O)OH, benzyl substituted with —C(═O)OH, C1₄alkoxy substituted with —C(═O)OH and C1₄alkyl substituted with —C(═O)OH;
each R¹⁵ is independently selected from H, —CH₃ and phenyl;
each m is independently selected from 1, 2, 3, 4, 5, 6, 7, 8, 9 and 10, and
each p is independently selected from 1, 2, 3, 4, 5, 6, 7, 8, 9, 10, 11, 12, 13 and 14.

Certain aspects and examples of the conjugates of the invention are provided in the following listing of additional, enumerated embodiments. It will be recognized that features specified in each embodiment may be combined with other specified features to provide further embodiments of the present invention.

Embodiment 69. The conjugate having the structure of Formula (E) is a conjugate having has the structure of Formula (E-1):

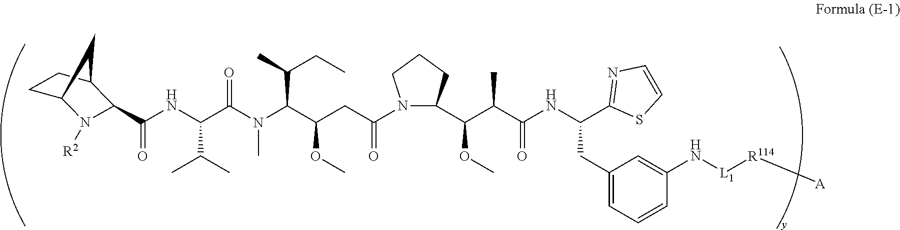

wherein: R², R¹¹⁴, A, y, and L₁ are as defined for conjugates of Formula (E) above.

Embodiment 70. The conjugate having the structure of Formula (F) is a conjugate having has the structure of Formula (F-1):

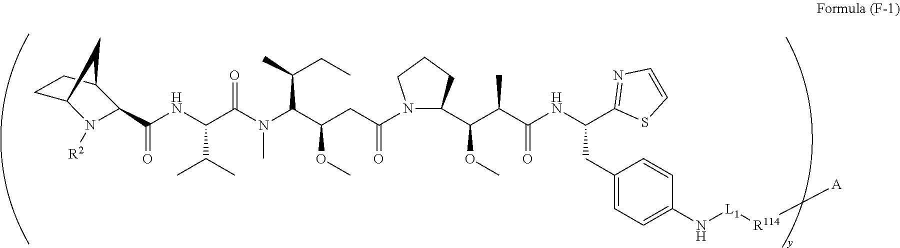

wherein: R², R¹¹⁴, A, y, and L₁ are as defined for conjugates of Formula (F) above.

Embodiment 71. The conjugate having the structure of Formula (G) is a conjugate having has the structure of Formula (G-1):

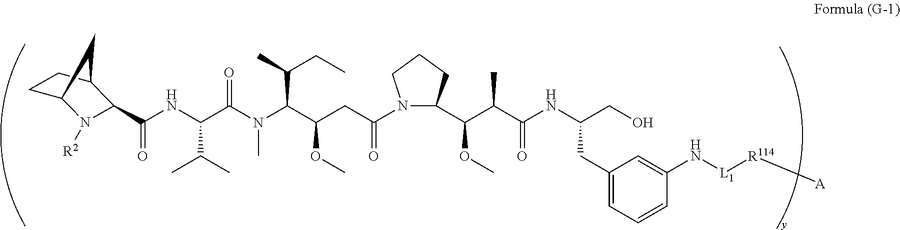

wherein: R², R¹¹⁴, A, y, and L₁ are as defined for conjugates of Formula (G) above.

Embodiment 72. The conjugate having the structure of Formula (H) is a conjugate having has the structure of Formula (H-1):

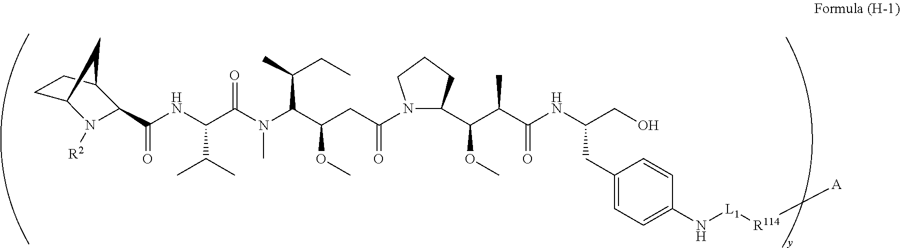

wherein: R², R¹¹⁴, A, y, and L₁ are as defined for conjugates of Formula (H) above.

Embodiment 73. The conjugate of Formula (E), Formula (F), Formula (G), Formula (H) or any one of Embodiments 69 to 72, wherein R² is H or C₁-C₆alkyl.

Embodiment 74. The conjugate of Formula (E), Formula (F), Formula (G), Formula (H) or any one of Embodiments 69 to 72, wherein R² is H or methyl.

Embodiment 75. The conjugate of Formula (E), Formula (F), Formula (G), Formula (H) or any one of Embodiments 69 to 72, wherein R² is H.

Embodiment 76. The conjugate of Formula (E), Formula (F), Formula (G), Formula (H) or any one of Embodiments 69 to 72, wherein R² is methyl.

Embodiment 77. The conjugate having the structure of Formula (E) is a conjugate having has the structure of Formula (E-2):

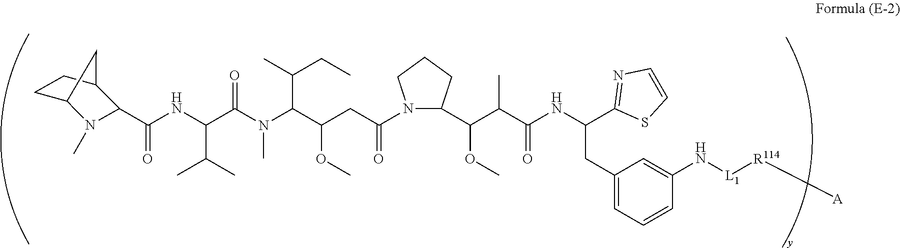

wherein: R¹¹⁴, A, y, and L₁ are as defined for conjugates of Formula (E) above.

Embodiment 78. The conjugate having the structure of Formula (E) is a conjugate having has the structure of Formula (E-3):

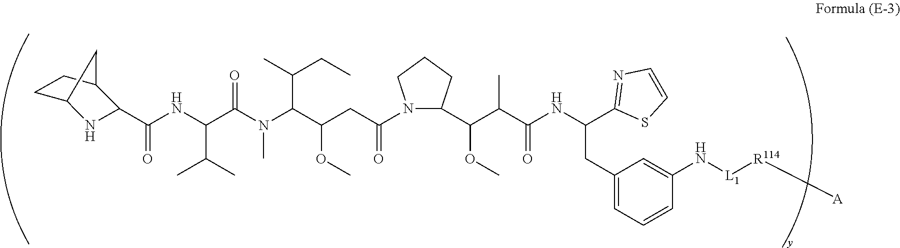

wherein: R¹¹⁴, A, y, and L₁ are as defined for conjugates of Formula (E) above.

Embodiment 79. The conjugate having the structure of Formula (E) or Formula (E-1) is a conjugate having has the structure of Formula (E-4):

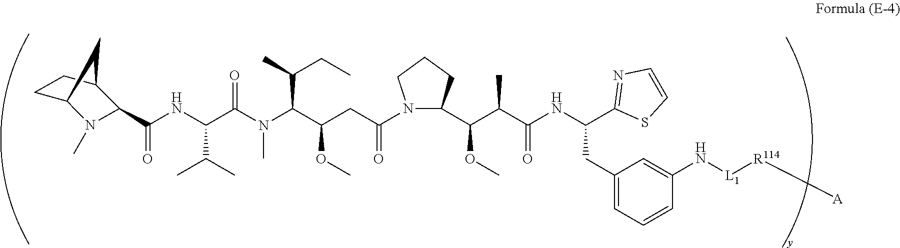

wherein: R¹¹⁴, A, y, and L₁ are as defined for conjugates of Formula (E) above.

Embodiment 80. The conjugate having the structure of Formula (E) or Formula (E-1) is a conjugate having has the structure of Formula (E-5):

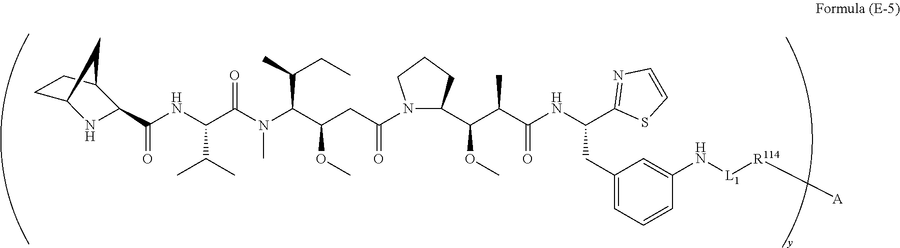

wherein: R¹¹⁴, A, y, and L₁ are as defined for conjugates of Formula (E) above.

Embodiment 81. The conjugate having the structure of Formula (F) is a conjugate having has the structure of Formula (F-2):

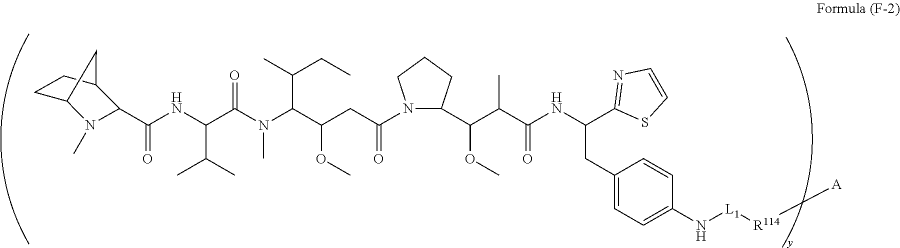

wherein: R¹¹⁴, A, y, and L₁ are as defined for conjugates of Formula (F) above.

Embodiment 82. The conjugate having the structure of Formula (F) is a conjugate having has the structure of Formula (F-3):

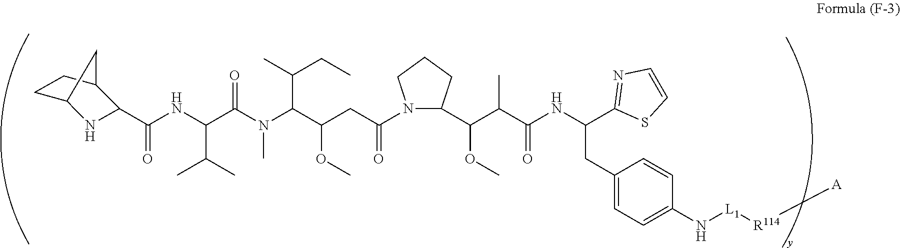

wherein: R¹¹⁴, A, y, and L₁ are as defined for conjugates of Formula (F) above.

Embodiment 83. The conjugate having the structure of Formula (F) or Formula (F-1) is a conjugate having has the structure of Formula (F-4):

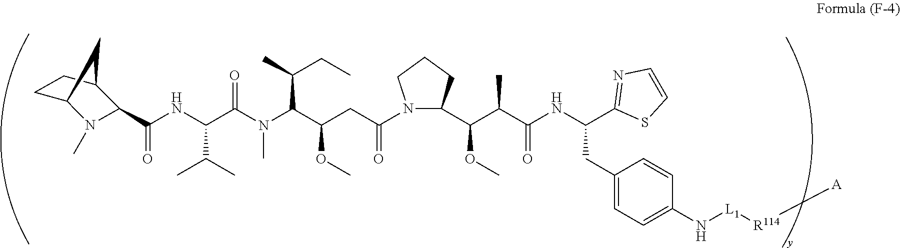

wherein: R¹¹⁴, A, y, and L₁ are as defined for conjugates of Formula (F) above.

Embodiment 84. The conjugate having the structure of Formula (F) or Formula (F-1) is a conjugate having has the structure of Formula (F-5):

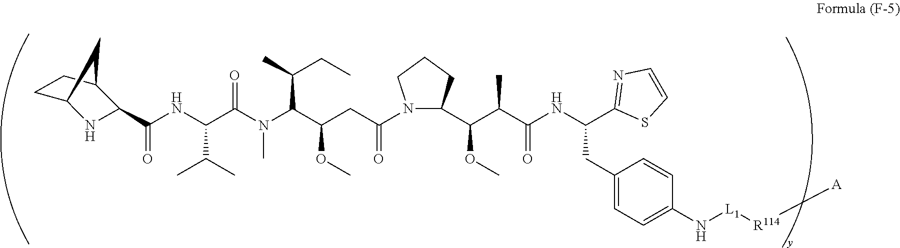

wherein: R¹¹⁴, A, y, and L₁ are as defined for conjugates of Formula (F) above.

Embodiment 85. The conjugate having the structure of Formula (G) is a conjugate having has the structure of Formula (G-2):

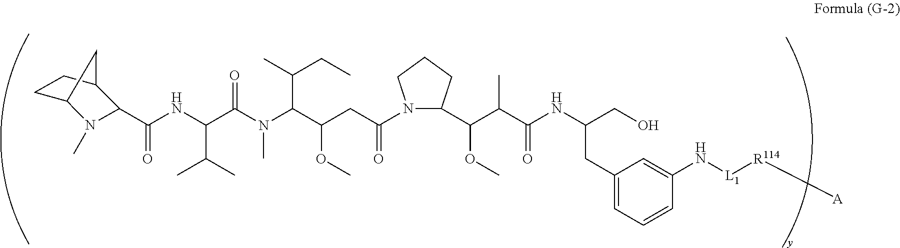

wherein: R¹¹⁴, A, y, and L₁ are as defined for conjugates of Formula (G) above.

Embodiment 86. The conjugate having the structure of Formula (G) is a conjugate having has the structure of Formula (G-3):

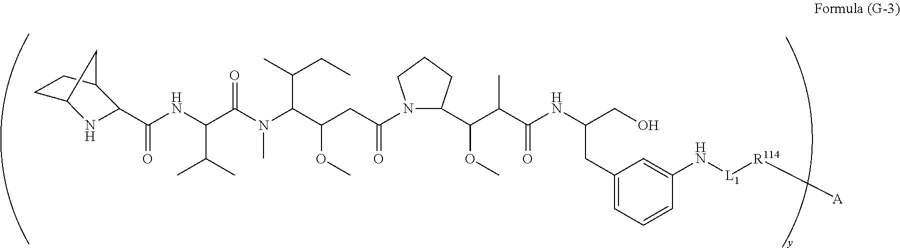

wherein: R¹¹⁴, A, y, and L₁ are as defined for conjugates of Formula (G) above.

Embodiment 87. The conjugate having the structure of Formula (G) or Formula (G-1) is a conjugate having has the structure of Formula (G-4):

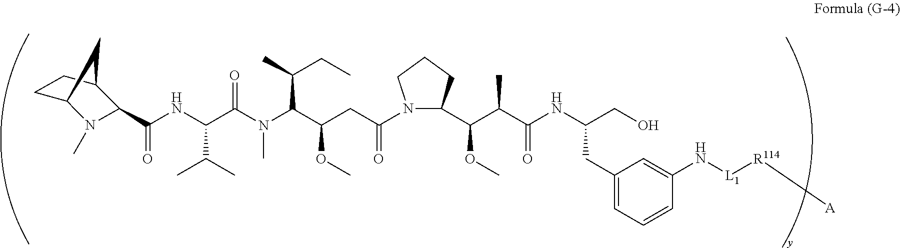

wherein: R¹¹⁴, A, y, and L₁ are as defined for conjugates of Formula (G) above.

Embodiment 88. The conjugate having the structure of Formula (G) or Formula (G-1) is a conjugate having has the structure of Formula (G-5):

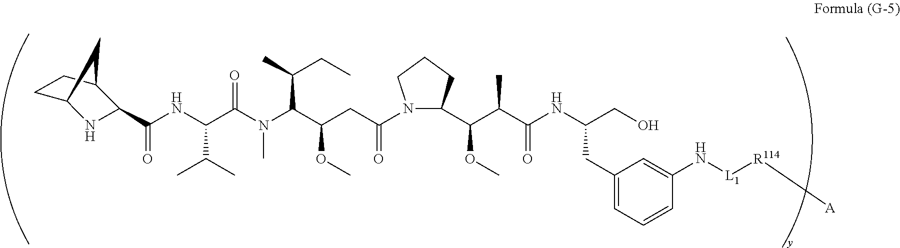

wherein: R¹¹⁴, A, y, and L₁ are as defined for conjugates of Formula (G) above.

Embodiment 89. The conjugate having the structure of Formula (H) is a conjugate having has the structure of Formula (H-2):

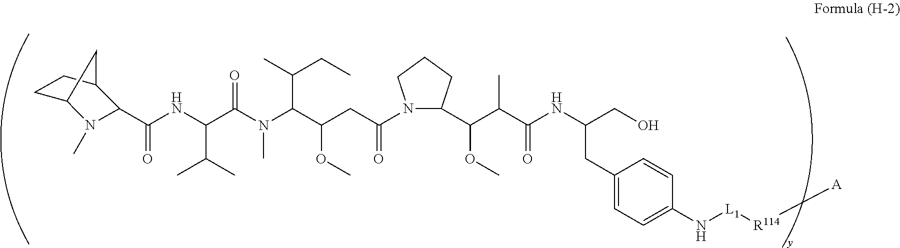

wherein: R¹¹⁴, A, y, and L₁ are as defined for conjugates of Formula (H) above.

Embodiment 90. The conjugate having the structure of Formula (H) is a conjugate having has the structure of Formula (H-3):

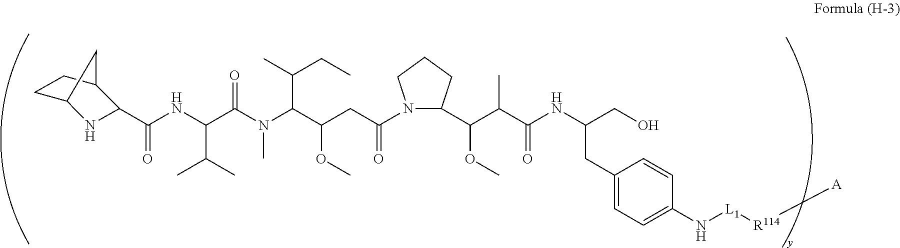

wherein: R¹¹⁴, A, y, and L₁ are as defined for conjugates of Formula (H) above.

Embodiment 91. The conjugate having the structure of Formula (H) or Formula (H-1) is a conjugate having has the structure of Formula (H-4):

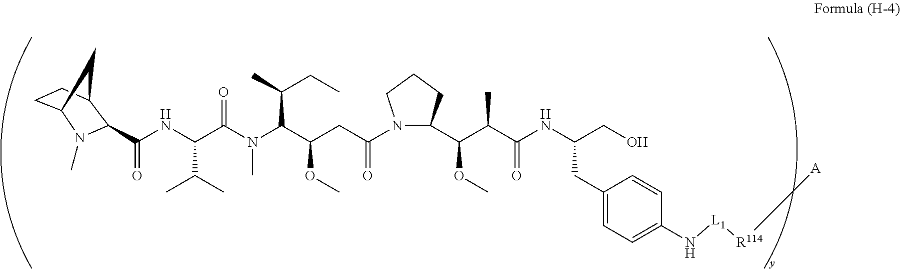

wherein: R¹¹⁴, A, y, and L₁ are as defined for conjugates of Formula (H) above.

Embodiment 92. The conjugate having the structure of Formula (H) or Formula (H-1) is a conjugate having has the structure of Formula (H-5):

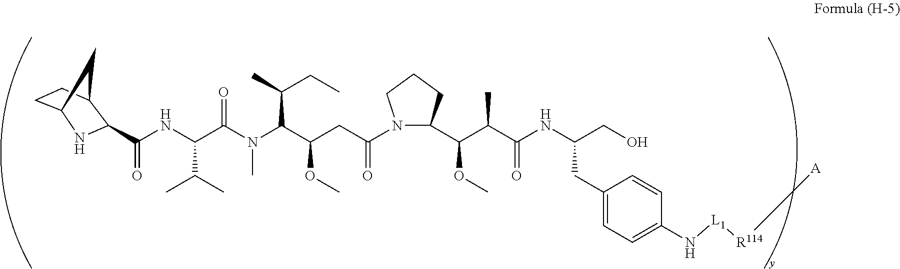

wherein: R¹¹⁴, A, y, and L₁ are as defined for conjugates of Formula (H) above.

Embodiment 93. The conjugate of Formula (E), Formula (F), Formula (G), Formula (H) or the conjugate of any one of Embodiments 69 to 92, wherein:
L₁ is —X₁C(═O)((CH₂)ₘO)ₚ(CH₂)ₘ—** or —X₁C(═O)(CH₂)ₘ—**, where ** indicates the point of attachment to R¹¹⁴;
and
X₁ is

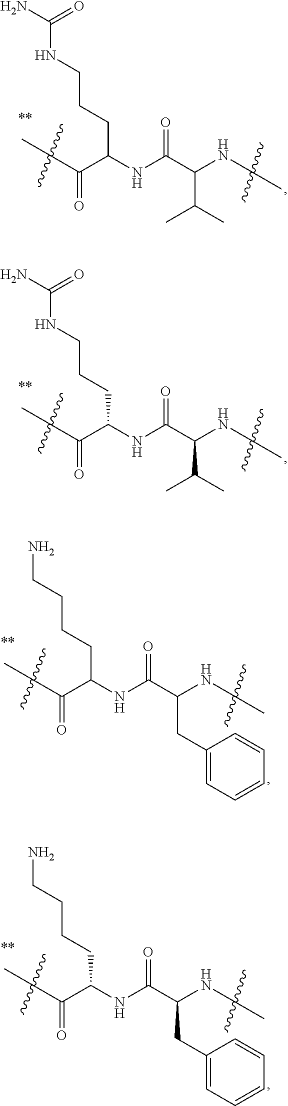 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O]

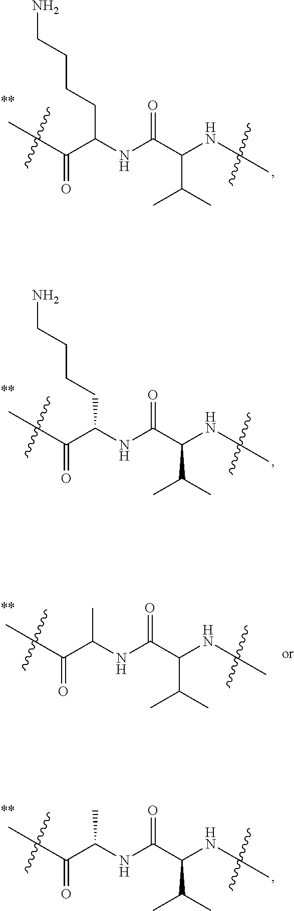 [SMILES: *NC(C(=O)NC(CCCCN)C(*)=O)C(C)C ; *N[C@H](C(=O)N[C@@H](CCCCN)C(*)=O)C(C)C ; *NC(C(=O)NC(C)C(*)=O)C(C)C ; *N[C@H](C(=O)N[C@@H](C)C(*)=O)C(C)C]

where ** indicates the point of attachment to the —NH— or to X₂.

Embodiment 94. The conjugate of Formula (E), Formula (F), Formula (G), Formula (H) or the conjugate of any one of Embodiments 69 to 92, wherein:
L₁ is —X₁C(═O)((CH₂)ₘO)ₚ(CH₂)ₘ—**, where ** indicates the point of attachment to R¹¹⁴
and
X₁ is

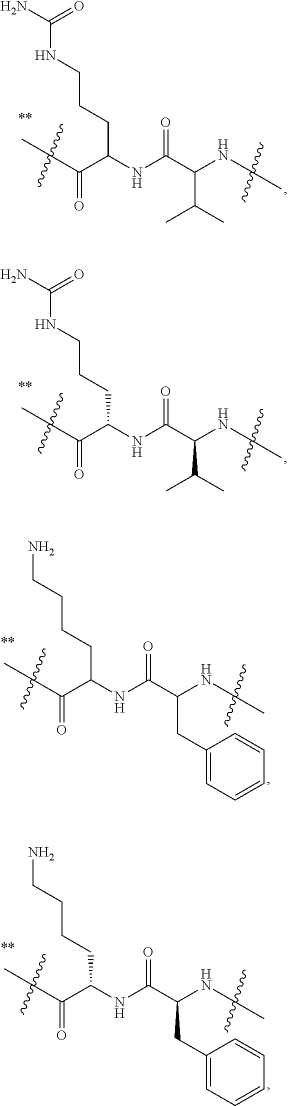 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O]

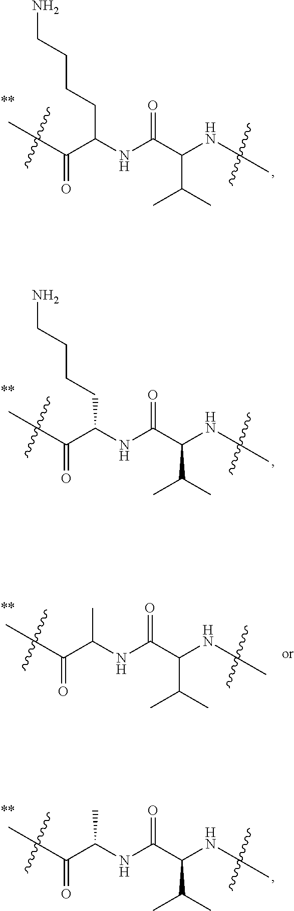 [SMILES: *NC(C(=O)NC(CCCCN)C(*)=O)C(C)C ; *N[C@H](C(=O)N[C@@H](CCCCN)C(*)=O)C(C)C ; *NC(C(=O)NC(C)C(*)=O)C(C)C ; *N[C@H](C(=O)N[C@@H](C)C(*)=O)C(C)C]

where ** indicates the point of attachment to the —NH— or to X₂.

Embodiment 95. The conjugate of Formula (E), Formula (F), Formula (G), Formula (H) or the conjugate of any one of Embodiments 69 to 94, each m is independently selected from 1, 2, 3, 4, 5 and 6.

Embodiment 96. The conjugate of Formula (E), Formula (F), Formula (G), Formula (H) or the conjugate of any one of Embodiments 69 to 94, each m is independently selected from 1, 2, 3, 4 and 5.

Embodiment 97. The conjugate of Formula (E), Formula (F), Formula (G), Formula (H) or the conjugate of any one of Embodiments 69 to 94, each m is independently selected from 1, 2, 3 and 4.

Embodiment 98. The conjugate of Formula (E), Formula (F), Formula (G), Formula (H) or the conjugate of any one of Embodiments 69 to 94, each m is independently selected from 1, 2 and 3.

Embodiment 99. The conjugate of Formula (E), Formula (F), Formula (G), Formula (H) or the conjugate of any one of Embodiments 69 to 94, each m is independently selected from 1 and 2.

Embodiment 100. The conjugate of Formula (E), Formula (F), Formula (G), Formula (H) or the conjugate of any one of Embodiments 69 to 99, each n is independently selected from 1, 2, 3, 4, 5, 6, 7, 8, 9, 10, 11 and 12.

Embodiment 101. The conjugate of Formula (E), Formula (F), Formula (G), Formula (H) or the conjugate of any one of Embodiments 69 to 99, each n is independently selected from 1, 2, 3, 4, 5, 6, 7, 8, 9, 10 and 11.

Embodiment 102. The conjugate of Formula (E), Formula (F), Formula (G), Formula (H) or the conjugate of any one of Embodiments 69 to 99, each n is independently selected from 1, 2, 3, 4, 5, 6, 7, 8, 9 and 10.

Embodiment 103. The conjugate of Formula (E), Formula (F), Formula (G), Formula (H) or the conjugate of any one of Embodiments 69 to 99, each n is independently selected from 1, 2, 3, 4, 5, 6, 7, 8 and 9.

Embodiment 104. The conjugate of Formula (E), Formula (F), Formula (G), Formula (H) or the conjugate of any one of Embodiments 69 to 99, each n is independently selected from 1, 2, 3, 4, 5, 6, 7 and 8.

Embodiment 105. The conjugate of Formula (E), Formula (F), Formula (G), Formula (H) or the conjugate of any one of Embodiments 69 to 99, each n is independently selected from 1, 2, 3, 4, 5, 6 and 7.

Embodiment 106. The conjugate of Formula (E), Formula (F), Formula (G), Formula (H) or the conjugate of any one of Embodiments 69 to 99, each n is independently selected from 1, 2, 3, 4, 5 and 6.

Embodiment 107. The conjugate of Formula (E), Formula (F), Formula (G), Formula (H) or the conjugate of any one of Embodiments 69 to 99, each n is independently selected from 1, 2, 3, 4 and 5.

Embodiment 108. The conjugate of Formula (E), Formula (F), Formula (G), Formula (H) or the conjugate of any one of Embodiments 69 to 99, each n is independently selected from 1, 2, 3 and 4.

Embodiment 109. The conjugate of Formula (E), Formula (F), Formula (G), Formula (H) or the conjugate of any one of Embodiments 69 to 99, each n is independently selected from 1, 2 and 3. In any of the above embodiments, each n is independently selected from 1 and 2.

Embodiment 110. The conjugate of Formula (E), Formula (F), Formula (G), Formula (H) or the conjugate of any one of Embodiments 69 to 109, each y is independently selected from 1, 2, 3, 4, 5, 6, 7, 8, 9, 10, 11 and 12.

Embodiment 111. The conjugate of Formula (E), Formula (F), Formula (G), Formula (H) or the conjugate of any one of Embodiments 69 to 109, each y is independently selected from 1, 2, 3, 4, 5, 6, 7, 8, 9, 10 and 11.

Embodiment 112. The conjugate of Formula (E), Formula (F), Formula (G), Formula (H) or the conjugate of any one of Embodiments 69 to 109, each y is independently selected from 1, 2, 3, 4, 5, 6, 7, 8, 9 and 10.

Embodiment 113. The conjugate of Formula (E), Formula (F), Formula (G), Formula (H) or the conjugate of any one of Embodiments 69 to 109, each y is independently selected from 1, 2, 3, 4, 5, 6, 7, 8 and 9.

Embodiment 114. The conjugate of Formula (E), Formula (F), Formula (G), Formula (H) or the conjugate of any one of Embodiments 69 to 109, each y is independently selected from 1, 2, 3, 4, 5, 6, 7 and 8.

Embodiment 115. The conjugate of Formula (E), Formula (F), Formula (G), Formula (H) or the conjugate of any one of Embodiments 69 to 109, each y is independently selected from 1, 2, 3, 4, 5, 6 and 7.

Embodiment 116. The conjugate of Formula (E), Formula (F), Formula (G), Formula (H) or the conjugate of any one of Embodiments 69 to 109, each y is independently selected from 1, 2, 3, 4, 5 and 6.

Embodiment 117. The conjugate of Formula (E), Formula (F), Formula (G), Formula (H) or the conjugate of any one of Embodiments 69 to 109, each y is independently selected from 1, 2, 3, 4 and 5.

Embodiment 118. The conjugate of Formula (E), Formula (F), Formula (G), Formula (H) or the conjugate of any one of Embodiments 69 to 109, each y is independently selected from 1, 2, 3 and 4.

Embodiment 119. The conjugate of Formula (E), Formula (F), Formula (G), Formula (H) or the conjugate of any one of Embodiments 69 to 109, each y is independently selected from 1, 2 and 3.

Embodiment 120. The conjugate of Formula (E), Formula (F), Formula (G), Formula (H) or the conjugate of any one of Embodiments 69 to 109, each y is independently selected from 1 and 2.

Embodiment 121. The conjugate of Formula (E), Formula (F), Formula (G), Formula (H) or the conjugate of any one of Embodiments 69 to 120, wherein:
L₁ is

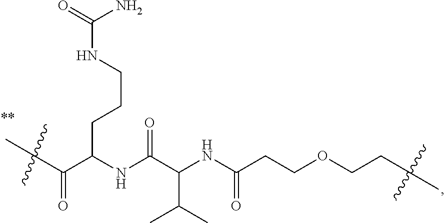 [SMILES: *CCOCCC(=O)NC(C(=O)NC(CCCNC(N)=O)C(*)=O)C(C)C]

where ** indicates the point of attachment to the —NH— or to X₂.

Embodiment 121. The conjugate of Formula (E), Formula (F), Formula (G), Formula (H) or the conjugate of any one of Embodiments 69 to 120, wherein:
L₁ is

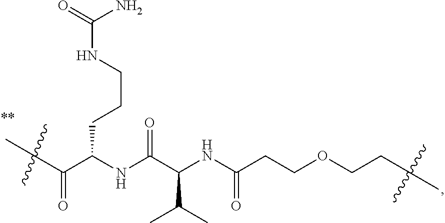 [SMILES: *CCOCCC(=O)N[C@H](C(=O)N[C@@H](CCCNC(N)=O)C(*)=O)C(C)C]

where ** indicates the point of attachment to the —NH— or to X₂.

Embodiment 122. The conjugate of Formula (E), Formula (F), Formula (G), Formula (H) or the conjugate of any one of Embodiments 69 to 121, wherein:
R¹¹⁴ is

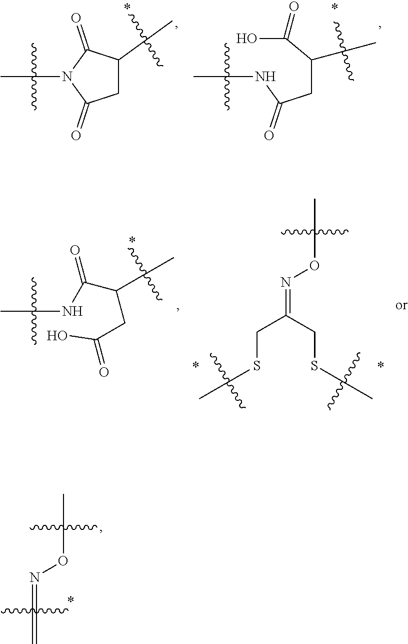

where the * indicates the point of attachment to A.

Embodiment 123. The conjugate of Formula (E), Formula (E-1), Formula (E-2) and Formula (E-4) selected from:

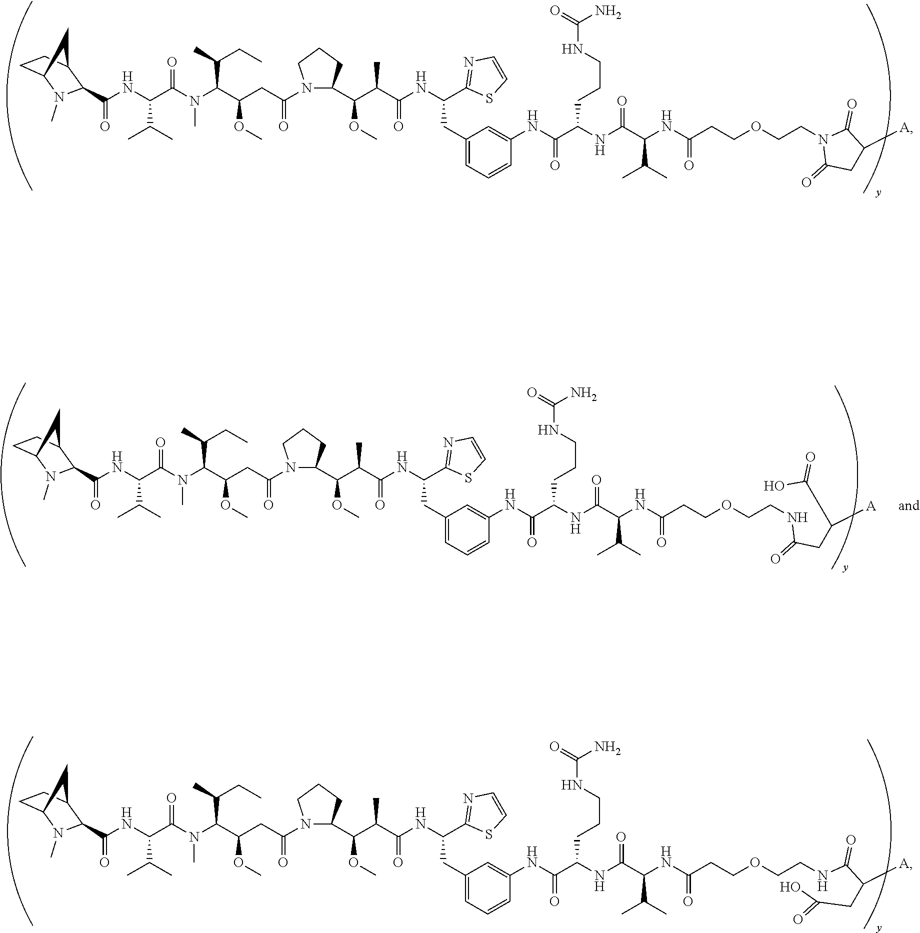

where y and A are as defined for conjugates of Formula (E) above.

Embodiment 124. The conjugate of Formula (E), Formula (E-1), Formula (E-3) and Formula (E-5) selected from:

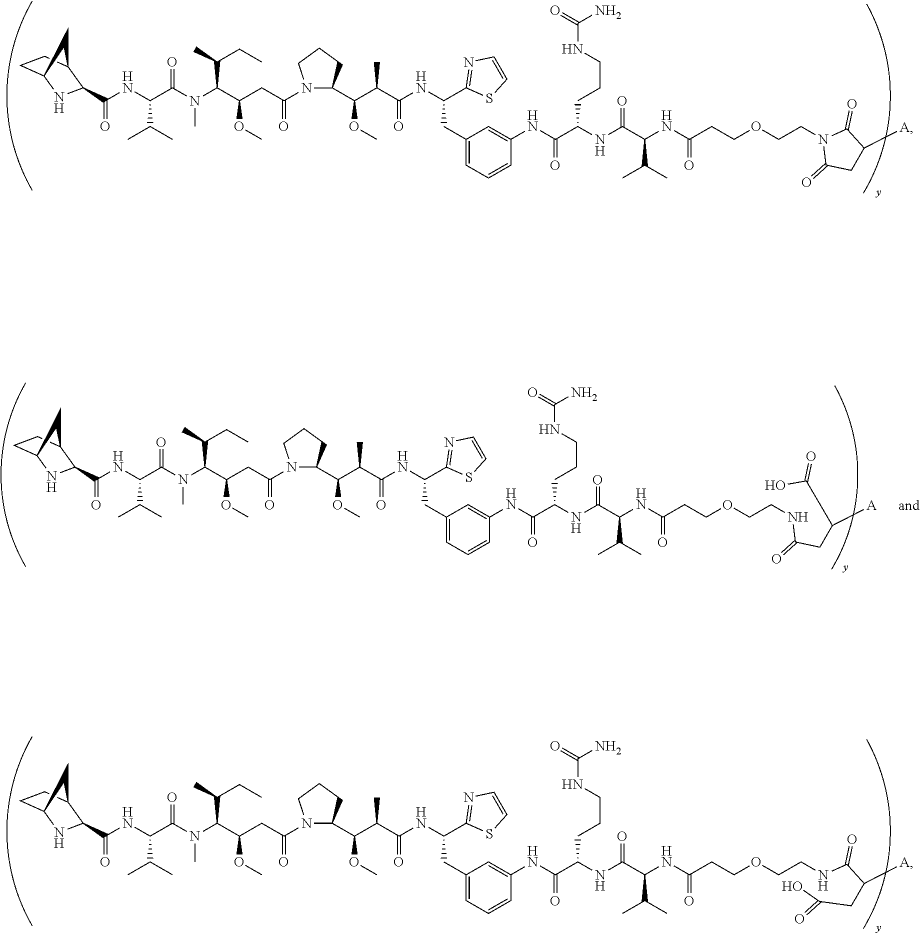

where y and A are as defined for conjugates of Formula (E) above.

Embodiment 125. The conjugate of Formula (F), Formula (F-1), Formula (F-2) and Formula (F-4) selected from:

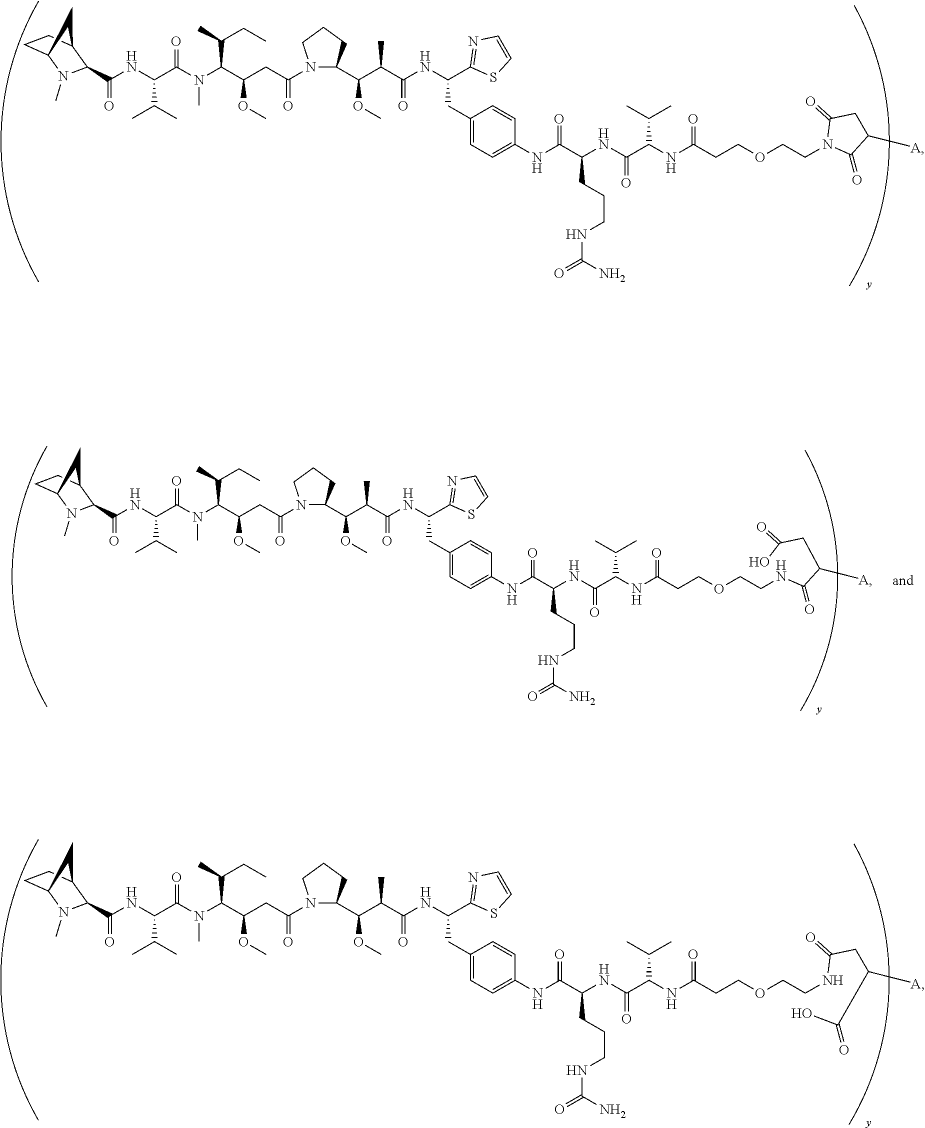

where y and A are as defined for conjugates of Formula (F) above.

Embodiment 126. The conjugate of Formula (F), Formula (F-1), Formula (F-3) and Formula (F-5) selected from:

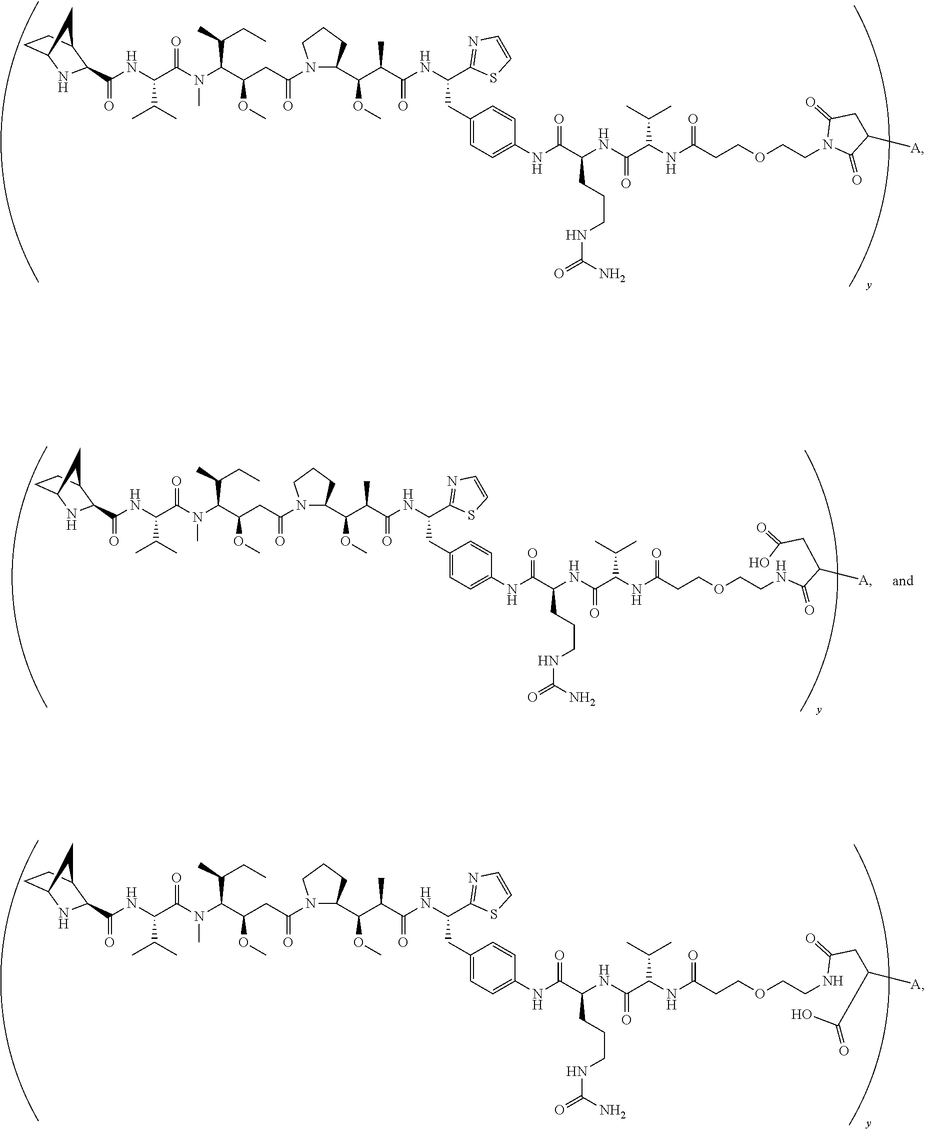

where y and A are as defined for conjugates of Formula (F) above.

Embodiment 127. The conjugate of Formula (G), Formula (G-1), Formula (G-2) and Formula (G-4) selected from:

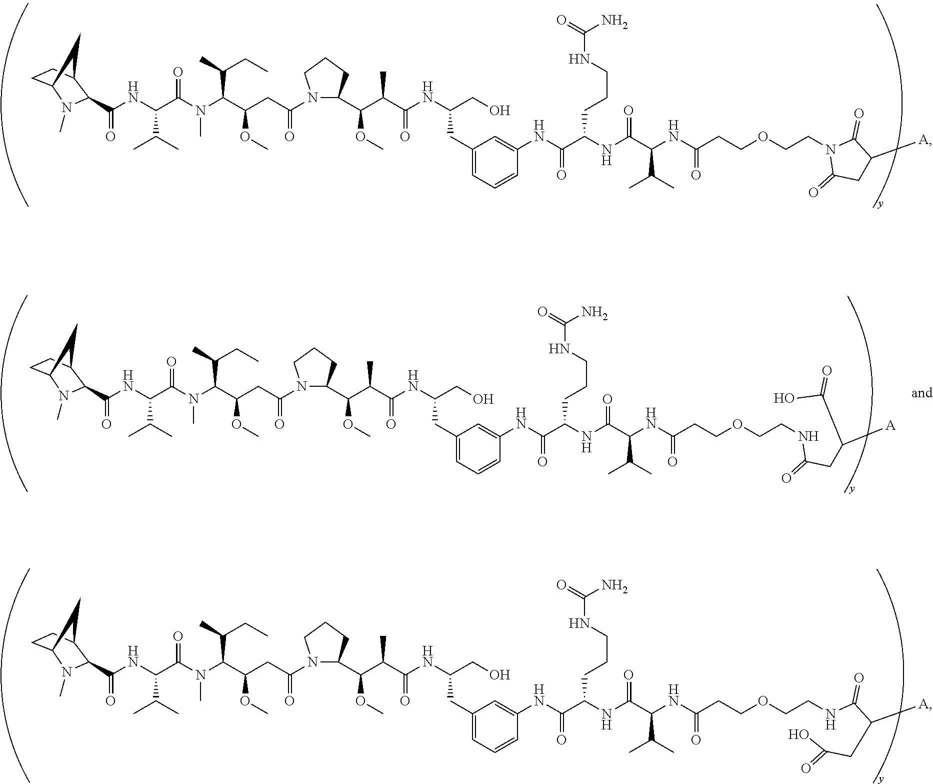

where y and A are as defined for conjugates of Formula (G) above.

Embodiment 128. The conjugate of Formula (G), Formula (G-1), Formula (G-3) and Formula (G-5) selected from:

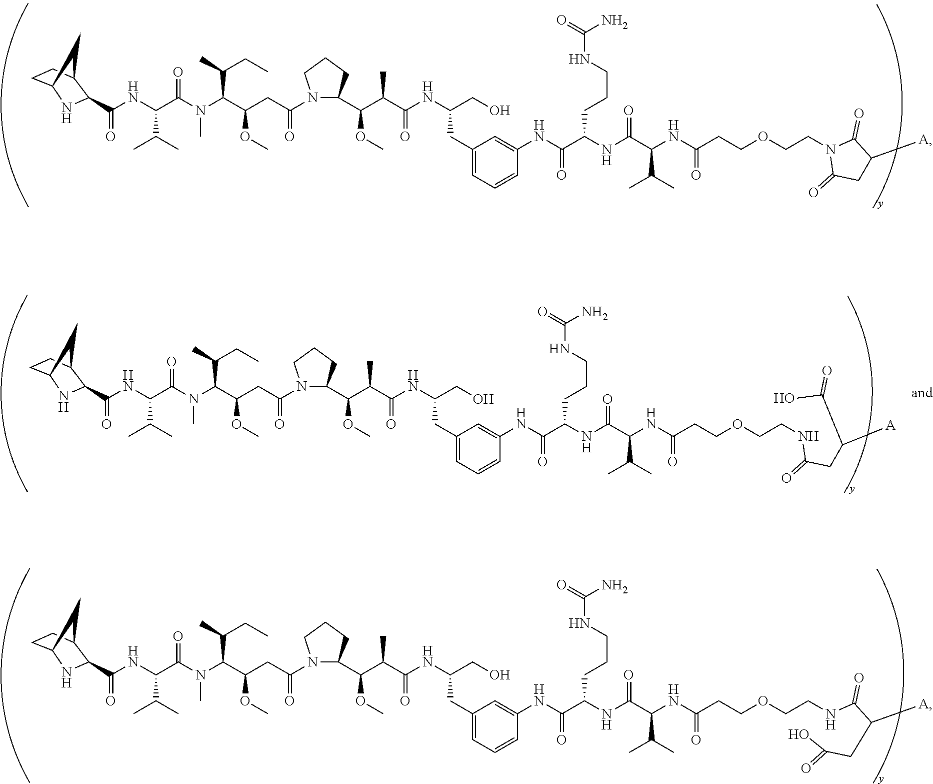

where y and A are as defined for conjugates of Formula (G) above.

Embodiment 129. The conjugate of Formula (H), Formula (H-1), Formula (H-2) and Formula (H-4) selected from:

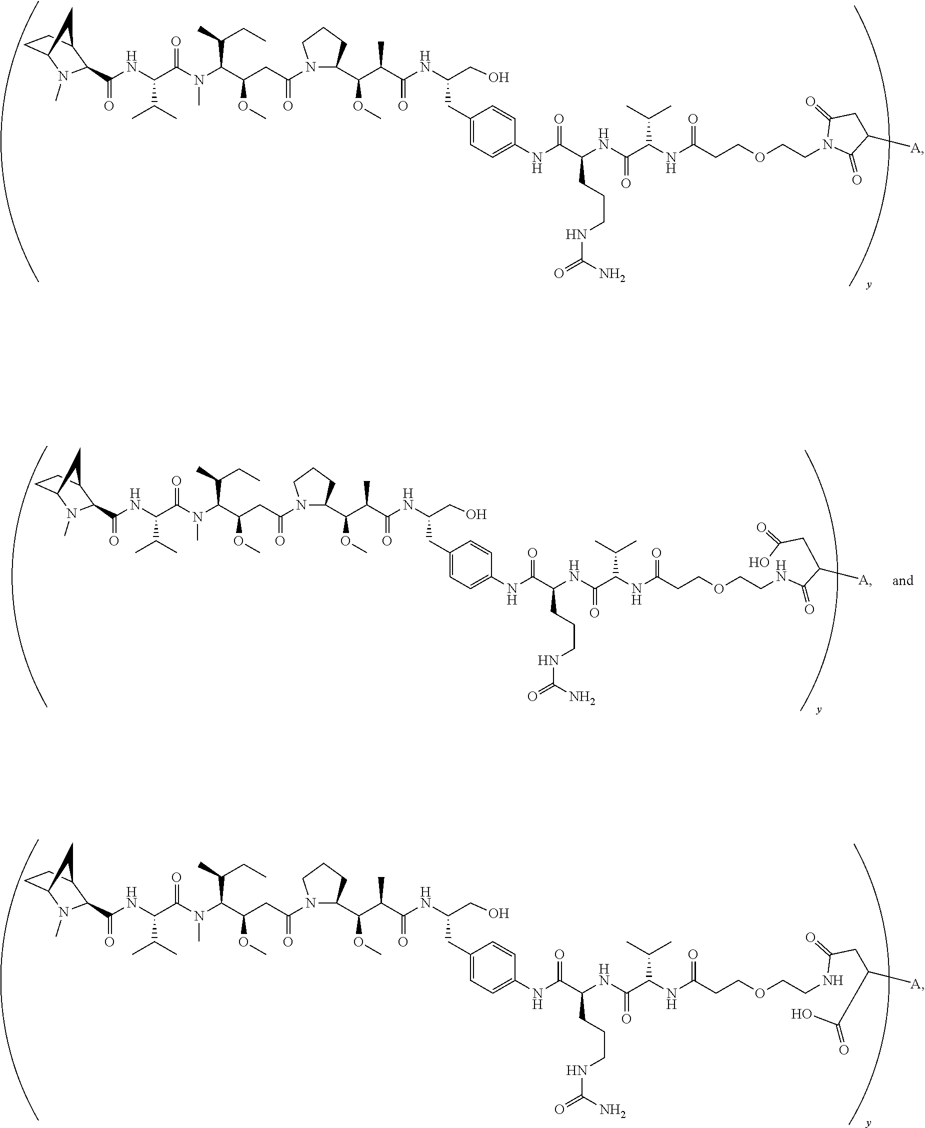

where y and A are as defined for conjugates of Formula (H) above.

Embodiment 130. The conjugate of Formula (H), Formula (H-1), Formula (H-3) and Formula (H-5) selected from:

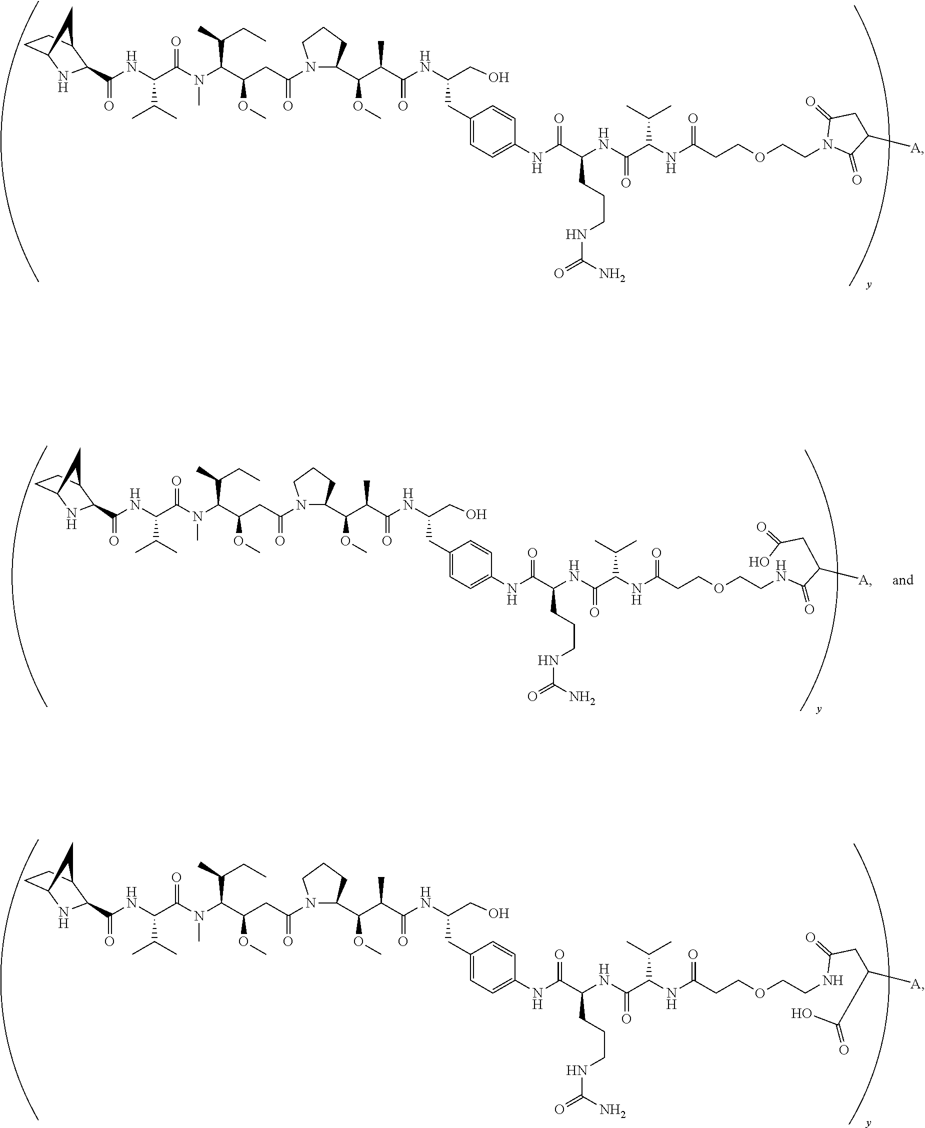

where y and A are as defined for conjugates of Formula (H) above.

The compounds of any of the Formulae disclosed herein, such as Formula (A), Formula (B), Formula (C), Formula (D), Formula (E), Formula (F), Formula (G) and Formula (H) can be produced using the methods described in the following examples. The following examples are intended to illustrate the invention and are not to be construed as being limitations thereon. Temperatures are given in degrees Celsius. If not mentioned otherwise, all evaporations are performed under reduced pressure, typically between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

### Abbreviations

| | |
|---|---|
| aq.: aqueous | Boc₂O: di-tert-butyl dicarbonate |
| br: broad | Cbz: Carboxybenzyl |
| d: doublet; dd: doublet of doublets | DCM: dichloromethane |
| m: multiplet | DEAD: Diethyl azodicarboxylate, |
| q: quartet | DIEA: N,N-Diisopropylethylamine |
| s: singlet | DIP-chloride ™: (+)-B- |
| | Chlorodiisopinocampheylborane |
| t: triplet | DIPEA: N,N-Diisopropylethylamine |
| h, hr: hour(s) | DMF: dimethyl formamide |
| ESI-MS: electrospray ionization mass | DMSO: dimethylsulfoxide |
| spectrometry | |
| HPLC: high pressure liquid chromatography | DPPA: Diphenyl phosphoryl azide |
| Isco, ISCO: Flash chromatography cartridge | EtOAc: ethyl acetate |
| containing silica gel provided by Teledyne Isco | |
| LC and LCMS: liquid chromatography and | EEDQ: 2-Ethoxy-1-ethoxycarbonyl- |
| liquid chromatography-mass spectrometry | 1,2-dihydroquinoline |
| MS: mass | HATU: 1- |
| | [Bis(dimethylamino)methylene]- |
| | 1H-1,2,3-triazolo[4,5-b]pyridinium 3- |
| | oxid hexafluorophosphate |
| min(s): minute(s) | MeCN: acetonitrile |
| m/z: mass to charge ratio | TFA: trifluoroacetic acid |
| M and mM: molar and millimolar | THF: tetrahydrofuran |
| mg: milligram | TMEDA: N,N,N′,N- |
| | Tetramethylethylenediamine |
| μL, mL and L: microliter, milliliter, and liter | RT and rt: room temperature |
| mmol and μmol: millimole and micromole | |
| NMR: nuclear magnetic resonance | |
| wt: weight | |

All starting materials, building blocks, reagents, acids, bases, dehydrating agents, solvents, and catalysts utilized to synthesis the compounds of the present invention are either commercially available or can be produced by organic synthesis methods known to one of ordinary skill in the art or can be produced by organic synthesis methods as described herein.

### Synthesis of Intermediates

### Synthesis of (S)-t-butyl (3-(2-amino-3-hydroxypropyl)phenyl)carbamate (i-1)

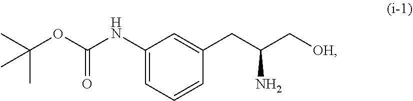 [SMILES: CC(C)(C)OC(=O)Nc1cccc(C[C@H](N)CO)c1]

Step 1: BH₃ in THE (1M, 10 ml) was added to (S)-2-((t-butoxycarbonyl)amino)-3-(3-nitrophenyl)propanoic acid (562 mg, 1.81 mmol) in THE (10 ml) with stirring at 0° C. Then the reaction was stirred at 50° C. for 1 h. The reaction mixture was cooled at 0° C., quenched with water, diluted with EtOAc and washed with 10% aqueous K₂CO₃, dried over MgSO4, filtered and concentrated. The crude was purified by a silica gel column (30-70% EtOAc-hexanes) to obtain (S)-t-butyl (1-hydroxy-3-(3-nitrophenyl)propan-2-yl)carbamate as a white solid. MS m/z 319.1 (M+Na). Retention time 1.183 minute. 1H NMR (600 MHz, Chloroform-d) δ 8.13-8.04 (m, 2H), 7.57 (d, J=7.7 Hz, 1H), 7.46 (dd, J=8.9, 7.6 Hz, 1H), 4.76 (s, 1H), 3.87 (dq, J=8.0, 4.6, 4.1 Hz, 1H), 3.69 (dd, J=10.9, 3.9 Hz, 1H), 3.58 (dd, J=10.8, 4.7 Hz, 1H), 2.97 (td, J=13.1, 12.5, 7.3 Hz, 2H), 1.37 (s, 9H).
Step 2: To (S)-t-butyl (1-hydroxy-3-(3-nitrophenyl)propan-2-yl)carbamate (0.31 g, 1.0 mmol) in acetonitrile (5 ml) was added 10% hydrochloric acid (5 ml). The reaction mixture was stirred at rt for 48 h and then concentrated to give (S)-2-amino-3-(3-nitrophenyl)propan-1-ol as HCl salt. MS m/z 197.2 (M+H). Retention time 0.775 min.
Step 3: (S)-2-Amino-3-(3-nitrophenyl)propan-1-ol HCl salt (0.243 g, 1.046 mmol) was dissolved in MeOH (10 ml) and 10% palladium on carbon (50 mg, 0.047 mmol) was added. A 2 L hydrogen balloon was attached. The reaction was flushed with H₂ three times and then stirred at rt for 1 h. LCMS indicated the reaction was complete. The reaction was filtered through a celite pad and concentrated to give (S)-2-amino-3-(3-aminophenyl)propan-1-ol as HCl salt. MS m/z 167.2 (M+H). Retention time 0.373 min.
Step 4: (S)-2-Amino-3-(3-aminophenyl)propan-1-ol HCl salt (0.212 g, 1.046 mmol) and Boc₂O (228 mg, 1.05 mmol) and dioxane-water-AcOH (10:9:1, 20 ml) were combined and stirred at rt for 3 days. LCMS indicated the reaction was 75% complete. Additional Boc₂O (150 mg) was added and the reaction was further stirred for 6 h. The reaction mixture was then concentrated and purified with preparative HPLC (10⁻⁴⁰% acetonitrile in water with 0.05% TFA) to give (S)-t-butyl (3-(2-amino-3-hydroxypropyl)phenyl)carbamate (i-1) as an oil. MS m/z 267.2 (M+H). Retention time 1.011 min.

### Synthesis of (3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoic Acid (i-2)

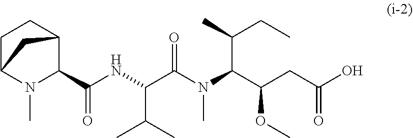

Step 1: Dil-OtBu HCl salt

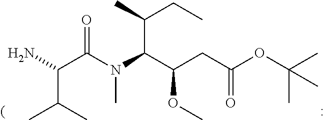

388 mg, 0.982 mmol), (1R,3S,4S)-2-(t-butoxycarbonyl)-2-azabicyclo[2.2.1]heptane-3-carboxylic acid

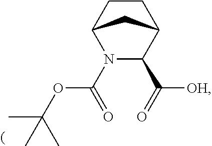 [SMILES: CC(C)(C)OC(=O)N1[C@@H]2CC[C@@H](C2)[C@H]1C(=O)O]

287 mg, 1.19 mmol), HATU (411 mg, 1.08 mmol) and DIEA (0.42 ml, 2.38 mmol) and DMF (5 ml) were combined and stirred at rt for 30 min. The reaction mixture was diluted with water (10 ml) and purified by RP-C18 ISCO to give tert-butyl (1R,3S,4S)-3-(((S)-1-(((3R,4S,5S)-1-(tert-butoxy)-3-methoxy-5-methyl-1-oxoheptan-4-yl)(methyl)amino)-3-methyl-1-oxobutan-2-yl)carbamoyl)-2-azabicyclo[2.2.1]heptane-2-carboxylate

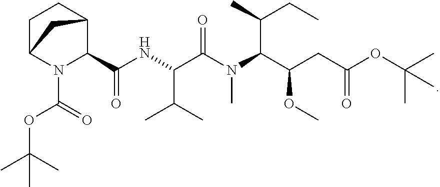

MS (m+1)=582.5, HPLC Peak RT=1.542 min
Step 2: The product obtained in step 1 (540 mg, 0.93 mmol) in 4M HCl in 1.4-dioxane (10 ml) was stirred at rt overnight. The reaction mixture was concentrated in to give (3R,4S,5S)-4-((S)-2-((1R,3S,4S)-2-azabicyclo[2.2.1]heptane-3-carboxamido)-N, 3-dimethylbutanamido)-3-methoxy-5-methylheptanoic acid,

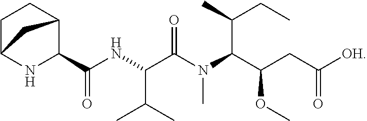

MS (m+1)=426.2, HPLC Peak RT=0.736 min
Step 3: The product obtained in step 2 (430 mg, 0.93 mmol), 37% formaldehyde solution (0.38 ml, 4.7 mmol), acetic acid (0.27 ml, 4.65 mmol), NaBH3CN (585 mg, 9.31 mmol) and MeOH (10 ml) were combined and stirred at rt for 30 min and then concentrated. The residue was purified by RP-C18 ISCO to give 450 mg of (3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoic acid (i-2), as a TFA salt. The TFA salt was treated with 10 ml of 12N HCl solution and concentrated twice to give (3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoic acid HCl salt. MS (m+1)=440.2, HPLC Peak RT=0.754 min.

### Synthesis of Dap-OMe: ((2R,3R)-methyl 3-methoxy-2-methyl-3-((S)-pyrrolidin-2-yl)propanoate) (i-3)

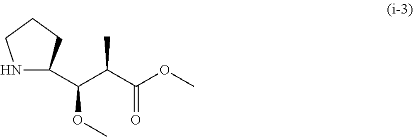

### Step 1: Boc-Dap-OH

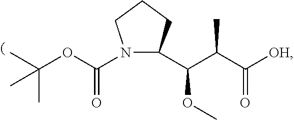 [SMILES: CO[C@H]([C@@H](C)C(=O)O)[C@@H]1CCCN1C(=O)OC(C)(C)C]

3.11 g, 10.8 mmol), K₂CO₃ (2.99 g, 21.6 mmol), iodomethane (2.95 g) and acetone (55 mL) were combined. The reaction was stirred at 20° C. for 2 h. An additonal methyliodide (2.28 g) was added to the reaction and the reaction was stirred at 40° C. for 3 h. The reaction mixture was concentrated. The residue was partitioned between 200 mL EtOAc and 100 mL H2O. The organic layer was separated, washed with 50 mL saturated aq NaCl, dryed over MgSO₄, filtered and concentrated, affording Boc-Dap-OMe,

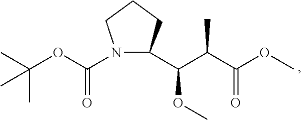 [SMILES: COC(=O)[C@H](C)[C@@H](OC)[C@@H]1CCCN1C(=O)OC(C)(C)C]

as a yellow oil. MS (ESI+) m/z calc 324.2, found 324.2 (M+23). Retention time 1.245 min.
Step 2: Boc-Dap-OMe (3.107 g, 10.3 mmol) was combined with HCl in diethyl ether (2 M, 10 mL) and concentrated. This operation was repeated. The reaction was complete after the 7^{th} treatment. HCl salt of Dap-OMe (i-3) was obtained as a white solid after being concentrated. MS (ESI+) m/z calc 202.1, found 202.2 (M+1). Retention time 0.486 min. ¹H NMR (400 MHz, CDCl₃): δ 4.065-4.041 (m, 1H), 3.732 (br.s, 1H), 3.706 (s, 3H), 3.615 (s, 3H), 3.368 (br.s, 1H), 3.314 (br.s, 1H), 2.795 (q, 1H, J=6.8 Hz), 2.085-1.900 (m, 4H), 1.287 (d, 3H, J=7.2 Hz).

### Synthesis of tert-butyl (S)-(3-(2-amino-2-(thiazo-2-yl)ethyl)phenyl)carbamate (i-4)

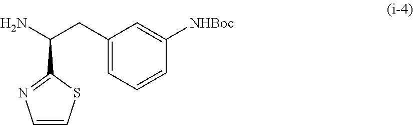 [SMILES: CC(C)(C)OC(=O)Nc1cccc(C[C@H](N)c2nccs2)c1]

Step 1: To a Solution of 2-(3-nitrophenyl)acetic acid (3 g, 16.56 mmol) in DMF (dry, 17 ml) was added HATU (6.93 g, 18.22 mmol), N,O-dimethylhydroxylamine hydrochloride (1.615 g, 16.56 mmol), and DIPEA (14.46 ml, 83 mmol) at RT. The reaction mixture was stirred overnight at RT. The reaction mixture was concentrated under high vacuum to remove most solvent. Then the residue was extracted between DCM and water. The aq. phase was extracted by DCM 2×. Combined DCM phases were concentrated under vacuum. The residue was separated by silica gel flash column (EtOAc/Heptane 0-70%, then 70%) to obtain 3.5 g N-methoxy-N-methyl-2-(3-nitrophenyl)acetamide as white solid. MS m/z 225.1 (M+1). Retention time 1.09 min. ¹H NMR (400 MHz, Chloroform-d) δ 8.28-8.09 (m, 2H), 7.67 (m, 1H), 7.63-7.46 (m, 1H), 3.90 (s, 2H), 3.74 (s, 3H), 3.24 (s, 3H).
Step 2: To a solution of TMEDA (2.63 mL, 17.39 mmol) in THE (dry, 30 ml) under N₂ atmosphere at −78° C. (Acetone-dry ice bath), was added dropwise n-butyllithium (2.5 M in hexane) (1.028 g, 16.06 mmol). Then at −78° C., 2-bromothiazole (2.63 g, 16.06 mmol) was also added dropwise to the reaction mixture. The reaction mixture was stirred at −78° C. for 1 h. The mixture of N-methoxy-N-methyl-2-(3-nitrophenyl)acetamide (3 g, 13.38 mmol) in THE (30 ml) was added dropwise to the reaction mixture at −78° C. The reaction mixture was stirred at −78° C. for 1 h, then at −10° C. (Acetone-ice bath) for 2 h. The reaction mixture was quenched by adding sat. KHSO₄ aq. solution, then extracted with EtOAc 3×. The combined EtOAc phases were dried over sat. NaCl, NaSO₄, and concentrated. The residue was separated by silica gel flash column (EtOAc/Heptane 0-30%, then 30%) to obtain 1.95 g 2-(3-nitrophenyl)-1-(thiazol-2-yl)ethan-1-one as light yellow oil. MS m/z 249.0 (M+1). Retention time 1.34 min. ¹H NMR (400 MHz, Chloroform-d) δ 8.33-8.22 (m, 1H), 8.17 (ddd, J=8.1, 2.3, 1.0 Hz, 1H), 8.10 (d, J=3.0 Hz, 1H), 7.79-7.67 (m, 2H), 7.54 (t, J=7.9 Hz, 1H), 4.62 (s, 2H).
Step 3: To the solution of (+)-DIP-Chloride™ (9.22 g, 28.8 mmol) in diethyl ether (7 ml) under N₂ atmosphere at 0° C. (ice-water bath) was added dropwise a solution of 2-(3-nitrophenyl)-1-(thiazol-2-yl)ethan-1-one (2.38 g, 9.59 mmol) in diethyl ether (37 ml). The reaction mixture was stirred at 0° C. for 24 h. Then the mixture was neutralized with 30 ml of (1:1) mixture of 10% NaOH and 30% H₂O₂ at 10° C. in a water-ice bath. The mixture was stirred for 1 h at RT. Then the mixture was diluted with water, extracted with EtOAc 3×. The combined EtOAc phases were washed with sat. K₂CO₃, sat NaCl, and dried over NaSO₄, and concentrated. The residue was separated by silica gel flash column (EtOAc/Heptane 0-60%, then 60%) to obtain 1.639 g (R)-2-(3-nitrophenyl)-1-(thiazol-2-yl)ethan-1-ol as light yellow solid. MS m/z 251.1 (M+1). Retention time 1.09 min. ¹H NMR (400 MHz, Chloroform-d) δ 8.33-8.07 (m, 2H), 8.07-7.80 (m, 1H), 7.74-7.55 (m, 1H), 7.55-7.36 (m, 2H), 5.55 (dd, J=7.9, 4.1 Hz, 1H), 4.48 (s, 1H), 3.53 (dd, J=13.9, 4.0 Hz, 1H), 3.32 (dd, J=13.9, 8.1 Hz, 1H). 92% e.e. determined by Chiral SFC.
Step 4: To a solution of (R)-2-(3-nitrophenyl)-1-(thiazol-2-yl)ethan-1-ol (1.636 g, 6.54 mmol) in MeOH (20 ml) was added Pd/C (10%, 0.696 g, 0.654 mmol). The reaction mixture was charged with H₂ (1 atm) after three vacuum/H₂ cycle, and stirred at RT. After overnight stirring, the reaction mixture was filtered through Celite and washed with MeOH. The filtrate was concentrated under vacuum to obtain 1.3 g (R)-2-(3-aminophenyl)-1-(thiazol-2-yl)ethan-1-ol as a solid which was directly used for the next step without further purification. MS m/z 221.1 (M+1). Retention time 0.50 min. ¹H NMR (400 MHz, DMSO-d₆) δ 7.72 (d, J=3.2 Hz, 1H), 7.59 (d, J=3.2 Hz, 1H), 6.88 (t, J=7.7 Hz, 1H), 6.46 (t, J=1.9 Hz, 1H), 6.42-6.29 (m, 2H), 6.14 (d, J=5.7 Hz, 1H), 5.03-4.78 (m, 3H), 3.03 (dd, J=13.7, 4.0 Hz, 1H), 2.72 (dd, J=13.7, 8.7 Hz, 1H).
Step 5: To the mixture of (R)-2-(3-aminophenyl)-1-(thiazol-2-yl)ethan-1-ol (1.3 g, 5.92 mmol) in Dioxane/Water (1/1, 16 ml/16 ml) was added Boc₂O (1.512 ml, 6.51 mmol) and NaOH (0.284 g, 7.10 mmol). The reaction mixture was stirred at RT overnight. The reaction mixture was added 10 ml water, then extracted with EtOAc (3*40 ml). The organic phases were combined, dried over Na₂SO₄, then concentrated under vacuum. The residue was then separated by silica gel flash column (EtOAc/Heptane 0 to 80% then 80%) to obtain 1.24 g tert-butyl (R)-(3-(2-hydroxy-2-(thiazol-2-yl)ethyl)phenyl)carbamate as solid. MS m/z 321.3 (M+1). Retention time 1.26 min. ¹H NMR (400 MHz, DMSO-d₆) δ 9.24 (s, 1H), 7.73 (d, J=3.2 Hz, 1H), 7.60 (d, J=3.3 Hz, 1H), 7.39 (t, J=1.8 Hz, 1H), 7.25 (ddd, J=8.2, 2.3, 1.1 Hz, 1H), 7.11 (t, J=7.8 Hz, 1H), 6.80 (dt, J=7.7, 1.2 Hz, 1H), 6.20 (d, J=5.7 Hz, 1H), 4.97 (ddd, J=8.6, 5.7, 4.0 Hz, 1H), 3.13 (dd, J=13.7, 4.0 Hz, 1H), 2.83 (dd, J=13.7, 8.7 Hz, 1H), 1.47 (s, 9H).
Step 6: To an ice-water bath cooled solution of tert-butyl (R)-(3-(2-hydroxy-2-(thiazol-2-yl)ethyl)phenyl)carbamate (1.2 g, 3.75 mmol) in THE (dry, 25 ml) under N₂ atmosphere, was added PPh₃ (1.670 g, 6.37 mmol). DEAD (40% wt in Toluene) (2.90 ml, 6.37 mmol) was then added dropwise at 0° C., followed by DPPA (1.372 ml, 6.37 mmol). Then the cold bath was removed. the reaction mixture was stirred at RT for overnight. The reaction mixture was concentrated under vacuum, and then subjected to flash silica gel column separation (EtOAc/Heptane 0 to 30%, then 30%) to obtain 1.03 g tert-butyl (S)-(3-(2-azido-2-(thiazol-2-yl)ethyl)phenyl)carbamate as oil. MS m/z 346.3 (M+1). Retention time 1.55 min. ¹H NMR (400 MHz, DMSO-d₆) δ 9.29 (s, 1H), 7.86 (d, J=3.2 Hz, 1H), 7.76 (d, J=3.2 Hz, 1H), 7.41 (t, J=1.9 Hz, 1H), 7.29 (ddd, J=8.3, 2.2, 1.1 Hz, 1H), 7.16 (t, J=7.8 Hz, 1H), 6.86 (dt, J=7.9, 1.2 Hz, 1H), 5.31 (dd, J=8.7, 5.7 Hz, 1H), 3.17 (d, J=5.3 Hz, 1H), 3.09 (dd, J=13.9, 8.7 Hz, 1H), 1.47 (s, 9H).
Step 7: To a solution of tert-butyl (S)-(3-(2-azido-2-(thiazol-2-yl)ethyl)phenyl)carbamate (861 mg, 2.493 mmol) in MeOH (4 ml) was added Pd/C (10% wet, 265 mg, 0.249 mmol). The reaction mixture was charged with H₂ (1 atm) after three vacuum/H₂ cycle, and stirred at RT. After overnight stirring, the reaction mixture was concentrated and then filtered through Celite and washed with MeOH. The filtrate was concentrated under vacuum. to obtain 781 mg tert-butyl (S)-(3-(2-amino-2-(thiazol-2-yl)ethyl)phenyl)carbamate (i-4) as sticky oil. MS m/z 320.2 (M+1). Retention time 0.91 min. ¹H NMR (400 MHz, DMSO-d₆) δ 9.26 (s, 1H), 7.71 (d, J=3.3 Hz, 1H), 7.55 (d, J=3.3 Hz, 1H), 7.36 (t, J=1.9 Hz, 1H), 7.26 (dt, J=8.3, 1.5 Hz, 1H), 7.13 (t, J=7.8 Hz, 1H), 6.78 (dt, J=7.6, 1.3 Hz, 1H), 4.31 (dd, J=8.7, 4.7 Hz, 1H), 3.14 (dd, J=21.2, 5.0 Hz, 1H), 2.73 (dd, J=13.4, 8.7 Hz, 1H), 2.11 (s, 2H), 1.47 (s, 9H).

### Synthesis of Exemplary Drug Moieties

### Example A: Synthesis of (1R,3S,4S)—N—((S)-1-(((3R,4S,5S)-1-((S)-2-((1R,2R)-3-(((S)-2-(3-aminophenyl)-1-(thiazol-2-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (C1)

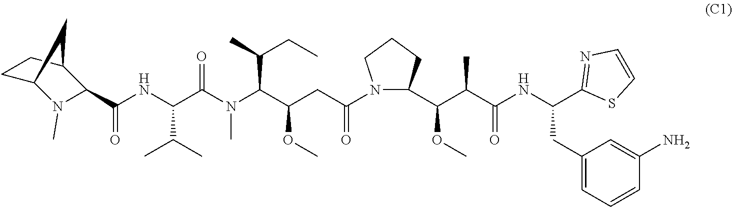 [SMILES: CC[C@H](C)[C@@H]([C@@H](CC(=O)N1CCC[C@H]1[C@H](OC)[C@@H](C)C(=O)N[C@@H](Cc1cccc(N)c1)c1nccs1)OC)N(C)C(=O)[C@@H](NC(=O)[C@@H]1[C@H]2CC[C@@H](C2)N1C)C(C)C]

Step 1: To a solution of (2R,3R)-3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoic acid (250 mg, 0.346 mmol) in DMF (4 ml) was added tert-butyl (S)-(3-(2-amino-2-(thiazol-2-yl)ethyl)phenyl)carbamate (i-4) (110 mg, 0.346 mmol), HATU (158 mg, 0.415 mmol), and DIPEA (362 μl, 2.075 mmol). The reaction mixture was stirred at RT overnight. The reaction mixture was concentrated under vacuum. The residue was then dissolved in MeOH, and was separated by ISCO gold C-18 100 gram reversed phase column (MeCN/H₂O 0-100%) to obtain 173 mg tert-butyl (3-((S)-2-((2R,3R)-3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-2-(thiazol-2-yl)ethyl)phenyl)carbamate as white powder. MS m/z 911.0 (M+1). Retention time 1.15 min.
Step 2: tert-butyl (3-((S)-2-((2R,3R)-3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-2-(thiazol-2-yl)ethyl)phenyl)carbamate (173 mg, 0.190 mmol) was dissolved in 1 ml Dioxane, then 10 ml 4N HCl in Dioxane was added to the mixture. The reaction mixture was stirred at room temperature for 30 min. The reaction mixture was concentrated under high vacumm, and then was partitioned between sat. NaHCO₃ and DCM to make the aq. phase pH as 8. The basic aq. phase was extracted with DCM 3×. The combined DCM phases were dried over sat NaCl and Na₂SO₄, and then concentrated under high vacuum to obtain 155 mg (1R,3S,4S)—N—((S)-1-(((3R,4S,5S)-1-((S)-2-((1R,2R)-3-(((S)-2-(3-aminophenyl)-1-(thiazol-2-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide as solid. MS m/z 810.5 (M+1). Retention time 0.90 min.

### Example B: Synthesis of (1R,3S,4S)—N—((S)-1-(((3R,4S,5S)-1-((S)-2-((1R,2R)-3-(((S)-1-(3-aminophenyl)-3-hydroxypropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (C2)

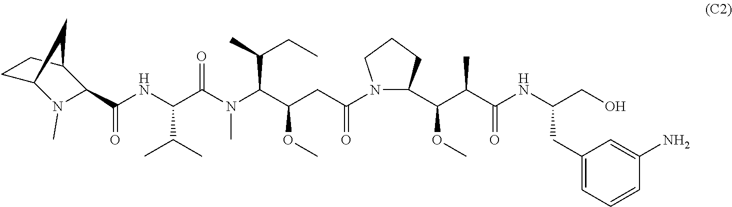 [SMILES: CC[C@H](C)[C@@H]([C@@H](CC(=O)N1CCC[C@H]1[C@H](OC)[C@@H](C)C(=O)N[C@H](CO)Cc1cccc(N)c1)OC)N(C)C(=O)[C@@H](NC(=O)[C@@H]1[C@H]2CC[C@@H](C2)N1C)C(C)C]

Step 1: DIEA (0.105 ml, 0.60 mmol) and HATU (45.5 mg, 0.12 mmol) were added to (3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoic acid (i-2) (57 mg, 0.12 mmol) in DMF (2 ml). The reaction mixture was stirred at rt for 5 min and then DapOMe (i-3) (28.5 mg, 0.12 mmol) in DMF (1 ml) was added. The reaction mixture was stirred at rt for 1 h and then purified by preparative HPLC (10⁻⁵⁰% acetonitrile-H₂O containing 0.05% TFA) to obtain (2R,3R)-methyl 3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoate. MS m/z 623.5 (M+H). Retention time 1.225 min.
Step 2: LiOH (30 mg, 1.25 mmol) was added to (2R,3R)-methyl 3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoate (43.2 mg, 0.059 mmol) in MeOH—H₂O (1:1, 4 ml). The reaction mixture was stirred at rt for 18 h, concentrated and acidified with HCl (1N, 1 ml). The crude was purified by preparative HPLC (10⁻³⁸% acetonitrile-H₂O containing 0.05% TFA) obtain (2R,3R)-3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoic acid as TFA salt. MS m/z 609.5 (M+H). Retention time 0.962 min.
Step 3: To (2R,3R)-3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanoic acid (45.7 mg, 0.063 mmol) in DMF (1 ml) were added DIEA (0.055 ml, 0.32 mmol) and HATU (24.0 mg, 0.063 mmol). The reaction mixture was stirred at rt for 10 min and then added to (S)-t-butyl (3-(2-amino-3-hydroxypropyl)phenyl)carbamate TFA salt (i-1) (24.1 mg, 0.063 mmol) in DMF (1 ml). The reaction mixture was stirred at rt for 1 h and then concentrated. The crude was purified by preparative HPLC (20-70% acetonitrile-H₂O containing 0.05% TFA) to obtain t-butyl (3-((S)-2-((2R,3R)-3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-3-hydroxypropyl)phenyl)carbamate as TFA salt. MS m/z 857.5 (M+H). Retention time 1.145 min.
Step 4: A solution of t-butyl (3-((S)-2-((2R,3R)-3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-3-hydroxypropyl)phenyl)carbamate (61.4 mg, 0.063 mmol) in acetonitrile-water (1:1, 4 ml) with 5% HCl was stirred at rt for 24 h. The reaction mixture was then concentrated and purified by preparative HPLC (10⁻³⁰% acetonitrile-H₂O containing 0.05% TFA) to give (1R,3S,4S)—N—((S)-1-(((3R,4S,5S)-1-((S)-2-((1R,2R)-3-(((S)-1-(3-aminophenyl)-3-hydroxypropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (C2) as TFA salt. MS m/z 757.5 (M+H). Retention time 0.744 min.

### Synthesis of Exemplary Linker-Drug Compounds

### Example C: Synthesis of (1R,3S,4S)—N—((S)-1-(((3R,4S,5S)-1-((S)-2-((1R,2R)-3-(((S)-2-(3-((S)-2-((S)-2-(3-(2-(2,5-dioxo-2,5-dihydro-1H-pyrrol-1-yl)ethoxy)propanamido)-3-methylbutanamido)-5-ureidopentanamido)phenyl)-1-(thiazol-2-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (LP1)

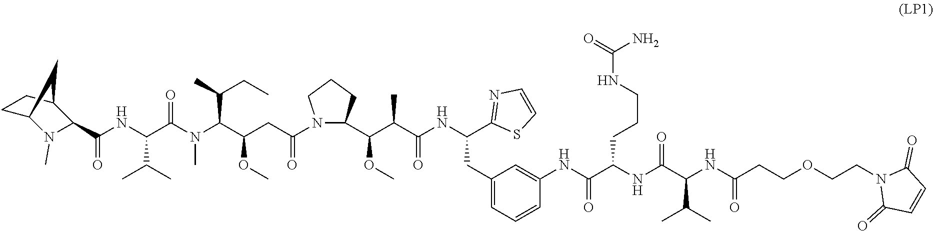 [SMILES: CC[C@H](C)[C@@H]([C@@H](CC(=O)N1CCC[C@H]1[C@H](OC)[C@@H](C)C(=O)N[C@@H](Cc1cccc(NC(=O)[C@H](CCCNC(N)=O)NC(=O)[C@@H](NC(=O)CCOCCN2C(=O)C=CC2=O)C(C)C)c1)c1nccs1)OC)N(C)C(=O)[C@@H](NC(=O)[C@@H]1[C@H]2CC[C@@H](C2)N1C)C(C)C]

Step 1: To the mixture of (1R,3S,4S)—N—((S)-1-(((3R,4S,5S)-1-((S)-2-((1R,2R)-3-(((S)-2-(3-aminophenyl)-1-(thiazol-2-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (C1) (154 mg, 0.190 mmol) and Boc-Val-Cit-OH (

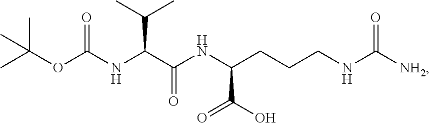 [SMILES: CC(C)[C@H](NC(=O)OC(C)(C)C)C(=O)N[C@@H](CCCNC(N)=O)C(=O)O]

92 mg, 0.247 mmol) in DCM (5 ml)/MeOH (0.1 ml) was added EEDQ (94 mg, 0.380 mmol). The reaction mixture was stirred at RT overnight. The reaction mixture was concentrated under vacuum. The residue was then dissolved in MeOH, and was separated by ISCO gold C-18 50 gram reversed phase column (MeCN/H₂O containing 0.05% TFA, 0-100%) to obtain 232 mg tert-butyl ((S)-1-(((S)-1-((3-((S)-2-((2R,3R)-3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-2-(thiazo-2-yl)ethyl)phenyl)amino)-1-oxo-5-ureidopentan-2-yl)amino)-3-methyl-1-oxobutan-2-yl)carbamate as TFA salt. MS m/z 1167.3 (M+1). Retention time 1.10 min.
Step 2: To tert-butyl ((S)-1-(((S)-1-((3-((S)-2-((2R,3R)-3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-2-(thiazol-2-yl)ethyl)phenyl)amino)-1-oxo-5-ureidopentan-2-yl)amino)-3-methyl-1-oxobutan-2-yl)carbamate (232 mg, 0.181 mmol) was added to a cold solution of TFA/DCM (25%, 6 ml) at 0° C. with ice-water bath, then the mixture was stirred at 0° C. for 15 min, then was allowed to warm to RT. The mixture was stirred at RT for 30 min. The reaction mixture was concentrated under vacuum. The residue was then dissolved in DMSO and was separated by ISCO gold C-18 50 gram reversed phase column (MeCN/H₂O containing 0.05% TFA, 0-100%) to obtain 219 mg ((1R,2R)-3-(((S)-2-(3-((S)-2-((S)-2-amino-3-methylbutanamido)-5-ureidopentanamido)phenyl)-1-(thiazol-2-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide as TFA salt. MS m/z 1067.2 (M+1). Retention time 0.88 min.
Step 3: ((1R,2R)-3-(((S)-2-(3-((S)-2-((S)-2-amino-3-methylbutanamido)-5-ureidopentanamido)phenyl)-1-(thiazol-2-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (219 mg 0.18 mmol) was dissolved in DMF (2 ml), then MAL-PEG1-NHS ester

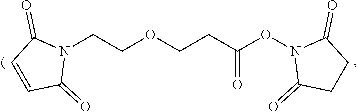 [SMILES: O=C(CCOCCN1C(=O)C=CC1=O)ON1C(=O)CCC1=O]

73.1 mg, 0.236 mmol) and DIPEA (190 μl, 1.087 mmol) were added. The reaction mixture was stirred at RT for 1 h. The reaction mixture was concentrated under vacuum. The residue was then dissolved in DMSO and was separated by ISCO gold C-18 50 gram reversed phase column (MeCN/H₂O containing 0.05% TFA, 0-100%) to obtain 174 mg (1R,3S,4S)—N—((S)-1-(((3R,4S,5S)-1-((S)-2-((1R,2R)-3-(((S)-2-(3-((S)-2-((S)-2-(3-(2-(2,5-dioxo-2,5-dihydro-1H-pyrrol-1-yl)ethoxy)propanamido)-3-methylbutanamido)-5-ureidopentanamido)phenyl)-1-(thiazol-2-yl)ethyl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (LP1) as TFA salt. MS m/z 1262.4 (M+1). Retention time 1.02 min.

### Example D: Synthesis of (1R,3S,4S)—N—((S)-1-(((3R,4S,5S)-1-((S)-2-((1R,2R)-3-(((S)-1-(3-((S)-2-((S)-2-(3-(2-(2,5-dioxo-2,5-dihydro-1H-pyrrol-1-yl)ethoxy)propanamido)-3-methylbutanamido)-5-ureidopentanamido)phenyl)-3-hydroxypropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (LP2)

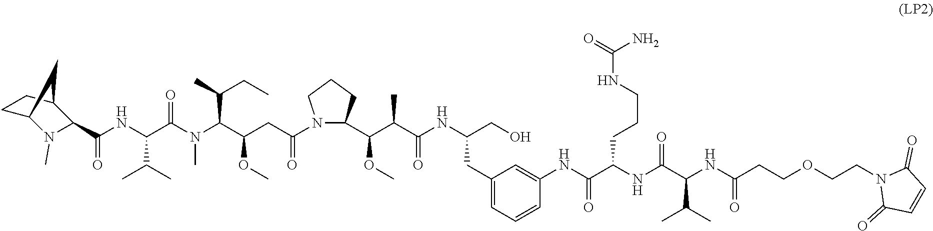 [SMILES: CC[C@H](C)[C@@H]([C@@H](CC(=O)N1CCC[C@H]1[C@H](OC)[C@@H](C)C(=O)N[C@H](CO)Cc1cccc(NC(=O)[C@H](CCCNC(N)=O)NC(=O)[C@@H](NC(=O)CCOCCN2C(=O)C=CC2=O)C(C)C)c1)OC)N(C)C(=O)[C@@H](NC(=O)[C@@H]1[C@H]2CC[C@@H](C2)N1C)C(C)C]

Step 1: To a solution of Fmoc-Cit-OH

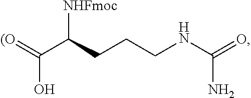

10.0 mg, 0.025 mmol) in DMF (1 ml) was added DIEA (13.0 mg, 0.10 mmol) and then HATU (9.6 mg, 0.025 mmol) and the reaction mixture was stirred at rt for 5 min and the solution was then added to (1R,3S,4S)—N—((S)-1-(((3R,4S,5S)-1-((S)-2-((1R,2R)-3-(((S)-1-(3-aminophenyl)-3-hydroxypropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (C2) (20 mg, 0.025 mmol). This reaction mixture was stirred at rt for 1 hour and then purified by reverse phase HPLC, using C18 column, eluted with 10⁻⁴⁵% acetonitrile-H₂O containing 0.05% TFA. The fractions containing the desired product were concentrated to obtain (9H-fluoren-9-yl)methyl ((S)-1-((3-((S)-2-((2R,3R)-3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-3-hydroxypropyl)phenyl)amino)-1-oxo-5-ureidopentan-2-yl)carbamate as TFA salt. LCMS MS m/z 1136.6 (M+1), Retention time 1.042 minutes.
Step 2: (9H-fluoren-9-yl)methyl ((S)-1-((3-((S)-2-((2R,3R)-3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-3-hydroxypropyl)phenyl)amino)-1-oxo-5-ureidopentan-2-yl)carbamate (31.5 mg, 0.025 mmol) TFA salt was dissolved in MeOH (1 mL). Then Pd/C (10 mg, 9.40 μmol) was added. A 2 L hydrogen balloon was attached and the reaction mixture was vacuum flushed three times with H₂ and then stirred under H₂ at rt for 30 min. The catalyst was then removed by filtration through celite and the mixture was concentrated and treated with 1N NaOH. The crude mixture was purified by reverse phase HPLC, using C18 column, eluted with 5-37% acetonitrile-H₂O containing 0.05% TFA. The fractions containing desired product were lyophilized to obtain (1R,3S,4S)—N—((S)-1-(((3R,4S,5S)-1-((S)-2-((1R,2R)-3-(((S)-1-(3-((S)-2-amino-5-ureidopentanamido)phenyl)-3-hydroxypropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide as TFA salt. LCMS m/z 914.6 (M+1), Retention time 0.773 min.
Step 3: To a solution of Cbz-Val-OH

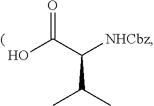

2.6 mg, 0.011 mmol) in DMF (1 ml) was added DIEA (0.011 ml, 0.061 mmol) and then HATU (3.86 mg, 0.011 mmol). The reaction mixture was stirred at rt for 5 min and then added to a solution of (1R,3S,4S)—N—((S)-1-(((3R,4S,5S)-1-((S)-2-((1R,2R)-3-(((S)-1-(3-((S)-2-amino-5-ureidopentanamido)phenyl)-3-hydroxypropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (11.6 mg, 0.011 mmol) TFA salt in DMF (1 ml). The reaction mixture was stirred at rt for 1 hour and then the crude was purified by reverse phase HPLC, using C18 column, eluted with 10⁻⁵⁰% acetonitrile-H₂O containing 0.05% TFA. The fractions containing desired product were lyophilized to obtain benzyl ((S)-1-(((S)-1-((3-((S)-2-((2R,3R)-3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-3-hydroxypropyl)phenyl)amino)-1-oxo-5-ureidopentan-2-yl)amino)-3-methyl-1-oxobutan-2-yl)carbamate as TFA salt. LCMS m/z 1147.6 (M+1), Retention time 0.986 min.
Step 4: Benzyl ((S)-1-(((S)-1-((3-((S)-2-((2R,3R)-3-((S)-1-((3R,4S,5S)-4-((S)—N, 3-dimethyl-2-((1R,3S,4S)-2-methyl-2-azabicyclo[2.2.1]heptane-3-carboxamido)butanamido)-3-methoxy-5-methylheptanoyl)pyrrolidin-2-yl)-3-methoxy-2-methylpropanamido)-3-hydroxypropyl)phenyl)amino)-1-oxo-5-ureidopentan-2-yl)amino)-3-methyl-1-oxobutan-2-yl)carbamate (7.7 mg, 0.006 mmol) TFA salt was dissolved in MeOH (2 ml) and then Pd/C (5 mg, 4.70 μmol) was added. A 2 L hydrogen balloon was attached and the reaction mixture was vacuum flushed with H₂ three times and then stirred under H₂ for 30 mins. LCMS indicated the reaction was complete. The catalyst was then removed by filtration through celite and the mixture was then concentrated to give (1R,3S,4S)—N—((S)-1-(((3R,4S,5S)-1-((S)-2-((1R,2R)-3-(((S)-1-(3-((S)-2-((S)-2-amino-3-methylbutanamido)-5-ureidopentanamido)phenyl)-3-hydroxypropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide as TFA salt. LCMS m/z 1013.6 (M+1) Retention time 0.774 min.
Step 5: To a solution of Mal-PEG1-acid

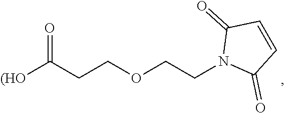

1.0 mg, 0.005 mmol) in DMF (0.5 ml) was added DIEA (2.8 mg, 0.022 mmol) and then HATU (1.8 mg, 0.005 mmol). The reaction was stirred at rt for 5 min and then added to a solution of (1R,3S,4S)—N—((S)-1-(((3R,4S,5S)-1-((S)-2-((1R,2R)-3-(((S)-1-(3-((S)-2-((S)-2-amino-3-methylbutanamido)-5-ureidopentanamido)phenyl)-3-hydroxypropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide (4.8 mg, 0.005 mmol) TFA salt in DMF (1 ml). The reaction was stirred at rt for 1 hour and then the crude was purified by reverse phase HPLC, using C18 column, eluted with 10⁻³⁸% acetonitrile-H₂O containing 0.05% TFA. The fractions containing desired product were lyophilized to obtain (1R,3S,4S)—N—((S)-1-(((3R,4S,5S)-1-((S)-2-((1R,2R)-3-(((S)-1-(3-((S)-2-((S)-2-(3-(2-(2,5-dioxo-2,5-dihydro-1H-pyrrol-1-yl)ethoxy)propanamido)-3-methylbutanamido)-5-ureidopentanamido)phenyl)-3-hydroxypropan-2-yl)amino)-1-methoxy-2-methyl-3-oxopropyl)pyrrolidin-1-yl)-3-methoxy-5-methyl-1-oxoheptan-4-yl)(methyl)amino)-3-methyl-1-oxobutan-2-yl)-2-methyl-2-azabicyclo[2.2.1]heptane-3-carboxamide as TFA salt (LP-2). LCMS m/z 1208.5 (M+1) Retention time 0.882 min. 3. Conjugation and Preparation of ADCs

### Processes for Making Antibody Conjugate of Formula (I)

A general reaction scheme for the formation of conjugates of Formula (I) is shown in Scheme 1 below:

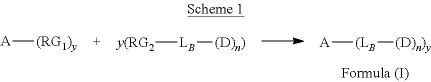

where: RG₁ is a reactive group, by way of example only a thiol or amine or ketone, which reacts with a compatible reactive group, RG₂, attached to the linker-drug moiety thereby covalently linking antibody fragment, A, to one or more linker-drug moieties. A non-limiting examples of such reactions of RG₁ and RG₂ groups is a maleimide (RG₂) reacting with a thiol (RG₁) to give a succinimide ring, or a hydroxylamine (RG₂) reacting with a ketone (RG₁) to give an oxime.

A general reaction scheme for the formation of conjugates of Formula (II) is shown in Scheme 2 below:

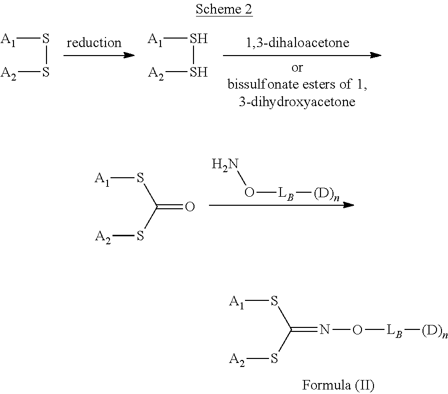

where: A₁, A₂, L_{B}, D and n are as defined herein, the 1,3-dihaloacetone is selected from 1,3-dichloroacetone, 1,3-dibromoacetone, and 1,3-diiodoacetone, and the reduction step is accomplished using a reducing agent selected from dithiothreitol (DTT) and Tris(2-carboxyethyl)phosphine hydrochloride (TCEP-HCl).

### Conjugation and Preparation of ADCs

### Processes for Making Antibody Conjugate of Formula (I)

A general reaction scheme for the formation of conjugates of Formula (I) is shown in Scheme 1 below:

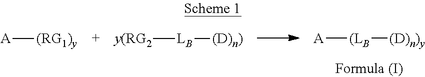

where: RG₁ is a reactive group, by way of example only a thiol or amine or ketone, which reacts with a compatible reactive group, RG₂, attached to the linker-drug moiety thereby covalently linking antibody fragment, A, to one or more linker-drug moieties. A non-limiting examples of such reactions of RG₁ and RG₂ groups is a maleimide (RG₂) reacting with a thiol (RG₁) to give a succinimide ring, or a hydroxylamine (RG₂) reacting with a ketone (RG₁) to give an oxime.

A general reaction scheme for the formation of conjugates of Formula (II) is shown in Scheme 2 below:

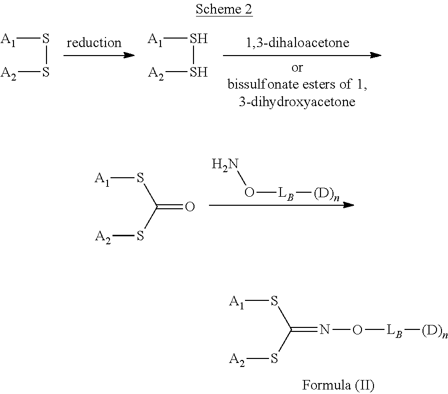

where: A₁, A₂, L_{B}, D and n are as defined herein, the 1,3-dihaloacetone is selected from 1,3-dichloroacetone, 1,3-dibromoacetone, and 1,3-diiodoacetone, and the reduction step is accomplished using a reducing agent selected from dithiothreitol (DTT) and Tris(2-carboxyethyl)phosphine hydrochloride (TCEP-HCl).

A general reaction scheme for the formation of conjugates of Formula (E) is shown in Scheme 3 below:

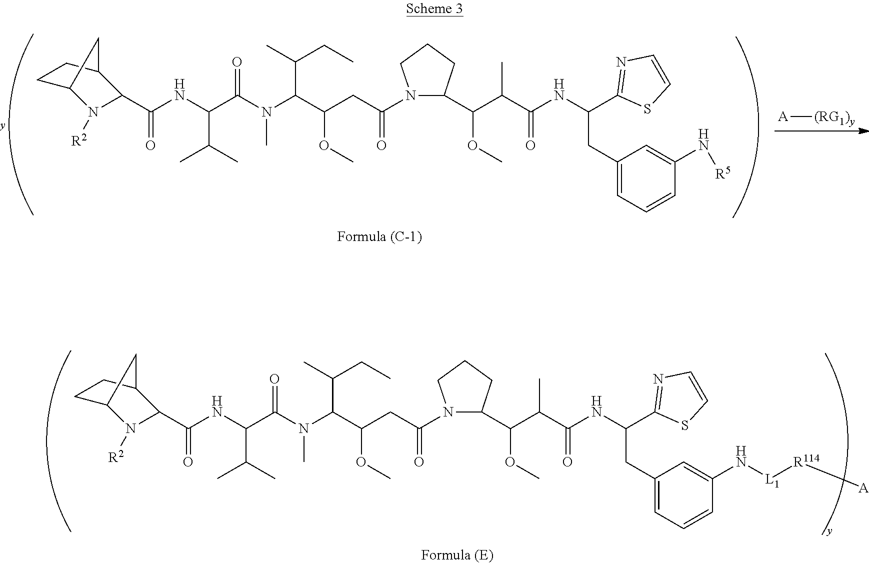

where: R⁵ is -L₁R¹⁴, -L₁R²⁴, -L₁R³⁴ or -L₁R⁴⁴ and RG₁ is a reactive group, by way of example only a thiol or amine or ketone, which reacts with a compatible R¹⁴, R²⁴, R³⁴ or R⁴⁴ group of a compound of Formula (C-1) to form a corresponding R¹¹⁴ group. By way of example, a maleimide reacting with a thiol to give a succinimide ring, or a hydroxylamine reacting with a ketone to give an oxime. A, y, L₁, R², R⁵ and R¹¹⁴ are as defined herein.

A general reaction scheme for the formation of conjugates of Formula (F) is shown in Scheme 4 below:

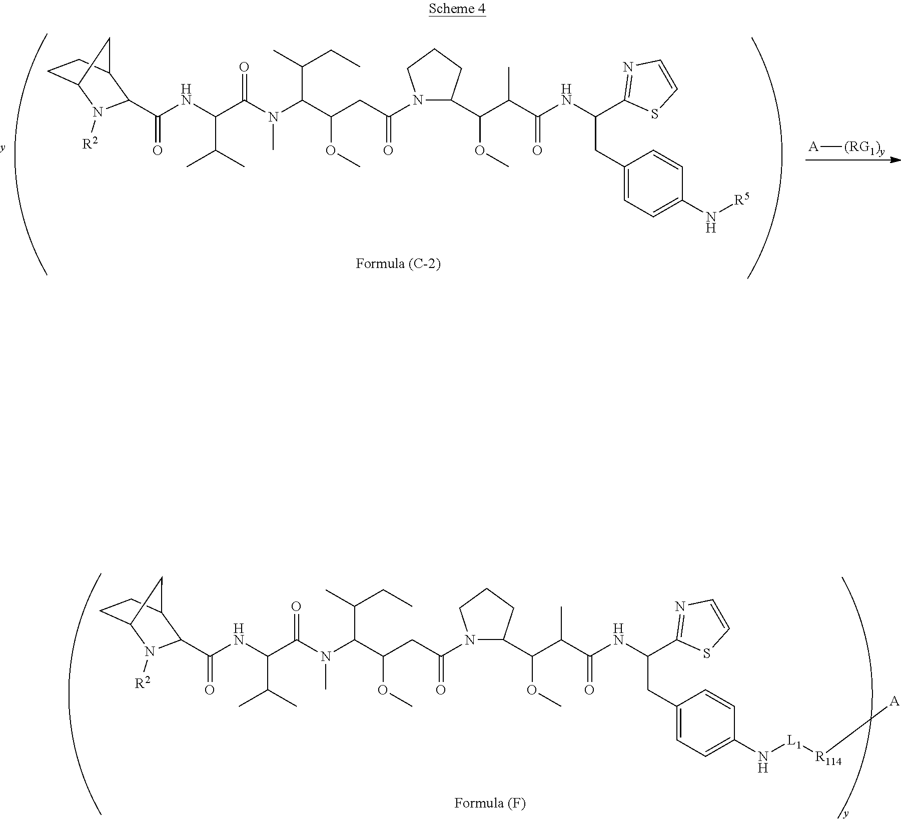

where: R⁵ is -L₁R¹⁴, -L₁R²⁴, -L₁R³⁴ or -L₁R⁴⁴ and RG₁ is a reactive group, by way of example only a thiol or amine or ketone, which reacts with a compatible R¹⁴, R²⁴, R³⁴ or R⁴⁴ group of a compound of Formula (C-2) to form a corresponding R¹¹⁴ group. By way of example, a maleimide reacting with a thiol to give a succinimide ring, or a hydroxylamine reacting with a ketone to give an oxime. A, y, L₁, R², R⁵ and R¹¹⁴ are as defined herein.

A general reaction scheme for the formation of conjugates of Formula (G) is shown in Scheme 5 below:

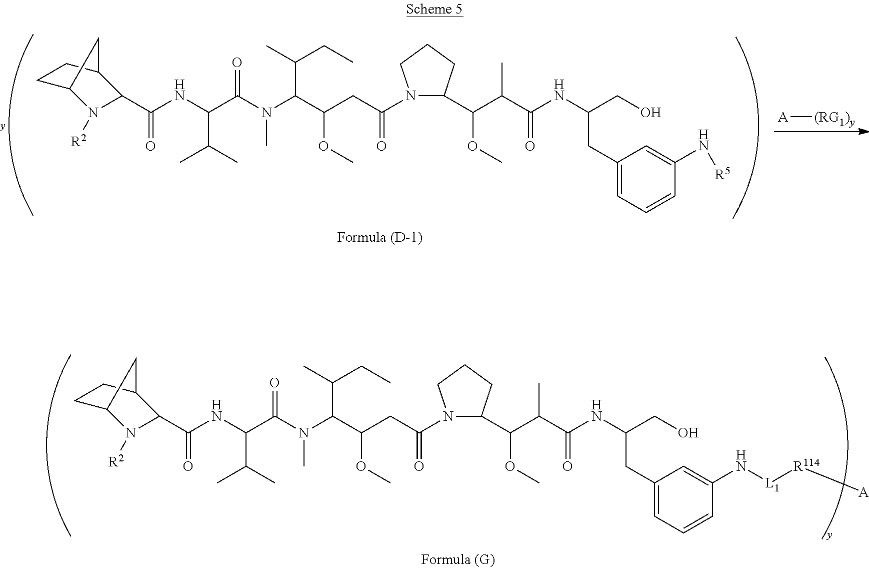

where: R⁵ is -L₁R¹⁴, -L₁R²⁴, -L₁R³⁴ or -L₁R⁴⁴ and RG₁ is a reactive group, by way of example only a thiol or amine or ketone, which reacts with a compatible R¹⁴, R²⁴, R³⁴ or R⁴⁴ group of a compound of Formula (D-1) to form a corresponding R¹¹⁴ group. By way of example, a maleimide reacting with a thiol to give a succinimide ring, or a hydroxylamine reacting with a ketone to give an oxime. A, y, L₁, R², R⁵ and R¹¹⁴ are as defined herein.

A general reaction scheme for the formation of conjugates of Formula (H) is shown in Scheme 6 below:

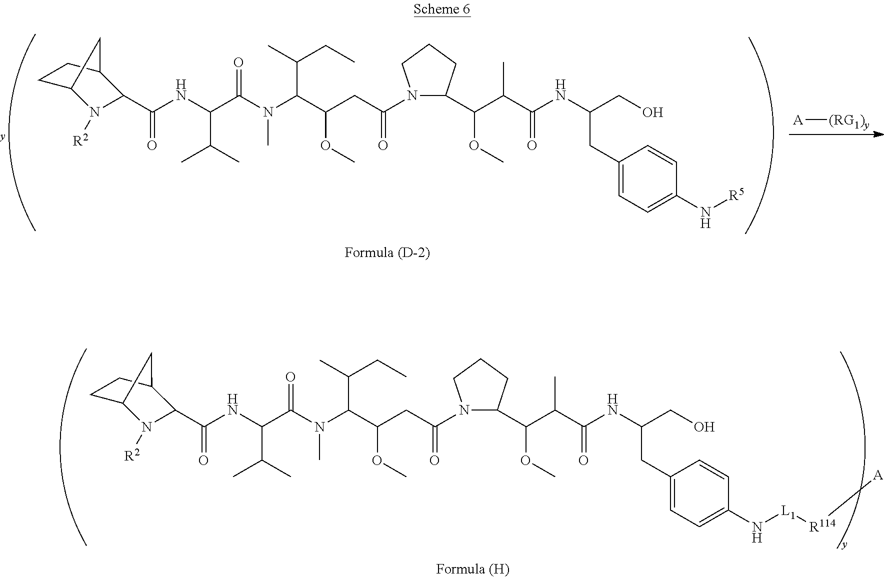

where: R⁵ is -L₁R¹⁴, -L₁R²⁴, -L₁R³⁴ or -L₁R⁴⁴ and RG₁ is a reactive group, by way of example only a thiol or amine or ketone, which reacts with a compatible R¹⁴, R²⁴, R³⁴ or R⁴⁴ group of a compound of Formula (D-2) to form a corresponding R¹¹⁴ group. By way of example, a maleimide reacting with a thiol to give a succinimide ring, or a hydroxylamine reacting with a ketone to give an oxime. A, y, L₁, R², R⁵ and R¹¹⁴ are as defined herein.

### 4. Characterization and Selection of Desirable Anti-cKIT ADCs

### Determination of DAR and Aggregation of the ADCs

DAR value of the cKIT ADC was evaluated by liquid chromatography-mass spectrometry (LC-MS). A compound-to-antibody ratio was extrapolated from LC-MS data for reduced and deglycosylated (when appropriate, i.e. when Fc is included) samples. LC-MS allows quantitation of the average number of molecules of linker-payload (compound) attached to an antibody in a conjugate sample.

Antibody drug conjugates of the invention were evaluated using analytical methods. Such analytical methodology and results can demonstrate that the conjugates have favorable properties, for example properties that would make them easier to manufacture, easier to administer to patients, more efficacious, and/or potentially safer for patients. One example is the determination of molecular size by size exclusion chromatography (SEC) wherein the amount of desired antibody species in a sample is determined relative to the amount of high molecular weight contaminants (e.g., dimer, multimer, or aggregated antibody) or low molecular weight contaminants (e.g., antibody fragments, degradation products, or individual antibody chains) present in the sample. In general, it is desirable to have higher amounts of monomer and lower amounts of, for example, aggregated antibody due to the impact of, for example, aggregates on other properties of the antibody sample such as but not limited to clearance rate, immunogenicity, and toxicity. A further example is the determination of the hydrophobicity by hydrophobic interaction chromatography (HIC) wherein the hydrophobicity of a sample is assessed relative to a set of standard antibodies of known properties. In general, it is desirable to have low hydrophobicity due to the impact of hydrophobicity on other properties of the antibody sample such as but not limited to aggregation, aggregation overtime, adherence to surfaces, hepatotoxicity, clearance rates, and pharmacokinetic exposure. See Damle, N. K., Nat Biotechnol. 2008; 26(8):884-885; Singh, S. K., Pharm Res. 2015; 32(11):3541-71.

### Selection of Anti-cKIT ADCs

To select anti-cKIT ADCs suitable for using in the methods described herein, an in vitro human hematopoietic stem cell killing assay can be used to screen the anti-cKIT ADCs for their efficacy and potency. For example, the methods described in Example 5 can be used to screen anti-cKIT ADCs. Suitable anti-cKIT ADCs can be selected based on EC50, e.g., anti-cKIT ADC with an EC50 less than 500 μg/ml, e.g., less than 100 μg/ml, less than 50 μg/ml, less than 10 μg/ml, or less than 5 g/ml.

Furthermore, it has been reported that cKIT expresses on mast cells, and stem-cell factor (SCF), the ligand of cKIT, induces direct degranulation of rat peritoneal mast cells in vitro and in vivo (Taylor et al., Immunology. 1995 November; 86(3):427-33). SCF also induces human mast cell degranulation in vivo (Costa et al., J Exp Med. 1996; 183(6): 2681-6). To avoid potential detrimental effects caused by mast cell degranulation in transplant recipients, selected cKIT ADCs can be tested for their ability to induce mast cell degranulation in vitro. For example, experiments described in Example 6 can be used to screen cKIT ADCs, and suitable anti-cKIT ADCs can be selected based on minimal mast cell degranulation, e.g., a baseline corrected O.D. readout of less than 0.25, e.g., less than 0.2, less than 0.15, or less than 0.1, in a beta-hexosaminidase release assay.

cKIT Antibody and Antibody Fragments

The present disclosure provides for antibodies or antibody fragments (e.g., antigen binding fragments) that specifically bind to human cKIT. Antibodies or antibody fragments (e.g., antigen binding fragments) of the present disclosure include, but are not limited to, the human monoclonal antibodies or fragments thereof described below.

In some embodiments, the presently disclosed anti-cKIT antibodies or antibody fragments (e.g., antigen binding fragments) have a reduced ability for causing mast cell degranulation, even when cross-linked and/or multimerized into larger complexes, in comparison to a full-length anti-cKIT antibody. In some embodiments, the anti-cKIT antibodies or antibody fragments (e.g., antigen binding fragments) disclosed herein are modified to have reduced ability to induce mast cell degranulation, even when cross-linked and/or multimerized into larger complexes. For example, the anti-cKIT antibodies or antibody fragments (e.g., antigen binding fragments) disclosed herein are modified to have an reduced ability to induce mast cell degranulation that is, is about, or is at least 10%, 20%, 30%, 40%, 50%, 60%, 70%, 80%, 90% reduced in comparison to a full-length anti-cKIT antibody, or an F(ab′)₂ or an F(ab)₂ fragment thereof, even when cross-linked and/or multimerized into larger complexes. In some embodiments, the anti-cKIT antibodies or antibody fragments (e.g., antigen binding fragments) disclosed herein may comprise an anti-cKIT Fab or Fab′ fragment. In some embodiments, the anti-cKIT antibodies or antibody fragments (e.g., antigen binding fragments) disclosed herein may have minimal ability to induce mast cell degranulation, e.g., a baseline corrected O.D. readout of less than 0.25, e.g., less than 0.2, less than 0.15, or less than 0.1, in a beta-hexosaminidase release assay, even when cross-linked and/or multimerized into larger complexes.

The antibody drug conjugates provided herein include a human cKIT-binding antibody fragment (e.g., Fab or Fab′). In some embodiments, antibody drug conjugates provided herein include a human or humanized antibody fragment (e.g., Fab or Fab′) that specifically binds to human cKIT. In some embodiments, antibody drug conjugates provided herein include a human or humanized Fab′ that specifically binds to human cKIT. In some embodiments, antibody drug conjugates provided herein include a human or humanized Fab that specifically binds to human cKIT.

In some embodiments, the antibody or antibody fragment (e.g., Fab or Fab′) that specifically binds to human cKIT comprises a VH domain having an amino acid sequence of any VH domain described in Table 1 (e.g., SEQ ID NO: 10, 36, 54, 69, 95). Other suitable antibody or antibody fragment (e.g., Fab or Fab′) can include a VH domain that has at least 80, 85, 90, 95, 96, 97, 98, or 99 percent sequence identity to any of the VH domains described in Table 1.

In some embodiments, the antibody or antibody fragment (e.g., Fab or Fab′) that specifically binds to human cKIT comprises a VH CDR (or HCDR) having an amino acid sequence of any one of the VH CDRs (or HCDR) listed in Table 1. In particular aspects, the present disclosure provides the antibody or antibody fragment (e.g., Fab or Fab′) comprising (or alternatively, consisting of) one, two, three, four, five or more VH CDRs (or HCDR) having an amino acid sequence of any of the VH CDRs (or HCDR) listed in Table 1.

In some embodiments, the antibody or antibody fragment (e.g., Fab or Fab′) that specifically binds to human cKIT comprises a VL domain having an amino acid sequence of any VL domain described in Table 1 (e.g., SEQ ID NO: 23, 47, 82, 108). Other suitable the antibody or antibody fragment (e.g., Fab or Fab′) can include a VL domain that has at least 80, 85, 90, 95, 96, 97, 98, or 99 percent sequence identity to any of the VL domains described in Table 1.

In some embodiments, the antibody or antibody fragment (e.g., Fab or Fab′) that specifically binds to human cKIT comprises a VL CDR (or LCDR) having an amino acid sequence of any one of the VL CDRs (or LCDR) listed in Table 1. In particular aspects, the present disclosure provides the antibody or antibody fragment (e.g., Fab or Fab′) comprising (or alternatively, consisting of) one, two, three, four, five or more VL CDRs (or LCDR) having an amino acid sequence of any of the VL CDRs (or LCDR) listed in Table 1.

Other anti-cKIT antibody or antibody fragment (e.g., Fab or Fab′) disclosed herein include amino acids that have been mutated, yet have at least 60, 70, 80, 90 or 95 percent sequence identity in the CDR regions with the CDR regions depicted in the sequences described in Table 1. In some aspects, it includes mutant amino acid sequences wherein no more than 1, 2, 3, 4 or amino acids have been mutated in the CDR regions when compared with the CDR regions depicted in the sequence described in Table 1.

The present disclosure also provides nucleic acid sequences that encode VH, VL, the heavy chain, and the light chain of the antibody or antibody fragment (e.g., Fab or Fab′) that specifically binds to human cKIT. Such nucleic acid sequences can be optimized for expression in mammalian cells.

TABLE 1
Sequences of exemplary anti-cKIT antibodies and antibody fragments
Anti-cKIT Ab1/Fab1/Fab′1

| | | |
|---|---|---|
| SEQ ID NO: 1 | HCDR1 (Kabat) | SYAIS |
| SEQ ID NO: 2 | HCDR2 (Kabat) | VIFPAEGAPGYAQKFQG |
| SEQ ID NO: 3 | HCDR3 (Kabat) | GGYISDFDV |
| SEQ ID NO: 4 | HCDR1 (Chothia) | GGTFSSY |
| SEQ ID NO: 5 | HCDR2 (Chothia) | FPAEGA |
| SEQ ID NO: 3 | HCDR3 (Chothia) | GGYISDFDV |
| SEQ ID NO: 6 | HCDR1 (Combined) | GGTFSSYAIS |
| SEQ ID NO: 2 | HCDR2 (Combined) | VIFPAEGAPGYAQKFQG |
| SEQ ID NO: 3 | HCDR3 (Combined) | GGYISDFDV |
| SEQ ID NO: 7 | HCDR1 (IMGT) | GGTFSSYA |
| SEQ ID NO: 8 | HCDR2 (IMGT) | IFPAEGAP |
| SEQ ID NO: 9 | HCDR3 (IMGT) | ARGGYISDFDV |
| SEQ ID NO: 10 | VH | QVQLVQSGAEVKKPGSSVKVSCKASGGTFSSYAI |
| | | SWVRQAPGQGLEWMGVIFPAEGAPGYAQKFQG |
| | | RVTITADESTSTAYMELSSLRSEDTAVYYCARGG |
| | | YISDFDVWGQGTLVTVSS |
| SEQ ID NO: 11 | VH DNA | CAGGTGCAATTGGTGCAGAGCGGTGCCGAAGT |
| | | GAAAAAACCGGGCAGCAGCGTGAAAGTTAGCT |
| | | GCAAAGCATCCGGAGGGACGTTTAGCAGCTAT |
| | | GCGATTAGCTGGGTGCGCCAGGCCCCGGGCC |
| | | AGGGCCTCGAGTGGATGGGCGTTATCTTCCCG |
| | | GCTGAAGGCGCTCCGGGTTACGCCCAGAAATT |
| | | TCAGGGCCGGGTGACCATTACCGCCGATGAAA |
| | | GCACCAGCACCGCCTATATGGAACTGAGCAGC |
| | | CTGCGCAGCGAAGATACGGCCGTGTATTATTG |
| | | CGCGCGTGGTGGTTACATCTCTGACTTCGATG |
| | | TTTGGGGCCAAGGCACCCTGGTGACTGTTAGC |
| | | TCA |
| SEQ ID NO: 12 | Ab HC | QVQLVQSGAEVKKPGSSVKVSCKASGGTFSSYAI |
| | | SWVRQAPGQGLEWMGVIFPAEGAPGYAQKFQG |
| | | RVTITADESTSTAYMELSSLRSEDTAVYYCARGG |
| | | YISDFDVWGQGTLVTVSSASTKGPSVFPLAPSSK |
| | | STSGGTAALGCLVKDYFPEPVTVSWNSGALTSG |
| | | VHTFPAVLQSSGLYSLSSVVTVPSSSLGTQTYICN |
| | | VNHKPSNTKVDKRVEPKSCDKTHTCPPCPAPELL |
| | | GGPSVFLFPPKPKDTLMISRTPEVTCVVVDVSHE |
| | | DPEVKFNWYVDGVEVHNAKTKPREEQYNSTYRV |
| | | VSVLTVLHQDWLNGKEYKCKVSNKALPAPIEKTIS |
| | | KAKGQPREPQVYTLPPSREEMTKNQVSLTCLVK |
| | | GFYPSDIAVEWESNGQPENNYKTTPPVLDSDGS |
| | | FFLYSKLTVDKSRWQQGNVFSCSVMHEALHNHY |
| | | TQKSLSLSPGK |
| SEQ ID NO: 13 | Ab HC DNA | CAGGTGCAATTGGTGCAGAGCGGTGCCGAAGT |
| | | GAAAAAACCGGGCAGCAGCGTGAAAGTTAGCT |
| | | GCAAAGCATCCGGAGGGACGTTTAGCAGCTAT |
| | | GCGATTAGCTGGGTGCGCCAGGCCCCGGGCC |
| | | AGGGCCTCGAGTGGATGGGCGTTATCTTCCCG |
| | | GCTGAAGGCGCTCCGGGTTACGCCCAGAAATT |
| | | TCAGGGCCGGGTGACCATTACCGCCGATGAAA |
| | | GCACCAGCACCGCCTATATGGAACTGAGCAGC |
| | | CTGCGCAGCGAAGATACGGCCGTGTATTATTG |
| | | CGCGCGTGGTGGTTACATCTCTGACTTCGATG |
| | | TTTGGGGCCAAGGCACCCTGGTGACTGTTAGC |
| | | TCAGCTAGCACCAAGGGCCCCAGCGTGTTCCC |
| | | CCTGGCCCCCAGCAGCAAGTCTACTTCCGGCG |
| | | GAACTGCTGCCCTGGGTTGCCTGGTGAAGGAC |
| | | TACTTCCCCGAGCCCGTGACAGTGTCCTGGAA |
| | | CTCTGGGGCTCTGACTTCCGGCGTGCACACCT |
| | | TCCCCGCCGTGCTGCAGAGCAGCGGCCTGTAC |
| | | AGCCTGAGCAGCGTGGTGACAGTGCCCTCCAG |
| | | CTCTCTGGGAACCCAGACCTATATCTGCAACGT |
| | | GAACCACAAGCCCAGCAACACCAAGGTGGACA |
| | | AGAGAGTGGAGCCCAAGAGCTGCGACAAGACC |
| | | CACACCTGCCCCCCCTGCCCAGCTCCAGAACT |
| | | GCTGGGAGGGCCTTCCGTGTTCCTGTTCCCCC |
| | | CCAAGCCCAAGGACACCCTGATGATCAGCAGG |
| | | ACCCCCGAGGTGACCTGCGTGGTGGTGGACG |
| | | TGTCCCACGAGGACCCAGAGGTGAAGTTCAAC |
| | | TGGTACGTGGACGGCGTGGAGGTGCACAACG |
| | | CCAAGACCAAGCCCAGAGAGGAGCAGTACAAC |
| | | AGCACCTACAGGGTGGTGTCCGTGCTGACCGT |
| | | GCTGCACCAGGACTGGCTGAACGGCAAAGAAT |
| | | ACAAGTGCAAAGTCTCCAACAAGGCCCTGCCA |
| | | GCCCCAATCGAAAAGACAATCAGCAAGGCCAA |
| | | GGGCCAGCCACGGGAGCCCCAGGTGTACACC |
| | | CTGCCCCCCAGCCGGGAGGAGATGACCAAGA |
| | | ACCAGGTGTCCCTGACCTGTCTGGTGAAGGGC |
| | | TTCTACCCCAGCGATATCGCCGTGGAGTGGGA |
| | | GAGCAACGGCCAGCCCGAGAACAACTACAAGA |
| | | CCACCCCCCCAGTGCTGGACAGCGACGGCAG |
| | | CTTCTTCCTGTACAGCAAGCTGACCGTGGACAA |
| | | GTCCAGGTGGCAGCAGGGCAACGTGTTCAGCT |
| | | GCAGCGTGATGCACGAGGCCCTGCACAACCAC |
| | | TACACCCAGAAGTCCCTGAGCCTGAGCCCCGG |
| | | CAAG |
| SEQ ID NO: 14 | Fab' HC(EU236) | QVQLVQSGAEVKKPGSSVKVSCKASGGTFSSYAI |
| | | SWVRQAPGQGLEWMGVIFPAEGAPGYAQKFQG |
| | | RVTITADESTSTAYMELSSLRSEDTAVYYCARGG |
| | | YISDFDVWGQGTLVTVSSASTKGPSVFPLAPSSK |
| | | STSGGTAALGCLVKDYFPEPVTVSWNSGALTSG |
| | | VHTFPAVLQSSGLYSLSSVVTVPSSSLGTQTYICN |
| | | VNHKPSNTKVDKRVEPKSCDKTHTCPPCPAPELL |
| | | G |
| SEQ ID NO: 15 | Fab' HC DNA | CAGGTGCAATTGGTGCAGAGCGGTGCCGAAGT |
| | | GAAAAAACCGGGCAGCAGCGTGAAAGTTAGCT |
| | | GCAAAGCATCCGGAGGGACGTTTAGCAGCTAT |
| | | GCGATTAGCTGGGTGCGCCAGGCCCCGGGCC |
| | | AGGGCCTCGAGTGGATGGGCGTTATCTTCCCG |
| | | GCTGAAGGCGCTCCGGGTTACGCCCAGAAATT |
| | | TCAGGGCCGGGTGACCATTACCGCCGATGAAA |
| | | GCACCAGCACCGCCTATATGGAACTGAGCAGC |
| | | CTGCGCAGCGAAGATACGGCCGTGTATTATTG |
| | | CGCGCGTGGTGGTTACATCTCTGACTTCGATG |
| | | TTTGGGGCCAAGGCACCCTGGTGACTGTTAGC |
| | | TCAGCTAGCACCAAGGGCCCCAGCGTGTTCCC |
| | | CCTGGCCCCCAGCAGCAAGTCTACTTCCGGCG |
| | | GAACTGCTGCCCTGGGTTGCCTGGTGAAGGAC |
| | | TACTTCCCCGAGCCCGTGACAGTGTCCTGGAA |
| | | CTCTGGGGCTCTGACTTCCGGCGTGCACACCT |
| | | TCCCCGCCGTGCTGCAGAGCAGCGGCCTGTAC |
| | | AGCCTGAGCAGCGTGGTGACAGTGCCCTCCAG |
| | | CTCTCTGGGAACCCAGACCTATATCTGCAACGT |
| | | GAACCACAAGCCCAGCAACACCAAGGTGGACA |
| | | AGAGAGTGGAGCCCAAGAGCTGCGACAAGACC |
| | | CACACCTGCCCCCCCTGCCCAGCTCCAGAACT |
| | | GCTGGGA |
| SEQ ID NO: 118 | Cys Fab- | QVQLVQSGAEVKKPGSSVKVSCKASGGTFSSYAI |
| | HC(EU221)-HC- | SWVRQAPGQGLEWMGVIFPAEGAPGYAQKFQG |
| | E152C (EU) | RVTITADESTSTAYMELSSLRSEDTAVYYCARGG |
| | | YISDFDVWGQGTLVTVSSASTKGPSVFPLAPSSK |
| | | STSGGTAALGCLVKDYFPCPVTVSWNSGALTSG |
| | | VHTFPAVLQSSGLYSLSSVVTVPSSSLGTQTYICN |
| | | VNHKPSNTKVDKRVEPKSCD |
| SEQ ID NO: 119 | Fab' HC(EU230) | QVQLVQSGAEVKKPGSSVKVSCKASGGTFSSYAI |
| | | SWVRQAPGQGLEWMGVIFPAEGAPGYAQKFQG |
| | | RVTITADESTSTAYMELSSLRSEDTAVYYCARGG |
| | | YISDFDVWGQGTLVTVSSASTKGPSVFPLAPSSK |
| | | STSGGTAALGCLVKDYFPEPVTVSWNSGALTSG |
| | | VHTFPAVLQSSGLYSLSSVVTVPSSSLGTQTYICN |
| | | VNHKPSNTKVDKRVEPKSCDKTHTCPPCP |
| SEQ ID NO: 120 | Fab' HC(EU232) | QVQLVQSGAEVKKPGSSVKVSCKASGGTFSSYAI |
| | | SWVRQAPGQGLEWMGVIFPAEGAPGYAQKFQG |
| | | RVTITADESTSTAYMELSSLRSEDTAVYYCARGG |
| | | YISDFDVWGQGTLVTVSSASTKGPSVFPLAPSSK |
| | | STSGGTAALGCLVKDYFPEPVTVSWNSGALTSG |
| | | VHTFPAVLQSSGLYSLSSVVTVPSSSLGTQTYICN |
| | | VNHKPSNTKVDKRVEPKSCDKTHTCPPCPAP |
| SEQ ID NO: 121 | Fab' HC(EU236)-Pro | QVQLVQSGAEVKKPGSSVKVSCKASGGTFSSYAI |
| | | SWVRQAPGQGLEWMGVIFPAEGAPGYAQKFQG |
| | | RVTITADESTSTAYMELSSLRSEDTAVYYCARGG |
| | | YISDFDVWGQGTLVTVSSASTKGPSVFPLAPSSK |
| | | STSGGTAALGCLVKDYFPEPVTVSWNSGALTSG |
| | | VHTFPAVLQSSGLYSLSSVVTVPSSSLGTQTYICN |
| | | VNHKPSNTKVDKRVEPKSCDKTHTCPPCPAPELL |
| | | GP |
| SEQ ID NO: 16 | LCDR1 (Kabat) | RASQSISNYLA |
| SEQ ID NO: 17 | LCDR2 (Kabat) | DASSLQS |
| SEQ ID NO: 18 | LCDR3 (Kabat) | QQYYYESIT |
| SEQ ID NO: 19 | LCDR1 (Chothia) | SQSISNY |
| SEQ ID NO: 20 | LCDR2 (Chothia) | DAS |
| SEQ ID NO: 21 | LCDR3 (Chothia) | YYYESI |
| SEQ ID NO: 16 | LCDR1 (Combined) | RASQSISNYLA |
| SEQ ID NO: 17 | LCDR2 (Combined) | DASSLQS |
| SEQ ID NO: 18 | LCDR3 (Combined) | QQYYYESIT |
| SEQ ID NO: 22 | LCDR1 (IMGT) | QSISNY |
| SEQ ID NO: 20 | LCDR2 (IMGT) | DAS |
| SEQ ID NO: 18 | LCDR3 (IMGT) | QQYYYESIT |
| SEQ ID NO: 23 | VL (kappa) | DIQMTQSPSSLSASVGDRVTITCRASQSISNYLA |
| | | WYQQKPGKAPKLLIYDASSLQSGVPSRFSGSGS |
| | | GTDFTLTISSLQPEDFATYYCQQYYYESITFGQGT |
| | | KVEIK |
| SEQ ID NO: 24 | VL DNA | GATATCCAGATGACCCAGAGCCCGAGCAGCCT |
| | | GAGCGCCAGCGTGGGCGATCGCGTGACCATTA |
| | | CCTGCAGAGCCAGCCAGTCTATTTCTAACTACC |
| | | TGGCTTGGTACCAGCAGAAACCGGGCAAAGCG |
| | | CCGAAACTATTAATCTACGACGCTTCTTCTCTG |
| | | CAAAGCGGCGTGCCGAGCCGCTTTAGCGGCA |
| | | GCGGATCCGGCACCGATTTCACCCTGACCATT |
| | | AGCTCTCTGCAACCGGAAGACTTTGCGACCTAT |
| | | TATTGCCAGCAGTACTACTACGAATCTATCACC |
| | | TTTGGCCAGGGCACGAAAGTTGAAATTAAA |
| SEQ ID NO: 25 | Ab/Fab' LC (kappa) | DIQMTQSPSSLSASVGDRVTITCRASQSISNYLA |
| | | WYQQKPGKAPKLLIYDASSLQSGVPSRFSGSGS |
| | | GTDFTLTISSLQPEDFATYYCQQYYYESITFGQGT |
| | | KVEIKRTVAAPSVFIFPPSDEQLKSGTASVVCLLN |
| | | NFYPREAKVQWKVDNALQSGNSQESVTEQDSK |
| | | DSTYSLSSTLTLSKADYEKHKVYACEVTHQGLSS |
| | | PVTKSFNRGEC |
| SEQ ID NO: 26 | Ab/Fab' LC DNA | GATATCCAGATGACCCAGAGCCCGAGCAGCCT |
| | | GAGCGCCAGCGTGGGCGATCGCGTGACCATTA |
| | | CCTGCAGAGCCAGCCAGTCTATTTCTAACTACC |
| | | TGGCTTGGTACCAGCAGAAACCGGGCAAAGCG |
| | | CCGAAACTATTAATCTACGACGCTTCTTCTCTG |
| | | CAAAGCGGCGTGCCGAGCCGCTTTAGCGGCA |
| | | GCGGATCCGGCACCGATTTCACCCTGACCATT |
| | | AGCTCTCTGCAACCGGAAGACTTTGCGACCTAT |
| | | TATTGCCAGCAGTACTACTACGAATCTATCACC |
| | | TTTGGCCAGGGCACGAAAGTTGAAATTAAACGT |
| | | ACGGTGGCCGCTCCCAGCGTGTTCATCTTCCC |
| | | CCCCAGCGACGAGCAGCTGAAGAGTGGCACC |
| | | GCCAGCGTGGTGTGCCTGCTGAACAACTTCTA |
| | | CCCCCGGGAGGCCAAGGTGCAGTGGAAGGTG |
| | | GACAACGCCCTGCAGAGCGGCAACAGCCAGG |
| | | AGAGCGTCACCGAGCAGGACAGCAAGGACTCC |
| | | ACCTACAGCCTGAGCAGCACCCTGACCCTGAG |
| | | CAAGGCCGACTACGAGAAGCATAAGGTGTACG |
| | | CCTGCGAGGTGACCCACCAGGGCCTGTCCAG |
| | | CCCCGTGACCAAGAGCTTCAACAGGGGCGAGT |
| | | GC |
| SEQ ID NO: 122 | Cys Fab-LC-E165C | DIQMTQSPSSLSASVGDRVTITCRASQSISNYLA |
| | (EU) | WYQQKPGKAPKLLIYDASSLQSGVPSRFSGSGS |
| | | GTDFTLTISSLQPEDFATYYCQQYYYESITFGQGT |
| | | KVEIKRTVAAPSVFIFPPSDEQLKSGTASVVCLLN |
| | | NFYPREAKVQWKVDNALQSGNSQESVTCQDSK |
| | | DSTYSLSSTLTLSKADYEKHKVYACEVTHQGLSS |
| | | PVTKSFNRGEC |
| SEQ ID NO: 123 | Cys Fab-LC-S114C | DIQMTQSPSSLSASVGDRVTITCRASQSISNYLA |
| | (EU) | WYQQKPGKAPKLLIYDASSLQSGVPSRFSGSGS |
| | | GTDFTLTISSLQPEDFATYYCQQYYYESITFGQGT |
| | | KVEIKRTVAAPCVFIFPPSDEQLKSGTASVVCLLN |
| | | NFYPREAKVQWKVDNALQSGNSQESVTEQDSK |
| | | DSTYSLSSTLTLSKADYEKHKVYACEVTHQGLSS |
| | | PVTKSFNRGEC |

Anti-cKIT Ab2/Fab2/Fab'2

| | | |
|---|---|---|
| SEQ ID NO: 27 | HCDR1 (Kabat) | SHALS |
| SEQ ID NO: 28 | HCDR2 (Kabat) | GIIPSFGTADYAQKFQG |
| SEQ ID NO: 29 | HCDR3 (Kabat) | GLYDFDY |
| SEQ ID NO: 30 | HCDR1 (Chothia) | GGTFSSH |
| SEQ ID NO: 31 | HCDR2 (Chothia) | IPSFGT |
| SEQ ID NO: 29 | HCDR3 (Chothia) | GLYDFDY |
| SEQ ID NO: 32 | HCDR1 (Combined) | GGTFSSHALS |
| SEQ ID NO: 28 | HCDR2 (Combined) | GIIPSFGTADYAQKFQG |
| SEQ ID NO: 29 | HCDR3 (Combined) | GLYDFDY |
| SEQ ID NO: 33 | HCDR1 (IMGT) | GGTFSSHA |
| SEQ ID NO: 34 | HCDR2 (IMGT) | IIPSFGTA |
| SEQ ID NO: 35 | HCDR3 (IMGT) | ARGLYDFDY |
| SEQ ID NO: 36 | VH | QVQLVQSGAEVKKPGSSVKVSCKASGGTFSSHA |
| | | LSWVRQAPGQGLEWMGGIIPSFGTADYAQKFQG |
| | | RVTITADESTSTAYMELSSLRSEDTAVYYCARGL |
| | | YDFDYWGQGTLVTVSS |
| SEQ ID NO: 37 | VH DNA | CAGGTGCAATTGGTGCAGAGCGGTGCCGAAGT |
| | | GAAAAAACCGGGCAGCAGCGTGAAAGTTAGCT |
| | | GCAAAGCATCCGGAGGGACGTTTTCTTCTCAT |
| | | GCTCTGTCTTGGGTGCGCCAGGCCCCGGGCC |
| | | AGGGCCTCGAGTGGATGGGCGGTATCATCCCG |
| | | TCTTTCGGCACTGCGGACTACGCCCAGAAATTT |
| | | CAGGGCCGGGTGACCATTACCGCCGATGAAAG |
| | | CACCAGCACCGCCTATATGGAACTGAGCAGCC |
| | | TGCGCAGCGAAGATACGGCCGTGTATTATTGC |
| | | GCGCGTGGTCTGTACGACTTCGACTACTGGGG |
| | | CCAAGGCACCCTGGTGACTGTTAGCTCA |
| SEQ ID NO: 38 | Ab HC | QVQLVQSGAEVKKPGSSVKVSCKASGGTFSSHA |
| | | LSWVRQAPGQGLEWMGGIIPSFGTADYAQKFQG |
| | | RVTITADESTSTAYMELSSLRSEDTAVYYCARGL |
| | | YDFDYWGQGTLVTVSSASTKGPSVFPLAPSSKS |
| | | TSGGTAALGCLVKDYFPEPVTVSWNSGALTSGV |
| | | HTFPAVLQSSGLYSLSSVVTVPSSSLGTQTYICNV |
| | | NHKPSNTKVDKRVEPKSCDKTHTCPPCPAPELL |
| | | GGPSVFLFPPKPKDTLMISRTPEVTCVVVDVSHE |
| | | DPEVKFNWYVDGVEVHNAKTKPREEQYNSTYRV |
| | | VSVLTVLHQDWLNGKEYKCKVSNKALPAPIEKTIS |
| | | KAKGQPREPQVYTLPPSREEMTKNQVSLTCLVK |
| | | GFYPSDIAVEWESNGQPENNYKTTPPVLDSDGS |
| | | FFLYSKLTVDKSRWQQGNVFSCSVMHEALHNHY |
| | | TQKSLSLSPGK |
| SEQ ID NO: 39 | Ab HC DNA | CAGGTGCAATTGGTGCAGAGCGGTGCCGAAGT |
| | | GAAAAAACCGGGCAGCAGCGTGAAAGTTAGCT |
| | | GCAAAGCATCCGGAGGGACGTTTTCTTCTCAT |
| | | GCTCTGTCTTGGGTGCGCCAGGCCCCGGGCC |
| | | AGGGCCTCGAGTGGATGGGCGGTATCATCCCG |
| | | TCTTTCGGCACTGCGGACTACGCCCAGAAATTT |
| | | CAGGGCCGGGTGACCATTACCGCCGATGAAAG |
| | | CACCAGCACCGCCTATATGGAACTGAGCAGCC |
| | | TGCGCAGCGAAGATACGGCCGTGTATTATTGC |
| | | GCGCGTGGTCTGTACGACTTCGACTACTGGGG |
| | | CCAAGGCACCCTGGTGACTGTTAGCTCAGCTA |
| | | GCACCAAGGGCCCCAGCGTGTTCCCCCTGGC |
| | | CCCCAGCAGCAAGTCTACTTCCGGCGGAACTG |
| | | CTGCCCTGGGTTGCCTGGTGAAGGACTACTTC |
| | | CCCGAGCCCGTGACAGTGTCCTGGAACTCTGG |
| | | GGCTCTGACTTCCGGCGTGCACACCTTCCCCG |
| | | CCGTGCTGCAGAGCAGCGGCCTGTACAGCCTG |
| | | AGCAGCGTGGTGACAGTGCCCTCCAGCTCTCT |
| | | GGGAACCCAGACCTATATCTGCAACGTGAACC |
| | | ACAAGCCCAGCAACACCAAGGTGGACAAGAGA |
| | | GTGGAGCCCAAGAGCTGCGACAAGACCCACAC |
| | | CTGCCCCCCCTGCCCAGCTCCAGAACTGCTGG |
| | | GAGGGCCTTCCGTGTTCCTGTTCCCCCCCAAG |
| | | CCCAAGGACACCCTGATGATCAGCAGGACCCC |
| | | CGAGGTGACCTGCGTGGTGGTGGACGTGTCC |
| | | CACGAGGACCCAGAGGTGAAGTTCAACTGGTA |
| | | CGTGGACGGCGTGGAGGTGCACAACGCCAAG |
| | | ACCAAGCCCAGAGAGGAGCAGTACAACAGCAC |
| | | CTACAGGGTGGTGTCCGTGCTGACCGTGCTGC |
| | | ACCAGGACTGGCTGAACGGCAAAGAATACAAG |
| | | TGCAAAGTCTCCAACAAGGCCCTGCCAGCCCC |
| | | AATCGAAAAGACAATCAGCAAGGCCAAGGGCC |
| | | AGCCACGGGAGCCCCAGGTGTACACCCTGCC |
| | | CCCCAGCCGGGAGGAGATGACCAAGAACCAG |
| | | GTGTCCCTGACCTGTCTGGTGAAGGGCTTCTA |
| | | CCCCAGCGATATCGCCGTGGAGTGGGAGAGC |
| | | AACGGCCAGCCCGAGAACAACTACAAGACCAC |
| | | CCCCCCAGTGCTGGACAGCGACGGCAGCTTCT |
| | | TCCTGTACAGCAAGCTGACCGTGGACAAGTCC |
| | | AGGTGGCAGCAGGGCAACGTGTTCAGCTGCAG |
| | | CGTGATGCACGAGGCCCTGCACAACCACTACA |
| | | CCCAGAAGTCCCTGAGCCTGAGCCCCGGCAAG |
| SEQ ID NO: 40 | Fab' HC(EU236) | QVQLVQSGAEVKKPGSSVKVSCKASGGTFSSHA |
| | | LSWVRQAPGQGLEWMGGIIPSFGTADYAQKFQG |
| | | RVTITADESTSTAYMELSSLRSEDTAVYYCARGL |
| | | YDFDYWGQGTLVTVSSASTKGPSVFPLAPSSKS |
| | | TSGGTAALGCLVKDYFPEPVTVSWNSGALTSGV |
| | | HTFPAVLQSSGLYSLSSVVTVPSSSLGTQTYICNV |
| | | NHKPSNTKVDKRVEPKSCDKTHTCPPCPAPELL |
| | | G |
| SEQ ID NO: 41 | Fab' HC DNA | CAGGTGCAATTGGTGCAGAGCGGTGCCGAAGT |
| | | GAAAAAACCGGGCAGCAGCGTGAAAGTTAGCT |
| | | GCAAAGCATCCGGAGGGACGTTTTCTTCTCAT |
| | | GCTCTGTCTTGGGTGCGCCAGGCCCCGGGCC |
| | | AGGGCCTCGAGTGGATGGGCGGTATCATCCCG |
| | | TCTTTCGGCACTGCGGACTACGCCCAGAAATTT |
| | | CAGGGCCGGGTGACCATTACCGCCGATGAAAG |
| | | CACCAGCACCGCCTATATGGAACTGAGCAGCC |
| | | TGCGCAGCGAAGATACGGCCGTGTATTATTGC |
| | | GCGCGTGGTCTGTACGACTTCGACTACTGGGG |
| | | CCAAGGCACCCTGGTGACTGTTAGCTCAGCTA |
| | | GCACCAAGGGCCCCAGCGTGTTCCCCCTGGC |
| | | CCCCAGCAGCAAGTCTACTTCCGGCGGAACTG |
| | | CTGCCCTGGGTTGCCTGGTGAAGGACTACTTC |
| | | CCCGAGCCCGTGACAGTGTCCTGGAACTCTGG |
| | | GGCTCTGACTTCCGGCGTGCACACCTTCCCCG |
| | | CCGTGCTGCAGAGCAGCGGCCTGTACAGCCTG |
| | | AGCAGCGTGGTGACAGTGCCCTCCAGCTCTCT |
| | | GGGAACCCAGACCTATATCTGCAACGTGAACC |
| | | ACAAGCCCAGCAACACCAAGGTGGACAAGAGA |
| | | GTGGAGCCCAAGAGCTGCGACAAGACCCACAC |
| | | CTGCCCCCCCTGCCCAGCTCCAGAACTGCTGG |
| | | GA |
| SEQ ID NO: 124 | Cys Fab- | QVQLVQSGAEVKKPGSSVKVSCKASGGTFSSHA |
| | HC(EU221)-HC- | LSWVRQAPGQGLEWMGGIIPSFGTADYAQKFQG |
| | E152C (EU) | RVTITADESTSTAYMELSSLRSEDTAVYYCARGL |
| | | YDFDYWGQGTLVTVSSASTKGPSVFPLAPSSKS |
| | | TSGGTAALGCLVKDYFPCPVTVSWNSGALTSGV |
| | | HTFPAVLQSSGLYSLSSVVTVPSSSLGTQTYICNV |
| | | NHKPSNTKVDKRVEPKSCD |
| SEQ ID NO: 125 | Fab' HC(EU230) | QVQLVQSGAEVKKPGSSVKVSCKASGGTFSSHA |
| | | LSWVRQAPGQGLEWMGGIIPSFGTADYAQKFQG |
| | | RVTITADESTSTAYMELSSLRSEDTAVYYCARGL |
| | | YDFDYWGQGTLVTVSSASTKGPSVFPLAPSSKS |
| | | TSGGTAALGCLVKDYFPEPVTVSWNSGALTSGV |
| | | HTFPAVLQSSGLYSLSSVVTVPSSSLGTQTYICNV |
| | | NHKPSNTKVDKRVEPKSCDKTHTCPPCP |
| SEQ ID NO: 126 | Fab' HC(EU232) | QVQLVQSGAEVKKPGSSVKVSCKASGGTFSSHA |
| | | LSWVRQAPGQGLEWMGGIIPSFGTADYAQKFQG |
| | | RVTITADESTSTAYMELSSLRSEDTAVYYCARGL |
| | | YDFDYWGQGTLVTVSSASTKGPSVFPLAPSSKS |
| | | TSGGTAALGCLVKDYFPEPVTVSWNSGALTSGV |
| | | HTFPAVLQSSGLYSLSSVVTVPSSSLGTQTYICNV |
| | | NHKPSNTKVDKRVEPKSCDKTHTCPPCPAP |
| SEQ ID NO: 127 | Fab' HC(EU236)-Pro | QVQLVQSGAEVKKPGSSVKVSCKASGGTFSSHA |
| | | LSWVRQAPGQGLEWMGGIIPSFGTADYAQKFQG |
| | | RVTITADESTSTAYMELSSLRSEDTAVYYCARGL |
| | | YDFDYWGQGTLVTVSSASTKGPSVFPLAPSSKS |
| | | TSGGTAALGCLVKDYFPEPVTVSWNSGALTSGV |
| | | HTFPAVLQSSGLYSLSSVVTVPSSSLGTQTYICNV |
| | | NHKPSNTKVDKRVEPKSCDKTHTCPPCPAPELL |
| | | GP |
| SEQ ID NO: 42 | LCDR1 (Kabat) | RASQDISQDLA |
| SEQ ID NO: 17 | LCDR2 (Kabat) | DASSLQS |
| SEQ ID NO: 43 | LCDR3 (Kabat) | QQYYYLPST |
| SEQ ID NO: 44 | LCDR1 (Chothia) | SQDISQD |
| SEQ ID NO: 20 | LCDR2 (Chothia) | DAS |
| SEQ ID NO: 45 | LCDR3 (Chothia) | YYYLPS |
| SEQ ID NO: 42 | LCDR1 (Combined) | RASQDISQDLA |
| SEQ ID NO: 17 | LCDR2 (Combined) | DASSLQS |
| SEQ ID NO: 43 | LCDR3 (Combined) | QQYYYLPST |
| SEQ ID NO: 46 | LCDR1 (IMGT) | QDISQD |
| SEQ ID NO: 20 | LCDR2 (IMGT) | DAS |
| SEQ ID NO: 43 | LCDR3 (IMGT) | QQYYYLPST |
| SEQ ID NO: 47 | VL (kappa) | DIQMTQSPSSLSASVGDRVTITCRASQDISQDLA |
| | | WYQQKPGKAPKLLIYDASSLQSGVPSRFSGSGS |
| | | GTDFTLTISSLQPEDFAVYYCQQYYYLPSTFGQG |
| | | TKVEIK |
| SEQ ID NO: 48 | VL DNA | GATATCCAGATGACCCAGAGCCCGAGCAGCCT |
| | | GAGCGCCAGCGTGGGCGATCGCGTGACCATTA |
| | | CCTGCAGAGCCAGCCAGGACATTTCTCAGGAC |
| | | CTGGCTTGGTACCAGCAGAAACCGGGCAAAGC |
| | | GCCGAAACTATTAATCTACGACGCTTCTTCTCT |
| | | GCAAAGCGGCGTGCCGAGCCGCTTTAGCGGC |
| | | AGCGGATCCGGCACCGATTTCACCCTGACCAT |
| | | TAGCTCTCTGCAACCGGAAGACTTTGCGGTGT |
| | | ATTATTGCCAGCAGTACTACTACCTGCCGTCTA |
| | | CCTTTGGCCAGGGCACGAAAGTTGAAATTAAA |
| SEQ ID NO: 49 | Ab/Fab' LC (kappa) | DIQMTQSPSSLSASVGDRVTITCRASQDISQDLA |
| | | WYQQKPGKAPKLLIYDASSLQSGVPSRFSGSGS |
| | | GTDFTLTISSLQPEDFAVYYCQQYYYLPSTFGQG |
| | | TKVEIKRTVAAPSVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGLS |
| | | SPVTKSFNRGEC |
| SEQ ID NO: 50 | Ab/Fab' LC DNA | GATATCCAGATGACCCAGAGCCCGAGCAGCCT |
| | | GAGCGCCAGCGTGGGCGATCGCGTGACCATTA |
| | | CCTGCAGAGCCAGCCAGGACATTTCTCAGGAC |
| | | CTGGCTTGGTACCAGCAGAAACCGGGCAAAGC |
| | | GCCGAAACTATTAATCTACGACGCTTCTTCTCT |
| | | GCAAAGCGGCGTGCCGAGCCGCTTTAGCGGC |
| | | AGCGGATCCGGCACCGATTTCACCCTGACCAT |
| | | TAGCTCTCTGCAACCGGAAGACTTTGCGGTGT |
| | | ATTATTGCCAGCAGTACTACTACCTGCCGTCTA |
| | | CCTTTGGCCAGGGCACGAAAGTTGAAATTAAAC |
| | | GTACGGTGGCCGCTCCCAGCGTGTTCATCTTC |
| | | CCCCCCAGCGACGAGCAGCTGAAGAGTGGCA |
| | | CCGCCAGCGTGGTGTGCCTGCTGAACAACTTC |
| | | TACCCCCGGGAGGCCAAGGTGCAGTGGAAGG |
| | | TGGACAACGCCCTGCAGAGCGGCAACAGCCA |
| | | GGAGAGCGTCACCGAGCAGGACAGCAAGGAC |
| | | TCCACCTACAGCCTGAGCAGCACCCTGACCCT |
| | | GAGCAAGGCCGACTACGAGAAGCATAAGGTGT |
| | | ACGCCTGCGAGGTGACCCACCAGGGCCTGTC |
| | | CAGCCCCGTGACCAAGAGCTTCAACAGGGGCG |
| | | AGTGC |
| SEQ ID NO: 128 | Cys Fab-LC-E165C | DIQMTQSPSSLSASVGDRVTITCRASQDISQDLA |
| | (EU) | WYQQKPGKAPKLLIYDASSLQSGVPSRFSGSGS |
| | | GTDFTLTISSLQPEDFAVYYCQQYYYLPSTFGQG |
| | | TKVEIKRTVAAPSVFIFPPSDEQLKSGTASVVCLL |
| | | NNFYPREAKVQWKVDNALQSGNSQESVTCQDS |
| | | KDSTYSLSSTLTLSKADYEKHKVYACEVTHQGLS |
| | | SPVTKSFNRGEC |
| SEQ ID NO: 129 | Cys Fab-LC-S114C | DIQMTQSPSSLSASVGDRVTITCRASQDISQDLA |
| | (EU) | WYQQKPGKAPKLLIYDASSLQSGVPSRFSGSGS |
| | | GTDFTLTISSLQPEDFAVYYCQQYYYLPSTFGQG |
| | | TKVEIKRTVAAPCVFIFPPSDEQLKSGTASVVCLL |
| | | NNFYPREAKVQWKVDNALQSGNSQESVTEQDS |
| | | KDSTYSLSSTLTLSKADYEKHKVYACEVTHQGLS |
| | | SPVTKSFNRGEC |

Anti-cKIT Ab3/Fab3/Fab'3

| | | |
|---|---|---|
| SEQ ID NO: 1 | HCDR1 (Kabat) | SYAIS |
| SEQ ID NO: 51 | HCDR2 (Kabat) | TIGPFEGQPRYAQKFQG |
| SEQ ID NO: 3 | HCDR3 (Kabat) | GGYISDFDV |
| SEQ ID NO: 4 | HCDR1 (Chothia) | GGTFSSY |
| SEQ ID NO: 52 | HCDR2 (Chothia) | GPFEGQ |
| SEQ ID NO: 3 | HCDR3 (Chothia) | GGYISDFDV |
| SEQ ID NO: 6 | HCDR1 (Combined) | GGTFSSYAIS |
| SEQ ID NO: 51 | HCDR2 (Combined) | TIGPFEGQPRYAQKFQG |
| SEQ ID NO: 3 | HCDR3 (Combined) | GGYISDFDV |
| SEQ ID NO: 7 | HCDR1 (IMGT) | GGTFSSYA |
| SEQ ID NO: 53 | HCDR2 (IMGT) | IGPFEGQP |
| SEQ ID NO: 9 | HCDR3 (IMGT) | ARGGYISDFDV |
| SEQ ID NO: 54 | VH | QVQLVQSGAEVKKPGSSVKVSCKASGGTFSSYAI |
| | | SWVRQAPGQGLEWMGTIGPFEGQPRYAQKFQG |
| | | RVTITADESTSTAYMELSSLRSEDTAVYYCARGG |
| | | YISDFDVWGQGTLVTVSS |
| SEQ ID NO: 55 | VH DNA | CAGGTGCAATTGGTGCAGAGCGGTGCCGAAGT |
| | | GAAAAAACCGGGCAGCAGCGTGAAAGTTAGCT |
| | | GCAAAGCATCCGGAGGGACGTTTAGCAGCTAT |
| | | GCGATTAGCTGGGTGCGCCAGGCCCCGGGCC |
| | | AGGGCCTCGAGTGGATGGGCACTATCGGTCCG |
| | | TTCGAAGGCCAGCCGCGTTACGCCCAGAAATT |
| | | TCAGGGCCGGGTGACCATTACCGCCGATGAAA |
| | | GCACCAGCACCGCCTATATGGAACTGAGCAGC |
| | | CTGCGCAGCGAAGATACGGCCGTGTATTATTG |
| | | CGCGCGTGGTGGTTACATCTCTGACTTCGATG |
| | | TTTGGGGCCAAGGCACCCTGGTGACTGTTAGC |
| | | TCA |
| SEQ ID NO: 56 | Ab HC | QVQLVQSGAEVKKPGSSVKVSCKASGGTFSSYAI |
| | | SWVRQAPGQGLEWMGTIGPFEGQPRYAQKFQG |
| | | RVTITADESTSTAYMELSSLRSEDTAVYYCARGG |
| | | YISDFDVWGQGTLVTVSSASTKGPSVFPLAPSSK |
| | | STSGGTAALGCLVKDYFPEPVTVSWNSGALTSG |
| | | VHTFPAVLQSSGLYSLSSVVTVPSSSLGTQTYICN |
| | | VNHKPSNTKVDKRVEPKSCDKTHTCPPCPAPELL |
| | | GGPSVFLFPPKPKDTLMISRTPEVTCVVVDVSHE |
| | | DPEVKFNWYVDGVEVHNAKTKPREEQYNSTYRV |
| | | VSVLTVLHQDWLNGKEYKCKVSNKALPAPIEKTIS |
| | | KAKGQPREPQVYTLPPSREEMTKNQVSLTCLVK |
| | | GFYPSDIAVEWESNGQPENNYKTTPPVLDSDGS |
| | | FFLYSKLTVDKSRWQQGNVFSCSVMHEALHNHY |
| | | TQKSLSLSPGK |
| SEQ ID NO: 57 | Ab HC DNA | CAGGTGCAATTGGTGCAGAGCGGTGCCGAAGT |
| | | GAAAAAACCGGGCAGCAGCGTGAAAGTTAGCT |
| | | GCAAAGCATCCGGAGGGACGTTTAGCAGCTAT |
| | | GCGATTAGCTGGGTGCGCCAGGCCCCGGGCC |
| | | AGGGCCTCGAGTGGATGGGCACTATCGGTCCG |
| | | TTCGAAGGCCAGCCGCGTTACGCCCAGAAATT |
| | | TCAGGGCCGGGTGACCATTACCGCCGATGAAA |
| | | GCACCAGCACCGCCTATATGGAACTGAGCAGC |
| | | CTGCGCAGCGAAGATACGGCCGTGTATTATTG |
| | | CGCGCGTGGTGGTTACATCTCTGACTTCGATG |
| | | TTTGGGGCCAAGGCACCCTGGTGACTGTTAGC |
| | | TCAGCTAGCACCAAGGGCCCAAGTGTGTTTCC |
| | | CCTGGCCCCCAGCAGCAAGTCTACTTCCGGCG |
| | | GAACTGCTGCCCTGGGTTGCCTGGTGAAGGAC |
| | | TACTTCCCCGAGCCCGTGACAGTGTCCTGGAA |
| | | CTCTGGGGCTCTGACTTCCGGCGTGCACACCT |
| | | TCCCCGCCGTGCTGCAGAGCAGCGGCCTGTAC |
| | | AGCCTGAGCAGCGTGGTGACAGTGCCCTCCAG |
| | | CTCTCTGGGAACCCAGACCTATATCTGCAACGT |
| | | GAACCACAAGCCCAGCAACACCAAGGTGGACA |
| | | AGAGAGTGGAGCCCAAGAGCTGCGACAAGACC |
| | | CACACCTGCCCCCCCTGCCCAGCTCCAGAACT |
| | | GCTGGGAGGGCCTTCCGTGTTCCTGTTCCCCC |
| | | CCAAGCCCAAGGACACCCTGATGATCAGCAGG |
| | | ACCCCCGAGGTGACCTGCGTGGTGGTGGACG |
| | | TGTCCCACGAGGACCCAGAGGTGAAGTTCAAC |
| | | TGGTACGTGGACGGCGTGGAGGTGCACAACG |
| | | CCAAGACCAAGCCCAGAGAGGAGCAGTACAAC |
| | | AGCACCTACAGGGTGGTGTCCGTGCTGACCGT |
| | | GCTGCACCAGGACTGGCTGAACGGCAAAGAAT |
| | | ACAAGTGCAAAGTCTCCAACAAGGCCCTGCCA |
| | | GCCCCAATCGAAAAGACAATCAGCAAGGCCAA |
| | | GGGCCAGCCACGGGAGCCCCAGGTGTACACC |
| | | CTGCCCCCCAGCCGGGAGGAGATGACCAAGA |
| | | ACCAGGTGTCCCTGACCTGTCTGGTGAAGGGC |
| | | TTCTACCCCAGCGATATCGCCGTGGAGTGGGA |
| | | GAGCAACGGCCAGCCCGAGAACAACTACAAGA |
| | | CCACCCCCCCAGTGCTGGACAGCGACGGCAG |
| | | CTTCTTCCTGTACAGCAAGCTGACCGTGGACAA |
| | | GTCCAGGTGGCAGCAGGGCAACGTGTTCAGCT |
| | | GCAGCGTGATGCACGAGGCCCTGCACAACCAC |
| | | TACACCCAGAAGTCCCTGAGCCTGAGCCCCGG |
| | | CAAG |
| SEQ ID NO: 58 | Fab' HC(EU236) | QVQLVQSGAEVKKPGSSVKVSCKASGGTFSSYAI |
| | | SWVRQAPGQGLEWMGTIGPFEGQPRYAQKFQG |
| | | RVTITADESTSTAYMELSSLRSEDTAVYYCARGG |
| | | YISDFDVWGQGTLVTVSSASTKGPSVFPLAPSSK |
| | | STSGGTAALGCLVKDYFPEPVTVSWNSGALTSG |
| | | VHTFPAVLQSSGLYSLSSVVTVPSSSLGTQTYICN |
| | | VNHKPSNTKVDKRVEPKSCDKTHTCPPCPAPELL |
| | | G |
| SEQ ID NO: 59 | Fab' HC DNA | CAGGTGCAATTGGTGCAGAGCGGTGCCGAAGT |
| | | GAAAAAACCGGGCAGCAGCGTGAAAGTTAGCT |
| | | GCAAAGCATCCGGAGGGACGTTTAGCAGCTAT |
| | | GCGATTAGCTGGGTGCGCCAGGCCCCGGGCC |
| | | AGGGCCTCGAGTGGATGGGCACTATCGGTCCG |
| | | TTCGAAGGCCAGCCGCGTTACGCCCAGAAATT |
| | | TCAGGGCCGGGTGACCATTACCGCCGATGAAA |
| | | GCACCAGCACCGCCTATATGGAACTGAGCAGC |
| | | CTGCGCAGCGAAGATACGGCCGTGTATTATTG |
| | | CGCGCGTGGTGGTTACATCTCTGACTTCGATG |
| | | TTTGGGGCCAAGGCACCCTGGTGACTGTTAGC |
| | | TCAGCTAGCACCAAGGGCCCAAGTGTGTTTCC |
| | | CCTGGCCCCCAGCAGCAAGTCTACTTCCGGCG |
| | | GAACTGCTGCCCTGGGTTGCCTGGTGAAGGAC |
| | | TACTTCCCCGAGCCCGTGACAGTGTCCTGGAA |
| | | CTCTGGGGCTCTGACTTCCGGCGTGCACACCT |
| | | TCCCCGCCGTGCTGCAGAGCAGCGGCCTGTAC |
| | | AGCCTGAGCAGCGTGGTGACAGTGCCCTCCAG |
| | | CTCTCTGGGAACCCAGACCTATATCTGCAACGT |
| | | GAACCACAAGCCCAGCAACACCAAGGTGGACA |
| | | AGAGAGTGGAGCCCAAGAGCTGCGACAAGACC |
| | | CACACCTGCCCCCCCTGCCCAGCTCCAGAACT |
| | | GCTGGGA |
| SEQ ID NO: 130 | Cys Fab | QVQLVQSGAEVKKPGSSVKVSCKASGGTFSSYAI |
| | HC(EU221)-HC- | SWVRQAPGQGLEWMGTIGPFEGQPRYAQKFQG |
| | E152C (EU) | RVTITADESTSTAYMELSSLRSEDTAVYYCARGG |
| | | YISDFDVWGQGTLVTVSSASTKGPSVFPLAPSSK |
| | | STSGGTAALGCLVKDYFPCPVTVSWNSGALTSG |
| | | VHTFPAVLQSSGLYSLSSVVTVPSSSLGTQTYICN |
| | | VNHKPSNTKVDKKVEPKSCD |
| SEQ ID NO: 131 | Fab' HC(EU230) | QVQLVQSGAEVKKPGSSVKVSCKASGGTFSSYAI |
| | | SWVRQAPGQGLEWMGTIGPFEGQPRYAQKFQG |
| | | RVTITADESTSTAYMELSSLRSEDTAVYYCARGG |
| | | YISDFDVWGQGTLVTVSSASTKGPSVFPLAPSSK |
| | | STSGGTAALGCLVKDYFPEPVTVSWNSGALTSG |
| | | VHTFPAVLQSSGLYSLSSVVTVPSSSLGTQTYICN |
| | | VNHKPSNTKVDKRVEPKSCDKTHTCPPCP |
| SEQ ID NO: 132 | Fab' HC(EU232) | QVQLVQSGAEVKKPGSSVKVSCKASGGTFSSYAI |
| | | SWVRQAPGQGLEWMGTIGPFEGQPRYAQKFQG |
| | | RVTITADESTSTAYMELSSLRSEDTAVYYCARGG |
| | | YISDFDVWGQGTLVTVSSASTKGPSVFPLAPSSK |
| | | STSGGTAALGCLVKDYFPEPVTVSWNSGALTSG |
| | | VHTFPAVLQSSGLYSLSSVVTVPSSSLGTQTYICN |
| | | VNHKPSNTKVDKRVEPKSCDKTHTCPPCPAP |
| SEQ ID NO: 133 | Fab' HC(EU236)-Pro | QVQLVQSGAEVKKPGSSVKVSCKASGGTFSSYAI |
| | | SWVRQAPGQGLEWMGTIGPFEGQPRYAQKFQG |
| | | RVTITADESTSTAYMELSSLRSEDTAVYYCARGG |
| | | YISDFDVWGQGTLVTVSSASTKGPSVFPLAPSSK |
| | | STSGGTAALGCLVKDYFPEPVTVSWNSGALTSG |
| | | VHTFPAVLQSSGLYSLSSVVTVPSSSLGTQTYICN |
| | | VNHKPSNTKVDKRVEPKSCDKTHTCPPCPAPELL |
| | | GP |
| SEQ ID NO: 16 | LCDR1 (Kabat) | RASQSISNYLA |
| SEQ ID NO: 17 | LCDR2 (Kabat) | DASSLQS |
| SEQ ID NO: 18 | LCDR3 (Kabat) | QQYYYESIT |
| SEQ ID NO: 19 | LCDR1 (Chothia) | SQSISNY |
| SEQ ID NO: 20 | LCDR2 (Chothia) | DAS |
| SEQ ID NO: 21 | LCDR3 (Chothia) | YYYESI |
| SEQ ID NO: 16 | LCDR1 (Combined) | RASQSISNYLA |
| SEQ ID NO: 17 | LCDR2 (Combined) | DASSLQS |
| SEQ ID NO: 18 | LCDR3 (Combined) | QQYYYESIT |
| SEQ ID NO: 22 | LCDR1 (IMGT) | QSISNY |
| SEQ ID NO: 20 | LCDR2 (IMGT) | DAS |
| SEQ ID NO: 18 | LCDR3 (IMGT) | QQYYYESIT |
| SEQ ID NO: 23 | VL (kappa) | DIQMTQSPSSLSASVGDRVTITCRASQSISNYLA |
| | | WYQQKPGKAPKLLIYDASSLQSGVPSRFSGSGS |
| | | GTDFTLTISSLQPEDFATYYCQQYYYESITFGQGT |
| | | KVEIK |
| SEQ ID NO: 24 | VL DNA | GATATCCAGATGACCCAGAGCCCGAGCAGCCT |
| | | GAGCGCCAGCGTGGGCGATCGCGTGACCATTA |
| | | CCTGCAGAGCCAGCCAGTCTATTTCTAACTACC |
| | | TGGCTTGGTACCAGCAGAAACCGGGCAAAGCG |
| | | CCGAAACTATTAATCTACGACGCTTCTTCTCTG |
| | | CAAAGCGGCGTGCCGAGCCGCTTTAGCGGCA |
| | | GCGGATCCGGCACCGATTTCACCCTGACCATT |
| | | AGCTCTCTGCAACCGGAAGACTTTGCGACCTAT |
| | | TATTGCCAGCAGTACTACTACGAATCTATCACC |
| | | TTTGGCCAGGGCACGAAAGTTGAAATTAAA |
| SEQ ID NO: 25 | Ab/Fab' LC (kappa) | DIQMTQSPSSLSASVGDRVTITCRASQSISNYLA |
| | | WYQQKPGKAPKLLIYDASSLQSGVPSRFSGSGS |
| | | GTDFTLTISSLQPEDFATYYCQQYYYESITFGQGT |
| | | KVEIKRTVAAPSVFIFPPSDEQLKSGTASVVCLLN |
| | | NFYPREAKVQWKVDNALQSGNSQESVTEQDSK |
| | | DSTYSLSSTLTLSKADYEKHKVYACEVTHQGLSS |
| | | PVTKSFNRGEC |
| SEQ ID NO: 26 | Ab/Fab' LC DNA | GATATCCAGATGACCCAGAGCCCGAGCAGCCT |
| | | GAGCGCCAGCGTGGGCGATCGCGTGACCATTA |
| | | CCTGCAGAGCCAGCCAGTCTATTTCTAACTACC |
| | | TGGCTTGGTACCAGCAGAAACCGGGCAAAGCG |
| | | CCGAAACTATTAATCTACGACGCTTCTTCTCTG |
| | | CAAAGCGGCGTGCCGAGCCGCTTTAGCGGCA |
| | | GCGGATCCGGCACCGATTTCACCCTGACCATT |
| | | AGCTCTCTGCAACCGGAAGACTTTGCGACCTAT |
| | | TATTGCCAGCAGTACTACTACGAATCTATCACC |
| | | TTTGGCCAGGGCACGAAAGTTGAAATTAAACGT |
| | | ACGGTGGCCGCTCCCAGCGTGTTCATCTTCCC |
| | | CCCCAGCGACGAGCAGCTGAAGAGTGGCACC |
| | | GCCAGCGTGGTGTGCCTGCTGAACAACTTCTA |
| | | CCCCCGGGAGGCCAAGGTGCAGTGGAAGGTG |
| | | GACAACGCCCTGCAGAGCGGCAACAGCCAGG |
| | | AGAGCGTCACCGAGCAGGACAGCAAGGACTCC |
| | | ACCTACAGCCTGAGCAGCACCCTGACCCTGAG |
| | | CAAGGCCGACTACGAGAAGCATAAGGTGTACG |
| | | CCTGCGAGGTGACCCACCAGGGCCTGTCCAG |
| | | CCCCGTGACCAAGAGCTTCAACAGGGGCGAGT |
| | | GC |
| SEQ ID NO: 134 | Cys Fab-LC-E165C | DIQMTQSPSSLSASVGDRVTITCRASQSISNYLA |
| | (EU) | WYQQKPGKAPKLLIYDASSLQSGVPSRFSGSGS |
| | | GTDFTLTISSLQPEDFATYYCQQYYYESITFGQGT |
| | | KVEIKRTVAAPSVFIFPPSDEQLKSGTASVVCLLN |
| | | NFYPREAKVQWKVDNALQSGNSQESVTCQDSK |
| | | DSTYSLSSTLTLSKADYEKHKVYACEVTHQGLSS |
| | | PVTKSFNRGEC |
| SEQ ID NO: 135 | Cys Fab-LC-S114C | DIQMTQSPSSLSASVGDRVTITCRASQSISNYLA |
| | (EU) | WYQQKPGKAPKLLIYDASSLQSGVPSRFSGSGS |
| | | GTDFTLTISSLQPEDFATYYCQQYYYESITFGQGT |
| | | KVEIKRTVAAPCVFIFPPSDEQLKSGTASVVCLLN |
| | | NFYPREAKVQWKVDNALQSGNSQESVTEQDSK |
| | | DSTYSLSSTLTLSKADYEKHKVYACEVTHQGLSS |
| | | PVTKSFNRGEC |

Anti-cKIT Ab4/Fab4/Fab'4

| | | |
|---|---|---|
| SEQ ID NO: 60 | HCDR1 (Kabat) | TNSAAWN |
| SEQ ID NO: 61 | HCDR2 (Kabat) | RIYYRSQWLNDYAVSVKS |
| SEQ ID NO: 62 | HCDR3 (Kabat) | QLTYPYTVYHKALDV |
| SEQ ID NO: 63 | HCDR1 (Chothia) | GDSVSTNSA |
| SEQ ID NO: 64 | HCDR2 (Chothia) | YYRSQWL |
| SEQ ID NO: 62 | HCDR3 (Chothia) | QLTYPYTVYHKALDV |
| SEQ ID NO: 65 | HCDR1 (Combined) | GDSVSTNSAAWN |
| SEQ ID NO: 61 | HCDR2 (Combined) | RIYYRSQWLNDYAVSVKS |
| SEQ ID NO: 62 | HCDR3 (Combined) | QLTYPYTVYHKALDV |
| SEQ ID NO: 66 | HCDR1 (IMGT) | GDSVSTNSAA |
| SEQ ID NO: 67 | HCDR2 (IMGT) | IYYRSQWLN |
| SEQ ID NO: 68 | HCDR3 (IMGT) | ARQLTYPYTVYHKALDV |
| SEQ ID NO: 69 | VH | QVQLQQSGPGLVKPSQTLSLTCAISGDSVSTNSA |
| | | AWNWIRQSPSRGLEWLGRIYYRSQWLNDYAVSV |
| | | KSRITINPDTSKNQFSLQLNSVTPEDTAVYYCARQ |
| | | LTYPYTVYHKALDVWGQGTLVTVSS |
| SEQ ID NO: 70 | VH DNA | CAGGTGCAATTGCAGCAGAGCGGTCCGGGCCT |
| | | GGTGAAACCGAGCCAGACCCTGAGCCTGACCT |
| | | GCGCGATTTCCGGAGATAGCGTGAGCACTAAC |
| | | TCTGCTGCTTGGAACTGGATTCGTCAGAGCCC |
| | | GAGCCGTGGCCTCGAGTGGCTGGGCCGTATCT |
| | | ACTACCGTAGCCAGTGGCTGAACGACTATGCC |
| | | GTGAGCGTGAAAAGCCGCATTACCATTAACCC |
| | | GGATACTTCGAAAAACCAGTTTAGCCTGCAACT |
| | | GAACAGCGTGACCCCGGAAGATACGGCCGTGT |
| | | ATTATTGCGCGCGTCAGCTGACTTACCCGTACA |
| | | CTGTTTACCATAAAGCTCTGGATGTTTGGGGTC |
| | | AAGGAACCCTGGTCACCGTCTCCTCG |
| SEQ ID NO: 71 | Ab HC | QVQLQQSGPGLVKPSQTLSLTCAISGDSVSTNSA |
| | | AWNWIRQSPSRGLEWLGRIYYRSQWLNDYAVSV |
| | | KSRITINPDTSKNQFSLQLNSVTPEDTAVYYCARQ |
| | | LTYPYTVYHKALDVWGQGTLVTVSSASTKGPSVF |
| | | PLAPSSKSTSGGTAALGCLVKDYFPEPVTVSWNS |
| | | GALTSGVHTFPAVLQSSGLYSLSSVVTVPSSSLG |
| | | TQTYICNVNHKPSNTKVDKRVEPKSCDKTHTCPP |
| | | CPAPELLGGPSVFLFPPKPKDTLMISRTPEVTCVV |
| | | VDVSHEDPEVKFNWYVDGVEVHNAKTKPREEQY |
| | | NSTYRVVSVLTVLHQDWLNGKEYKCKVSNKALP |
| | | APIEKTISKAKGQPREPQVYTLPPSREEMTKNQV |
| | | SLTCLVKGFYPSDIAVEWESNGQPENNYKTTPPV |
| | | LDSDGSFFLYSKLTVDKSRWQQGNVFSCSVMHE |
| | | ALHNHYTQKSLSLSPGK |
| SEQ ID NO: 72 | Ab HC DNA | CAGGTGCAATTGCAGCAGAGCGGTCCGGGCCT |
| | | GGTGAAACCGAGCCAGACCCTGAGCCTGACCT |
| | | GCGCGATTTCCGGAGATAGCGTGAGCACTAAC |
| | | TCTGCTGCTTGGAACTGGATTCGTCAGAGCCC |
| | | GAGCCGTGGCCTCGAGTGGCTGGGCCGTATCT |
| | | ACTACCGTAGCCAGTGGCTGAACGACTATGCC |
| | | GTGAGCGTGAAAAGCCGCATTACCATTAACCC |
| | | GGATACTTCGAAAAACCAGTTTAGCCTGCAACT |
| | | GAACAGCGTGACCCCGGAAGATACGGCCGTGT |
| | | ATTATTGCGCGCGTCAGCTGACTTACCCGTACA |
| | | CTGTTTACCATAAAGCTCTGGATGTTTGGGGTC |
| | | AAGGAACCCTGGTCACCGTCTCCTCGGCTAGC |
| | | ACCAAGGGCCCCAGCGTGTTCCCCCTGGCCCC |
| | | CAGCAGCAAGTCTACTTCCGGCGGAACTGCTG |
| | | CCCTGGGTTGCCTGGTGAAGGACTACTTCCCC |
| | | GAGCCCGTGACAGTGTCCTGGAACTCTGGGGC |
| | | TCTGACTTCCGGCGTGCACACCTTCCCCGCCG |
| | | TGCTGCAGAGCAGCGGCCTGTACAGCCTGAGC |
| | | AGCGTGGTGACAGTGCCCTCCAGCTCTCTGGG |
| | | AACCCAGACCTATATCTGCAACGTGAACCACAA |
| | | GCCCAGCAACACCAAGGTGGACAAGAGAGTGG |
| | | AGCCCAAGAGCTGCGACAAGACCCACACCTGC |
| | | CCCCCCTGCCCAGCTCCAGAACTGCTGGGAGG |
| | | GCCTTCCGTGTTCCTGTTCCCCCCCAAGCCCA |
| | | AGGACACCCTGATGATCAGCAGGACCCCCGAG |
| | | GTGACCTGCGTGGTGGTGGACGTGTCCCACGA |
| | | GGACCCAGAGGTGAAGTTCAACTGGTACGTGG |
| SEQ ID NO: 136 | Cys Fab | QVQLQQSGPGLVKPSQTLSLTCAISGDSVSTNSA |
| | HC(EU221)-HC- | AWNWIRQSPSRGLEWLGRIYYRSQWLNDYAVSV |
| | E152C (EU) | KSRITINPDTSKNQFSLQLNSVTPEDTAVYYCARQ |
| | | LTYPYTVYHKALDVWGQGTLVTVSSASTKGPSVF |
| | | PLAPSSKSTSGGTAALGCLVKDYFPCPVTVSWN |
| | | SGALTSGVHTFPAVLQSSGLYSLSSVVTVPSSSL |
| | | GTQTYICNVNHKPSNTKVDKRVEPKSCD |
| SEQ ID NO: 137 | Fab' HC(EU230) | QVQLQQSGPGLVKPSQTLSLTCAISGDSVSTNSA |
| | | AWNWIRQSPSRGLEWLGRIYYRSQWLNDYAVSV |
| | | KSRITINPDTSKNQFSLQLNSVTPEDTAVYYCARQ |
| | | LTYPYTVYHKALDVWGQGTLVTVSSASTKGPSVF |
| | | PLAPSSKSTSGGTAALGCLVKDYFPEPVTVSWNS |
| | | GALTSGVHTFPAVLQSSGLYSLSSVVTVPSSSLG |
| | | TQTYICNVNHKPSNTKVDKRVEPKSCDKTHTCPP |
| | | CP |
| SEQ ID NO: 138 | Fab' HC(EU232) | QVQLQQSGPGLVKPSQTLSLTCAISGDSVSTNSA |
| | | AWNWIRQSPSRGLEWLGRIYYRSQWLNDYAVSV |
| | | KSRITINPDTSKNQFSLQLNSVTPEDTAVYYCARQ |
| | | LTYPYTVYHKALDVWGQGTLVTVSSASTKGPSVF |
| | | PLAPSSKSTSGGTAALGCLVKDYFPEPVTVSWNS |
| | | GALTSGVHTFPAVLQSSGLYSLSSVVTVPSSSLG |
| | | TQTYICNVNHKPSNTKVDKRVEPKSCDKTHTCPP |
| | | CPAP |
| SEQ ID NO: 139 | Fab' HC(EU236)-Pro | QVQLQQSGPGLVKPSQTLSLTCAISGDSVSTNSA |
| | | AWNWIRQSPSRGLEWLGRIYYRSQWLNDYAVSV |
| | | KSRITINPDTSKNQFSLQLNSVTPEDTAVYYCARQ |
| | | LTYPYTVYHKALDVWGQGTLVTVSSASTKGPSVF |
| | | PLAPSSKSTSGGTAALGCLVKDYFPEPVTVSWNS |
| | | GALTSGVHTFPAVLQSSGLYSLSSVVTVPSSSLG |
| | | TQTYICNVNHKPSNTKVDKRVEPKSCDKTHTCPP |
| | | CPAPELLGP |
| SEQ ID NO: 75 | LCDR1 (Kabat) | SGDNLGDQYVS |
| SEQ ID NO: 76 | LCDR2 (Kabat) | DDTDRPS |
| SEQ ID NO: 77 | LCDR3 (Kabat) | QSTDSKSVV |
| SEQ ID NO: 78 | LCDR1 (Chothia) | DNLGDQY |
| SEQ ID NO: 79 | LCDR2 (Chothia) | DDT |
| SEQ ID NO: 80 | LCDR3 (Chothia) | TDSKSV |
| SEQ ID NO: 75 | LCDR1 (Combined) | SGDNLGDQYVS |
| SEQ ID NO: 76 | LCDR2 (Combined) | DDTDRPS |
| SEQ ID NO: 77 | LCDR3 (Combined) | QSTDSKSVV |
| SEQ ID NO: 81 | LCDR1 (IMGT) | NLGDQY |
| SEQ ID NO: 79 | LCDR2 (IMGT) | DDT |
| SEQ ID NO: 77 | LCDR3 (IMGT) | QSTDSKSVV |
| SEQ ID NO: 82 | VL (lambda) | DIELTQPPSVSVSPGQTASITCSGDNLGDQYVSW |
| | | YQQKPGQAPVLVIYDDTDRPSGIPERFSGSNSGN |
| | | TATLTISGTQAEDEADYYCQSTDSKSVVFGGGTK |
| | | LTVL |
| SEQ ID NO: 83 | VL DNA | GATATCGAACTGACCCAGCCGCCGAGCGTGAG |
| | | CGTGAGCCCGGGCCAGACCGCGAGCATTACCT |
| | | GTAGCGGCGATAACCTGGGTGACCAATACGTT |
| | | TCTTGGTACCAGCAGAAACCGGGCCAGGCGCC |
| | | GGTGCTGGTGATCTACGACGACACTGACCGTC |
| | | CGAGCGGCATCCCGGAACGTTTTAGCGGATCC |
| | | AACAGCGGCAACACCGCGACCCTGACCATTAG |
| | | CGGCACCCAGGCGGAAGACGAAGCGGATTATT |
| | | ACTGCCAGTCTACTGACTCTAAATCTGTTGTGT |
| | | TTGGCGGCGGCACGAAGTTAACCGTCCTA |
| SEQ ID NO: 84 | Ab/Fab' LC (lambda) | DIELTQPPSVSVSPGQTASITCSGDNLGDQYVSW |
| | | YQQKPGQAPVLVIYDDTDRPSGIPERFSGSNSGN |
| | | TATLTISGTQAEDEADYYCQSTDSKSVVFGGGTK |
| | | LTVLGQPKAAPSVTLFPPSSEELQANKATLVCLIS |
| | | DFYPGAVTVAWKADSSPVKAGVETTTPSKQSNN |
| | | KYAASSYLSLTPEQWKSHRSYSCQVTHEGSTVE |
| | | KTVAPTECS |
| SEQ ID NO: 85 | Ab/Fab' LC DNA | GATATCGAACTGACCCAGCCGCCGAGCGTGAG |
| | | CGTGAGCCCGGGCCAGACCGCGAGCATTACCT |
| | | GTAGCGGCGATAACCTGGGTGACCAATACGTT |
| | | TCTTGGTACCAGCAGAAACCGGGCCAGGCGCC |
| | | GGTGCTGGTGATCTACGACGACACTGACCGTC |
| | | CGAGCGGCATCCCGGAACGTTTTAGCGGATCC |
| | | AACAGCGGCAACACCGCGACCCTGACCATTAG |
| | | CGGCACCCAGGCGGAAGACGAAGCGGATTATT |
| | | ACTGCCAGTCTACTGACTCTAAATCTGTTGTGT |
| | | TTGGCGGCGGCACGAAGTTAACCGTCCTAGGC |
| | | CAGCCTAAGGCCGCTCCCTCCGTGACCCTGTT |
| | | CCCCCCCAGCTCCGAGGAACTGCAGGCCAACA |
| | | AGGCCACCCTGGTGTGCCTGATCAGCGACTTC |
| | | TACCCTGGCGCCGTGACCGTGGCCTGGAAGG |
| | | CCGACAGCAGCCCCGTGAAGGCCGGCGTGGA |
| | | GACAACCACCCCCAGCAAGCAGAGCAACAACA |
| | | AGTACGCCGCCAGCAGCTACCTGAGCCTGACC |
| | | CCCGAGCAGTGGAAGAGCCACAGAAGCTACAG |
| | | CTGCCAGGTCACCCACGAGGGCAGCACCGTG |
| | | GAGAAAACCGTGGCCCCCACCGAGTGCAGC |
| SEQ ID NO: 140 | Cys Fab-LC | DIELTQPPSVSVSPGQTASITCSGDNLGDQYVSW |
| | (lambda)-A143C | YQQKPGQAPVLVIYDDTDRPSGIPERFSGSNSGN |
| | (EU) | TATLTISGTQAEDEADYYCQSTDSKSVVFGGGTK |
| | | LTVLGQPKAAPSVTLFPPSSEELQANKATLVCLIS |
| | | DFYPGCVTVAWKADSSPVKAGVETTTPSKQSNN |
| | | KYAASSYLSLTPEQVVKSHRSYSCQVTHEGSTVE |
| | | KTVAPTECS |

Anti-cKIT Ab5/Fab5/Fab'5

| | | |
|---|---|---|
| SEQ ID NO: 86 | HCDR1 (Kabat) | NYWIA |
| SEQ ID NO: 87 | HCDR2 (Kabat) | IIYPSNSYTLYSPSFQG |
| SEQ ID NO: 88 | HCDR3 (Kabat) | VPPGGSISYPAFDH |
| SEQ ID NO: 89 | HCDR1 (Chothia) | GYSFTNY |
| SEQ ID NO: 90 | HCDR2 (Chothia) | YPSNSY |
| SEQ ID NO: 88 | HCDR3 (Chothia) | VPPGGSISYPAFDH |
| SEQ ID NO: 91 | HCDR1 (Combined) | GYSFTNYWIA |
| SEQ ID NO: 87 | HCDR2 (Combined) | IIYPSNSYTLYSPSFQG |
| SEQ ID NO: 88 | HCDR3 (Combined) | VPPGGSISYPAFDH |
| SEQ ID NO: 92 | HCDR1 (IMGT) | GYSFTNYW |
| SEQ ID NO: 93 | HCDR2 (IMGT) | IYPSNSYT |
| SEQ ID NO: 94 | HCDR3 (IMGT) | ARVPPGGSISYPAFDH |
| SEQ ID NO: 95 | VH | QVQLVQSGAEVKKPGESLKISCKGSGYSFTNYWI |
| | | AWVRQMPGKGLEWMGIIYPSNSYTLYSPSFQGQ |
| | | VTISADKSISTAYLQWSSLKASDTAMYYCARVPP |
| | | GGSISYPAFDHWGQGTLVTVSS |
| SEQ ID NO: 96 | VH DNA | CAGGTGCAATTGGTGCAGAGCGGTGCGGAAGT |
| | | GAAAAAACCGGGCGAAAGCCTGAAAATTAGCT |
| | | GCAAAGGCTCCGGATATAGCTTCACTAACTACT |
| | | GGATCGCTTGGGTGCGCCAGATGCCGGGCAA |
| | | AGGTCTCGAGTGGATGGGCATCATCTACCCGT |
| | | CTAACAGCTACACCCTGTATAGCCCGAGCTTTC |
| | | AGGGCCAGGTGACCATTAGCGCGGATAAAAGC |
| | | ATCAGCACCGCGTATCTGCAATGGAGCAGCCT |
| | | GAAAGCGAGCGATACCGCGATGTATTATTGCG |
| | | CGCGTGTTCCGCCGGGTGGTTCTATCTCTTAC |
| | | CCGGCTTTCGATCATTGGGGCCAAGGCACCCT |
| | | GGTGACTGTTAGCTCA |
| SEQ ID NO: 97 | Ab HC | QVQLVQSGAEVKKPGESLKISCKGSGYSFTNYWI |
| | | AWVRQMPGKGLEWMGIIYPSNSYTLYSPSFQGQ |
| | | VTISADKSISTAYLQWSSLKASDTAMYYCARVPP |
| | | GGSISYPAFDHWGQGTLVTVSSASTKGPSVFPLA |
| | | PSSKSTSGGTAALGCLVKDYFPEPVTVSWNSGA |
| | | LTSGVHTFPAVLQSSGLYSLSSVVTVPSSSLGTQ |
| | | TYICNVNHKPSNTKVDKRVEPKSCDKTHTCPPCP |
| | | APELLGGPSVFLFPPKPKDTLMISRTPEVTCVVVD |
| | | VSHEDPEVKFNWYVDGVEVHNAKTKPREEQYNS |
| | | TYRVVSVLTVLHQDWLNGKEYKCKVSNKALPAPI |
| | | EKTISKAKGQPREPQVYTLPPSREEMTKNQVSLT |
| | | CLVKGFYPSDIAVEWESNGQPENNYKTTPPVLDS |
| | | DGSFFLYSKLTVDKSRWQQGNVFSCSVMHEALH |
| | | NHYTQKSLSLSPGK |
| SEQ ID NO: 98 | Ab HC DNA | CAGGTGCAATTGGTGCAGAGCGGTGCGGAAGT |
| | | GAAAAAACCGGGCGAAAGCCTGAAAATTAGCT |
| | | GCAAAGGCTCCGGATATAGCTTCACTAACTACT |
| | | GGATCGCTTGGGTGCGCCAGATGCCGGGCAA |
| | | AGGTCTCGAGTGGATGGGCATCATCTACCCGT |
| | | CTAACAGCTACACCCTGTATAGCCCGAGCTTTC |
| | | AGGGCCAGGTGACCATTAGCGCGGATAAAAGC |
| | | ATCAGCACCGCGTATCTGCAATGGAGCAGCCT |
| | | GAAAGCGAGCGATACCGCGATGTATTATTGCG |
| | | CGCGTGTTCCGCCGGGTGGTTCTATCTCTTAC |
| | | CCGGCTTTCGATCATTGGGGCCAAGGCACCCT |
| | | GGTGACTGTTAGCTCAGCTAGCACCAAGGGCC |
| | | CCAGCGTGTTCCCCCTGGCCCCCAGCAGCAAG |
| | | TCTACTTCCGGCGGAACTGCTGCCCTGGGTTG |
| | | CCTGGTGAAGGACTACTTCCCCGAGCCCGTGA |
| | | CAGTGTCCTGGAACTCTGGGGCTCTGACTTCC |
| | | GGCGTGCACACCTTCCCCGCCGTGCTGCAGAG |
| | | CAGCGGCCTGTACAGCCTGAGCAGCGTGGTGA |
| | | CAGTGCCCTCCAGCTCTCTGGGAACCCAGACC |
| | | TATATCTGCAACGTGAACCACAAGCCCAGCAAC |
| | | ACCAAGGTGGACAAGAGAGTGGAGCCCAAGAG |
| | | CTGCGACAAGACCCACACCTGCCCCCCCTGCC |
| | | CAGCTCCAGAACTGCTGGGAGGGCCTTCCGTG |
| | | TTCCTGTTCCCCCCCAAGCCCAAGGACACCCT |
| | | GATGATCAGCAGGACCCCCGAGGTGACCTGCG |
| | | TGGTGGTGGACGTGTCCCACGAGGACCCAGA |
| | | GGTGAAGTTCAACTGGTACGTGGACGGCGTGG |
| | | AGGTGCACAACGCCAAGACCAAGCCCAGAGAG |
| | | GAGCAGTACAACAGCACCTACAGGGTGGTGTC |
| | | CGTGCTGACCGTGCTGCACCAGGACTGGCTGA |
| | | ACGGCAAAGAATACAAGTGCAAAGTCTCCAACA |
| | | AGGCCCTGCCAGCCCCAATCGAAAAGACAATC |
| | | AGCAAGGCCAAGGGCCAGCCACGGGAGCCCC |
| | | AGGTGTACACCCTGCCCCCCAGCCGGGAGGA |
| | | GATGACCAAGAACCAGGTGTCCCTGACCTGTC |
| | | TGGTGAAGGGCTTCTACCCCAGCGATATCGCC |
| | | GTGGAGTGGGAGAGCAACGGCCAGCCCGAGA |
| | | ACAACTACAAGACCACCCCCCCAGTGCTGGAC |
| | | AGCGACGGCAGCTTCTTCCTGTACAGCAAGCT |
| | | GACCGTGGACAAGTCCAGGTGGCAGCAGGGC |
| | | AACGTGTTCAGCTGCAGCGTGATGCACGAGGC |
| | | CCTGCACAACCACTACACCCAGAAGTCCCTGA |
| | | GCCTGAGCCCCGGCAAG |
| SEQ ID NO: 99 | Fab' HC (EU236) | QVQLVQSGAEVKKPGESLKISCKGSGYSFTNYWI |
| | | AWVRQMPGKGLEWMGIIYPSNSYTLYSPSFQGQ |
| | | VTISADKSISTAYLQWSSLKASDTAMYYCARVPP |
| | | GGSISYPAFDHWGQGTLVTVSSASTKGPSVFPLA |
| | | PSSKSTSGGTAALGCLVKDYFPEPVTVSWNSGA |
| | | LTSGVHTFPAVLQSSGLYSLSSVVTVPSSSLGTQ |
| | | TYICNVNHKPSNTKVDKRVEPKSCDKTHTCPPCP |
| | | APELLG |
| SEQ ID NO: 100 | Fab' HC DNA | CAGGTGCAATTGGTGCAGAGCGGTGCGGAAGT |
| | | GAAAAAACCGGGCGAAAGCCTGAAAATTAGCT |
| | | GCAAAGGCTCCGGATATAGCTTCACTAACTACT |
| | | GGATCGCTTGGGTGCGCCAGATGCCGGGCAA |
| | | AGGTCTCGAGTGGATGGGCATCATCTACCCGT |
| | | CTAACAGCTACACCCTGTATAGCCCGAGCTTTC |
| | | AGGGCCAGGTGACCATTAGCGCGGATAAAAGC |
| | | ATCAGCACCGCGTATCTGCAATGGAGCAGCCT |
| | | GAAAGCGAGCGATACCGCGATGTATTATTGCG |
| | | CGCGTGTTCCGCCGGGTGGTTCTATCTCTTAC |
| | | CCGGCTTTCGATCATTGGGGCCAAGGCACCCT |
| | | GGTGACTGTTAGCTCAGCTAGCACCAAGGGCC |
| | | CCAGCGTGTTCCCCCTGGCCCCCAGCAGCAAG |
| | | TCTACTTCCGGCGGAACTGCTGCCCTGGGTTG |
| | | CCTGGTGAAGGACTACTTCCCCGAGCCCGTGA |
| | | CAGTGTCCTGGAACTCTGGGGCTCTGACTTCC |
| | | GGCGTGCACACCTTCCCCGCCGTGCTGCAGAG |
| | | CAGCGGCCTGTACAGCCTGAGCAGCGTGGTGA |
| | | CAGTGCCCTCCAGCTCTCTGGGAACCCAGACC |
| | | TATATCTGCAACGTGAACCACAAGCCCAGCAAC |
| | | ACCAAGGTGGACAAGAGAGTGGAGCCCAAGAG |
| | | CTGCGACAAGACCCACACCTGCCCCCCCTGCC |
| | | CAGCTCCAGAACTGCTGGGA |
| SEQ ID NO: 141 | Cys Fab | QVQLVQSGAEVKKPGESLKISCKGSGYSFTNYWI |
| | HC(EU221)-HC- | AWVRQMPGKGLEWMGIIYPSNSYTLYSPSFQGQ |
| | E152C (EU) | VTISADKSISTAYLQWSSLKASDTAMYYCARVPP |
| | | GGSISYPAFDHWGQGTLVTVSSASTKGPSVFPLA |
| | | PSSKSTSGGTAALGCLVKDYFPCPVTVSWNSGA |
| | | LTSGVHTFPAVLQSSGLYSLSSVVTVPSSSLGTQ |
| | | TYICNVNHKPSNTKVDKRVEPKSCD |
| SEQ ID NO: 142 | Fab' HC(EU230) | QVQLVQSGAEVKKPGESLKISCKGSGYSFTNYWI |
| | | AWVRQMPGKGLEWMGIIYPSNSYTLYSPSFQGQ |
| | | VTISADKSISTAYLQWSSLKASDTAMYYCARVPP |
| | | GGSISYPAFDHWGQGTLVTVSSASTKGPSVFPLA |
| | | PSSKSTSGGTAALGCLVKDYFPEPVTVSWNSGA |
| | | LTSGVHTFPAVLQSSGLYSLSSVVTVPSSSLGTQ |
| | | TYICNVNHKPSNTKVDKRVEPKSCDKTHTCPPCP |
| SEQ ID NO: 143 | Fab' HC(EU232) | QVQLVQSGAEVKKPGESLKISCKGSGYSFTNYWI |
| | | AWVRQMPGKGLEWMGIIYPSNSYTLYSPSFQGQ |
| | | VTISADKSISTAYLQWSSLKASDTAMYYCARVPP |
| | | GGSISYPAFDHWGQGTLVTVSSASTKGPSVFPLA |
| | | PSSKSTSGGTAALGCLVKDYFPEPVTVSWNSGA |
| | | LTSGVHTFPAVLQSSGLYSLSSVVTVPSSSLGTQ |
| | | TYICNVNHKPSNTKVDKRVEPKSCDKTHTCPPCP |
| | | AP |
| SEQ ID NO: 144 | Fab' HC(EU236)-Pro | QVQLVQSGAEVKKPGESLKISCKGSGYSFTNYWI |
| | | AWVRQMPGKGLEWMGIIYPSNSYTLYSPSFQGQ |
| | | VTISADKSISTAYLQWSSLKASDTAMYYCARVPP |
| | | GGSISYPAFDHWGQGTLVTVSSASTKGPSVFPLA |
| | | PSSKSTSGGTAALGCLVKDYFPEPVTVSWNSGA |
| | | LTSGVHTFPAVLQSSGLYSLSSVVTVPSSSLGTQ |
| | | TYICNVNHKPSNTKVDKRVEPKSCDKTHTCPPCP |
| | | APELLGP |
| SEQ ID NO: 101 | LCDR1 (Kabat) | SGDNIGSIYAS |
| SEQ ID NO: 102 | LCDR2 (Kabat) | RDNKRPS |
| SEQ ID NO: 103 | LCDR3 (Kabat) | SVTDMEQHSV |
| SEQ ID NO: 104 | LCDR1 (Chothia) | DNIGSIY |
| SEQ ID NO: 105 | LCDR2 (Chothia) | RDN |
| SEQ ID NO: 106 | LCDR3 (Chothia) | TDMEQHS |
| SEQ ID NO: 101 | LCDR1 (Combined) | SGDNIGSIYAS |
| SEQ ID NO: 102 | LCDR2 (Combined) | RDNKRPS |
| SEQ ID NO: 103 | LCDR3 (Combined) | SVTDMEQHSV |
| SEQ ID NO: 107 | LCDR1 (IMGT) | NIGSIY |
| SEQ ID NO: 105 | LCDR2 (IMGT) | RDN |
| SEQ ID NO: 103 | LCDR3 (IMGT) | SVTDMEQHSV |
| SEQ ID NO: 108 | VL (lambda) | DIELTQPPSVSVSPGQTASITCSGDNIGSIYASWY |
| | | QQKPGQAPVLVIYRDNKRPSGIPERFSGSNSGNT |
| | | ATLTISGTQAEDEADYYCSVTDMEQHSVFGGGT |
| | | KLTVL |
| SEQ ID NO: 109 | VL DNA | GATATCGAACTGACCCAGCCGCCGAGCGTGAG |
| | | CGTGAGCCCGGGCCAGACCGCGAGCATTACCT |
| | | GTAGCGGCGATAACATCGGTTCTATCTACGCTT |
| | | CTTGGTACCAGCAGAAACCGGGCCAGGCGCC |
| | | GGTGCTGGTGATCTACCGTGACAACAAACGTC |
| | | CGAGCGGCATCCCGGAACGTTTTAGCGGATCC |
| | | AACAGCGGCAACACCGCGACCCTGACCATTAG |
| | | CGGCACCCAGGCGGAAGACGAAGCGGATTATT |
| | | ACTGCTCCGTTACTGACATGGAACAGCATTCTG |
| | | TGTTTGGCGGCGGCACGAAGTTAACCGTCCTA |
| SEQ ID NO: 110 | Ab/Fab' LC (lambda) | DIELTQPPSVSVSPGQTASITCSGDNIGSIYASWY |
| | | QQKPGQAPVLVIYRDNKRPSGIPERFSGSNSGNT |
| | | ATLTISGTQAEDEADYYCSVTDMEQHSVFGGGT |
| | | KLTVLGQPKAAPSVTLFPPSSEELQANKATLVCLI |
| | | SDFYPGAVTVAWKADSSPVKAGVETTTPSKQSN |
| | | NKYAASSYLSLTPEQWKSHRSYSCQVTHEGSTV |
| | | EKTVAPTECS |
| SEQ ID NO: 111 | Ab/Fab' LC DNA | GATATCGAACTGACCCAGCCGCCGAGCGTGAG |
| | | CGTGAGCCCGGGCCAGACCGCGAGCATTACCT |
| | | GTAGCGGCGATAACATCGGTTCTATCTACGCTT |
| | | CTTGGTACCAGCAGAAACCGGGCCAGGCGCC |
| | | GGTGCTGGTGATCTACCGTGACAACAAACGTC |
| | | CGAGCGGCATCCCGGAACGTTTTAGCGGATCC |
| | | AACAGCGGCAACACCGCGACCCTGACCATTAG |
| | | CGGCACCCAGGCGGAAGACGAAGCGGATTATT |
| | | ACTGCTCCGTTACTGACATGGAACAGCATTCTG |
| | | TGTTTGGCGGCGGCACGAAGTTAACCGTCCTA |
| | | GGCCAGCCTAAGGCCGCTCCCTCCGTGACCCT |
| | | GTTCCCCCCCAGCTCCGAGGAACTGCAGGCCA |
| | | ACAAGGCCACCCTGGTGTGCCTGATCAGCGAC |
| | | TTCTACCCTGGCGCCGTGACCGTGGCCTGGAA |
| | | GGCCGACAGCAGCCCCGTGAAGGCCGGCGTG |
| | | GAGACAACCACCCCCAGCAAGCAGAGCAACAA |
| | | CAAGTACGCCGCCAGCAGCTACCTGAGCCTGA |
| | | CCCCCGAGCAGTGGAAGAGCCACAGAAGCTAC |
| | | AGCTGCCAGGTCACCCACGAGGGCAGCACCG |
| | | TGGAGAAAACCGTGGCCCCCACCGAGTGCAGC |
| SEQ ID NO: 145 | Cys Fab-LC | DIELTQPPSVSVSPGQTASITCSGDNIGSIYASWY |
| | (lambda)-A144C | QQKPGQAPVLVIYRDNKRPSGIPERFSGSNSGNT |
| | (EU) | ATLTISGTQAEDEADYYCSVTDMEQHSVFGGGT |
| | | KLTVLGQPKAAPSVTLFPPSSEELQANKATLVCLI |
| | | SDFYPGCVTVAWKADSSPVKAGVETTTPSKQSN |
| | | NKYAASSYLSLTPEQWKSHRSYSCQVTHEGSTV |
| | | EKTVAPTECS |

Other anti-cKIT antibody or antibody fragment (e.g., Fab or Fab′) disclosed herein include those where the amino acids or nucleic acids encoding the amino acids have been mutated, yet have at least 60, 70, 80, 90 or 95 percent identity to the sequences described in Table 1. In some aspects, it includes mutant amino acid sequences wherein no more than 1, 2, 3, 4 or 5 amino acids have been mutated in the variable regions when compared with the variable regions depicted in the sequence described in Table 1, while retaining substantially the same therapeutic activity.

Since each of these antibody or antibody fragment (e.g., Fab or Fab′) can bind to cKIT, the VH, VL, heavy chain, and light chain sequences (amino acid sequences and the nucleotide sequences encoding the amino acid sequences) can be “mixed and matched” to create other cKIT-binding antibody or antibody fragment (e.g., Fab or Fab′). Such “mixed and matched” cKIT-binding antibody or antibody fragment (e.g., Fab or Fab′) can be tested using the binding assays known in the art (e.g., ELISAs, and other assays described in the Example section). When these chains are mixed and matched, a VH sequence from a particular VH/VL pairing should be replaced with a structurally similar VH sequence. Likewise a heavy chain sequence from a particular heavy chain/light chain pairing should be replaced with a structurally similar heavy chain sequence. Likewise, a VL sequence from a particular VH/VL pairing should be replaced with a structurally similar VL sequence. Likewise, a light chain sequence from a particular heavy chain/light chain pairing should be replaced with a structurally similar light chain sequence.

Accordingly, in one aspect, the disclosure provides for an isolated antibody or antibody fragment (e.g., Fab or Fab′) having: a heavy chain variable region comprising an amino acid sequence selected from the group consisting of SEQ ID NOs: 10, 36, 54, 69, and 95 (Table 1); and a light chain variable region comprising an amino acid sequence selected from the group consisting of SEQ ID NOs: 23, 47, 82, and 108 (Table 1); wherein the antibody or antibody fragment (e.g., Fab or Fab′) specifically binds to human cKIT.

In another aspect, the disclosure provides an isolated antibody having: a heavy chain comprising an amino acid sequence selected from the group consisting of SEQ ID NOs: 12, 38, 56, 71, and 97; and a light chain comprising an amino acid sequence selected from the group consisting of SEQ ID NOs: 25, 49, 84, and 110.

In another aspect, the disclosure provides an isolated antibody fragment (e.g., Fab′) having: a heavy chain comprising an amino acid sequence selected from the group consisting of SEQ ID NOs: 14, 40, 58, 73, and 99; and a light chain comprising an amino acid sequence selected from the group consisting of SEQ ID NOs: 25, 49, 84, and 110.

In another aspect, the present disclosure provides cKIT-binding antibody or antibody fragment (e.g., Fab or Fab′) that comprises the heavy chain and light chain CDR1s, CDR2s and CDR3s as described in Table 1, or combinations thereof. The amino acid sequences of the VH CDR1s (or HCDR1) of the antibodies or antibody fragments (e.g., Fab or Fab′) are shown in SEQ ID NOs: 1, 4, 6, 7, 27, 30, 32, 33, 60, 63, 65, 66, 86, 89, 91, and 92. The amino acid sequences of the VH CDR2s (or HCDR2) of the antibodies or antibody fragments (e.g., Fab or Fab′) and are shown in SEQ ID NOs: 2, 5, 8, 28, 31, 34, 51, 52, 53, 61, 64, 67, 87, 90, and 93. The amino acid sequences of the VH CDR3s (or HCDR3) of the antibodies or antibody fragments (e.g., Fab or Fab′) are shown in SEQ ID NOs: 3, 9, 29, 35, 62, 68, 88, and 94. The amino acid sequences of the VL CDR1s (or LCDR1) of the antibodies or antibody fragments (e.g., Fab or Fab′) are shown in SEQ ID NOs: 16, 19, 22, 42, 44, 46, 75, 78, 81, 101, 104, and 107. The amino acid sequences of the VL CDR2s (or LCDR2) of the antibodies or antibody fragments (e.g., Fab or Fab′) are shown in SEQ ID NOs: 17, 20, 76, 79, 102, and 105. The amino acid sequences of the VL CDR3s (or LCDR3) of the antibodies or antibody fragments (e.g., Fab or Fab′) are shown in SEQ ID NOs: 18, 21, 43, 45, 77, 80, 103, and 106.

Given that each of these antibodies or antibody fragments (e.g., Fab or Fab′) can bind to human cKIT and that antigen-binding specificity is provided primarily by the CDR1, 2 and 3 regions, the VH CDR1, 2 and 3 sequences (or HCDR1, 2, 3) and VL CDR1, 2 and 3 sequences (or LCDR1, 2, 3) can be “mixed and matched” (i.e., CDRs from different antibodies can be mixed and match, although each antibody must contain a VH CDR1, 2 and 3 and a VL CDR1, 2 and 3 to create a cKIT-binding antibody or antibody fragment (e.g., Fab or Fab′). Such “mixed and matched” cKIT-binding antibody or antibody fragment (e.g., Fab or Fab′) can be tested using the binding assays known in the art. When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences shown herein.

Accordingly, the present disclosure provides an isolated antibody or antibody fragment (e.g., Fab or Fab′) comprising a heavy chain CDR1 (HCDR1) comprising an amino acid sequence selected from the group consisting of SEQ ID NOs: 1, 4, 6, 7, 27, 30, 32, 33, 60, 63, 65, 66, 86, 89, 91, and 92; a heavy chain CDR2 (HCDR2) comprising an amino acid sequence selected from the group consisting of SEQ ID NOs: 2, 5, 8, 28, 31, 34, 51, 52, 53, 61, 64, 67, 87, 90, and 93; a heavy chain CDR3 (HCDR3) comprising an amino acid sequence selected from the group consisting of SEQ ID NOs: 3, 9, 29, 35, 62, 68, 88, and 94; a light chain CDR1 (LCDR1) comprising an amino acid sequence selected from the group consisting of SEQ ID NOs: 16, 19, 22, 42, 44, 46, 75, 78, 81, 101, 104, and 107; a light chain CDR2 (LCDR2) comprising an amino acid sequence selected from the group consisting of SEQ ID NOs: 17, 20, 76, 79, 102, and 105; and a light chain CDR3 (LCDR3) comprising an amino acid sequence selected from the group consisting of SEQ ID NOs: 18, 21, 43, 45, 77, 80, 103, and 106; wherein the antibody specifically binds cKIT.

In some embodiments, the antibody or antibody fragment (e.g., Fab or Fab′) that specifically binds to human cKIT comprises a HCDR1 of SEQ ID NO: 1, a HCDR2 of SEQ ID NO: 2; a HCDR3 of SEQ ID NO: 3; a LCDR1 of SEQ ID NO:16; a LCDR2 of SEQ ID NO: 17; and a LCDR3 of SEQ ID NO: 18.

In some embodiments, the antibody or antibody fragment (e.g., Fab or Fab′) that specifically binds to human cKIT comprises a HCDR1 of SEQ ID NO: 4, a HCDR2 of SEQ ID NO: 5; a HCDR3 of SEQ ID NO: 3; a LCDR1 of SEQ ID NO:19; a LCDR2 of SEQ ID NO: 20; and a LCDR3 of SEQ ID NO: 21.

In some embodiments, the antibody or antibody fragment (e.g., Fab or Fab′) that specifically binds to human cKIT comprises a HCDR1 of SEQ ID NO: 6, a HCDR2 of SEQ ID NO: 2; a HCDR3 of SEQ ID NO: 3; a LCDR1 of SEQ ID NO:16; a LCDR2 of SEQ ID NO: 17; and a LCDR3 of SEQ ID NO: 18.

In some embodiments, the antibody or antibody fragment (e.g., Fab or Fab′) that specifically binds to human cKIT comprises a HCDR1 of SEQ ID NO: 7, a HCDR2 of SEQ ID NO: 8; a HCDR3 of SEQ ID NO: 9; a LCDR1 of SEQ ID NO: 22; a LCDR2 of SEQ ID NO: 20; and a LCDR3 of SEQ ID NO: 18.

In some embodiments, the antibody or antibody fragment (e.g., Fab or Fab′) that specifically binds to human cKIT comprises a HCDR1 of SEQ ID NO: 27, a HCDR2 of SEQ ID NO: 28; a HCDR3 of SEQ ID NO: 29; a LCDR1 of SEQ ID NO: 42; a LCDR2 of SEQ ID NO: 17; and a LCDR3 of SEQ ID NO: 43.

In some embodiments, the antibody or antibody fragment (e.g., Fab or Fab′) that specifically binds to human cKIT comprises a HCDR1 of SEQ ID NO: 30, a HCDR2 of SEQ ID NO: 31; a HCDR3 of SEQ ID NO: 29; a LCDR1 of SEQ ID NO: 44; a LCDR2 of SEQ ID NO: 20; and a LCDR3 of SEQ ID NO: 45.

In some embodiments, the antibody or antibody fragment (e.g., Fab or Fab′) that specifically binds to human cKIT comprises a HCDR1 of SEQ ID NO: 32, a HCDR2 of SEQ ID NO: 28; a HCDR3 of SEQ ID NO: 29; a LCDR1 of SEQ ID NO: 42; a LCDR2 of SEQ ID NO: 17; and a LCDR3 of SEQ ID NO: 43.

In some embodiments, the antibody or antibody fragment (e.g., Fab or Fab′) that specifically binds to human cKIT comprises a HCDR1 of SEQ ID NO: 33, a HCDR2 of SEQ ID NO: 34; a HCDR3 of SEQ ID NO: 35; a LCDR1 of SEQ ID NO: 46; a LCDR2 of SEQ ID NO: 20; and a LCDR3 of SEQ ID NO: 43.

In some embodiments, the antibody or antibody fragment (e.g., Fab or Fab′) that specifically binds to human cKIT comprises a HCDR1 of SEQ ID NO: 1, a HCDR2 of SEQ ID NO: 51; a HCDR3 of SEQ ID NO: 3; a LCDR1 of SEQ ID NO:16; a LCDR2 of SEQ ID NO: 17; and a LCDR3 of SEQ ID NO: 18.

In some embodiments, the antibody or antibody fragment (e.g., Fab or Fab′) that specifically binds to human cKIT comprises a HCDR1 of SEQ ID NO: 4, a HCDR2 of SEQ ID NO: 52; a HCDR3 of SEQ ID NO: 3; a LCDR1 of SEQ ID NO:19; a LCDR2 of SEQ ID NO: 20; and a LCDR3 of SEQ ID NO: 21.

In some embodiments, the antibody or antibody fragment (e.g., Fab or Fab′) that specifically binds to human cKIT comprises a HCDR1 of SEQ ID NO: 6, a HCDR2 of SEQ ID NO: 51; a HCDR3 of SEQ ID NO: 3; a LCDR1 of SEQ ID NO:16; a LCDR2 of SEQ ID NO: 17; and a LCDR3 of SEQ ID NO: 18.

In some embodiments, the antibody or antibody fragment (e.g., Fab or Fab′) that specifically binds to human cKIT comprises a HCDR1 of SEQ ID NO: 7, a HCDR2 of SEQ ID NO: 53; a HCDR3 of SEQ ID NO: 9; a LCDR1 of SEQ ID NO: 22; a LCDR2 of SEQ ID NO: 20; and a LCDR3 of SEQ ID NO: 18.

In some embodiments, the antibody or antibody fragment (e.g., Fab or Fab′) that specifically binds to human cKIT comprises a HCDR1 of SEQ ID NO: 60, a HCDR2 of SEQ ID NO: 61; a HCDR3 of SEQ ID NO: 62; a LCDR1 of SEQ ID NO: 75; a LCDR2 of SEQ ID NO: 76; and a LCDR3 of SEQ ID NO: 77.

In some embodiments, the antibody or antibody fragment (e.g., Fab or Fab′) that specifically binds to human cKIT comprises a HCDR1 of SEQ ID NO: 63, a HCDR2 of SEQ ID NO: 64; a HCDR3 of SEQ ID NO: 62; a LCDR1 of SEQ ID NO: 78; a LCDR2 of SEQ ID NO: 79; and a LCDR3 of SEQ ID NO: 80.

In some embodiments, the antibody or antibody fragment (e.g., Fab or Fab′) that specifically binds to human cKIT comprises a HCDR1 of SEQ ID NO: 65, a HCDR2 of SEQ ID NO: 61; a HCDR3 of SEQ ID NO: 62; a LCDR1 of SEQ ID NO:75; a LCDR2 of SEQ ID NO: 76; and a LCDR3 of SEQ ID NO: 77.

In some embodiments, the antibody or antibody fragment (e.g., Fab or Fab′) that specifically binds to human cKIT comprises a HCDR1 of SEQ ID NO: 66, a HCDR2 of SEQ ID NO: 67; a HCDR3 of SEQ ID NO: 68; a LCDR1 of SEQ ID NO: 81; a LCDR2 of SEQ ID NO: 79; and a LCDR3 of SEQ ID NO: 77.

In some embodiments, the antibody or antibody fragment (e.g., Fab or Fab′) that specifically binds to human cKIT comprises a HCDR1 of SEQ ID NO: 86, a HCDR2 of SEQ ID NO: 87; a HCDR3 of SEQ ID NO: 88; a LCDR1 of SEQ ID NO: 101; a LCDR2 of SEQ ID NO: 102; and a LCDR3 of SEQ ID NO: 103.

In some embodiments, the antibody or antibody fragment (e.g., Fab or Fab′) that specifically binds to human cKIT comprises a HCDR1 of SEQ ID NO: 89, a HCDR2 of SEQ ID NO: 90; a HCDR3 of SEQ ID NO: 88; a LCDR1 of SEQ ID NO: 104; a LCDR2 of SEQ ID NO: 105; and a LCDR3 of SEQ ID NO: 106.

In some embodiments, the antibody or antibody fragment (e.g., Fab or Fab′) that specifically binds to human cKIT comprises a HCDR1 of SEQ ID NO: 91, a HCDR2 of SEQ ID NO: 87; a HCDR3 of SEQ ID NO: 88; a LCDR1 of SEQ ID NO: 101; a LCDR2 of SEQ ID NO: 102; and a LCDR3 of SEQ ID NO: 103.

In some embodiments, the antibody or antibody fragment (e.g., Fab or Fab′) that specifically binds to human cKIT comprises a HCDR1 of SEQ ID NO: 92, a HCDR2 of SEQ ID NO: 93; a HCDR3 of SEQ ID NO: 94; a LCDR1 of SEQ ID NO: 107; a LCDR2 of SEQ ID NO: 105; and a LCDR3 of SEQ ID NO: 103.

In some embodiments, the antibody or antibody fragment (e.g., Fab or Fab′) that specifically binds to human cKIT comprises a heavy chain variable region (VH) comprising the amino acid sequence of SEQ ID NO: 10, and a light chain variable region (VL) comprising the amino acid sequence of SEQ ID NO: 23.

In some embodiments, the antibody or antibody fragment (e.g., Fab or Fab′) that specifically binds to human cKIT comprises a VH comprising the amino acid sequence of SEQ ID NO: 36, and a VL comprising the amino acid sequence of SEQ ID NO: 47.

In some embodiments, the antibody or antibody fragment (e.g., Fab or Fab′) that specifically binds to human cKIT comprises a VH comprising the amino acid sequence of SEQ ID NO: 54, and a VL comprising the amino acid sequence of SEQ ID NO: 23.

In some embodiments, the antibody or antibody fragment (e.g., Fab or Fab′) that specifically binds to human cKIT comprises a VH comprising the amino acid sequence of SEQ ID NO: 69, and a VL comprising the amino acid sequence of SEQ ID NO: 82.

In some embodiments, the antibody or antibody fragment (e.g., Fab or Fab′) that specifically binds to human cKIT comprises a VH comprising the amino acid sequence of SEQ ID NO: 95, and a VL comprising the amino acid sequence of SEQ ID NO: 108.

In some embodiments, the antibody fragment (e.g., Fab′) that specifically binds to human cKIT comprises a heavy chain comprising the amino acid sequence of SEQ ID NO: 14, and a light chain comprising the amino acid sequence of SEQ ID NO: 25.

In some embodiments, the antibody fragment (e.g., Fab′) that specifically binds to human cKIT comprises a heavy chain comprising the amino acid sequence of SEQ ID NO: 40, and a light chain comprising the amino acid sequence of SEQ ID NO: 49.

In some embodiments, the antibody fragment (e.g., Fab′) that specifically binds to human cKIT comprises a heavy chain comprising the amino acid sequence of SEQ ID NO: 58, and a light chain comprising the amino acid sequence of SEQ ID NO: 25.

In some embodiments, the antibody fragment (e.g., Fab′) that specifically binds to human cKIT comprises a heavy chain comprising the amino acid sequence of SEQ ID NO: 73, and a light chain comprising the amino acid sequence of SEQ ID NO: 84.

In some embodiments, the antibody fragment (e.g., Fab′) that specifically binds to human cKIT comprises a heavy chain comprising the amino acid sequence of SEQ ID NO: 99, and a light chain comprising the amino acid sequence of SEQ ID NO: 110.

In some embodiments, the antibody fragment (e.g., Fab′) that specifically binds to human cKIT comprises a heavy chain comprising an amino acid sequence selected from SEQ ID NO: 119, 120 or 121, and a light chain comprising the amino acid sequence of SEQ ID NO: 25.

In some embodiments, the antibody fragment (e.g., Fab′) that specifically binds to human cKIT comprises a heavy chain comprising an amino acid sequence selected from SEQ ID NO: 125, 126, or 127, and a light chain comprising the amino acid sequence of SEQ ID NO: 49.

In some embodiments, the antibody fragment (e.g., Fab′) that specifically binds to human cKIT comprises a heavy chain comprising an amino acid sequence selected from SEQ ID NO: 131, 132, or 133, and a light chain comprising the amino acid sequence of SEQ ID NO: 25.

In some embodiments, the antibody fragment (e.g., Fab′) that specifically binds to human cKIT comprises a heavy chain comprising an amino acid sequence selected from SEQ ID NO: 137, 138, or 139, and a light chain comprising the amino acid sequence of SEQ ID NO: 84.

In some embodiments, the antibody fragment (e.g., Fab′) that specifically binds to human cKIT comprises a heavy chain comprising an amino acid sequence selected from SEQ ID NO: 142, 143, or 144, and a light chain comprising the amino acid sequence of SEQ ID NO: 110.

In some embodiments, the antibody that specifically binds to human cKIT comprises a heavy chain comprising the amino acid sequence of SEQ ID NO: 12, and a light chain comprising the amino acid sequence of SEQ ID NO: 25.

In some embodiments, the antibody that specifically binds to human cKIT comprises a heavy chain comprising the amino acid sequence of SEQ ID NO: 38, and a light chain comprising the amino acid sequence of SEQ ID NO: 49.

In some embodiments, the antibody that specifically binds to human cKIT comprises a heavy chain comprising the amino acid sequence of SEQ ID NO: 56, and a light chain comprising the amino acid sequence of SEQ ID NO: 25.

In some embodiments, the antibody that specifically binds to human cKIT comprises a heavy chain comprising the amino acid sequence of SEQ ID NO: 71, and a light chain comprising the amino acid sequence of SEQ ID NO: 84.

In some embodiments, the antibody that specifically binds to human cKIT comprises a heavy chain comprising the amino acid sequence of SEQ ID NO: 97, and a light chain comprising the amino acid sequence of SEQ ID NO: 110.

In certain aspects, the antibody or antibody fragment (e.g., Fab or Fab′) that specifically binds to human cKIT is an antibody or antibody fragment (e.g., Fab or Fab′) described in Table 1.

1. Antibodies that Bind to the Same Epitope

The present disclosure provides the antibody or antibody fragment (e.g., Fab or Fab′) that specifically binds to an epitope within the extracellular domain of the human cKIT receptor. In certain aspects the antibody or antibody fragment (e.g., Fab or Fab′) can bind to an epitope within domains 1-3 of the human cKIT extracellular domain.

The present disclosure also provides antibody or antibody fragment (e.g., Fab or Fab′) that binds to the same epitope as the anti-cKIT antibody or antibody fragment (e.g., Fab or Fab′) described in Table 1. Additional antibody or antibody fragment (e.g., Fab or Fab′) can therefore be identified based on their ability to cross-compete (e.g., to competitively inhibit the binding of, in a statistically significant manner) with other antibody or antibody fragment (e.g., Fab or Fab′) in cKIT binding assays. A high throughput process for “binning” antibodies based upon their cross-competition is described in International Patent Application No. WO 2003/48731. The ability of a test antibody or antibody fragment (e.g., Fab or Fab′) to inhibit the binding of antibody or antibody fragment (e.g., Fab or Fab′) disclosed herein to a cKIT protein (e.g., human cKIT) demonstrates that the test antibody or antibody fragment (e.g., Fab or Fab′) can compete with that antibody or antibody fragment (e.g., Fab or Fab′) for binding to cKIT; such an antibody or antibody fragment (e.g., Fab or Fab′) may, according to non-limiting theory, bind to the same or a related (e.g., a structurally similar or spatially proximal) epitope on the cKIT protein as the antibody or antibody fragment (e.g., Fab or Fab′) with which it competes. In a certain aspect, the antibody or antibody fragment (e.g., Fab or Fab′) that binds to the same epitope on cKIT as the antibody or antibody fragment (e.g., Fab or Fab′) disclosed herein is a human or humanized antibody or antibody fragment (e.g., Fab or Fab′). Such human or humanized antibody or antibody fragment (e.g., Fab or Fab′) can be prepared and isolated as described herein.

### 2. Modification of the Framework

Antibody drug conjugates disclosed herein may comprise modified cKIT-binding antibody or antibody fragment (e.g., Fab or Fab′) that comprises modifications to framework residues within VH and/or VL, e.g. to improve the properties of the antibody drug conjugate.

In some embodiments, framework modifications are made to decrease immunogenicity of an antibody or antibody drug conjugate. For example, one approach is to “back-mutate” one or more framework residues to a corresponding germline sequence. Such residues can be identified by comparing antibody framework sequences to germline sequences from which the antibody is derived. To “match” framework region sequences to desired germline configuration, residues can be “back-mutated” to a corresponding germline sequence by, for example, site-directed mutagenesis. Such “back-mutated” antibodies or antibody drug conjugates are also intended to be encompassed by the invention.

Another type of framework modification involves mutating one or more residues within the framework region, or even within one or more CDR regions, to remove T-cell epitopes to thereby reduce the potential immunogenicity of the antibody or antibody drug conjugate. This approach is also referred to as “deimmunization” and is described in further detail in U.S. Patent Publication No. 2003/0153043 by Carr et al.

In addition or alternative to modifications made within the framework or CDR regions, antibodies can be engineered to alter one or more functional properties of the antibody, such as serum half-life, complement fixation. Furthermore, an antibody can be chemically modified (e.g., one or more chemical moieties can be attached to the antibody) or be modified to alter its glycosylation, again to alter one or more functional properties of the antibody. Each of these aspects is described in further detail below.

In one aspect, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains, to increase or decrease the stability of the antibody, or to allow conjugation to another molecule.

In some embodiments, the antibody or antibody fragment (e.g., Fab or Fab′) disclosed herein include modified or engineered amino acid residues, e.g., one or more cysteine residues, as sites for conjugation to a drug moiety (Junutula J R, et al.: Nat Biotechnol 2008, 26:925-932). In one embodiment, the invention provides a modified antibody or antibody fragment (e.g., Fab or Fab′) comprising a substitution of one or more amino acids with cysteine at the positions described herein. Sites for cysteine substitution are in the constant regions of the antibody or antibody fragment (e.g., Fab or Fab′) and are thus applicable to a variety of antibody or antibody fragment (e.g., Fab or Fab′), and the sites are selected to provide stable and homogeneous conjugates. A modified antibody or fragment can have one, two or more cysteine substitutions, and these substitutions can be used in combination with other modification and conjugation methods as described herein. Methods for inserting cysteine at specific locations of an antibody are known in the art, see, e.g., Lyons et al, (1990) Protein Eng., 3:703-708, WO 2011/005481, WO2014/124316, WO 2015/138615. In certain embodiments, a modified antibody comprises a substitution of one or more amino acids with cysteine on its constant region selected from positions 117, 119, 121, 124, 139, 152, 153, 155, 157, 164, 169, 171, 174, 189, 191, 195, 197, 205, 207, 246, 258, 269, 274, 286, 288, 290, 292, 293, 320, 322, 326, 333, 334, 335, 337, 344, 355, 360, 375, 382, 390, 392, 398, 400 and 422 of a heavy chain of the antibody, and wherein the positions are numbered according to the EU system. In certain embodiments, a modified antibody fragment (e.g., Fab or Fab′) comprises a substitution of one or more amino acids with cysteine on its constant region selected from positions 121, 124, 152, 153, 155, 157, 164, 169, 171, 174, 189, and 207 of a heavy chain of the antibody fragment (e.g., Fab or Fab′), and wherein the positions are numbered according to the EU system. In certain embodiments, a modified antibody fragment (e.g., Fab or Fab′) comprises a substitution of one or more amino acids with cysteine on its constant region selected from positions 124, 152, 153, 155, 157, 164, 174, 189, and 207 of a heavy chain of the antibody fragment (e.g., Fab or Fab′), and wherein the positions are numbered according to the EU system.

In some embodiments, a modified antibody or antibody fragment (e.g., Fab or Fab′) comprises a substitution of one or more amino acids with cysteine on its constant region selected from positions 107, 108, 109, 114, 126, 127, 129, 142, 143, 145, 152, 154, 156, 157, 159, 161, 165, 168, 169, 170, 182, 183, 188, 197, 199, and 203 of a light chain of the antibody or antibody fragment (e.g., Fab or Fab′), wherein the positions are numbered according to the EU system, and wherein the light chain is a human kappa light chain. In some embodiments, a modified antibody or antibody fragment (e.g., Fab or Fab′) comprises a substitution of one or more amino acids with cysteine on its constant region selected from positions 107, 108, 114, 126, 127, 129, 142, 159, 161, 165, 183, and 203 of a light chain of the antibody or antibody fragment (e.g., Fab or Fab′), wherein the positions are numbered according to the EU system, and wherein the light chain is a human kappa light chain. In some embodiments, a modified antibody or antibody fragment (e.g., Fab or Fab′) comprises a substitution of one or more amino acids with cysteine on its constant region selected from positions 114, 129, 142, 145, 152, 159, 161, 165, and 197 of a light chain of the antibody or antibody fragment (e.g., Fab or Fab′), wherein the positions are numbered according to the EU system, and wherein the light chain is a human kappa light chain. In some embodiments, a modified antibody or antibody fragment (e.g., Fab or Fab′) comprises a substitution of one or more amino acids with cysteine on its constant region selected from positions 107, 108, 109, 126, 143, 145, 152, 154, 156, 157, 159, 182, 183, 188, 197, 199, and 203 of a light chain of the antibody or antibody fragment (e.g., Fab or Fab′), wherein the positions are numbered according to the EU system, and wherein the light chain is a human kappa light chain. In some embodiments, a modified antibody or antibody fragment (e.g., Fab or Fab′) comprises a substitution of one or more amino acids with cysteine on its constant region selected from positions 145, 152, and 197 of a light chain of the antibody or antibody fragment (e.g., Fab or Fab′), wherein the positions are numbered according to the EU system, and wherein the light chain is a human kappa light chain. In some embodiments, a modified antibody or antibody fragment (e.g., Fab or Fab′) comprises a substitution of one or more amino acids with cysteine on its constant region selected from positions 114 and 165 of a light chain of the antibody or antibody fragment (e.g., Fab or Fab′), wherein the positions are numbered according to the EU system, and wherein the light chain is a human kappa light chain.

In some embodiments, a modified antibody or antibody fragment (e.g., Fab or Fab′) comprises a substitution of one or more amino acids with cysteine on its constant region selected from positions 143, 145, 147, 156, 159, 163, 168 of a light chain of the antibody or antibody fragment (e.g., Fab or Fab′), wherein the positions are numbered according to the EU system, and wherein the light chain is a human lambda light chain. In some embodiments, a modified antibody or antibody fragment (e.g., Fab or Fab′) comprises cysteine at position 143 (by EU numbering) of a light chain of the antibody or antibody fragment (e.g., Fab or Fab′), wherein the light chain is a human lambda light chain.

In certain embodiments, a modified antibody or antibody fragment (e.g., Fab or Fab′) comprises a combination of substitution of two or more amino acids with cysteine on its constant regions and the combination of positions can be selected from any of the positions listed above.

In some embodiments, a modified antibody or antibody fragment (e.g., Fab or Fab′) comprises cysteine at one or more of the following positions: position 124 of the heavy chain, position 152 of the heavy chain, position 153 of the heavy chain, position 155 of the heavy chain, position 157 of the heavy chain, position 164 of the heavy chain, position 174 of the heavy chain, position 114 of the light chain, position 129 of the light chain, position 142 of the light chain, position 159 of the light chain, position 161 of the light chain, or position 165 of the light chain, and wherein the positions are numbered according to the EU system, and wherein the light chain is a kappa chain. In some embodiments, a modified antibody or antibody fragment (e.g., Fab or Fab′) comprises cysteine at four of the following positions: position 124 of the heavy chain, position 152 of the heavy chain, position 153 of the heavy chain, position 155 of the heavy chain, position 157 of the heavy chain, position 164 of the heavy chain, position 174 of the heavy chain, position 114 of the light chain, position 129 of the light chain, position 142 of the light chain, position 159 of the light chain, position 161 of the light chain, or position 165 of the light chain, and wherein the positions are numbered according to the EU system, and wherein the light chain is a kappa chain.

In some embodiments, a modified antibody or antibody fragment (e.g., Fab or Fab′) comprises cysteine at position 152 of the heavy chain, wherein the position is numbered according to the EU system. In some embodiments, a modified antibody or antibody fragment (e.g., Fab or Fab′) comprises cysteine at position 124 of the heavy chain, wherein the position is numbered according to the EU system. In some embodiments, a modified antibody or antibody fragment (e.g., Fab or Fab′) comprises cysteine at position 165 of the light chain, wherein the position is numbered according to the EU system and wherein the light chain is a kappa chain. In some embodiments, a modified antibody or antibody fragment (e.g., Fab or Fab′) comprises cysteine at position 114 of the light chain, wherein the position is numbered according to the EU system and wherein the light chain is a kappa chain. In some embodiments, a modified antibody or antibody fragment (e.g., Fab or Fab′) comprises cysteine at position 143 of the light chain, wherein the position is numbered according to the EU system and wherein the light chain is a lambda chain.

In some embodiments, a modified antibody or antibody fragment (e.g., Fab or Fab′) comprises cysteines at position 152 of the heavy chain and position 165 of the light chain and wherein the positions are numbered according to the EU system, and wherein the light chain is a kappa chain. In some embodiments, a modified antibody or antibody fragment (e.g., Fab or Fab′) comprises cysteines at position 152 of the heavy chain and position 114 of the light chain and wherein the positions are numbered according to the EU system, and wherein the light chain is a kappa chain. In some embodiments, a modified antibody or antibody fragment (e.g., Fab or Fab′) comprises cysteines at position 152 of the heavy chain and position 143 of the light chain and wherein the positions are numbered according to the EU system, and wherein the light chain is a lambda chain. In some embodiments, a modified antibody or antibody fragment (e.g., Fab or Fab′) comprises cysteines at position 124 and position 152 of the heavy chain and wherein the positions are numbered according to the EU system.

In some embodiments, a modified antibody or antibody fragment (e.g., Fab or Fab′) comprises cysteine at one or more of the following positions: position 155 of the heavy chain, position 189 of the heavy chain, position 207 of the heavy chain, position 145 of the light chain, position 152 of the light chain, or position 197 of the light chain, and wherein the positions are numbered according to the EU system, and wherein the light chain is a kappa chain. In some embodiments, a modified antibody or antibody fragment (e.g., Fab or Fab′) comprises cysteine at two or more (e.g., 2, 3, 4) of the following positions: position 155 of the heavy chain, position 189 of the heavy chain, position 207 of the heavy chain, position 145 of the light chain, position 152 of the light chain, or position 197 of the light chain, and wherein the positions are numbered according to the EU system, and wherein the light chain is a kappa chain.

In some embodiments, a modified antibody or antibody fragment (e.g., Fab or Fab′) comprises cysteine at one or more of the following positions: position 124 of the heavy chain, position 152 of the heavy chain, position 153 of the heavy chain, position 155 of the heavy chain, position 157 of the heavy chain, position 164 of the heavy chain, position 174 of the heavy chain, position 114 of the light chain, position 129 of the light chain, position 142 of the light chain, position 159 of the light chain, position 161 of the light chain, or position 165 of the light chain, and wherein the positions are numbered according to the EU system, and wherein the light chain is a kappa chain. In some embodiments, a modified antibody or antibody fragment (e.g., Fab or Fab′) comprises cysteine at two or more (e.g., 2, 3, 4) of the following positions: position 124 of the heavy chain, position 152 of the heavy chain, position 153 of the heavy chain, position 155 of the heavy chain, position 157 of the heavy chain, position 164 of the heavy chain, position 174 of the heavy chain, position 114 of the light chain, position 129 of the light chain, position 142 of the light chain, position 159 of the light chain, position 161 of the light chain, or position 165 of the light chain, and wherein the positions are numbered according to the EU system, and wherein the light chain is a kappa chain.

In some embodiments, a modified antibody fragment (e.g., Fab) that specifically binds to human cKIT comprises a heavy chain comprising the amino acid sequence of SEQ ID NO: 118, and a light chain comprising the amino acid sequence of SEQ ID NO: 122.

In some embodiments, a modified antibody fragment (e.g., Fab) that specifically binds to human cKIT comprises a heavy chain comprising the amino acid sequence of SEQ ID NO: 118, and a light chain comprising the amino acid sequence of SEQ ID NO: 123.

In some embodiments, a modified antibody fragment (e.g., Fab) that specifically binds to human cKIT comprises a heavy chain comprising the amino acid sequence of SEQ ID NO: 124, and a light chain comprising the amino acid sequence of SEQ ID NO: 128.

In some embodiments, a modified antibody fragment (e.g., Fab) that specifically binds to human cKIT comprises a heavy chain comprising the amino acid sequence of SEQ ID NO: 124, and a light chain comprising the amino acid sequence of SEQ ID NO: 129.

In some embodiments, a modified antibody fragment (e.g., Fab) that specifically binds to human cKIT comprises a heavy chain comprising the amino acid sequence of SEQ ID NO: 130, and a light chain comprising the amino acid sequence of SEQ ID NO: 134.

In some embodiments, a modified antibody fragment (e.g., Fab) that specifically binds to human cKIT comprises a heavy chain comprising the amino acid sequence of SEQ ID NO: 130, and a light chain comprising the amino acid sequence of SEQ ID NO: 135.

In some embodiments, a modified antibody fragment (e.g., Fab) that specifically binds to human cKIT comprises a heavy chain comprising the amino acid sequence of SEQ ID NO: 136, and a light chain comprising the amino acid sequence of SEQ ID NO: 140.

In some embodiments, a modified antibody fragment (e.g., Fab) that specifically binds to human cKIT comprises a heavy chain comprising the amino acid sequence of SEQ ID NO: 141, and a light chain comprising the amino acid sequence of SEQ ID NO: 145.

3. Production of the cKIT Antibodies or Antibody Fragments

Anti-cKIT antibody or antibody fragment (e.g., Fab or Fab′) can be produced by any means known in the art, including but not limited to, recombinant expression, chemical synthesis, or enzymatic digestion of full-length monoclonal antibodies, which can be obtained by, e.g., hybridoma or recombinant production. Recombinant expression can be from any appropriate host cells known in the art, for example, mammalian host cells, bacterial host cells, yeast host cells, insect host cells, or made by a cell-free system (e.g., Sutro's Xpress CF™ Platform, http://www.sutrobio.com/technology/.

The disclosure further provides polynucleotides encoding the antibody or antibody fragment (e.g., Fab or Fab′) described herein, e.g., polynucleotides encoding heavy or light chain variable regions or segments comprising the complementarity determining regions as described herein. In some aspects, the polynucleotide encoding the heavy chain variable regions (VH) has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NOs: 11, 37, 55, 70, and 96. In some aspects, the polynucleotide encoding the light chain variable regions (VL) has at least 85%, 89%, 90%, 91%, 92%, 93%, 94%, 95%, 96%, 97%, 98%, 99%, or 100% nucleic acid sequence identity with a polynucleotide selected from the group consisting of SEQ ID NOs: 24, 48, 83, and 109.

In some aspects, the polynucleotide encoding the antibody heavy chain has at least 85%, 89%, 90%, 91%, 92%, 93%, 94%, 95%, 96%, 97%, 98%, 99%, or 100% nucleic acid sequence identity with a polynucleotide of SEQ ID NOs: 13, 39, 57, 72, and 98. In some aspects, the polynucleotide encoding the antibody light chain has at least 85%, 89%, 90%, 91%, 92%, 93%, 94%, 95%, 96%, 97%, 98%, 99%, or 100% nucleic acid sequence identity with a polynucleotide of SEQ ID NOs: 26, 50, 85, and 111.

In some aspects, the polynucleotide encoding the Fab′ heavy chain has at least 85%, 89%, 90%, 91%, 92%, 93%, 94%, 95%, 96%, 97%, 98%, 99%, or 100% nucleic acid sequence identity with a polynucleotide of SEQ ID NOs: 15, 41, 59, 74, and 100. In some aspects, the polynucleotide encoding the Fab′ light chain has at least 85%, 89%, 90%, 91%, 92%, 93%, 94%, 95%, 96%, 97%, 98%, 99%, or 100% nucleic acid sequence identity with a polynucleotide of SEQ ID NOs: 26, 50, 85, and 111.

The polynucleotides of the present disclosure can encode only the variable region sequence of an anti-cKIT antibody or antibody fragment (e.g., Fab or Fab′). They can also encode both a variable region and a constant region of the antibody or antibody fragment (e.g., Fab or Fab′). Some of the polynucleotide sequences encode a polypeptide that comprises variable regions of both the heavy chain and the light chain of one of an exemplified anti-cKIT antibody or antibody fragment (e.g., Fab or Fab′).

The polynucleotide sequences can be produced by de novo solid-phase DNA synthesis or by PCR mutagenesis of an existing sequence (e.g., sequences as described in the Examples below) encoding an anti-cKIT antibody or its binding fragment. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided in the present disclosure are expression vectors and host cells for producing the anti-cKIT antibody or antibody fragment (e.g., Fab or Fab′) described above. Various expression vectors can be employed to express the polynucleotides encoding the anti-cKIT antibody or antibody fragment (e.g., Fab or Fab′). Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Genet. 15:345, 1997). For example, nonviral vectors useful for expression of the anti-cKIT polynucleotides and polypeptides in mammalian (e.g., human) cells include pThioHis A, B & C, pcDNA3.1/His, pEBVHis A, B & C (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding an anti-cKIT antibody or antibody fragment (e.g., Fab or Fab′). In some aspects, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of an anti-cKIT antibody or antibody fragment (e.g., Fab or Fab′). These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted anti-cKIT antibody or antibody fragment (e.g., Fab or Fab′) sequences. More often, the inserted anti-cKIT antibody or antibody fragment (e.g., Fab or Fab′) sequences are linked to a signal sequences before inclusion in the vector. Vectors to be used to receive sequences encoding anti-cKIT antibody or antibody fragment (e.g., Fab or Fab′) light and heavy chain variable domains sometimes also encode constant regions or parts thereof. Such vectors allow expression of the variable regions as fusion proteins with the constant regions thereby leading to production of intact antibodies or fragments thereof.

The host cells for harboring and expressing the anti-cKIT antibody or antibody fragment (e.g., Fab or Fab′) chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-cKIT antibody or antibody fragment (e.g., Fab or Fab′) polypeptides. Insect cells in combination with baculovirus vectors can also be used.

In other aspects, mammalian host cells are used to express and produce the anti-cKIT antibody or antibody fragment (e.g., Fab or Fab′) polypeptides of the present disclosure. For example, they can be either a hybridoma cell line expressing endogenous immunoglobulin genes (e.g., the myeloma hybridoma clones as described in the Examples) or a mammalian cell line harboring an exogenous expression vector (e.g., the SP2/0 myeloma cells exemplified below). These include any normal mortal or normal or abnormal immortal animal or human cell. For example, a number of suitable host cell lines capable of secreting intact immunoglobulins have been developed, including the CHO cell lines, various COS cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express anti-cKIT antibody or antibody fragment (e.g., Fab or Fab′) chains can be prepared using expression vectors which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

Antibody fragments, such as Fab or Fab′ may be produced by proteolytic cleavage of immunoglobulin molecules, using enzymes such as papain (to produce Fab fragments), or pepsin (to produce Fab′ fragments), etc. Compared to Fab fragments, Fab′ fragments also contain the hinge region which includes the two natural cysteines that form disulfide bonds between two heavy chains of an immunoglobulin molecule.

### Therapeutic Uses

The conjugates of the present disclosure are useful in a variety of applications including, but not limited to, for ablating hematopoietic stem cells in a patient in need thereof, e.g., a hematopoietic stem cell transplantation recipient. Accordingly, provided herein are methods of ablating hematopoietic stem cells in a patient in need thereof by administering to the patient an effective amount of any of the conjugates described herein. Provided herein are also methods of conditioning a hematopoietic stem cell transplantation patient (e.g., a transplant recipient) by administering to the patient an effective amount of any of the conjugates described herein, and allowing a sufficient period of time for the conjugates to clear from the patient's circulation before performing hematopoietic stem cell transplantation to the patient. The conjugates can be administered to the patient intravenously. Also provided are use of any of the conjugates or pharmaceutical compositions described herein for ablating hematopoietic stem cells in a patient in need thereof. Further provided are use of any of the conjugates or pharmaceutical compositions described herein in the manufacture of a medicament for ablating hematopoietic stem cells in a patient in need thereof.

Endogenous hematopoietic stem cells usually reside within bone marrow sinusoids. This physical environment in which stem cells reside is referred to as the stem cell microenvironment, or stem cell niche. The stromal and other cells involved in this niche provide soluble and bound factors, which have a multitude of effects. Various models have been proposed for the interaction between hematopoietic stem cells and their niche. For example, a model has been suggested where, when a stem cell divides, only one daughter remains in the niche and the other daughter cell leaves the niche to differentiate. It has been proposed that the efficiency of engraftment can be enhanced by selective depletion of endogenous hematopoietic stem cells, thereby opening the stem cell niches for the engraftment of donor stem cells (see e.g., WO 2008/067115).

Hematopoietic stem cell (HSC) transplantation, or bone marrow transplantation (as called earlier), is an established treatment for a wide range of diseases that affect the body's blood stem cells such as leukemia, severe anemia, immune defects, and some enzyme deficiency diseases. These illnesses often lead to the patient needing to have his bone marrow replaced by new, healthy blood cells.

HSC transplantation is often allogeneic, which means that the patient receives stem cells from another individual of the same species, either a sibling, matched related, haploidentical related or unrelated, volunteer donor. It is estimated that about 30% of patients in need of hematopoietic stem cell transplantation have access to a sibling whose tissue type is suitable. The other 70% of patients must rely on the matching of an unrelated, volunteer donor or the availability of a haploidentical, related donor. It is important that the characteristics of donor and patient cells are comparable. The hematopoietic stem cell transplantation could also be autologous, in which the transplanted cells are originating from the subject itself, i.e., the donor and the recipient are the same individual. Further, the transplantations could be syngeneic, i.e., from a genetically identical individual such as a twin. In an additional aspect the transplantations could be xenogeneic, i.e., originating from a different species, which is of interest when there are not sufficient donors of the same species, such as for organ transplantations.

Before the HSC transplantation, patients usually undergo a pre-treatment or conditioning method. The purpose of this pre-treatment or conditioning is to remove as many undesired cells (e.g., malignant/cancer cells) in the body as possible, to minimize rejection, and/or to open up stem cell niches by depletion of endogenous HSCs for efficient engraftment of donor stem cells into those niches. Donor's healthy HSCs are then given to the patient intravenously, or in some cases intraosseously. Many risks, however, are associated with HSC transplantation, including poor engraftment, immunological rejection, graft-versus-host disease (GVHD), or infection. Although the donor and the patient's cells appear to be equal in terms of tissue type, e.g., the MHC molecules are matched (or haploidentical); there are still minor differences between these individuals that immune cells can perceive as dangerous. This means that the new immune system (white blood cells from the new stem cells) perceive the new body as “foreign”, which provokes an immune attack. This reaction, called graft-versus-host disease (GVHD), can become life-threatening to the patient. Patients after HSC transplantation also have an increased risk of infections due to absence of white blood cells before the new marrow begins to function. This period can in some cases last for many months until the new immune system have matured. Some of these opportunistic infections may be life-threatening.

Thus, there is a need for improving the conditioning and transplantation methods and decreasing the risks associated with HSC transplantation and increasing its effectiveness for various disorders. Provided herein are new antibody drug conjugates that, by specifically killing the recipient's endogenous HSCs prior to transplantation but not all other immune cells, keep a partially active immune defense to combat infections right after transplantation, but at the same time provide an indirect immunosuppressive effect due to the subject's inability to form new immune cells from its own HSCs. Since the pre-treatment can be milder than chemotherapy or radiation, and with less serious side effects, it might induce less GVHD in transplant patients.

The antibody drug conjugates described herein could be used to ablate endogenous hematopoietic stem cell, e.g., in a pre-treatment/conditioning method before hematopoietic stem cell transplantation. For example, the conjugates of the invention could be used to treat any non-malignant condition/disorder wherein stem cell transplantation could be beneficial, such as Severe aplastic anemia (SAA), Wiskott Aldrich Syndrome, Hurlers Syndrome, familial haemophagocytic lymphohistiocytosis (FHL), Chronic granulomatous disease (CGD), Kostmanns syndrome, Severe immunodeficiency syndrome (SCID), other autoimmune disorders such as SLE, Multiple sclerosis, IBD, Crohns Disease, Ulcerative colitis, Sjogrens syndrome, vasculitis, Lupus, Myasthenia Gravis, Wegeners disease, inborn errors of metabolism and/or other immunodeficiencies.

Further, the conjugates of the invention could be used to treat any malignant condition/disorder wherein stem cell transplantation could be beneficial, such as hematologic diseases, hematological malignancies or solid tumors (e.g., renal cancer, hepatic cancer, pancreatic cancer). Common types of hematological diseases/malignancies that could be treated with the claimed methods and antibodies are leukemias, lymphomas and myelodysplastic syndromes. Leukemia is a type of cancer of the blood or bone marrow characterized by an abnormal increase of immature white blood cells called blast cells, and the term leukemia includes: acute lymphoblastic leukemia (ALL), acute myelogenous leukemia (AML), acute monocytic leukemia (AMoL), chronic lymphocytic leukemia (CLL), chronic myelogenous leukemia (CML) and other leukemias such as hairy cell leukemia (HCL), T-cell prolymphocytic leukemia (T-PLL), large granular lymphocytic leukemia and adult T-cell leukemia. In one aspect of the invention, the leukemia treated is acute leukemia. In a further aspect, the leukemia is ALL, AML or AMoL. Lymphomas include precursor T-cell leukemia/lymphoma, Burkitt lymphoma, follicular lymphoma, diffuse large B cell lymphoma, mantle cell lymphoma, B-cell chronic lymphocytic leukemia/lymphoma, MALT lymphoma, Mycosis fungoides, Peripheral T-cell lymphoma not otherwise specified, Nodular sclerosis form of Hodgkin lymphoma Mixed-cellularity subtype of Hodgkin lymphoma. Myelodysplastic syndrome (MDS) is the name of a group of conditions that occur when the blood-forming cells in the bone marrow are damaged. This damage leads to low numbers of one or more type of blood cells. MDS is subdivided into 7 categories; Refractory cytopenia with unilineage dysplasia (RCUD), Refractory anemia with ringed sideroblasts (RARS), Refractory cytopenia with multilineage dysplasia (RCMD), Refractory anemia with excess blasts-1 (RAEB-1), Refractory anemia with excess blasts-2 (RAEB-2), Myelodysplastic syndrome, unclassified (MDS-U), and Myelodysplastic syndrome associated with isolated del (5q).

In some embodiments, a patient in need of ablating hematopoietic stem cells (e.g., a hematopoietic stem cell transplantation recipient) may have an inherited immunodeficient disease, an autoimmune disorder, a hematopoietic disorder, or inborn errors of metabolism.

In some embodiments, the hematopoietic disorder can be selected from any of the following: Acute myeloid leukemia (AML), Acute lymphoblastic leukemia (ALL), acute monocytic leukemia (AMoL), Chronic myeloid leukemia (CML), Chronic lymphocytic leukemia (CLL), Myeloproliferative disorders, Myelodysplastic syndromes, Multiple myeloma, Non-Hodgkin lymphoma, Hodgkin disease, Aplastic anemia, Pure red cell aplasia, Paroxysmal nocturnal hemoglobinuria, Fanconi anemi, Thalassemia major, Sickle cell anemia, Severe combined immunodeficiency, Wiskott-Aldrich syndrome, Hemophagocytic lymphohistiocytosis.

Inborn errors of metabolism are also known as inherited metabolic diseases (IMB) or congenital metabolic diseases, which are a class of genetic diseases that include congenital disorders of carbohydrate metabolism, amino acid metabolism, organic acid metabolism, or lysosomal storage diseases. In some embodiments, inborn errors of metabolism are selected from mucopolysaccharidosis, Gaucher disease, metachromatic leukodystrophies, or adrenoleukodystrophies.

In some embodiments, the antibody drug conjugates described herein may be used to ablate endogenous hematopoietic stem cell as a reduced-intensity conditioning method before allogeneic stem cell transplantation in a patient who have previously been treated with autologous stem cell transplantation for a disease or condition disclosed herein. For example, the antibody drug conjugates described herein may be used in allogeneic stem cell transplantation to patients who have previously been treated with autologous stem cell transplantation, as described in Chen et al., Biol Blood Marrow Transplant 21 (2015) 1583e1588. In some embodiments, the allogeneic stem cell transplantation may be performed 1 month, 2 months, 3 months, 4 months, 5 months, 6 months, or longer, after the patient has received autologous stem cell transplantation.

Further, the conjugates of the invention could be used to treat a gastrointestinal stromal tumor (GIST), such as GIST that is cKIT positive. In some embodiments, the conjugates of the invention could be used to treat GIST that expresses wild-type cKIT. In some embodiments, the conjugates of the invention could be used to treat GIST that is resistant to a treatment, e.g., imatinib (Glivec®/Gleevec®).

### Combination Therapy

In certain instances, an antibody drug conjugate of the present disclosure can be used in combination with another conditioning regiment such as radiation therapy or chemotherapy.

In certain instances, an antibody drug conjugate of the present disclosure can be used in combination with another therapeutic agent, such as an anti-cancer agent, anti-nausea agent (or anti-emetic), pain reliever, mobilizing agent, or combinations thereof.

General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclosporine (Sandimmune, Neoral® or Restasis®), cyclophosphamide (Cytoxan® or Neosars), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

In some embodiments, the antibody drug conjugate of the present disclosure can be used in combination with a CD47 blocker, e.g., an anti-CD47 antibody or fragment thereof. It was reported that an anti-CD47 microbody that blocks the interaction between CD47 and signal regulatory protein alpha (SIRPa) can enhance depletion of endogenous HSCs by a naked anti-c-Kit antibody (Chhabra et al., Science Translational Medicine 8 (351), 351ra105).

In some embodiments, the antibody drug conjugate of the present disclosure can be used in combination with another antibody or fragment thereof that specifically binds to hematopoietic stem cells or hematopoietic progenitor cells, e.g., anti-CD45 antibody or fragment thereof, anti-CD34 antibody or fragment thereof, anti-CD133 antibody or fragment thereof, anti-CD59 antibody or fragment thereof, or anti-CD90 antibody or fragment thereof. In some embodiments, the antibody drug conjugates of the present disclosure can be used in combination with a Dyrk1a inhibitor, such as Harmine, INDY, ML 315 hydrochloride, ProINDY, Tocris™ TC-S 7044, Tocris™ TG 003, FINDY, TBB, DMAT, CaNDY, etc.

In some embodiments, the antibody drug conjugate of the present disclosure can be used in combination with one or more immune suppressors, such as glucocorticoids, e.g., prednisone, dexamethasone, and hydrocortisone; cytostatics, e.g., alkylating agents, antimetabolites, methotrexate, azathioprine, mercaptopurine, dactinomycin, etc.; drugs acting on immunophilins, e.g., tacrolimus (Prograf®, Astograf XL® or Envarsus XR®), sirolimus (rapamycin or Rapamune®) and everolimus; interferons; opoids; TNF binding proteins; mycophenolate; fingolimod; myriocin; etc. In some embodiments, the antibody drug conjugate of the present disclosure can be used in combination with one or more agents that specifically deplete T cells, such as Fludarabine, Ciclosporin, anti-CD52 antibody, e.g., Alemtuzumab, Antithymocyte globulin (ATG), anti-CD3 antibody or fragment thereof, anti-CD4 antibody or fragment thereof, anti-CD8 antibody or fragment thereof, or anti-human TCR α/β antibody or fragment thereof. T cell depletion therapies can reduce host versus graft reaction, which could lead to rejection of a transplant.

In some embodiments, the antibody drug conjugate of the present disclosure can be used in combination with one or more agents selected from plerixafor (also known as AMD3100, Mozobil®), granulocyte-macrophage colony stimulating factor (GM-CSF), e.g., sargramostim (Leukine®), or granulocyte-colony stimulating factor (G-CSF), e.g., filgrastim or pegfilgrastim (Zarzio®, Zarxio®, Neupogen®, Neulasta®, Nufil®, Religrast®, Emgrast®, Neukine®, Grafeel®, Imumax, Filcad®).

In one aspect, an antibody drug conjugate of the present disclosure is combined in a pharmaceutical combination formulation, or dosing regimen as combination therapy, with a second compound having anti-cancer properties. The second compound of the pharmaceutical combination formulation or dosing regimen can have complementary activities to the conjugate of the combination such that they do not adversely affect each other.

The term “pharmaceutical combination” as used herein refers to either a fixed combination in one dosage unit form, or non-fixed combination or a kit of parts for the combined administration where two or more therapeutic agents may be administered independently at the same time or separately within time intervals, especially where these time intervals allow that the combination partners show a cooperative, e.g. synergistic effect.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients. Alternatively, such administration encompasses co-administration in multiple, or in separate containers (e.g., capsules, powders, and liquids) for each active ingredient. Powders and/or liquids may be reconstituted or diluted to a desired dose prior to administration. In addition, such administration also encompasses use of each type of therapeutic agent in a sequential manner, either at approximately the same time or at different times. In either case, the treatment regimen will provide beneficial effects of the drug combination in treating the conditions or disorders described herein.

The combination therapy can provide “synergy” and prove “synergistic”, i.e., the effect achieved when the active ingredients used together is greater than the sum of the effects that results from using the compounds separately. A synergistic effect can be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect can be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

### Pharmaceutical Compositions

To prepare pharmaceutical or sterile compositions including one or more antibody drug conjugates described herein, the provided conjugate(s) can be mixed with a pharmaceutically acceptable carrier or excipient.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., Goodman and Gilman's The Pharmacological Basis of Therapeutics, McGraw-Hill, New York, N.Y., 2001; Gennaro, Remington: The Science and Practice of Pharmacy, Lippincott, Williams, and Wilkins, New York, N.Y., 2000; Avis, et al. (eds.), Pharmaceutical Dosage Forms: Parenteral Medications, Marcel Dekker, N Y, 1993; Lieberman, et al. (eds.), Pharmaceutical Dosage Forms: Tablets, Marcel Dekker, N Y, 1990; Lieberman, et al. (eds.) Pharmaceutical Dosage Forms: Disperse Systems, Marcel Dekker, N Y, 1990; Weiner and Kotkoskie, Excipient Toxicity and Safety, Marcel Dekker, Inc., New York, N.Y., 2000).

In some embodiments, the pharmaceutical composition comprising the antibody conjugate of the present invention is a lyophilisate preparation. In certain embodiments a pharmaceutical composition comprising the antibody conjugate is a lyophilisate in a vial containing an antibody conjugate, histidine, sucrose, and polysorbate 20. In certain embodiments the pharmaceutical composition comprising the antibody conjugate is a lyophilisate in a vial containing an antibody conjugate, sodium succinate, and polysorbate 20. In certain embodiments the pharmaceutical composition comprising the antibody conjugate is a lyophilisate in a vial containing an antibody conjugate, trehalose, citrate, and polysorbate 8. The lyophilisate can be reconstituted, e.g., with water, saline, for injection. In a specific embodiment, the solution comprises the antibody conjugate, histidine, sucrose, and polysorbate 20 at a pH of about 5.0. In another specific embodiment the solution comprises the antibody conjugate, sodium succinate, and polysorbate 20. In another specific embodiment, the solution comprises the antibody conjugate, trehalose dehydrate, citrate dehydrate, citric acid, and polysorbate 8 at a pH of about 6.6. For intravenous administration, the obtained solution will usually be further diluted into a carrier solution.

Selecting an administration regimen for a therapeutic depends on several factors, including the serum or tissue turnover rate of the entity, the level of symptoms, the immunogenicity of the entity, and the accessibility of the target cells in the biological matrix. In certain embodiments, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak, Antibody Therapy, Bios Scientific Pub. Ltd, Oxfordshire, U K, 1996; Kresina (ed.), Monoclonal Antibodies, Cytokines and Arthritis, Marcel Dekker, New York, N.Y., 1991; Bach (ed.), Monoclonal Antibodies and Peptide Therapy in Autoimmune Diseases, Marcel Dekker, New York, N.Y., 1993; Baert et al., New Engl. J. Med. 348:601-608, 2003; Milgrom et al., New Engl. J. Med. 341:1966-1973, 1999; Slamon et al., New Engl. J. Med. 344:783-792, 2001; Beniaminovitz et al., New Engl. J. Med. 342:613-619, 2000; Ghosh et al., New Engl. J. Med. 348:24-32, 2003; Lipsky et al., New Engl. J. Med. 343:1594-1602, 2000).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors known in the medical arts.

Compositions comprising the antibody conjugate of the invention can be provided by continuous infusion, or by doses at intervals of, e.g., one day, one week, or 1-7 times per week, once every other week, once every three weeks, once every four weeks, once every five weeks, once every six weeks, once every seven weeks, or once every eight weeks. Doses may be provided intravenously, subcutaneously, or intraosseously. A specific dose protocol is one involving the maximal dose or dose frequency that avoids significant undesirable side effects.

For the antibody conjugates of the invention, the dosage administered to a patient may be 0.0001 mg/kg to 100 mg/kg of the patient's body weight. The dosage may be between 0.001 mg/kg and 50 mg/kg, 0.005 mg/kg and 20 mg/kg, 0.01 mg/kg and 20 mg/kg, 0.02 mg/kg and 10 mg/kg, 0.05 and 5 mg/kg, 0.1 mg/kg and 10 mg/kg, 0.1 mg/kg and 8 mg/kg, 0.1 mg/kg and 5 mg/kg, 0.1 mg/kg and 2 mg/kg, 0.1 mg/kg and 1 mg/kg of the patient's body weight. The dosage of the antibody conjugate may be calculated using the patient's weight in kilograms (kg) multiplied by the dose to be administered in mg/kg.

Doses of the antibody conjugates the invention may be repeated and the administrations may be separated by less than 1 day, at least 1 day, 2 days, 3 days, 5 days, 10 days, 15 days, 30 days, 45 days, 2 months, 75 days, 3 months, 4 months, 5 months, or at least 6 months. In some embodiments, an antibody conjugate of the invention is administered twice weekly, once weekly, once every two weeks, once every three weeks, once every four weeks, or less frequently.

An effective amount for a particular patient may vary depending on factors such as the condition being treated, the overall health of the patient, the method, route and dose of administration and the severity of side effects (see, e.g., Maynard et al., A Handbook of SOPs for Good Clinical Practice, Interpharm Press, Boca Raton, Fla., 1996; Dent, Good Laboratory and Good Clinical Practice, Urch Publ., London, U K, 2001).

The route of administration may be by, e.g., topical or cutaneous application, injection or infusion by subcutaneous, intravenous, intraperitoneal, intracerebral, intramuscular, intraocular, intraarterial, intracerebrospinal, intralesional administration, or by sustained release systems or an implant (see, e.g., Sidman et al., Biopolymers 22:547-556, 1983; Langer et al., J. Biomed. Mater. Res. 15:167-277, 1981; Langer, Chem. Tech. 12:98-105, 1982; Epstein et al., Proc. Natl. Acad. Sci. USA 82:3688-3692, 1985; Hwang et al., Proc. Natl. Acad. Sci. USA 77:4030-4034, 1980; U.S. Pat. Nos. 6,350,466 and 6,316,024). Where necessary, the composition may also include a solubilizing agent or a local anesthetic such as lidocaine to ease pain at the site of the injection, or both. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

Methods for co-administration or treatment with a second therapeutic agent, e.g., a cytokine, steroid, chemotherapeutic agent, antibiotic, or radiation (such as total body irradiation (TBI)), are known in the art (see, e.g., Hardman et al., (eds.) (2001) Goodman and Gilman's The Pharmacological Basis of Therapeutics, 10.sup.th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic may decrease the symptoms by at least 10%; by at least 20%; at least about 30%; at least 40%, or at least 50%.

Additional therapies, which can be administered in combination with the antibody conjugates of the invention may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the antibody conjugates of the invention. The two or more therapies may be administered within one same patient visit.

The invention provides protocols for the administration of pharmaceutical composition comprising antibody conjugates of the invention alone or in combination with other therapies to a subject in need thereof. The therapies of the combination therapies of the present invention can be administered concomitantly or sequentially to a subject. The therapy of the combination therapies of the present invention can also be cyclically administered. Cycling therapy involves the administration of a first therapy for a period of time, followed by the administration of a second therapy for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies of the combination therapies of the invention can be administered to a subject concurrently.

The term “concurrently” is not limited to the administration of therapies at exactly the same time, but rather it is meant that a pharmaceutical composition comprising antibodies or fragments thereof the invention are administered to a subject in a sequence and within a time interval such that the antibodies or antibody conjugates of the invention can act together with the other therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic effect. Each therapy can be administered to a subject separately, in any appropriate form and by any suitable route. In various embodiments, the therapies are administered to a subject less than 5 minutes apart, less than 15 minutes apart, less than 30 minutes apart, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In other embodiments, two or more therapies are administered within the same patient visit.

The combination therapies can be administered to a subject in the same pharmaceutical composition. Alternatively, the therapeutic agents of the combination therapies can be administered concurrently to a subject in separate pharmaceutical compositions. The therapeutic agents may be administered to a subject by the same or different routes of administration.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1: Generation of Anti-cKIT ADC

Preparation of Anti-cKit Antibodies and Antibody Fragments with or without Site-Specific Cysteine Mutations

Human anti-cKIT antibodies and antibody fragments were generated as described previously in WO2014150937 and WO2016020791.

DNA encoding variable regions of the heavy and light chains of an anti-cKit antibody were amplified from a vector isolated in a phage display based screen and cloned into mammalian expression vectors that contain the constant regions of human IgG1 heavy chain and human kappa light chain or lambda light chain. Vectors contain a CMV promoter and a signal peptide (MPLLLLLPLLWAGALA (SEQ ID NO: 149) for heavy chain and MSVLTQVLALLLLWLTGTRC (SEQ ID NO: 150) for light chain, and appropriate signal and selection sequences for amplification of DNA in a bacterial host, e.g. E. coli DH5alpha cells, transient expression in mammalian cells, e.g. HEK293 cells, or stable transfection into mammalian cells, e.g. CHO cells. To introduce Cys mutations, site-directed mutagenesis PCR was conducted with oligos designed to substitute single Cys residues at certain site in the constant regions of the heavy chain or light chain coding sequences. Examples of Cys substitution mutations are E152C or S375C of heavy chain; E165C or S114C of kappa light chain; or A143C of the lambda light chain (all EU numbering). In some cases, two or more Cys mutations were combined to make an antibody with multiple Cys substitutions, for example HC-E152C-S375C, lambda LC-A143C-HC-E152C, kappa LC-E165C-HC-E152C, or kappa LC-S114C-HC-E152C (all EU numbering). To generate plasmids encoding antibody fragments, mutagenesis PCR was conducted with oligos designed to remove or modify a portion of the heavy chain constant region. For example, a PCR was performed to remove residues 222-447 (EU numbering) of the heavy chain constant region such that a stop codon was encoded directly after residue 221 (EU number) in order to make an expression construct for a Fab fragment. For example, a PCR was performed to remove residues 233-447 (EU numbering) of the heavy chain constant region such that a stop codon was encoded directly after residue 232 (EU number) in order to make an expression construct for a Fab′ fragment including the two Cys residues of the IgG1 hinge.

Anti-cKit antibodies, antibody fragments, and Cys mutant antibodies or antibody fragments were expressed in 293 Freestyle™ cells by co-transfecting heavy chain and light chain plasmids using transient transfection methods as described previously (Meissner, et al., Biotechnol Bioeng. 75:197-203 (2001)). The expressed antibodies were purified from the cell supernatants by standard affinity chromatography methods using an appropriate resin such as Protein A, Protein G, Capto-L or LambdaFabSelect resins. Alternatively, anti-cKit antibodies, antibody fragments, and Cys mutant antibodies or antibody fragments were expressed in a CHO by co-transfecting a heavy chain vector and a light chain vector into CHO cells. Cells underwent selection, and stably transfected cells were then cultured under conditions optimized for antibody production. Antibodies were purified from the cell supernatants as above.

### Reduction, Re-Oxidation and Conjugation of Anti-cKit Antibodies and Antibody Fragments to Toxins

Compounds comprised of a reactive moiety, e.g. a maleimide group, for reaction to a thiol group (Cys side chain) on the antibody or antibody fragment, a linker as described, and a functional moiety, such as an auristatin or other toxin, were conjugated to Cys residues, native or engineered into the antibody using methods described previously (e.g., in WO2014124316, WO2015138615, Junutula J R, et al., Nature Biotechnology 26:925-932 (2008)).

Because engineered Cys residues in antibodies expressed in mammalian cells are modified by adducts (disulfides) such as glutathione (GSH) and/or cysteine during biosynthesis (Chen et al. 2009), the modified Cys as initially expressed is unreactive to thiol reactive reagents such as maleimido or bromo-acetamide or iodo-acetamide groups. To conjugate engineered Cys residues, glutathione or cysteine adducts need to be removed by reducing disulfides, which generally entails reducing all disulfides in the expressed antibody. Because native Cys residues in antibodies and antibody fragments generally form disulfide bonds to other Cys residues in the antibody or antibody fragment, these are also unreactive to thiol reactive reagents until the disulfides are reduced. Reduction of disulfides can be accomplished by first exposing antibody to a reducing agent such as dithiothreitol (DTT), cysteine, or Tris(2-carboxyethyl)phosphine hydrochloride (TCEP-HCl). Optionally, the reducing agent can be removed to allow re-oxidation of all native disulfide bonds of the antibody or antibody fragment to restore and/or stabilize the functional antibody structure.

In cases where an antibody or antibody fragment was conjugated only at engineered Cys residues, in order to reduce native disulfide bonds and disulfide bond between the cysteine or GSH adducts of engineered Cys residue(s), freshly prepared DTT was added to purified Cys mutant antibodies, to a final concentration of 10 mM or 20 mM. After antibody incubation with DTT at 37° C. for 1 hour, mixtures were dialyzed against PBS for three days with daily buffer exchange to remove DTT and re-oxidize native disulfide bonds. The re-oxidation process was monitored by reverse-phase HPLC, which is able to separate antibody tetramer from individual heavy and light chain molecules. Reactions were analyzed on a PRLP-S 4000A column (50 mm×2.1 mm, Agilent) heated to 80° C. and column elution was carried out by a linear gradient of 30-60% acetonitrile in water containing 0.1% TFA at a flow rate of 1.5 ml/min. The elution of proteins from the column was monitored at 280 nm. Dialysis was allowed to continue until reoxidation was complete. Reoxidation restores intra-chain and interchain disulfides, while dialysis allows cysteines and glutathiones connected to the newly-introduced Cys residue(s) to dialyze away. After re-oxidation, maleimide-containing compounds were added to re-oxidized antibodies or antibody fragments in PBS buffer (pH 7.2) at ratios of typically 1.5:1, 2:1, or 5:1 to engineered Cys, and incubations were carried out for 1 hour. Typically, excess free compound was removed by purification over Protein A or other appropriate resin by standard methods followed by buffer exchange into PBS.

Alternatively, antibodies or antibody fragments with engineered Cys sites were reduced and re-oxidized using an on-resin method. Protein A Sepharose beads (1 ml per 10 mg antibody) were equilibrated in PBS (no calcium or magnesium salts) and then added to an antibody sample in batch mode. A stock of 0.5 M cysteine was prepared by dissolving 850 mg of cysteine HCl in 10 ml of a solution prepared by adding 3.4 g of NaOH to 250 ml of 0.5 M sodium phosphate pH 8.0 and then 20 mM cysteine was added to the antibody/bead slurry, and mixed gently at room temperature for 30-60 minutes. Beads were loaded to a gravity column and washed with 50 bed volumes of PBS in less than 30 minutes. Then the column was capped with beads resuspended in one bed volume of PBS. To modulate the rate of re-oxidation, 50 nM to 1 μM copper chloride was optionally added. The re-oxidation progress was monitored by removing a small test sample of the resin, eluting in IgG Elution buffer (Thermo), and analyzing by RP-HPLC as described above. Once re-oxidation progressed to desired completeness, conjugation could be initiated immediately by addition of 2-3 molar excess of compound over engineered cysteines, and allowing the mixture to react for 5-10 minutes at room temperature before the column was washed with at least 20 column volumes of PBS. Antibody conjugates were eluted with IgG elution buffer and neutralized with 0.1 volumes 0.5 M sodium phosphate pH 8.0 and buffer exchanged to PBS. In some instances, instead of initiating conjugation with antibody on the resin, the column was washed with at least 20 column volumes of PBS, and antibody was eluted with IgG elution buffer and neutralized with buffer pH 8.0. Antibodies were then either used for conjugation reactions or flash frozen for future use.

In some instances, it is desired to conjugate to native Cys residues, such as those that usually form the heavy chain to light chain interchain disulfide bond and the Cys residues in the hinge region of the antibody that usually form heavy chain to heavy chain interchain disulfide bonds, in the absence of engineered Cys residues or at the same time as conjugation was also directed to engineered Cys residues. In these cases, the antibody or antibody fragment was reduced by adding 5-fold excess of TCEP to disulfide bonds and incubated the sample at 37° C. for 1 hour. The samples were then immediately conjugated or frozen at <−60° C. for future conjugation. Maleimide-containing compounds were added to antibodies or antibody fragments in PBS buffer (pH 7.2) at ratios of typically 2:1 to Cys residues used for conjugation, and incubations were carried out for 1 hour. Typically, excess free compound was removed by desalting column followed by more extensive buffer exchange to PBS.

Generation of Antibody Fragments from Full-Length Antibodies

In some instances, antibody fragments were generated by genetic manipulation of the antibody heavy chain coding sequence, as described above, such that the product of expression was a fragment of an antibody. In other instances, antibodies were generated by enzymatic digest of full-length antibodies.

To generate Fab fragments comprising residues 1-222 (EU numbering) of a starting antibody, the full antibody was treated with immobilized papain resin (ThermoFisher Scientific) according to manufacturer's protocol. Briefly, the immobilized papain resin is prepared by equilibrating in a digestion buffer of freshly dissolved 20 mM cysteine-HCl adjusted to pH 7.0. The antibody is adjusted to approximately 10 mg/ml and buffer exchanged into the digestion buffer and added to resin at a ratio of 4 mg IgG per ml resin and incubated at 37° C. for 5-7 hours. The resin is then removed, and the antibody fragment is purified by either an appropriate affinity resin, for example the intact IgG and Fc fragment are separated from the Fab fragment by binding to Protein A resin, or the separation is conducted by size exclusion chromatography.

To generate F(ab′)₂ fragments comprising residues 1-236 (EU numbering) of the starting antibody, the full antibody was treated with a proteolytic enzyme. Briefly, the antibody is prepared in PBS at approximately 10 mg/ml. The enzyme is added at a 1:100 weight/weight ratio and incubated for 2 hours at 37° C. The antibody fragment is purified by either an appropriate affinity resin, for example the intact IgG and Fc fragment are separated from the Fab′ fragment by binding to Protein A resin, or the separation is conducted by size exclusion chromatography.

### Properties of Anti-cKit-Toxin Antibody and Antibody Fragment Conjugates

Antibody and antibody fragment conjugates were analyzed to determine extent of conjugation. A compound-to-antibody ratio was extrapolated from LC-MS data for reduced and deglycosylated (where appropriate) samples. LC/MS allows quantitation of the average number of molecules of linker-payload (compound) attached to an antibody in a conjugate sample. High pressure liquid chromatography (HPLC) separates antibody into light and heavy chains, and under reducing conditions, separates heavy chain (HC) and light chain (LC) according to the number of linker-payload groups per chain. Mass spectral data enables identification of the component species in the mixture, e.g., LC, LC+1, LC+2, HC, HC+1, HC+2, etc. From the average loading on the LC and HC chains, the average compound to antibody ratio can be calculated for an antibody conjugate. A compound-to-antibody ratio for a given conjugate sample represents the average number of compound (linker-payload) molecules attached to a tetrameric antibody containing two light chains and two heavy chains.

Conjugates were profiled using analytical size-exclusion chromatography (AnSEC) on Superdex 200 10/300 GL (GE Healthcare) and/or Protein KW-803 5 μm 300×8 mm (Shodex) columns; aggregation was analyzed based on analytical size exclusion chromatography.

### Preparation of Exemplary Anti-cKIT Fab-Toxin Conjugates

To generate anti-cKIT Fab′-toxin DAR4 conjugates or anti-Her2 Fab-toxin DAR4 control conjugate, 50 mg full IgG (WT, without introduced cysteines) was digested with a proteolytic enzyme. The F(ab′)₂ fragment was purified by SEC on a Superdex-S200 (GE Healthcare) column. Alternatively, to generate anti-HER2 control conjugates or anti-cKit Fab′-toxin DAR4 conjugates, a vector encoding the Fab′ HC was co-transfected with a vector encoding the Fab′ LC in CHO. The expressed Fab′ was purified by capture on Protein G resin. The F(ab′)₂ or Fab′ was reduced by addition of TCEP (5× excess to interchain disulfides) and immediately reacted with a compound of the invention (2.5× excess to free Cys residues). Reaction was monitored by RP-HPLC, and additional 1× equivalents of compound were added until reaction was completed. Free compound was removed by PD10 desalting column (GE Healthcare). DAR were experimentally determined to be >3.9. Specific conjugates studied further in the provided examples are listed in Table 2.

To generate anti-cKIT Fab-toxin DAR2 conjugates, a vector encoding the Fab HC with an introduced Cys residue (HC 1-221 with E152C by EU numbering) was co-transfected with a vector encoding the Fab LC with an introduced Cys residue (kappa LC K107C, kappa LC S114C, or kappa LC E165C by EU numbering) in HEK293. To generate anti-Her2 Fab-toxin DAR2 control conjugates, a vector encoding the Fab HC with an introduced Cys residue (HC 1-222 with E152C by EU numbering, and a C-terminal His₆ tag (SEQ ID NO: 151)) was co-transfected with a vector encoding the Fab LC with an introduced Cys residue (kappa LC K107C, kappa LC S114C, or kappa LC E165C by EU numbering) in HEK293. The expressed Fabs were purified by capture on Capto-L resin (GE Healthcare) and elution with standard IgG Elution Buffer (Thermo). Fabs were buffer exchanged to PBS using Amicon ultra devices. Fabs were reduced with DTT and allowed to reoxidize at room temperature. After reformation of the interchain disulfide bond, the Fabs were conjugated to Compound 6 (3× excess to free Cys residues). Reaction was allowed to proceed for 30 min at room temperature and monitored by RP-HPLC with detection at 310 nm. Conjugated Fabs were purified over protein A (anti-her2) or capto-L (anti-cKit) resins and were washed with PBS+1% Triton X-100 and washed with extensive PBS before elution in IgG Elution Buffer. Fabs were then buffer exchanged to PBS using Amicon Ultra devices. Specific conjugates studied further in the provided examples are listed in Table 2 below with experimentally determined DAR values.

To generate anti-cKIT F(ab′)₂-toxin DAR2 conjugates, a vector encoding the HC with introduced Cys residues (E152C and S375C by EU numbering) was co-transfected with a vector encoding the Fab LC in CHO. To generate anti-Her2 F(ab′)₂-toxin DAR2 control conjugates, a vector encoding the HC with introduced Cys residues (E152C and S375C by EU numbering) was co-transfected with a vector encoding the Fab LC in HEK293. The expressed IgGs were purified by capture on protein A or mabselectsure resin (GE Healthcare) and elution with standard IgG Elution Buffer (Thermo). Full IgGs were reduced with DTT at room temperature and reoxidized following removal of DTT as monitored by RP-HPLC. The reoxidized IgGs were then digested with a proteolytic enzyme to generate F(ab′)₂ fragments. For anti-cKIT fragments, F(ab′)₂'s were buffer exchanged to PBS using Amicon ultra devices. For anti-HER2 fragment, F(ab′)₂ fraction was enriched by preparative HIC and then buffer exchanged to PBS using Amicon ultra devices. The F(ab′)₂'s were conjugated to Compound (LP) or Compound (LP2) (4× excess to free Cys residues). Reaction was allowed to proceed for 30 min at room temperature and monitored by RP-HPLC with detection at 310 nm. Conjugated F(ab′)'s were purified over capto-L (anti-cKit Ab3) resins and were washed with PBS+1% Triton X-100 and washed with extensive PBS before elution in IgG Elution Buffer or by preparative SEC (anti-her2 and anti-cKIT Ab4). F(ab′)'s were then concentrated and buffer exchanged to PBS using Amicon Ultra devices. Specific conjugates studied further in the provided examples are listed in Table 2 below with experimentally determined DAR values.

TABLE 2
Exemplary anti-cKIT or control conjugates

| | | | Antibody | Antibody | | |
|---|---|---|---|---|---|---|
| Conjug. | Antibody | Conjugation | fragment HC | fragment LC | Linker- | |
| No. | fragment | Method | sequence | sequence | Payload | DAR |
| J1 | Anti-cKIT | Native | SEQ ID NO: 99 | SEQ ID NO: 110 | LP2 | 4.0 |
| | Fab′5 | Cysteine | | | | |
| | | conjugation | | | | |
| J2 | Anti-cKIT | Native | SEQ ID NO: 99 | SEQ ID NO: 110 | LP1 | 4.0 |
| | Fab′5 | Cysteine | | | | |
| | | conjugation | | | | |
| J3 | Anti-cKIT | Native | SEQ ID NO: 99 | SEQ ID NO: 110 | LP1 | 3.9 |
| | Fab′5 | Cysteine | | | | |
| | | conjugation | | | | |
| J4 | Anti-cKIT | Native | SEQ ID NO: 120 | SEQ ID NO: 25 | LP1 | 3.8 |
| | Fab′1 | Cysteine | | | | |
| | | conjugation | | | | |
| J5 | Anti-cKIT | Native | SEQ ID NO: 120 | SEQ ID NO: 25 | LP2 | 3.9 |
| | Fab′1 | Cysteine | | | | |
| | | conjugation | | | | |
| J6 | Anti-cKIT | Engineered | SEQ ID NO: 130 | SEQ ID NO: 134 | LP1 | 1.7 |
| | Fab3 | Cysteines at | | | | |
| | | HC-E152C & | | | | |
| | | LC-E165C | | | | |
| | | (EU) | | | | |
| J7 | Anti-cKIT | Engineered | SEQ ID NO: 130 | SEQ ID NO: 134 | LP2 | 1.7 |
| | Fab3 | Cysteines at | | | | |
| | | HC-E152C & | | | | |
| | | LC-E165C | | | | |
| | | (EU) | | | | |
| J8 | Anti-cKIT | Native | SEQ ID NO: 14 | SEQ ID NO: 25 | LP1 | 3.8 |
| | Fab′1 | Cysteine | | | | |
| | | conjugation | | | | |
| J9 | Anti-cKIT | Native | SEQ ID NO: 14 | SEQ ID NO: 25 | LP2 | 3.8 |
| | Fab′1 | Cysteine | | | | |
| | | conjugation | | | | |
| J10 | Anti-cKIT | Native | SEQ ID NO: 40 | SEQ ID NO: 49 | LP1 | 3.6 |
| | Fab′2 | Cysteine | | | | |
| | | conjugation | | | | |
| J11 | Anti-cKIT | Native | SEQ ID NO: 40 | SEQ ID NO: 49 | LP2 | 3.8 |
| | Fab′2 | Cysteine | | | | |
| | | conjugation | | | | |
| J12 | Anti-cKIT | Native | SEQ ID NO: 126 | SEQ ID NO: 49 | LP1 | 3.9 |
| | Fab′2 | Cysteine | | | | |
| | | conjugation | | | | |
| J13 | Anti-cKIT | Native | SEQ ID NO: 132 | SEQ ID NO: 25 | LP1 | 3.9 |
| | Fab′3 | Cysteine | | | | |
| | | conjugation | | | | |
| J14 | Anti-cKIT | Native | SEQ ID NO: 143 | SEQ ID NO: 110 | LP1 | 3.8 |
| | Fab′5 | Cysteine | | | | |
| | | conjugation | | | | |
| J15 | Anti-cKIT | Native | SEQ ID NO: 120 | SEQ ID NO: 25 | LP1 | 3.9 |
| | Fab′1 | Cysteine | | | | |
| | | conjugation | | | | |
| J16 | Anti-cKIT | Native | SEQ ID NO: 120 | SEQ ID NO: 25 | LP2 | 3.9 |
| | Fab′1 | Cysteine | | | | |
| | | conjugation | | | | |
| J17 | Anti-cKIT | Native | SEQ ID NO: 126 | SEQ ID NO: 49 | LP1 | 3.9 |
| | Fab′2 | Cysteine | | | | |
| | | conjugation | | | | |
| J18 | Anti-cKIT | Native | SEQ ID NO: 126 | SEQ ID NO: 49 | LP2 | 3.9 |
| | Fab′2 | Cysteine | | | | |
| | | conjugation | | | | |
| J19 | Anti-HER2 | Native | EVQLVESGGGLVQ | DIQMTQSPSSLSAS | LP1 | 3.9 |
| | Fab′ | Cysteine | PGGSLRLSCAASGF | VGDRVTITCRASQD | | |
| | | conjugation | NIKDTYIHWVRQAP | VNTAVAWYQQKP | | |
| | | | GKGLEWVARIYPTN | GKAPKLLIYSASFLYS | | |
| | | | GYTRYADSVKGRFT | GVPSRFSGSRSGTD | | |
| | | | ISADTSKNTAYLQM | FTLTISSLQPEDFAT | | |
| | | | NSLRAEDTAVYYCS | YYCQQHYTTPPTFG | | |
| | | | RWGGDGFYAMDY | QGTKVEIKRTVAAP | | |
| | | | WGQGTLVTVSSAS | SVFIFPPSDEQLKSG | | |
| | | | TKGPSVFPLAPSSKS | TASVVCLLNNFYPR | | |
| | | | TSGGTAALGCLVKD | EAKVQWKVDNAL | | |
| | | | YFPEPVTVSWNSG | QSGNSQESVTEQD | | |
| | | | ALTSGVHTFPAVLQ | SKDSTYSLSSTLTLSK | | |
| | | | SSGLYSLSSVVTVPS | ADYEKHKVYACEVT | | |
| | | | SSLGTQTYICNVNH | HQGLSSPVTKSFNR | | |
| | | | KPSNTKVDKKVEPK | GEC | | |
| | | | SCDKTHTCPPCPAP | (SEQ ID NO: 147) | | |
| | | | (SEQ ID NO: 146) | | | |
| J20 | Anti-HER2 | Native | SEQ ID NO: 146 | SEQ ID NO: 147 | LP2 | 3.8 |
| | Fab′ | Cysteine | | | | |
| | | conjugation | | | | |
| J21 | Anti-cKIT | Native | SEQ NO 120 | SEQ ID NO: 25 | LP1 | 3.9 |
| | Fab′1 | Cysteine | | | | |
| | | conjugation | | | | |
| J22 | Anti-cKIT | Native | SEQ NO 120 | SEQ ID NO: 25 | LP2 | 3.8 |
| | Fab′1 | Cysteine | | | | |
| | | conjugation | | | | |
| J23 | Anti-cKIT | Native | SEQ NO: 132 | SEQ ID NO: 25 | LP1 | 3.9 |
| | Fab′3 | Cysteine | | | | |
| | | conjugation | | | | |
| J24 | Anti-cKIT | Native | SEQ NO: 132 | SEQ ID NO: 25 | LP2 | 3.8 |
| | Fab′3 | Cysteine | | | | |
| | | conjugation | | | | |
| J25 | Anti-cKIT | Native | SEQ NO 120 | SEQ ID NO: 25 | LP2 | 4.0 |
| | Fab′1 | Cysteine | | | | |
| | | conjugation | | | | |
| J26 | Anti-cKIT | Native | SEQ NO: 126 | SEQ ID NO: 49 | LP2 | 4.0 |
| | Fab′2 | Cysteine | | | | |
| | | conjugation | | | | |
| J27 | Anti-HER2 | Native | SEQ ID NO: 146 | SEQ ID NO: 147 | LP2 | 4.0 |
| | Fab′ | Cysteine | | | | |
| | | conjugation | | | | |
| J28 | Anti-HER2 | Native | SEQ ID NO: 146 | SEQ ID NO: 147 | LP2 | 4.0 |
| | Fab′ | Cysteine | | | | |
| | | conjugation | | | | |
| J29 | Anti-cKIT | Native | SEQ NO: 132 | SEQ ID NO: 25 | LP2 | 4.0 |
| | Fab′3 | Cysteine | | | | |
| | | conjugation | | | | |
| J30 | Anti-cKIT | Native | SEQ NO 120 | SEQ ID NO: 25 | LP2 | 4.0 |
| | Fab′1 | Cysteine | | | | |
| | | conjugation | | | | |
| J31 | Anti-HER2 | Native | SEQ ID NO: 146 | SEQ ID NO: 147 | LP2 | 4.0 |
| | Fab′ | Cysteine | | | | |
| | | conjugation | | | | |

### Example 2: Generation of Human, Cyno, Mouse and Rat cKIT Extracellular Domain Proteins, and of cKIT Subdomains 1-3 and 4-5 for Binding Assays

Human, mouse and rat cKIT extracellular domains (ECD) were gene synthesized based on amino acid sequences from the GenBank or Uniprot databases (see Table 3 below). Cynomolgus cKIT and 1ECD cDNA template were gene synthesized based on amino acid sequences information generated using mRNA from various cyno tissues (e.g. Zyagen Laboratories; Table 4 below). All synthesized DNA fragments were cloned into appropriate expression vectors e.g. hEF-HTLV based vector (pFUSE-mlgG2A-Fc2) with C-terminal tags to allow for purification.

TABLE 3
Sequences of human, mouse, rat cKIT constructs

| | | Accession | SEQ ID |
|---|---|---|---|
| Name | Description | Number | NO: |
| Human cKIT | Human cKIT tr. variant 2. | NM_ | 112 |
| D1-5 | residues 26-520-TAG | 001093772 | |
| (extracellular | QPSVSPGEPSPPSIHPGKSDLIVRVGDEI | | |
| domain) | RLLCTDPGFVKWTFEILDETNENKQNEW | | |
| | ITEKAEATNTGKYTCTNKHGLSNSIYVFV | | |
| | RDPAKLFLVDRSLYGKEDNDTLVRCPLT | | |
| | DPEVTNYSLKGCQGKPLPKDLRFIPDPK | | |
| | AGIMIKSVKRAYHRLCLHCSVDQEGKSV | | |
| | LSEKFILKVRPAFKAVPVVSVSKASYLLR | | |
| | EGEEFTVTCTIKDVSSSVYSTWKRENSQ | | |
| | TKLQEKYNSWHHGDFNYERQATLTISSA | | |
| | RVNDSGVFMCYANNTFGSANVTTTLEVVD | | |
| | KGFINIFPMINTTVFVNDGENVDLIVEYE | | |
| | AFPKPEHQQWIYMNRTFTDKWEDYPKSE | | |
| | NESNIRYVSELHLTRLKGTEGGTYTFLV | | |
| | SNSDVNAAIAFNVYVNTKPEILTYDRLVN | | |
| | GMLQCVAAGFPEPTIDWYFCPGTEQRC | | |
| | SASVLPVDVQTLNSSGPPFGKLVVQSSI | | |
| | DSSAFKHNGTVECKAYNDVGKTSAYFNF | | |
| | AFKEQIHPHTLFTPRSHHHHHH | | |
| Human cKIT | Human cKIT tr. Variant 1. | NM_000222 | 113 |
| D1-3 | residues 26-311-TAG | | |
| | QPSVSPGEPSPPSIHPGKSDLIVRVGDEI | | |
| | RLLCTDPGFVKWTFEILDETNENKQNEW | | |
| | ITEKAEATNTGKYTCTNKHGLSNSIYVFV | | |
| | RDPAKLFLVDRSLYGKEDNDTLVRCPLT | | |
| | DPEVTNYSLKGCQGKPLPKDLRFIPDPK | | |
| | AGIMIKSVKRAYHRLCLHCSVDQEGKSV | | |
| | LSEKFILKVRPAFKAVPWSVSKASYLLR | | |
| | EGEEFTVTCTIKDVSSSVYSTWKRENSQ | | |
| | TKLQEKYNSWHHGDFNYERQATLTISSA | | |
| | RVNDSGVFMCYANNTFGSANVTTTLEVV | | |
| | DKGRSHHHHHH | | |
| Human cKIT | Human cKIT tr. variant 1. | NM_000222 | 114 |
| D4-5 | residues 311-524-TAG | | |
| | GFINIFPMINTTVFVNDGENVDLIVEYEA | | |
| | FPKPEHQQWIYMNRTFTDKWEDYPKSEN | | |
| | ESNIRYVSELHLTRLKGTEGGTYTFLVSN | | |
| | SDVNAAIAFNVYVNTKPEILTYDRLVNGM | | |
| | LQCVAAGFPEPTIDWYFCPGTEQRCSAS | | |
| | VLPVDVQTLNSSGPPFGKLVVQSSIDSS | | |
| | AFKHNGTVECKAYNDVGKTSAYFNFAFK | | |
| | GNNKEQIHPHTLFTPRSHHHHHH | | |
| Mouse cKIT | Mouse cKIT tr. variant 1. | NM_ | 115 |
| D1-5 | residues 26-527-TAG | 001122733 | |
| | SQPSASPGEPSPPSIHPAQSELIVEAGDT | | |
| | LSLTCIDPDFVRWTFKTYFNEMVENKKN | | |
| | EWIQEKAEATRTGTYTCSNSNGLTSSIYV | | |
| | FVRDPAKLFLVGLPLFGKEDSDALVRCPL | | |
| | TDPQVSNYSLIECDGKSLPTDLTFVPNPK | | |
| | AGITIKNVKRAYHRLCVRCAAQRDGTWLH | | |
| | SDKFTLKVRAAIKAIPVVSVPETSHLLKK | | |
| | GDTFTVVCTIKDVSTSVNSMWLKMNPQP | | |
| | QHIAQVKHNSWHRGDFNYERQETLTISS | | |
| | ARVDDSGVFMCYANNTFGSANVTTTLKV | | |
| | VEKGFINISPVKNTTVFVTDGENVDLWE | | |
| | YEAYPKPEHQQWIYMNRTSANKGKDYV | | |
| | KSDNKSNIRYVNQLRLTRLKGTEGGTYT | | |
| | FLVSNSDASASVTFNVYVNTKPEILTYDR | | |
| | LINGMLQCVAEGFPEPTIDWYFCTGAEQ | | |
| | RCTTPVSPVDVQVQNVSVSPFGKLVVQS | | |
| | SIDSSVFRHNGTVECKASNDVGKSSAFF | | |
| | NFAFKEQIQAHTLFTPLEVLFQGPRSPRG | | |
| | PTIKPCPPCKCPAPNLLGGPSVFIFPPKI | | |
| | KDVLMISLSPIVTCWVDVSEDDPDVQISW | | |
| | FVNNVEVHTAQTQTHREDYNSTLRVVSA | | |
| | LPIQHQDWMSGKEFKCKVNNKDLPAPIE | | |
| | RTISKPKGSVRAPQVYVLPPPEEEMTKK | | |
| | QVTLTCMVTDFMPEDIYVEWTNNGKTEL | | |
| | NYKNTEPVLDSDGSYFMYSKLRVEKKN | | |
| | WVERNSYSCSVVHEGLHNHHTTKSFSR | | |
| | TPGK | | |
| Rat cKIT D1-5 | Rat cKIT. residues 25-526-TAG | NM_022264 | 116 |
| | SQPSASPGEPSPPSIQPAQSELIVEAGDT | | |
| | IRLTCTDPAFVKWTFEILDVRIENKQSEWI | | |
| | REKAEATHTGKYTCVSGSGLRSSIYVFV | | |
| | RDPAVLFLVGLPLFGKEDNDALVRCPLT | | |
| | DPQVSNYSLIECDGKSLPTDLKFVPNPKA | | |
| | GITIKNVKRAYHRLCIRCAAQREGKWMR | | |
| | SDKFTLKVRAAIKAIPWSVPETSHLLKEG | | |
| | DTFTVICTIKDVSTSVDSMWIKLNPQPQS | | |
| | KAQVKRNSWHQGDFNYERQETLTISSA | | |
| | RVNDSGVFMCYANNTFGSANVTTTLKVV | | |
| | EKGFINIFPVKNTTVFVTDGENVDLWEF | | |
| | EAYPKPEHQQWIYMNRTPTNRGEDYVK | | |
| | SDNQSNIRYVNELRLTRLKGTEGGTYTFL | | |
| | VSNSDVSASVTFDVYVNTKPEILTYDRLM | | |
| | NGRLQCVAAGFPEPTIDWYFCTGAEQR | | |
| | CTVPVPPVDVQIQNASVSPFGKLWQSSI | | |
| | DSSVFRHNGTVECKASNAVGKSSAFFNF | | |
| | AFKGNSKEQIQPHTLFTPRSLEVLFQGP | | |
| | GSPPLKECPPCAAPDLLGGPSVFIFPPKI | | |
| | KDVLMISLSPMVTCVWDVSEDDPDVQIS | | |
| | WFVNNVEVHTAQTQTHREDYNSTLRW | | |
| | SALPIQHQDWMSGKEFKCKVNNRALPS | | |
| | PIEKTISKPRGPVRAPQVYVLPPPAEEMT | | |
| | KKEFSLTCMITGFLPAEIAVDWTSNGRTE | | |
| | QNYKNTATVLDSDGSYFMYSKLRVQKST | | |
| | WERGSLFACSWHEGLHNHLTTKTISRS | | |
| | LGK | | |

TABLE 4
Sequences of cynomolgus cKIT protein

| | Amino acid sequence in | |
|---|---|---|
| | one letter code, signal | |
| | peptide underlined | SEQ |
| | Cynomolgus monkey cKIT, | ID |
| Construct | residues 25-520-TAG | NO |
| Cynomolgus | MYRMQLLSCIALSLALVTNSQ | 117 |
| monkey | PSVSPGEPSPPSIHPAKSELI | |
| cKIT D1-5 | VRVGNEIRLLCIDPGFVKWTF | |
| | EILDETNENKQNEWITEKAEA | |
| | TNTGKYTCTNKHGLSSSIYVF | |
| | VRDPAKLFLVDRSLYGKEDND | |
| | TLVRCPLTDPEVTSYSLKGCQ | |
| | GKPLPKDLRFVPDPKAGITIK | |
| | SVKRAYHRLCLHCSADQEGKS | |
| | VLSDKFILKVRPAFKAVPVVS | |
| | VSKASYLLREGEEFTVTCTIK | |
| | DVSSSVYSTWKRENSQTKLQE | |
| | KYNSWHHGDFNYERQATLTIS | |
| | SARVNDSGVFMCYANNTFGSA | |
| | NVTTTLEWDKGFINIFPMINT | |
| | TVFVNDGENVDLIVEYEAFPK | |
| | PEHQQWIYMNRTFTDKWEDYP | |
| | KSENESNIRYVSELHLTRLKG | |
| | TEGGTYTFLVSNSDVNASIAF | |
| | NVYVNTKPEILTYDRLVNGML | |
| | QCVAAGFPEPTIDWYFCPGTE | |
| | QRCSASVLPVDVQTLNASGPP | |
| | FGKLVVQSSIDSSAFKHNGTV | |
| | ECKAYNDVGKTSAYFNFAFKG | |
| | NNKEQIHPHTLFTPRSHHHHHH | |

Expression of Recombinant cKIT ECD Proteins

The desired cKIT recombinant proteins were expressed in HEK293 derived cell lines (293FS) previously adapted to suspension culture and grown in serum-free medium FreeStyle-293 (Gibco, catalogue #12338018). Both small scale and large scale protein production were via transient transfection and was performed in multiple shaker flasks (Nalgene), up to 1 L each, with 293Fectin® (Life Technologies, catalogue #12347019) as a plasmid carrier. Total DNA and 293Fectin was used at a ratio of 1:1.5 (w:v). DNA to culture ratio was 1 mg/L. The cell culture supernatants were harvested 3-4 days post transfection, centrifuged and sterile filtered prior to purification.

### Tagged ECD Protein Purification

Recombinant Fc-tagged cKIT extracellular domain proteins (e.g., human cKIT ECD-Fc, human cKIT (ECD subdomains 1-3, 4-5)-Fc, cyno cKIT-mFc, rat cKIT-mFc, mouse cKIT-mFc) were purified from the cell culture supernatant. The clarified supernatant was passed over a Protein A Sepharose® column which had been equilibrated with PBS. After washing to baseline, the bound material was eluted with Pierce Immunopure® low pH Elution Buffer, or 100 mM glycine (pH 2.7) and immediately neutralized with ⅛^{th} the elution volume of 1 M Tris pH 9.0. The pooled protein was concentrated if necessary using Amicon® Ultra 15 mL centrifugal concentrators with 10 kD or 30 kD nominal molecular weight cut-offs. The pools were then purified by SEC using a Superdex® 200 26/60 column to remove aggregates. The purified protein was then characterized by SDS-PAGE and SEC-MALLS (Multi-angle laser light scattering). Concentration was determined by absorbance at 280 nm, using the theoretical absorption coefficients calculated from the sequence by Vector NTI.

### Example 3: Binding of cKIT Fabs to cKIT ECD Subdomains

To help define the binding sites of the cKIT Abs, the human cKIT ECD was divided into subdomains 1-3 (ligand binding domain) and subdomains 4-5 (dimerization domain). To determine which subdomains were bound, a sandwich ELISA assay was employed. 1 μg/ml of ECD diluted in 1× Phosphate buffered saline corresponding to cKIT subdomains 1-3, subdomains 4-5 or full-length cKIT ECD were coated on 96 well Immulon® 4-HBX plates (Thermo Scientific Cat #3855, Rockford, Ill.) and incubated overnight at 4° C. Plates were washed three times with wash buffer (1× Phosphate buffered saline (PBS) with 0.01% Tween-20 (Bio-Rad 101-0781)). Plates were blocked with 280 μl/well 3% Bovine Serum Albumin diluted in 1×PBS for 2 hrs at room temperature. Plates were washed three times with wash buffer. Antibodies were prepared at 2 μg/ml in wash buffer with 5-fold dilutions for 8 points and added to ELISA plates at 100 μl/well in triplicate. Plates were incubated on an orbital shaker shaking at 200 rpm for 1 hr at room temperature. Assay plates were washed three times with wash buffer. Secondary antibody F(ab′)₂ Fragment Goat anti-human IgG (H+L) (Jackson Immunoresearch Cat #109-036-088, West Grove, Pa.) was prepared 1:10,000 in wash buffer and added to ELISA plates at 100 μl/well. Plates were incubated with secondary antibody for 1 hr at room temperature shaking at 200 rpm on an orbital shaker. Assay plates were washed three times with wash buffer. To develop the ELISA signal, 100 μl/well of Sure Blue® TMB substrate (KPL Cat #52-00-03, Gaithersburg, Md.) was added to plates and allowed to incubate for 10 mins at room temperature. To stop the reaction 50 μl of 1N Hydrochloric Acid was added to each well. Absorbance was measured at 450 nm using a Molecular Devices SpectraMax® M5 plate reader. To determine the binding response of each antibody the optical density measurements were averaged, standard deviation values generated and graphed using Excel. The binding characteristics of individual anti-cKIT antibody to cKIT can be found in Table 6.

### Example 4: Affinity Measurements of cKIT Antibodies

Affinity of the antibodies to cKIT species orthologues and also to human cKIT was determined using SPR technology using a Biacore® 2000 instrument (GE Healthcare, Pittsburgh, Pa.) and with CM5 sensor chips.

Briefly, HBS-P (0.01 M HEPES, pH 7.4, 0.15 M NaCl, 0.005% Surfactant P20) supplemented with 2% Odyssey® blocking buffer (Li-Cor Biosciences, Lincoln, Nebr.) was used as the running buffer for all the experiments. The immobilization level and analyte interactions were measured by response unit (RU). Pilot experiments were performed to test and confirm the feasibility of the immobilization of the anti-human Fc antibody (Catalog number BR100839, GE Healthcare, Pittsburgh, Pa.) and the capture of the test antibodies.

For kinetic measurements, the experiments were performed in which the antibodies were captured to the sensor chip surface via the immobilized anti-human Fc antibody and the ability of the cKIT proteins to bind in free solution was determined. Briefly, g/ml of anti-human Fc antibody at pH 5 was immobilized on a CM5 sensor chip through amine coupling at flow rate of 5 μl/min on both flow cells to reach 10,500 RUs. 0.1-1 μg/ml of test antibodies were then injected at 10 μl/min for 1 minute. Captured levels of the antibodies were generally kept below 200 RUs. Subsequently, 3.125-50 nM of cKIT receptor extracellular domains (ECD) were diluted in a 2-fold series and injected at a flow rate of I/min for 3 min over both reference and test flow cells. A table of tested ECDs is listed below (Table 5). Dissociation of ECD binding was followed for 10 min. After each injection cycle, the chip surface was regenerated with 3 M MgCl₂ at 10 μl/min for 30 seconds. All experiments were performed at 25° C. and the response data were globally fitted with a simple 1:1 interaction model (using Scrubber 2® software version 2.0b (BioLogic Software) to obtain estimates of on rate (kₐ), off-rate (k_{d}) and affinity (K_{D}). Table 6 lists the domain binding and affinity of selected anti-cKIT antibodies.

TABLE 5
cKIT ECD isotype and source

| ECD | | |
|---|---|---|
| Isotype | Tag | Source |
| Human | C-terminal 6x His | Novartis construct |
| | (SEQ ID NO: 151) | |
| Cyno | C-terminal 6x His | Novartis construct |
| | (SEQ ID NO: 151) | |
| Mouse | C-terminal 6x His | Sino Biological Inc (Catalog number: 50530- |
| | (SEQ ID NO: 151) | M08H) |
| Rat | C-terminal mFc | Novartis construct |

TABLE 6
Antibody affinity and cross reactivity

| | | KD (pM) | KD (pM) | Reac- | Reac- |
|---|---|---|---|---|---|
| | cKIT | to human | to cyno | tivity | tivity |
| | domain | cKIT | cKIT | to | to |
| | | ECD in | ECD in | mouse | rat |
| Ab | binding | SET | SET | cKIT | cKIT |
| Anti-cKIT Ab1 | D1-3 | 94 | 170 | Not | Not |
| | | | | reactive | reactive |
| Anti-cKIT Ab2 | D1-3 | 7 | 10 | Not | Not |
| | | | | reactive | reactive |
| Anti-cKIT Ab3 | D1-3 | 160 | 52 | Not | Not |
| | | | | reactive | reactive |
| Anti-cKIT Ab4 | D4-5 | 2400 | 140 | Yes | Yes |
| Anti-cKIT Ab5 | D1-3 | 110 | 180 | Yes | Yes |

### Example 5 In Vitro Human HSC Cell Killing Assays by cKIT ADCs

### In Vitro HSC Viability Assays

Human mobilized peripheral blood hematopoietic stem cells (HSCs) were obtained from HemaCare (catalog number M001F-GCSF-3). Each vial of ˜1 million cells was thawed and diluted into 10 ml of 1×HBSS and centrifuged for 7 minutes at 1200 rpm. The cell pellet was resuspended in 18 ml of growth medium containing three growth factors (StemSpan SFEM (StemCell Technologies, catalog number 09650) with 50 ng/ml each of TPO (R&D Systems, catalog number 288-TP) Flt3 ligand (Life Technologies, catalog number PHC9413), and IL-6 (Life Technologies, catalog number PHC0063), supplemented with amino acids (Gibco, catalog number 10378-016)).

Test agents were diluted in duplicate into a 384-well black assay plate at a final volume of 5 μl, starting at 10 μg/ml and with 1:3 serial dilutions. Cells from above were added to each well at a final volume of 45 μl. Cells were incubated at 37° C. and 5% oxygen for 7 days. At the end of culture, cells were harvested for staining by centrifuging the assay plate for 4 minutes at 1200 rpm. Supernatants were then aspirated and the cells were washed and transferred to a different 384-well plate (Greiner Bio-One TC-treated, black clear flat, catalog number 781092).

For human cell assays, each well was stained with anti-CD34-PerCP (Becton Dickinson, catalog number 340666) and anti-CD90-APC (Becton Dickinson, catalog number 559869), washed, and resuspended in FACS buffer to a final volume of 50 μl. Cells were then analyzed on a Becton Dickinson Fortessa flow cytometer and quantified for analysis.

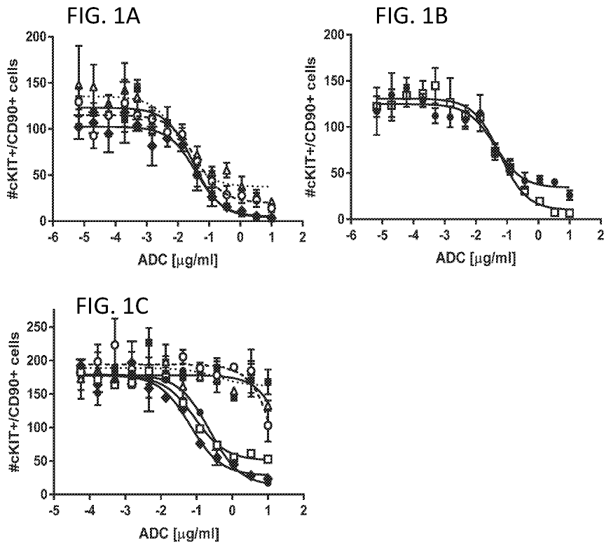

Toxin conjugates of antibodies and antibody fragments recognizing cKIT killed HSCs as determined in this assay. Quantitation of cells by FACS showed fewer viable cells in wells treated with anti-cKIT-toxin conjugates than in control wells treated with PBS or with isotype control toxin conjugates of antibody or antibody fragment. Data are shown in FIG. 1 and summarized in Table 7. The naming convention used herein is J #, correspond to the specific Conjugate No. described in Table 2.

TABLE 7
Cell viability after treatment of anti-cKIT Fab-toxin conjugates

| Conjug. No. | Cell population | EC50 (ng/ml) |
|---|---|---|
| J4 | Human total nucleated cells | 16 |
| | Human CD34 + cells | 25 |
| | Human CD90 + cells | 37 |
| J5 | Human total nucleated cells | 8.8 |
| | Human CD34 + cells | 13 |
| | Human CD90 + cells | 39 |
| J8 | Human total nucleated cells | 10 |
| | Human CD34 + cells | 16 |
| | Human CD90 + cells | 31 |
| J9 | Human total nucleated cells | 1.8 |
| | Human CD34 + cells | 3.6 |
| | Human CD90 + cells | 12 |
| J10 | Human total nucleated cells | 35 |
| | Human CD34 + cells | 57 |
| | Human CD90 + cells | 59 |
| J11 | Human total nucleated cells | 16 |
| | Human CD34 + cells | 27 |
| | Human CD90 + cells | 41 |
| J15 | Human total nucleated cells | 115 |
| | Human CD34 + cells | 151 |
| | Human CD90 + cells | 229 |
| J16 | Human total nucleated cells | 29 |
| | Human CD34 + cells | 43 |
| | Human CD90 + cells | 89 |

### Example 6 In Vitro Assay of Human Mast Cell Degranulation

Mature mast cells were generated using CD34+ progenitors from mobilized peripheral blood. CD34+ cells were cultured in StemSpan SFEM (StemCell Technologies) supplemented with recombinant human stem cell factor (rhSCF, 50 ng/ml, Gibco), recombinant human interleukin 6 (rhIL-6, 50 ng/ml, Gibco), recombinant human IL-3 (30 ng/ml, Peprotech), GlutaMAX (2 nM, Gibco), penicillin (100 U/ml, Hyclone) and streptomycin (100 μg/ml, Hyclone). Recombinant hll-3 was added only during the first week of the culture. After the third week, half of the medium was replaced weekly with fresh medium containing rhIL-6 (50 ng/ml) and rhSCF (50 ng/ml). Mature mast cell purity was evaluated by surface staining of high-affinity IgE receptor (FCεRI, eBioscience) and CD117 (BD). Cells were used between week 8 and 12 of the culture.

The derived mast cells were washed once to remove SCF, and the required amount of cells was incubated overnight in mast cell medium containing rhIL-6 (50 ng/ml) with or without rhSCF (50 ng/ml). As positive control for mast cell degranulation, a portion of the cells were sensitized with human myeloma IgE (100 ng/ml, EMD Millipore). The following day, anti-cKIT antibody or antibody fragments or toxin conjugates thereof, mouse monoclonal anti-human IgG1 (Fab specific, Sigma), goat anti-human IgE (Abcam) and compound 48/80 (Sigma) dilutions were prepared in HEPES degranulation buffer (10 mM HEPES, 137 mM NaCl, 2.7 mM KCl, 0.4 mM sodium phosphate dibasic, 5.6 mM glucose, pH adjusted at 7.4 and mixed with 1.8 mM calcium chloride and 1.3 mM magnesium sulfate) supplemented with 0.04% bovine serum albumin (BSA, Sigma). Test agents and anti-IgG1 were mixed together in a V-bottom 384-well assay plate while anti-IgE and compound 48/80 were tested alone. The assay plate was incubated 30 min at 37° C. During the incubation, cells were washed 3 times with HEPES degranulation buffer+0.04% BSA to remove medium and unbound IgE. Cells were resuspended in HEPES degranulation buffer+0.04% BSA and seeded at 3000 cells per well in the assay plate for a final reaction volume of 50 μl. Cells that were sensitized with IgE were used only with anti-IgE as a positive control for degranulation. The assay plate was incubated 30 min at 37° C. for degranulation to occur. During this incubation, p-nitro-N-acetyl-pi-D-gluosamine (pNAG, Sigma) buffer was prepared by sonicating 3.5 mg/ml of pNAG in citrate buffer (40 mM citric acid, 20 mM sodium phosphate dibasic, pH 4.5). β-hexosaminidase release was measured by mixing 20 μl of cell supernatant with 40 μl of pNAG solution in a flat-bottom 384-well plate. This plate was incubated for 1.5 hour at 3700, and the reaction was stopped by the addition of 40 μl of stop solution (400 mM glycine, pH 10.7). Absorbance was read using a plate reader at λ=405 nm with reference filter at λ=620 nm.

Full-length IgG controls used in the mast cell degranulation assays are described in Table 8.

TABLE 8
Full-length IgG controls used in the
mast cell degranulation assays

| Name | HC sequence | LC sequence |
|---|---|---|
| Anti- | SEQ ID NO: 12 | SEQ ID NO: 25 |
| cKIT | | |
| Ab1 | | |
| Anti- | SEQ ID NO: 38 | SEQ ID NO: 49 |
| cKIT | | |
| Ab2 | | |
| Anti- | SEQ ID NO: 56 | SEQ ID NO: 25 |
| cKIT | | |
| Ab3 | | |
| Anti- | SEQ ID NO: 71 | SEQ ID NO: 84 |
| cKIT | | |
| Ab4 | | |
| Anti- | EVQLVESGGGLVQPG | SEQ ID NO: 147 |
| Her2 | GSLRLSCAASGFNIK | |
| | DTYIHWVRQAPGKGL | |
| | EWVARIYPTNGYTRY | |
| | ADSVKGRFTISADTS | |
| | KNTAYLQMNSLRAED | |
| | TAVYYCSRWGGDGFY | |
| | AMDYWGQGTLVTVSS | |
| | ASTKGPSVFPLAPSS | |
| | KSTSGGTAALGCLVK | |
| | DYFPEPVTVSWNSGA | |
| | LTSGVHTFPAVLQSS | |
| | GLYSLSSWTVPSSSL | |
| | GTQTYICNVNHKPSN | |
| | TKVDKKVEPKSCDKT | |
| | HTCPPCPAPELLGGP | |
| | SVFLFPPKPKDTLMI | |
| | SRTPEVTCWVDVSHE | |
| | DPEVKFNWYVDGVEV | |
| | HNAKTKPREEQYNST | |
| | YRWSVLTVLHQDWLN | |
| | GKEYKCKVSNKALPA | |
| | PIEKTISKAKGQPRE | |
| | PQVYTLPPSREEMTK | |
| | NQVSLTCLVKGFYPS | |
| | DIAVEWESNGQPENN | |
| | YKTTPPVLDSDGSFF | |
| | LYSKLTVDKSRWQQG | |
| | NVFSCSVMHEALHNH | |
| | YTQKSLSLSPGK | |
| | (SEQ ID NO: 148) | |

### Example 7 In Vitro Assay of Human Mast Cell Degranulation by Full-Length Anti-cKIT Antibody and F(Ab′) and Fab Fragments Thereof

Mature mast cells were generated and tested with anti-cKIT antibody and F(ab′)₂ and Fab fragments as described in Example 6.

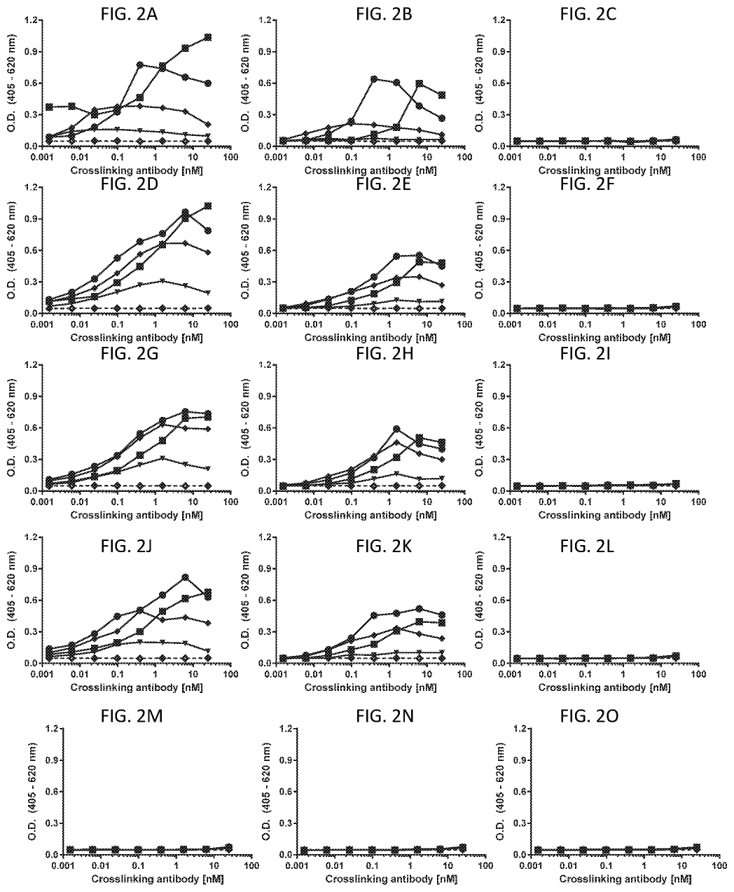

As shown in FIGS. 2A-2C, full length anti-cKIT Ab4 and F(ab′4)₂ fragment caused mast cell degranulation when cross-linked, while no mast cell degranulation was triggered by Fab4 (HC-E152C) fragment at all tested concentrations. FIGS. 2D-2F show that full length anti-cKIT Ab1 and F(ab′1)₂ fragment caused mast cell degranulation when cross-linked, while no mast cell degranulation was triggered by Fab1 (HC-E152C) fragment at all tested concentrations. FIGS. 2G-2I show that full length anti-cKIT Ab2 and F(ab′2)₂ fragment caused mast cell degranulation when cross-linked, while no mast cell degranulation was triggered by Fab2 (HC-E152C) fragment at all tested concentrations. FIGS. 2J-2L show that full length anti-cKIT Ab3 and F(ab′3)₂ fragment caused mast cell degranulation when cross-linked, while no mast cell degranulation was triggered by Fab3 (HC-E152C) fragment at all tested concentrations. This suggests that the Fab fragments do not cause mast cell degranulation even when bound and multimerized into larger complexes as could be observed if a patient developed or had pre-existing anti-drug antibodies recognizing Fab fragments. On the other hand, F(ab′)₂ fragments do cause mast cell degranulation at a level similar to the full-length anti-cKIT antibody when bound and multimerized into larger complexes.

### Example 8 In Vivo Ablation of Human HSCs from Mouse Host

To assess test agents for in vivo efficacy against human HSCs, severely immune compromised NOD.Cg-Prkdc^{scid} IL2^{tm1wjl}/SzJ mice that are humanized with human CD34+ cells were purchased from Jackson Laboratory. Percent human chimerism was determined by flow cytometry of blood samples. For this, blood was stained with the following antibodies: anti-human CD45-e450 (eBioscience, catalog #48-0459-42), anti-mouse CD45-APC (Becton Dickinson, catalog #559864 anti-human anti-human CD33-Pe (Becton Dickinson, catalog #347787), anti-human CD19-FITC (Becton Dickinson, catalog #555422), and anti-human CD3-PeCy7 (Becton Dickinson, catalog #557851). Once human chimerism was confirmed, humanized NSG mice were dosed with a test agent intraperitoneally b.i.d. The degree of human chimerism was re-assessed after dosing. To assess presence or absence of human HSCs, mice were euthanized and bone marrow was isolated and stained with the following antibodies: anti-human CD45-e450 (eBioscience, catalog #48-0459-42), anti-mouse CD45-APC (Becton Dickinson, catalog #559864), anti-human CD34-PE (Becton Dickinson, catalog #348057), anti-human CD38-FITC (Becton Dickinson, catalog #340926), anti-human CD11b-PE (Becton Dickinson, catalog #555388), anti-human CD33-PeCy7 (Becton Dickinson, catalog #333946), anti-human CD19-FITC (Becton Dickinson, catalog #555412), and anti-human CD3-PeCy7 (Becton Dickinson, catalog #557851). Cell populations were assessed via flow cytometry and analyzed with FlowJo.

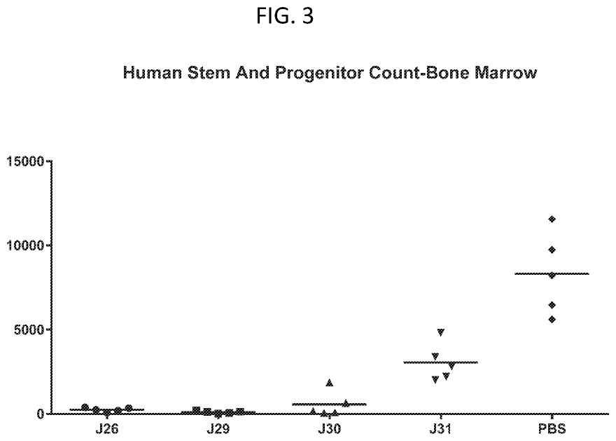

In one particular experiment, mice were dosed with 10 mg/kg of anti-cKIT conjugate J26, J29, or J30, or isotype control conjugate J31 twice per day for 2 days. Mice were euthanized on day 21 and their bone marrow was analyzed. As shown in FIG. 3, mice treated with an anti-cKIT conjugate J26, J29, or J30 showed reduced human HSCs (human CD45+, human CD34+, human CD38-), while mice treated with isotype control conjugate J31 showed variable chimerism.

This experiment shows that anti-cKit Fab′-toxin conjugates were able to deplete HSCs from bone marrow. The anti-cKIT Fab′-auristatin conjugates (e.g., J26, J29, J30) were able to ablate human HSCs in vivo.

Unless defined otherwise, the technical and scientific terms used herein have the same meaning as that usually understood by a specialist familiar with the field to which the disclosure belongs.

Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein. Unless indicated otherwise, each of the references cited herein is incorporated in its entirety by reference.

Claims to the invention are non-limiting and are provided below.

Although particular aspects and claims have been disclosed herein in detail, this has been done by way of example for purposes of illustration only, and is not intended to be limiting with respect to the scope of the appended claims, or the scope of subject matter of claims of any corresponding future application. In particular, it is contemplated by the inventors that various substitutions, alterations, and modifications may be made to the disclosure without departing from the spirit and scope of the disclosure as defined by the claims. The choice of nucleic acid starting material, clone of interest, or library type is believed to be a matter of routine for a person of ordinary skill in the art with knowledge of the aspects described herein. Other aspects, advantages, and modifications considered to be within the scope of the following claims. Those skilled in the art will recognize or be able to ascertain, using no more than routine experimentation, many equivalents of the specific aspects of the invention described herein. Such equivalents are intended to be encompassed by the following claims. Redrafting of claim scope in later filed corresponding applications may be due to limitations by the patent laws of various countries and should not be interpreted as giving up subject matter of the claims.

What is claimed is:

1. A conjugate of Formula (I) or a pharmaceutically acceptable salt thereof;
A-(L_{B}-(D)ₙ)y Formula (I)
wherein:
A is an antibody fragment that specifically binds to human cKIT;
L_{B} is a linker;
n is an integer from 1 to 10;
y is an integer from 1 to 10, and
D is a cytotoxic agent selected from a compound of Formula (A): 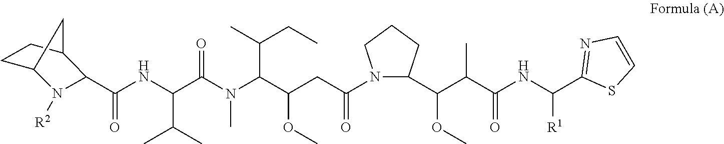
wherein:
R¹ is 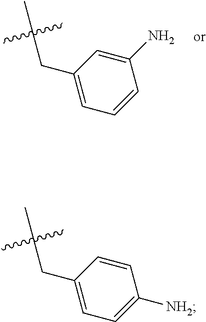 [SMILES: *Cc1cccc(N)c1 ; *Cc1ccc(N)cc1]
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)ₙR⁴ or C₁-C₆alkyl which is optionally substituted with —CN, —C(═O)NH₂ or 1 to 5 hydroxyl;
each R³ is independently selected from C₁-C₈alkyl and C₁-C₈alkyl which is optionally substituted with 1 to 5 hydroxyl;
and
each R⁴ is independently selected from H and C₁-C₆alkyl;
or D is a cytotoxic agent selected from a compound of Formula (B): 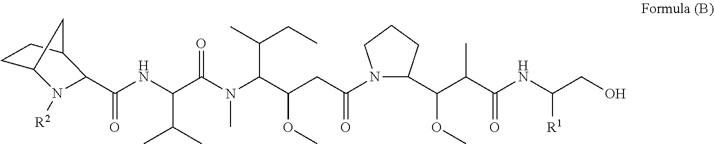
wherein:
R¹ is 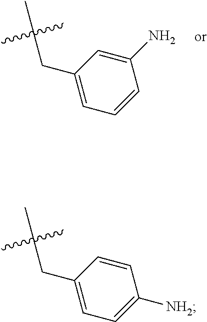 [SMILES: *Cc1cccc(N)c1 ; *Cc1ccc(N)cc1]
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)ₙR⁴ or C₁-C₆alkyl which is optionally substituted with —CN, —C(═O)NH₂ or 1 to 5 hydroxyl;
each R³ is independently selected from C₁-C₆alkyl and C₁-C₆alkyl which is optionally substituted with 1 to 5 hydroxyl;
and
each R⁴ is independently selected from H and C₁-C₆alkyl.

2. The conjugate of claim 1, wherein n is 1, 2, 3, 4, 5, 6, 7 or 8.

3. The conjugate of claim 1 or 2, wherein y is 1, 2, 3 or 4.

4. The conjugate of any one of claims 1 to 3, wherein each L_{B} is independently selected from a cleavable linker or a non-cleavable linker.

5. The conjugate of any one of claims 1 to 4, wherein each L_{B} is a cleavable linker.

6. The conjugate of any one of claims 1 to 4, wherein each L_{B} is a non-cleavable linker.

7. A conjugate having the structure of Formula (E): 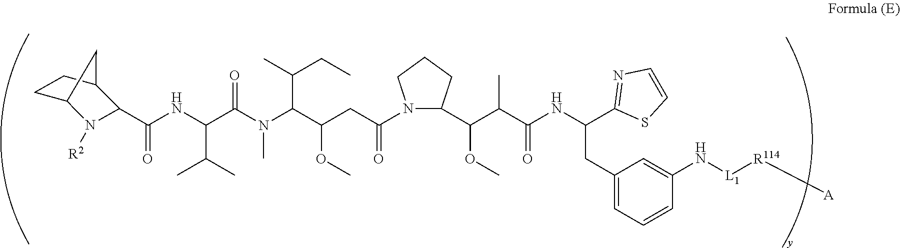
wherein:
A represents an antibody fragment (e.g., Fab or Fab′) that specifically binds to human cKIT;
y is an integer from 1 to 10;
R² is H, C₁-C₆alkyl, —C(═O)R³, —(CH₂)ₘOH, —C(═O)(CH₂)ₘOH, —C(═O)((CH₂)ₘO)ₙR⁴, —((CH₂)ₘO)ₙR⁴ or C₁-C₆alkyl which is optionally substituted with —CN, —C(═O)NH₂ or 1 to 5 hydroxy;
each R³ is independently selected from C₁-C₆alkyl and C₁-C₆alkyl which is optionally substituted with 1 to 5 hydroxyl;
each R⁴ is independently selected from H and C₁-C₆alkyl;
L₁ is —X₁C(═O)((CH₂)ₘO)ₚ(CH₂)ₘ—**, —X₁C(═O)(CH₂)ₘ—**, —X₂X₁C(═O)((CH₂)ₘO)ₚ(CH₂)ₘ—**, —X₂X₁C(═O)(CH₂)ₘ—**, —X₃C(═O)((CH₂)ₘO)ₚ(CH₂)ₘ—**, —X₃C(═O)(CH₂)ₘ—**, —X₃C(═O)(CH₂)ₘNHC(═O)((CH₂)ₘO)ₚ(CH₂)ₘ—**, —X₃C(═O)(CH₂)ₘNHC(═O)(CH₂)ₘ—**, —X₃C(═O)(CH₂)ₘ—**, —X₁C(═O)((CH₂)ₘO)ₚ(CH₂)ₘX₄(CH₂)ₘ—**, —X₁C(═O)(CH₂)ₘX₄(CH₂)ₘ—**, —X₂X₁C(═O)((CH₂)ₘO)ₚ(CH₂)ₘX₄(CH₂)ₘ—** or —X₂X₁C(═O)(CH₂)ₘX₄(CH₂)ₘ—**, where ** indicates the point of attachment to R¹¹⁴;
X₁ is 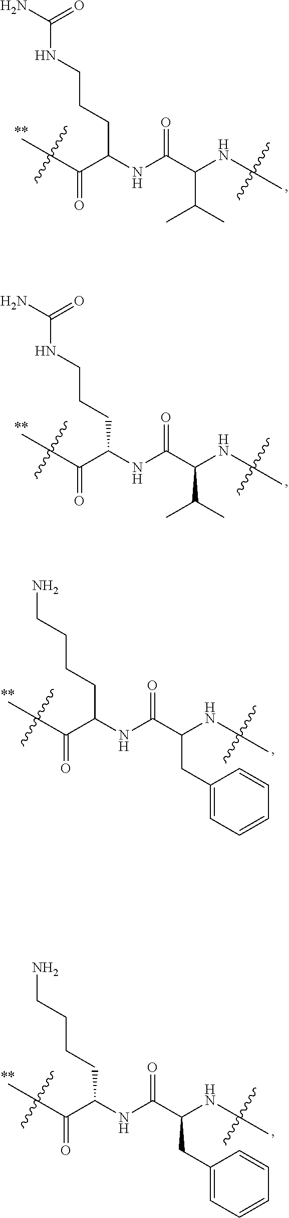 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O] 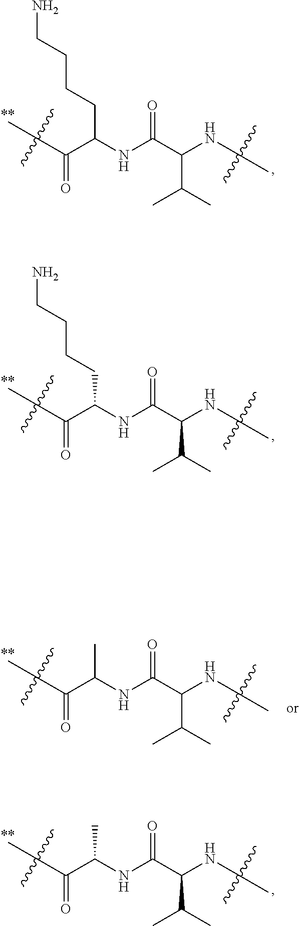 [SMILES: *NC(C(=O)NC(CCCCN)C(*)=O)C(C)C ; *N[C@H](C(=O)N[C@@H](CCCCN)C(*)=O)C(C)C ; *NC(C(=O)NC(C)C(*)=O)C(C)C ; *N[C@H](C(=O)N[C@@H](C)C(*)=O)C(C)C]
where ** indicates the point of attachment to the —NH— or to X₂;
X₂ is 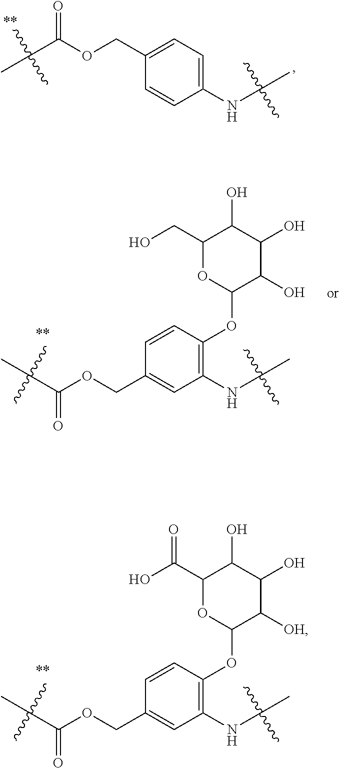 [SMILES: *Nc1ccc(COC(*)=O)cc1 ; *Nc1cc(COC(*)=O)ccc1OC1OC(CO)C(O)C(O)C1O ; *Nc1cc(COC(*)=O)ccc1OC1OC(C(=O)O)C(O)C(O)C1O]
where ** indicates the point of attachment to the —NH—;
X₃ is 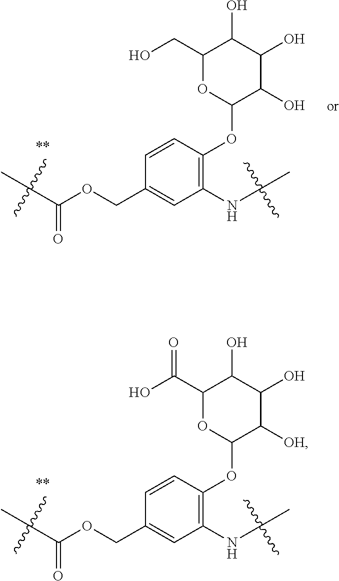 [SMILES: *Nc1cc(COC(*)=O)ccc1OC1OC(CO)C(O)C(O)C1O ; *Nc1cc(COC(*)=O)ccc1OC1OC(C(=O)O)C(O)C(O)C1O]
where ** indicates the point of attachment to the —NH—;
X₄ is 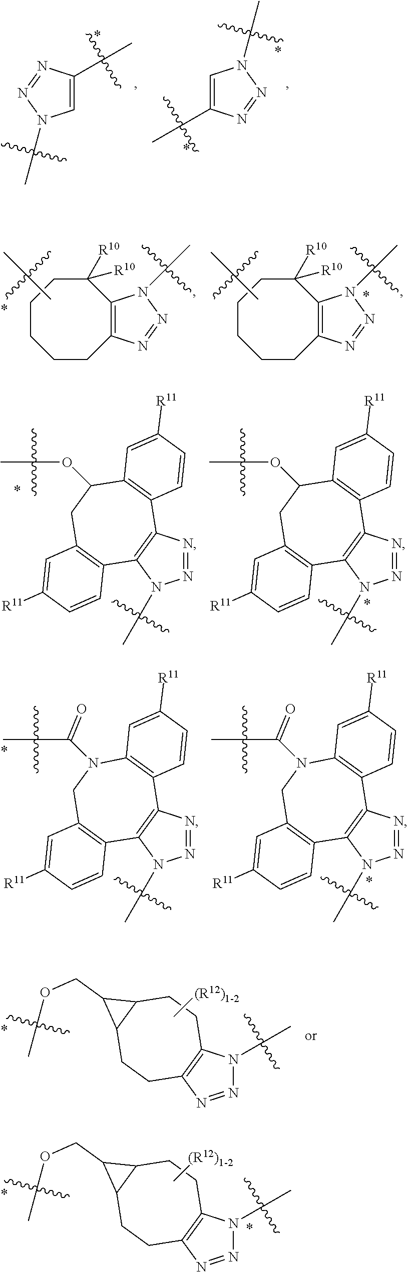
where the * indicates the point of attachment is toward R¹¹⁴;
R¹¹⁴ is 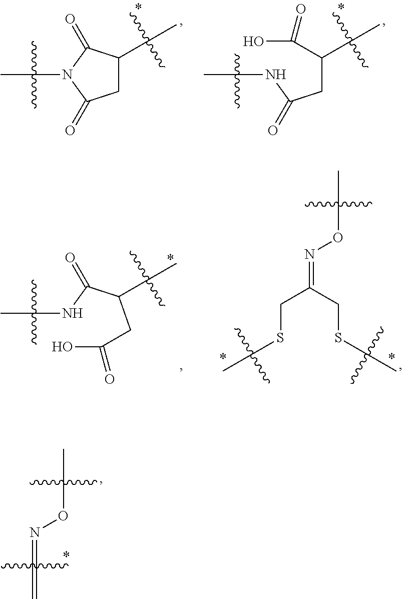
—NR⁶C(═O)CH₂—*, —NHC(═O)CH₂—*, —S(═O)₂CH₂CH₂—*, —(CH₂)₂S(═O)₂CH₂CH₂—*, —NR⁶S(═O)₂CH₂CH₂*, —NR⁶C(═O)CH₂CH₂*, —NH—, —C(═O)—, —NHC(═O)—*, —CH₂NHCH₂CH₂—*, —NHCH₂CH₂—*, —S—, 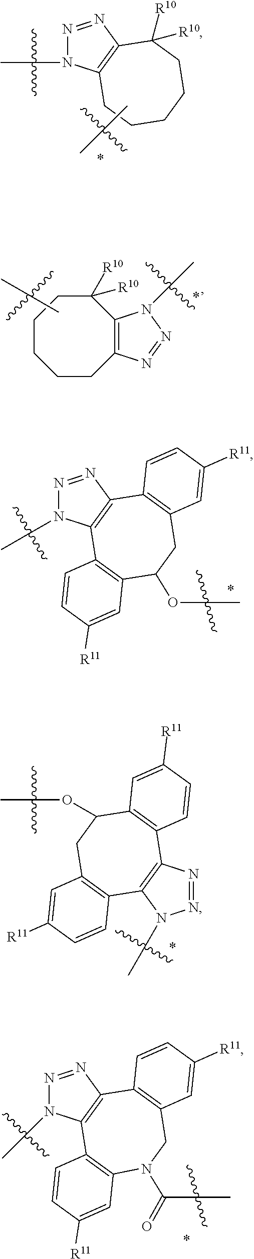 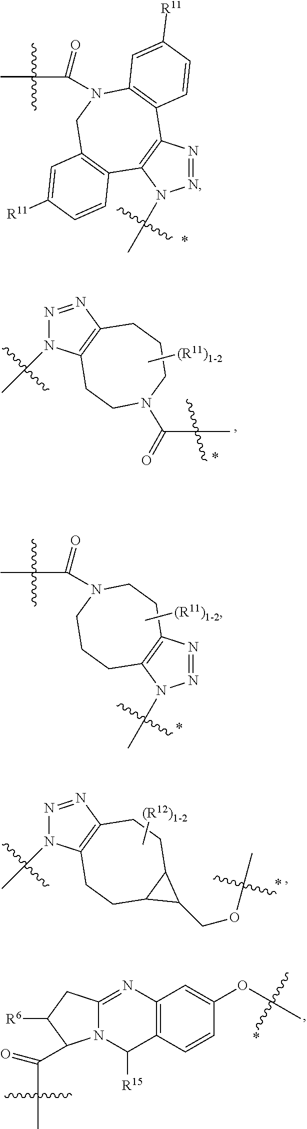 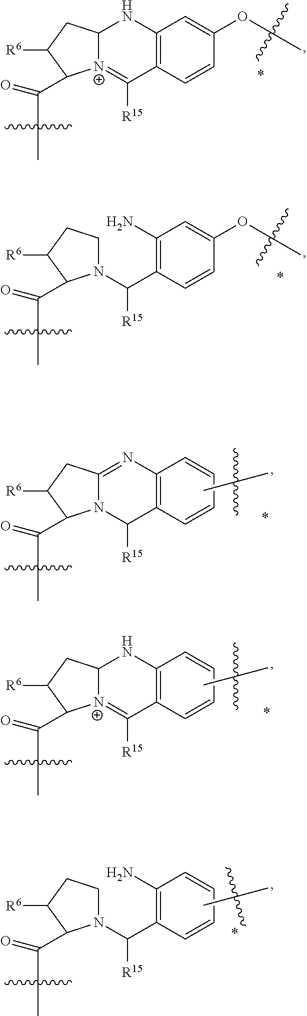 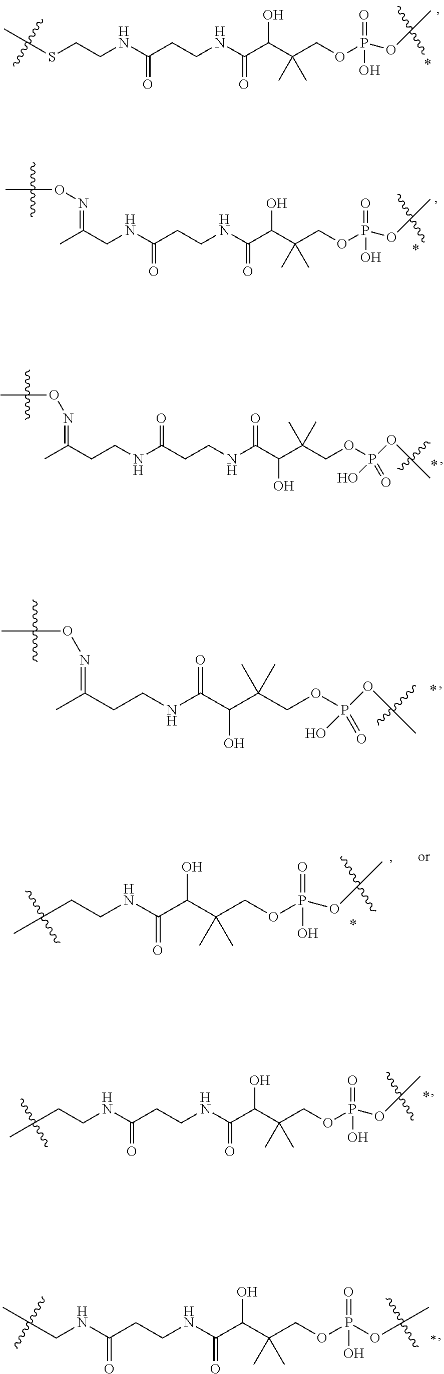
where the * indicates the point of attachment to A;
each R⁶ is independently selected from H and C₁-C₆alkyl;
each R¹⁰ is independently selected from H, C₁-C₆alkyl, F, Cl, and —OH;
each R¹¹ is independently selected from H, C₁-C₆alkyl, F, Cl, —NH₂, —OCH₃, —OCH₂CH₃, —N(CH₃)₂, —CN, —NO₂ and —OH;
each R¹² is independently selected from H, C1₆alkyl, fluoro, benzyloxy substituted with —C(═O)OH, benzyl substituted with —C(═O)OH, C1₄alkoxy substituted with —C(═O)OH and C1₄alkyl substituted with —C(═O)OH;
each R¹⁵ is independently selected from H, —CH₃ and phenyl;
each m is independently selected from 1, 2, 3, 4, 5, 6, 7, 8, 9 and 10, and
each p is independently selected from 1, 2, 3, 4, 5, 6, 7, 8, 9, 10, 11, 12, 13 and 14.

8. The conjugate of claim 7, selected from 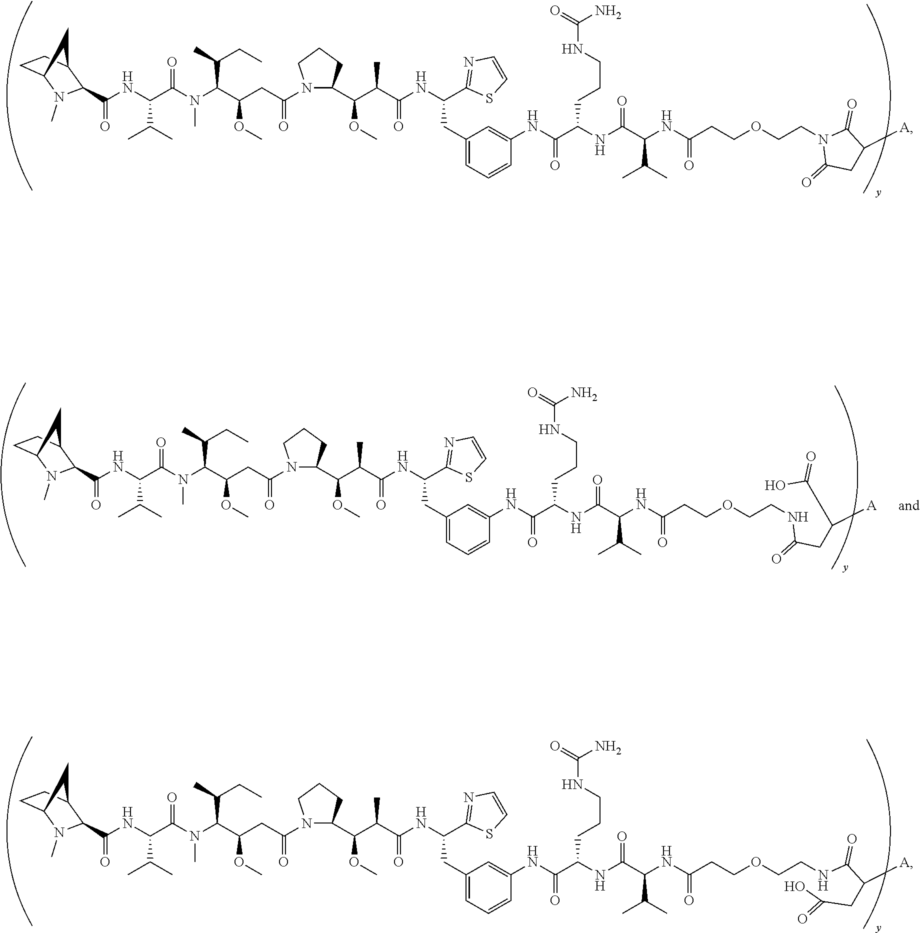
wherein:
A represents an antibody fragment (e.g., Fab or Fab′) that specifically binds to human cKIT, and
y is an integer from 1 to 10.

9. A conjugate having the structure of Formula (G): 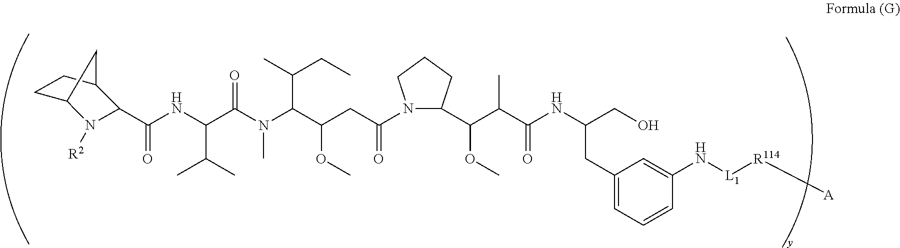
wherein:
A represents an antibody fragment (e.g., Fab or Fab′) that specifically binds to human cKIT;
y is an integer from 1 to 10;
R² is H, C₁-C₆alkyl, —C(═O)R³, (CH₂)ₘOH, —C(═O)(CH)ₘOH, —C(═O)((CH₂)ₘO)ₙR⁴, —((CH₂)ₘO)ₙR⁴ or C₁-C₆alkyl which is optionally substituted with —CN, —C(═O)NH₂ or 1 to 5 hydroxy;
each R³ is independently selected from C₁-C₆alkyl and C₁-C₆alkyl which is optionally substituted with 1 to 5 hydroxyl;
each R⁴ is independently selected from H and C₁-C₆alkyl;
L₁ is —X₁C(═O)((CH₂)ₘO)ₚ(CH₂)ₘ—**, —X₁C(═O)(CH₂)ₘ—**, —X₂X₁C(═O)((CH₂)ₘO)ₚ(CH₂)ₘ—**, —X₂X₁C(═O)(CH₂)ₘ—**, —X₃C(═O)((CH₂)ₘO)ₚ(CH₂)ₘ—**, —X₃C(═O)(CH₂)ₘ—**, —X₃C(═O)(CH₂)ₘNHC(═O)((CH₂)ₘO)ₚ(CH₂)ₘ—**, —X₃C(═O)(CH₂)ₘNHC(═O)(CH₂)ₘ—**, —X₃C(═O)(CH₂)ₘ—**, —X₁C(═O)((CH₂)ₘO)ₚ(CH₂)ₘX₄(CH₂)ₘ—**, —X₁C(═O)(CH₂)ₘX₄(CH₂)ₘ—**, —X₂X₁C(═O)((CH₂)ₘO)ₚ(CH₂)ₘX₄(CH₂)ₘ—** or —X₂X₁C(═O)(CH₂)ₘX₄(CH₂)ₘ—**, where ** indicates the point of attachment to R⁴;
X₁ is 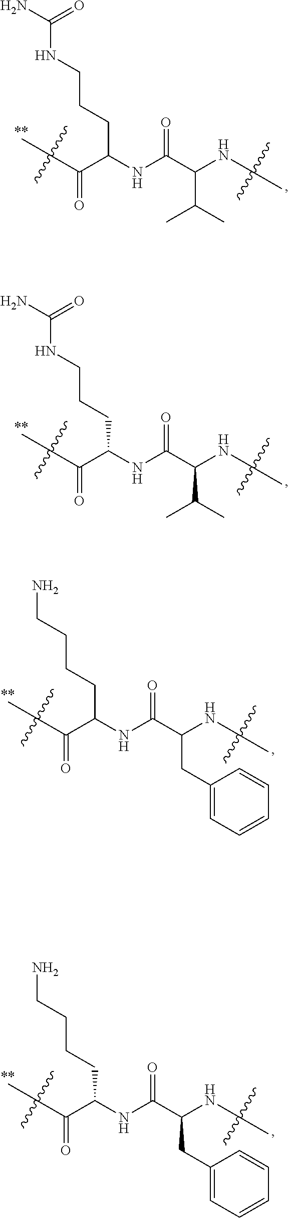 [SMILES: *NC(C(=O)NC(CCCNC(N)=O)C(*)=O)C(C)C ; *N[C@H](C(=O)N[C@@H](CCCNC(N)=O)C(*)=O)C(C)C ; *NC(Cc1ccccc1)C(=O)NC(CCCCN)C(*)=O ; *N[C@@H](Cc1ccccc1)C(=O)N[C@@H](CCCCN)C(*)=O] 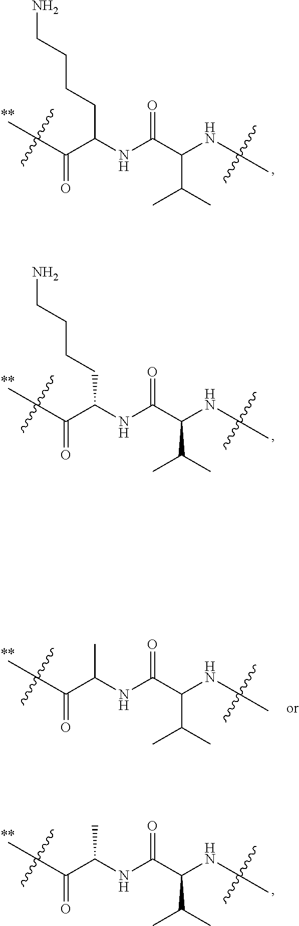 [SMILES: *NC(C(=O)NC(CCCCN)C(*)=O)C(C)C ; *N[C@H](C(=O)N[C@@H](CCCCN)C(*)=O)C(C)C ; *NC(C(=O)NC(C)C(*)=O)C(C)C ; *N[C@H](C(=O)N[C@@H](C)C(*)=O)C(C)C]
where ** indicates the point of attachment to the —NH— or to X₂;
X₂ is 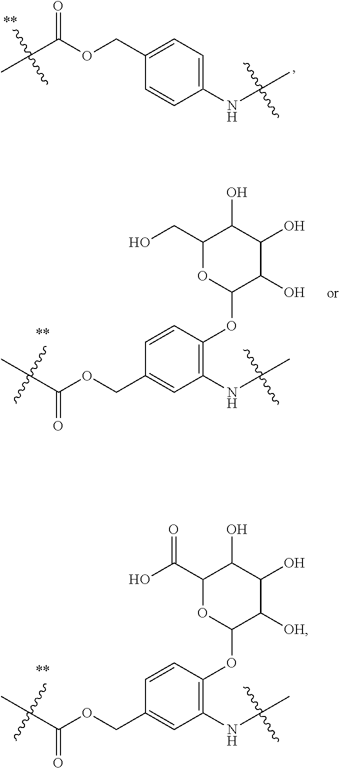 [SMILES: *Nc1ccc(COC(*)=O)cc1 ; *Nc1cc(COC(*)=O)ccc1OC1OC(CO)C(O)C(O)C1O ; *Nc1cc(COC(*)=O)ccc1OC1OC(C(=O)O)C(O)C(O)C1O]
where ** indicates the point of attachment to the —NH—;
X₃ is 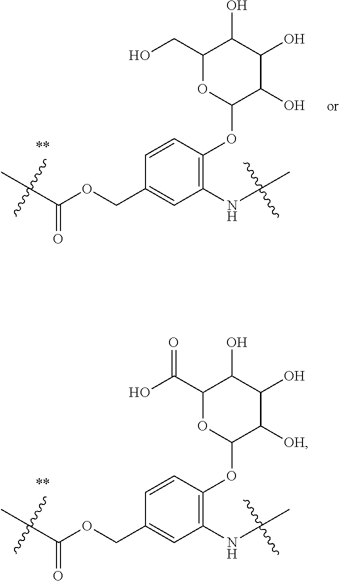 [SMILES: *Nc1cc(COC(*)=O)ccc1OC1OC(CO)C(O)C(O)C1O ; *Nc1cc(COC(*)=O)ccc1OC1OC(C(=O)O)C(O)C(O)C1O]
where ** indicates the point of attachment to the —NH—;
X₄ is 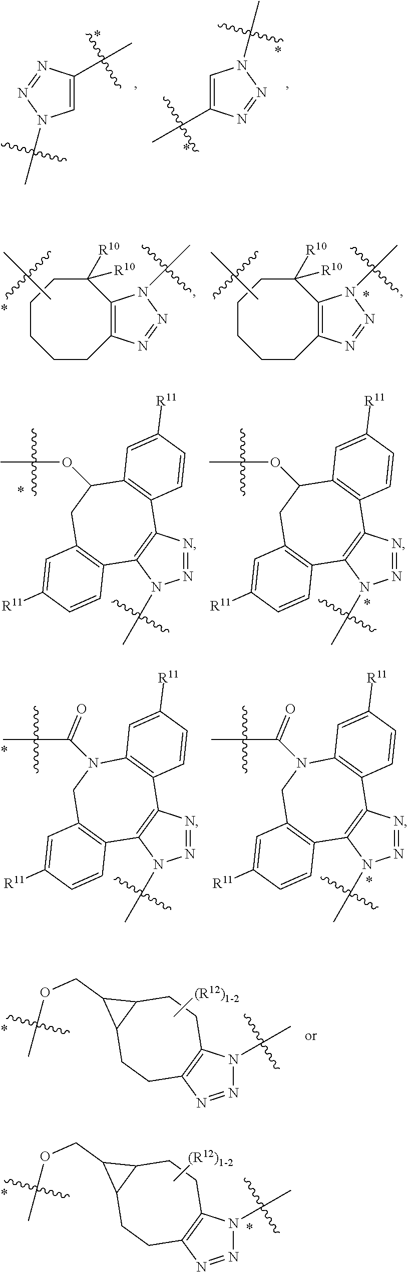
where the * indicates the point of attachment is toward R¹¹⁴;
R¹¹⁴ is 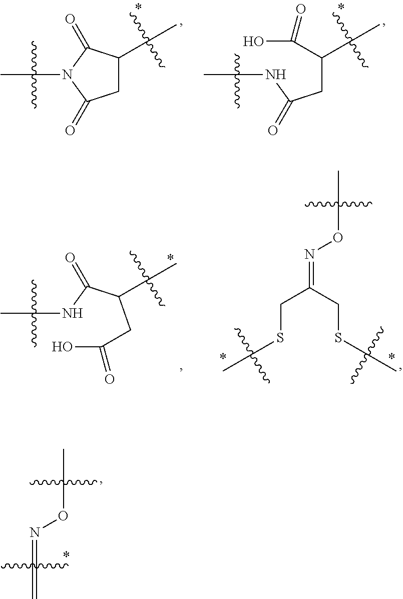
—NR⁶C(═O)CH₂—*, —NHC(═O)CH₂—*, —S(═O)₂CH₂CH₂—*, —(CH₂)₂S(═O)₂CH₂CH₂—*, —NR⁶S(═O)₂CH₂CH₂—*, —NR⁶C(═O)CH₂CH₂—*, —NH—, —C(═O)—, —NHC(═O)—*, —CH₂NHCH₂CH₂—*, —NHCH₂CH₂—*, —S—, 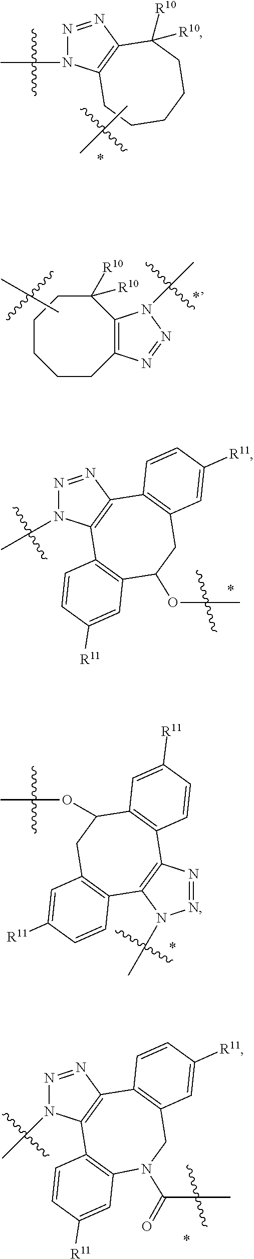 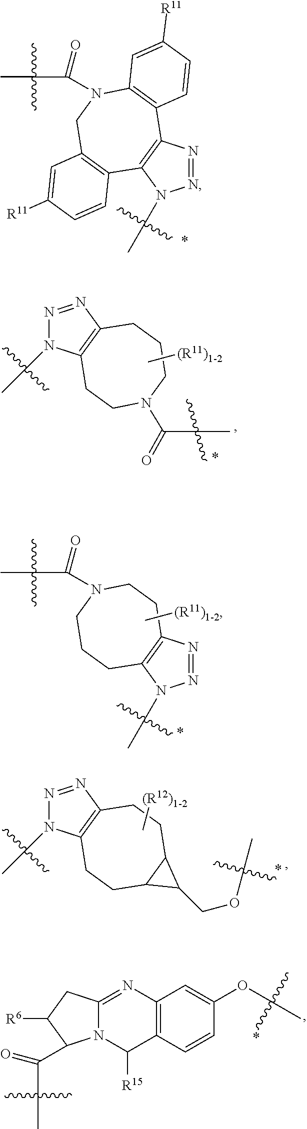 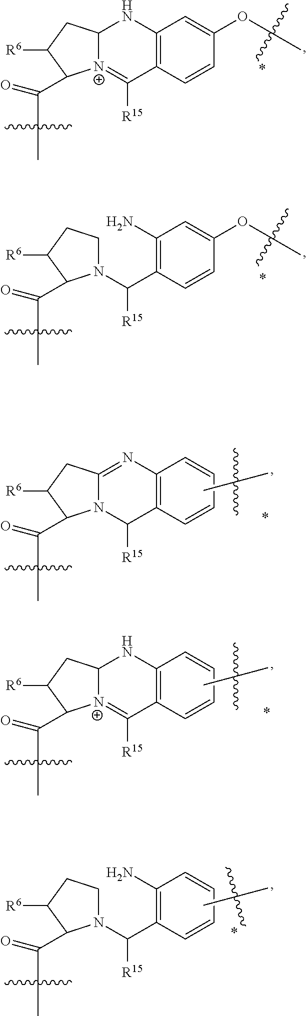 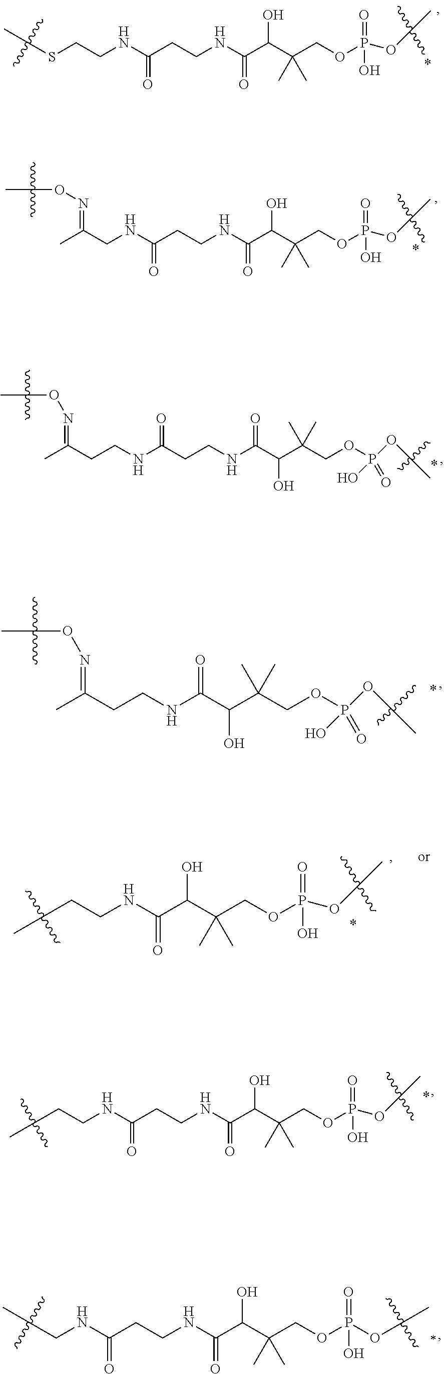
where the * indicates the point of attachment to A;
each R⁶ is independently selected from H and C₁-C₆alkyl;
each R¹⁰ is independently selected from H, C₁-C₆alkyl, F, Cl, and —OH;
each R¹¹ is independently selected from H, C₁-C₆alkyl, F, Cl, —NH₂, —OCH₃, —OCH₂CH₃, —N(CH₃)₂, —CN, —NO₂ and —OH;
each R¹² is independently selected from H, C₁₋₆alkyl, fluoro, benzyloxy substituted with —C(═O)OH, benzyl substituted with —C(═O)OH, C₁₋₄alkoxy substituted with —C(═O)OH and C1₄alkyl substituted with —C(═O)OH;
each R¹⁵ is independently selected from H, —CH₃ and phenyl;
each m is independently selected from 1, 2, 3, 4, 5, 6, 7, 8, 9 and 10, and
each p is independently selected from 1, 2, 3, 4, 5, 6, 7, 8, 9, 10, 11, 12, 13 and 14.

10. The conjugate of claim 9 selected from 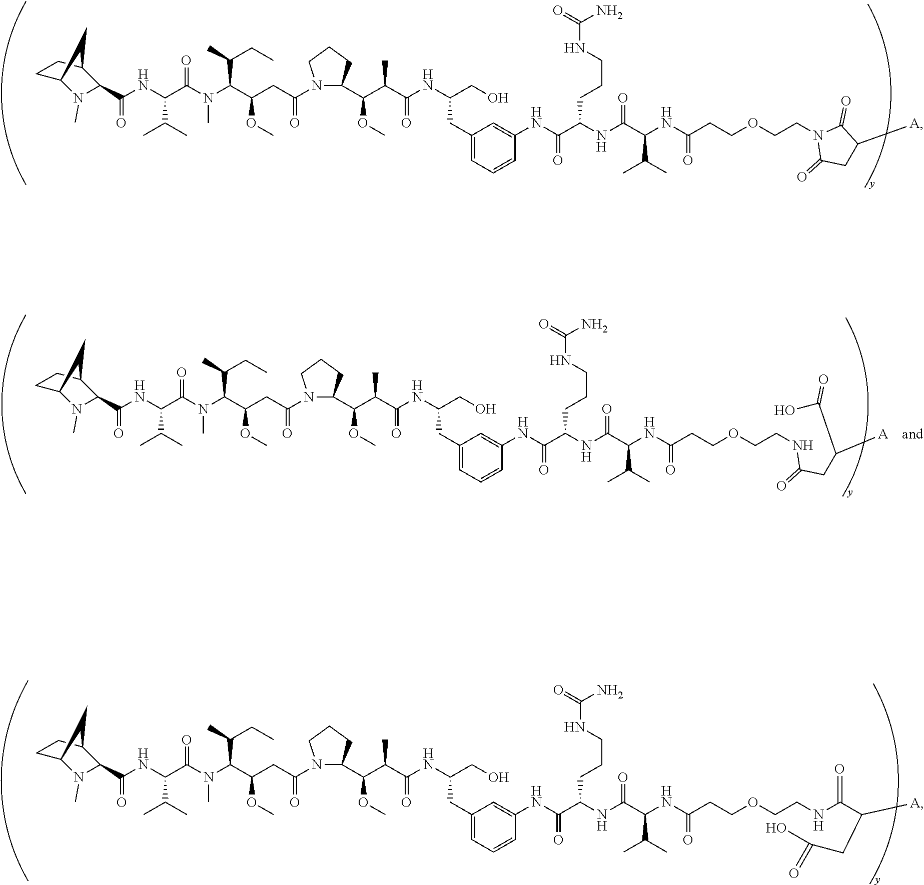
wherein:
A represents an antibody fragment (e.g., Fab or Fab′) that specifically binds to human cKIT, and
y is an integer from 1 to 10.

11. The conjugate of any one of claims 1 to 10, wherein the antibody fragment specifically binds to the extracellular domain of human cKIT (SEQ ID NO: 112).

12. The conjugate of any one of claims 1 to 10, wherein the antibody fragment specifically binds to an epitope in domains 1-3 of human cKIT (SEQ ID NO: 113).

13. The conjugate of any one of claims 1 to 12, wherein the antibody fragment is a Fab or Fab′.

14. The conjugate of any of claims 1 to 10 or claim 13, wherein the antibody fragment is selected from any of the following:
(1) a Fab or Fab′ comprising (i) a heavy chain variable region that comprises (a) a HCDR1 (Heavy Chain Complementarity Determining Region 1) of SEQ ID NO: 1, (b) a HCDR2 (Heavy Chain Complementarity Determining Region 2) of SEQ ID NO: 2, and (c) a HCDR3 (Heavy Chain Complementarity Determining Region 3) of SEQ ID NO: 3; and (ii) a light chain variable region that comprises: (d) a LCDR1 (Light Chain Complementarity Determining Region 1) of SEQ ID NO: 16, (e) a LCDR2 (Light Chain Complementarity Determining Region 2) of SEQ ID NO: 17, and (f) a LCDR3 (Light Chain Complementarity Determining Region 3) of SEQ ID NO: 18;
(2) a Fab or Fab′ comprising (i) a heavy chain variable region that comprises (a) a HCDR1 of SEQ ID NO: 4, (b) a HCDR2 of SEQ ID NO: 5, (c) a HCDR3 of SEQ ID NO: 3; and (ii) a light chain variable region that comprises: (d) a LCDR1 of SEQ ID NO: 19, (e) a LCDR2 of SEQ ID NO: 20, and (f a LCDR3 of SEQ ID NO: 21;
(3) a Fab or Fab′ comprising (i) a heavy chain variable region that comprises (a) a HCDR1 of SEQ ID NO: 6, (b) a HCDR2 of SEQ ID NO: 2, (c) a HCDR3 of SEQ ID NO: 3; and (ii) a light chain variable region that comprises: (d) a LCDR1 of SEQ ID NO: 16, (e) a LCDR2 of SEQ ID NO: 17, and (f a LCDR3 of SEQ ID NO: 18;
(4) a Fab or Fab′ comprising (i) a heavy chain variable region that comprises (a) a HCDR1 of SEQ ID NO: 7, (b) a HCDR2 of SEQ ID NO: 8, (c) a HCDR3 of SEQ ID NO: 9; and (ii) a light chain variable region that comprises: (d) a LCDR1 of SEQ ID NO: 22, (e) a LCDR2 of SEQ ID NO: 20, and (f a LCDR3 of SEQ ID NO: 18;
(5) a Fab or Fab′ comprising (i) a heavy chain variable region that comprises (a) a HCDR1 of SEQ ID NO: 27, (b) a HCDR2 of SEQ ID NO: 28, (c) a HCDR3 of SEQ ID NO: 29; and (ii) a light chain variable region that comprises: (d) a LCDR1 of SEQ ID NO: 42, (e) a LCDR2 of SEQ ID NO: 17, and (f) a LCDR3 of SEQ ID NO: 43;
(6) a Fab or Fab′ comprising (i) a heavy chain variable region that comprises (a) a HCDR1 of SEQ ID NO: 30, (b) a HCDR2 of SEQ ID NO: 31, (c) a HCDR3 of SEQ ID NO: 29; and (ii) a light chain variable region that comprises: (d) a LCDR1 of SEQ ID NO: 44, (e) a LCDR2 of SEQ ID NO: 20, and (f) a LCDR3 of SEQ ID NO: 45;
(7) a Fab or Fab′ comprising (i) a heavy chain variable region that comprises (a) a HCDR1 of SEQ ID NO: 32, (b) a HCDR2 of SEQ ID NO: 28, (c) a HCDR3 of SEQ ID NO: 29; and (ii) a light chain variable region that comprises: (d) a LCDR1 of SEQ ID NO: 42, (e) a LCDR2 of SEQ ID NO: 17, and (f) a LCDR3 of SEQ ID NO: 43;
(8) a Fab or Fab′ comprising (i) a heavy chain variable region that comprises (a) a HCDR1 of SEQ ID NO: 33, (b) a HCDR2 of SEQ ID NO: 34, (c) a HCDR3 of SEQ ID NO: 35; and (ii) a light chain variable region that comprises: (d) a LCDR1 of SEQ ID NO: 46, (e) a LCDR2 of SEQ ID NO: 20, and (f) a LCDR3 of SEQ ID NO: 43;
(9) a Fab or Fab′ comprising (i) a heavy chain variable region that comprises (a) a HCDR1 of SEQ ID NO: 1, (b) a HCDR2 of SEQ ID NO: 51, (c) a HCDR3 of SEQ ID NO: 3; and (ii) a light chain variable region that comprises: (d) a LCDR1 of SEQ ID NO: 16, (e) a LCDR2 of SEQ ID NO: 17, and (f) a LCDR3 of SEQ ID NO: 18;
(10) a Fab or Fab′ comprising (i) a heavy chain variable region that comprises (a) a HCDR1 of SEQ ID NO: 4, (b) a HCDR2 of SEQ ID NO: 52, (c) a HCDR3 of SEQ ID NO: 3; and (ii) a light chain variable region that comprises: (d) a LCDR1 of SEQ ID NO: 19, (e) a LCDR2 of SEQ ID NO: 20, and (f) a LCDR3 of SEQ ID NO: 21;
(11) a Fab or Fab′ comprising (i) a heavy chain variable region that comprises (a) a HCDR1 of SEQ ID NO: 6, (b) a HCDR2 of SEQ ID NO: 51, (c) a HCDR3 of SEQ ID NO: 3; and (ii) a light chain variable region that comprises: (d) a LCDR1 of SEQ ID NO: 16, (e) a LCDR2 of SEQ ID NO: 17, and (f) a LCDR3 of SEQ ID NO: 18;
(12) a Fab or Fab′ comprising (i) a heavy chain variable region that comprises (a) a HCDR1 of SEQ ID NO: 7, (b) a HCDR2 of SEQ ID NO: 53, (c) a HCDR3 of SEQ ID NO: 9; and (ii) a light chain variable region that comprises: (d) a LCDR1 of SEQ ID NO: 22, (e) a LCDR2 of SEQ ID NO: 20, and (f) a LCDR3 of SEQ ID NO: 18;
(13) a Fab or Fab′ comprising (i) a heavy chain variable region that comprises (a) a HCDR1 of SEQ ID NO: 60, (b) a HCDR2 of SEQ ID NO: 61, (c) a HCDR3 of SEQ ID NO: 62; and (ii) a light chain variable region that comprises: (d) a LCDR1 of SEQ ID NO: 75, (e) a LCDR2 of SEQ ID NO: 76, and (f) a LCDR3 of SEQ ID NO: 77;
(14) a Fab or Fab′ comprising (i) a heavy chain variable region that comprises (a) a HCDR1 of SEQ ID NO: 63, (b) a HCDR2 of SEQ ID NO: 64, (c) a HCDR3 of SEQ ID NO: 62; and (ii) a light chain variable region that comprises: (d) a LCDR1 of SEQ ID NO: 78, (e) a LCDR2 of SEQ ID NO: 79, and (f) a LCDR3 of SEQ ID NO: 80;
(15) a Fab or Fab′ comprising (i) a heavy chain variable region that comprises (a) a HCDR1 of SEQ ID NO: 65, (b) a HCDR2 of SEQ ID NO: 61, (c) a HCDR3 of SEQ ID NO: 62; and (ii) a light chain variable region that comprises: (d) a LCDR1 of SEQ ID NO: 75, (e) a LCDR2 of SEQ ID NO: 76, and (f) a LCDR3 of SEQ ID NO: 77;
(16) a Fab or Fab′ comprising (i) a heavy chain variable region that comprises (a) a HCDR1 of SEQ ID NO: 66, (b) a HCDR2 of SEQ ID NO: 67, (c) a HCDR3 of SEQ ID NO: 68; and (ii) a light chain variable region that comprises: (d) a LCDR1 of SEQ ID NO: 81, (e) a LCDR2 of SEQ ID NO: 79, and (f) a LCDR3 of SEQ ID NO: 77;
(17) a Fab or Fab′ comprising (i) a heavy chain variable region that comprises (a) a HCDR1 of SEQ ID NO: 86, (b) a HCDR2 of SEQ ID NO: 87, (c) a HCDR3 of SEQ ID NO: 88; and (ii) a light chain variable region that comprises: (d) a LCDR1 of SEQ ID NO: 101, (e) a LCDR2 of SEQ ID NO: 102, and (f a LCDR3 of SEQ ID NO: 103;
(18) a Fab or Fab′ comprising (i) a heavy chain variable region that comprises (a) a HCDR1 of SEQ ID NO: 89, (b) a HCDR2 of SEQ ID NO: 90, (c) a HCDR3 of SEQ ID NO: 88; and (ii) a light chain variable region that comprises: (d) a LCDR1 of SEQ ID NO: 104, (e) a LCDR2 of SEQ ID NO: 105, and (f a LCDR3 of SEQ ID NO: 106;
(19) a Fab or Fab′ comprising (i) a heavy chain variable region that comprises (a) a HCDR1 of SEQ ID NO: 91, (b) a HCDR2 of SEQ ID NO: 87, (c) a HCDR3 of SEQ ID NO: 88; and (ii) a light chain variable region that comprises: (d) a LCDR1 of SEQ ID NO: 101, (e) a LCDR2 of SEQ ID NO: 102, and (f a LCDR3 of SEQ ID NO: 103;
(20) a Fab or Fab′ comprising (i) a heavy chain variable region that comprises (a) a HCDR1 of SEQ ID NO: 92, (b) a HCDR2 of SEQ ID NO: 93, (c) a HCDR3 of SEQ ID NO: 94; and (ii) a light chain variable region that comprises: (d) a LCDR1 of SEQ ID NO: 107, (e) a LCDR2 of SEQ ID NO: 105, and (f a LCDR3 of SEQ ID NO: 103;
(21) a Fab or Fab′ comprising a heavy chain variable region (VH) that comprises SEQ ID NO: 10, and a light chain variable region (VL) that comprises SEQ ID NO: 23;
(22) a Fab or Fab′ comprising a VH that comprises SEQ ID NO: 36, and a VL that comprises SEQ ID NO: 47;
(23) a Fab or Fab′ comprising a VH that comprises SEQ ID NO: 54, and a VL that comprises SEQ ID NO: 23;
(24) a Fab or Fab′ comprising a VH that comprises SEQ ID NO: 69, and a VL that comprises SEQ ID NO: 82;
(25) a Fab or Fab′ comprising a VH that comprises SEQ ID NO: 95, and a VL that comprises SEQ ID NO: 108;
(26) a Fab′ comprising a heavy chain that that comprises SEQ ID NO: 14, and a light chain that comprises SEQ ID NO: 25;
(27) a Fab′ comprising a heavy chain that that comprises SEQ ID NO: 40, and a light chain that comprises SEQ ID NO: 49;
(28) a Fab′ comprising a heavy chain that that comprises SEQ ID NO: 58, and a light chain that comprises SEQ ID NO: 25;
(29) a Fab′ comprising a heavy chain that that comprises SEQ ID NO: 73, and a light chain that comprises SEQ ID NO: 84;
(30) a Fab′ comprising a heavy chain that that comprises SEQ ID NO: 99, and a light chain that comprises SEQ ID NO:110;
(31) a Fab comprising a heavy chain that comprises SEQ ID NO: 118, and a light chain that comprises SEQ ID NO: 122;
(32) a Fab comprising a heavy chain that comprises SEQ ID NO: 118, and a light chain that comprises SEQ ID NO: 123;
(33) a Fab comprising a heavy chain that comprises SEQ ID NO: 124, and a light chain that comprises SEQ ID NO: 128;
(34) a Fab comprising a heavy chain that comprises SEQ ID NO: 124, and a light chain that comprises SEQ ID NO: 129;
(35) a Fab comprising a heavy chain that comprises SEQ ID NO: 130, and a light chain that comprises SEQ ID NO: 134;
(36) a Fab comprising a heavy chain that comprises SEQ ID NO: 130, and a light chain that comprises SEQ ID NO: 135;
(37) a Fab comprising a heavy chain that comprises SEQ ID NO: 136, and a light chain that comprises SEQ ID NO: 140;
(38) a Fab comprising a heavy chain that comprises SEQ ID NO: 141, and a light chain that comprises SEQ ID NO: 145;
(39) a Fab comprising a heavy chain that comprises an amino acid sequence selected from SEQ ID NO: 119, 120 or 121, and a light chain comprising the amino acid sequence of SEQ ID NO: 25;
(40) a Fab comprising a heavy chain that comprises an amino acid sequence selected from SEQ ID NO: 125, 126, or 127, and a light chain comprising the amino acid sequence of SEQ ID NO: 49;
(41) a Fab comprising a heavy chain that comprises an amino acid sequence selected from SEQ ID NO: 131, 132, or 133, and a light chain comprising the amino acid sequence of SEQ ID NO: 25;
(42) a Fab comprising a heavy chain that comprises an amino acid sequence selected from SEQ ID NO: 137, 138, or 139, and a light chain comprising the amino acid sequence of SEQ ID NO: 84; or
(43) a Fab comprising a heavy chain that comprises an amino acid sequence selected from SEQ ID NO: 142, 143, or 144, and a light chain comprising the amino acid sequence of SEQ ID NO: 110.

15. The conjugate of any one of claims 1 to 14, wherein the antibody fragment is a human or humanized Fab or Fab′.

16. The conjugate of any one of claims 1 to 6, wherein the antibody fragment is a Fab′ and the linker (L_{B}) is attached to a native cysteine residue in the hinge region of the Fab′.

17. The conjugate of any one of claims 1 to 6, wherein the antibody fragment comprises at least one non-native cysteine introduced into a constant region, and the linker (L_{B}) is attached to the non-native cysteine.

18. The conjugate of any one of claims 7 to 10, wherein the antibody fragment is a Fab′ and R¹¹⁴ is attached to a native cysteine residue in the hinge region of the Fab′.

19. The conjugate of any one of claims 7 to 10, wherein the antibody fragment comprises at least one non-native cysteine introduced into a constant region, and R¹¹⁴ is attached to the non-native cysteine.

20. The conjugate of any one of claim 17 or 19, wherein the antibody fragment comprises cysteine at one or more of the following positions (all positions by EU numbering):
(a) position 152 of the heavy chain,
(b) position 114 or 165 of the kappa light chain, or
(c) position 143 of the lambda light chain.

21. The conjugate of any one of claims 1 to 20, wherein the half-life of the conjugate is less than about 24-48 hours.

22. The conjugate of any one of claims 1 to 21, wherein the conjugate does not induce mast cell degranulation.

23. A pharmaceutical composition comprising the conjugate of any one of claims 1 to 22 and a pharmaceutically acceptable carrier.

24. The pharmaceutical composition of claim 23 further comprising another therapeutic agent.

25. The pharmaceutical composition of claim 23 or 24, wherein the composition is a lyophilisate.

26. A method of ablating hematopoietic stem cells in a patient in need thereof, the method comprising administering to the patient an effective amount of the conjugate of any of claims 1-22, or the pharmaceutical composition of claim 23 or 24.

27. The method of claim 26, wherein the patient is a hematopoietic stem cell transplantation recipient.

28. The method of claim 27, wherein the method is performed before hematopoietic stem cell transplantation to the patient.

29. A method of conditioning a hematopoietic stem cell transplantation patient, the method comprising: administering to the patient an effective amount of the conjugate of any of claims 1-22, or the pharmaceutical composition of claim 23 or 24, and allowing a sufficient period of time for the conjugates to clear from the patient's circulation before performing hematopoietic stem cell transplantation to the patient.

30. The method of any one of claims 26-29, wherein the patient has an inherited immunodeficient disease, an autoimmune disorder, a hematopoietic disorder, an inborn error of metabolism, or previously been treated with autologous stem cell transplantation.

31. The method of claim 30, wherein the hematopoietic disorder is selected from: Acute myeloid leukemia (AML), Acute lymphoblastic leukemia (ALL), acute monocytic leukemia (AMoL), Chronic myeloid leukemia (CML), Chronic lymphocytic leukemia (CLL), Myeloproliferative disorders, Myelodysplastic syndromes, Multiple myeloma, Non-Hodgkin lymphoma, Hodgkin disease, Aplastic anemia, Pure red cell aplasia, Paroxysmal nocturnal hemoglobinuria, Fanconi anemi, Thalassemia major, Sickle cell anemia, Severe combined immunodeficiency, Wiskott-Aldrich syndrome, Hemophagocytic lymphohistiocytosis.

32. The method of claim 30, wherein the inborn error of metabolism is selected from mucopolysaccharidosis, Gaucher disease, metachromatic leukodystrophies, or adrenoleukodystrophies.

33. The method of any of claims 26-29, wherein the patient has a non-malignant disease or condition selected from Severe aplastic anemia (SAA), Wiskott Aldrich Syndrome, Hurlers Syndrome, FHL, CGD, Kostmanns syndrome, Severe immunodeficiency syndrome (SCID), other autoimmune disorders such as SLE, Multiple sclerosis, IBD, Crolms Disease, Sjogrens syndrome, vasculitis, Lupus, Myasthenia Gravis, Wegeners disease, inborn errors of metabolism and/or other immunodeficiencies.

34. The method of any of claims 26-29, wherein the patient has a malignant disease or condition selected from myelodysplastic syndromes (MDS), acute lymphoblastic leukemia (ALL), acute myelogenous leukemia (AML), acute monocytic leukemia (AMoL), chronic lymphocytic leukemia (CLL), chronic myelogenous leukemia (CML), hairy cell leukemia (HCL), T-cell prolymphocytic leukemia (T-PLL), large granular lymphocytic leukemia, adult T-cell leukemia, Precursor T-cell leukemia/lymphoma, Burkitt lymphoma, follicular lymphoma, diffuse large B cell lymphoma, mantle cell lymphoma, B-cell chronic lymphocytic leukemia/lymphoma, MALT lymphoma, Mycosis fungoides, Peripheral T-cell lymphoma not otherwise specified, Nodular sclerosis form of Hodgkin lymphoma Mixed-cellularity subtype of Hodgkin lymphoma.

35. Use of the conjugate of any of claims 1-22, or the pharmaceutical composition of claim 23 or 24, for ablating hematopoietic stem cells in a patient in need thereof.

36. Use of the conjugate of any of claims 1-22, or the pharmaceutical composition of claim 23 or 24, in the manufacture of a medicament for ablating hematopoietic stem cells in a patient in need thereof.

37. An antibody or antibody fragment that specifically binds to human cKIT, wherein the antibody or antibody fragment is selected from any of the following:
(1) an antibody or antibody fragment comprising (i) a heavy chain variable region that comprises (a) a HCDR1 (Heavy Chain Complementarity Determining Region 1) of SEQ ID NO: 1, (b) a HCDR2 (Heavy Chain Complementarity Determining Region 2) of SEQ ID NO: 2, and (c) a HCDR3 (Heavy Chain Complementarity Determining Region 3) of SEQ ID NO: 3; and (ii) a light chain variable region that comprises: (d) a LCDR1 (Light Chain Complementarity Determining Region 1) of SEQ ID NO: 16, (e) a LCDR2 (Light Chain Complementarity Determining Region 2) of SEQ ID NO: 17, and (f) a LCDR3 (Light Chain Complementarity Determining Region 3) of SEQ ID NO: 18;
(2) an antibody or antibody fragment comprising (i) a heavy chain variable region that comprises (a) a HCDR1 of SEQ ID NO: 4, (b) a HCDR2 of SEQ ID NO: 5, (c) a HCDR3 of SEQ ID NO: 3; and (ii) a light chain variable region that comprises: (d) a LCDR1 of SEQ ID NO: 19, (e) a LCDR2 of SEQ ID NO: 20, and (f) a LCDR3 of SEQ ID NO: 21;
(3) an antibody or antibody fragment comprising (i) a heavy chain variable region that comprises (a) a HCDR1 of SEQ ID NO: 6, (b) a HCDR2 of SEQ ID NO: 2, (c) a HCDR3 of SEQ ID NO: 3; and (ii) a light chain variable region that comprises: (d) a LCDR1 of SEQ ID NO: 16, (e) a LCDR2 of SEQ ID NO: 17, and (f) a LCDR3 of SEQ ID NO: 18;
(4) an antibody or antibody fragment comprising (i) a heavy chain variable region that comprises (a) a HCDR1 of SEQ ID NO: 7, (b) a HCDR2 of SEQ ID NO: 8, (c) a HCDR3 of SEQ ID NO: 9; and (ii) a light chain variable region that comprises: (d) a LCDR1 of SEQ ID NO: 22, (e) a LCDR2 of SEQ ID NO: 20, and (f) a LCDR3 of SEQ ID NO: 18;
(5) an antibody or antibody fragment comprising (i) a heavy chain variable region that comprises (a) a HCDR1 of SEQ ID NO: 27, (b) a HCDR2 of SEQ ID NO: 28, (c) a HCDR3 of SEQ ID NO: 29; and (ii) a light chain variable region that comprises: (d) a LCDR1 of SEQ ID NO: 42, (e) a LCDR2 of SEQ ID NO: 17, and (f) a LCDR3 of SEQ ID NO: 43;
(6) an antibody or antibody fragment comprising (i) a heavy chain variable region that comprises (a) a HCDR1 of SEQ ID NO: 30, (b) a HCDR2 of SEQ ID NO: 31, (c) a HCDR3 of SEQ ID NO: 29; and (ii) a light chain variable region that comprises: (d) a LCDR1 of SEQ ID NO: 44, (e) a LCDR2 of SEQ ID NO: 20, and (f) a LCDR3 of SEQ ID NO: 45;
(7) an antibody or antibody fragment comprising (i) a heavy chain variable region that comprises (a) a HCDR1 of SEQ ID NO: 32, (b) a HCDR2 of SEQ ID NO: 28, (c) a HCDR3 of SEQ ID NO: 29; and (ii) a light chain variable region that comprises: (d) a LCDR1 of SEQ ID NO: 42, (e) a LCDR2 of SEQ ID NO: 17, and (f) a LCDR3 of SEQ ID NO: 43;
(8) an antibody or antibody fragment comprising (i) a heavy chain variable region that comprises (a) a HCDR1 of SEQ ID NO: 33, (b) a HCDR2 of SEQ ID NO: 34, (c) a HCDR3 of SEQ ID NO: 35; and (ii) a light chain variable region that comprises: (d) a LCDR1 of SEQ ID NO: 46, (e) a LCDR2 of SEQ ID NO: 20, and (f) a LCDR3 of SEQ ID NO: 43;
(9) an antibody or antibody fragment comprising (i) a heavy chain variable region that comprises (a) a HCDR1 of SEQ ID NO: 60, (b) a HCDR2 of SEQ ID NO: 61, (c) a HCDR3 of SEQ ID NO: 62; and (ii) a light chain variable region that comprises: (d) a LCDR1 of SEQ ID NO: 75, (e) a LCDR2 of SEQ ID NO: 76, and (f) a LCDR3 of SEQ ID NO: 77;
(10) an antibody or antibody fragment comprising (i) a heavy chain variable region that comprises (a) a HCDR1 of SEQ ID NO: 63, (b) a HCDR2 of SEQ ID NO: 64, (c) a HCDR3 of SEQ ID NO: 62; and (ii) a light chain variable region that comprises: (d) a LCDR1 of SEQ ID NO: 78, (e) a LCDR2 of SEQ ID NO: 79, and (f) a LCDR3 of SEQ ID NO: 80;
(11) an antibody or antibody fragment comprising (i) a heavy chain variable region that comprises (a) a HCDR1 of SEQ ID NO: 65, (b) a HCDR2 of SEQ ID NO: 61, (c) a HCDR3 of SEQ ID NO: 62; and (ii) a light chain variable region that comprises: (d) a LCDR1 of SEQ ID NO: 75, (e) a LCDR2 of SEQ ID NO: 76, and (f) a LCDR3 of SEQ ID NO: 77;
(12) an antibody or antibody fragment comprising (i) a heavy chain variable region that comprises (a) a HCDR1 of SEQ ID NO: 66, (b) a HCDR2 of SEQ ID NO: 67, (c) a HCDR3 of SEQ ID NO: 68; and (ii) a light chain variable region that comprises: (d) a LCDR1 of SEQ ID NO: 81, (e) a LCDR2 of SEQ ID NO: 79, and (f) a LCDR3 of SEQ ID NO: 77;
(13) an antibody or antibody fragment comprising (i) a heavy chain variable region that comprises (a) a HCDR1 of SEQ ID NO: 86, (b) a HCDR2 of SEQ ID NO: 87, (c) a HCDR3 of SEQ ID NO: 88; and (ii) a light chain variable region that comprises: (d) a LCDR1 of SEQ ID NO: 101, (e) a LCDR2 of SEQ ID NO: 102, and (f) a LCDR3 of SEQ ID NO: 103;
(14) an antibody or antibody fragment comprising (i) a heavy chain variable region that comprises (a) a HCDR1 of SEQ ID NO: 89, (b) a HCDR2 of SEQ ID NO: 90, (c) a HCDR3 of SEQ ID NO: 88; and (ii) a light chain variable region that comprises: (d) a LCDR1 of SEQ ID NO: 104, (e) a LCDR2 of SEQ ID NO: 105, and (f) a LCDR3 of SEQ ID NO: 106;
(15) an antibody or antibody fragment comprising (i) a heavy chain variable region that comprises (a) a HCDR1 of SEQ ID NO: 91, (b) a HCDR2 of SEQ ID NO: 87, (c) a HCDR3 of SEQ ID NO: 88; and (ii) a light chain variable region that comprises: (d) a LCDR1 of SEQ ID NO: 101, (e) a LCDR2 of SEQ ID NO: 102, and (f) a LCDR3 of SEQ ID NO: 103;
(16) an antibody or antibody fragment comprising (i) a heavy chain variable region that comprises (a) a HCDR1 of SEQ ID NO: 92, (b) a HCDR2 of SEQ ID NO: 93, (c) a HCDR3 of SEQ ID NO: 94; and (ii) a light chain variable region that comprises: (d) a LCDR1 of SEQ ID NO: 107, (e) a LCDR2 of SEQ ID NO: 105, and (f) a LCDR3 of SEQ ID NO: 103;
(17) an antibody or antibody fragment comprising a heavy chain variable region (VH) that comprises SEQ ID NO: 10, and a light chain variable region (VL) that comprises SEQ ID NO: 23;
(18) an antibody or antibody fragment comprising a VH that comprises SEQ ID NO: 36, and a VL that comprises SEQ ID NO: 47;
(19) an antibody or antibody fragment comprising a VH that comprises SEQ ID NO: 69, and a VL that comprises SEQ ID NO: 82;
(20) an antibody or antibody fragment comprising a VH that comprises SEQ ID NO: 95, and a VL that comprises SEQ ID NO: 108;
(21) an antibody or antibody fragment comprising a heavy chain that that comprises SEQ ID NO: 14, and a light chain that comprises SEQ ID NO: 25;
(22) an antibody or antibody fragment comprising a heavy chain that that comprises SEQ ID NO: 40, and a light chain that comprises SEQ ID NO: 49;
(23) an antibody or antibody fragment comprising a heavy chain that that comprises SEQ ID NO: 73, and a light chain that comprises SEQ ID NO: 84;
(24) an antibody or antibody fragment comprising a heavy chain that that comprises SEQ ID NO: 99, and a light chain that comprises SEQ ID NO: 110;
(25) an antibody or antibody fragment comprising a heavy chain that comprises SEQ ID NO: 118, and a light chain that comprises SEQ ID NO: 122;
(26) an antibody or antibody fragment comprising a heavy chain that comprises SEQ ID NO: 118, and a light chain that comprises SEQ ID NO: 123;
(27) an antibody or antibody fragment comprising a heavy chain that comprises SEQ ID NO: 124, and a light chain that comprises SEQ ID NO: 128;
(28) an antibody or antibody fragment comprising a heavy chain that comprises SEQ ID NO: 124, and a light chain that comprises SEQ ID NO: 129;
(29) an antibody or antibody fragment comprising a heavy chain that comprises SEQ ID NO: 130, and a light chain that comprises SEQ ID NO: 134;
(30) an antibody or antibody fragment comprising a heavy chain that comprises SEQ ID NO: 130, and a light chain that comprises SEQ ID NO: 135;
(31) an antibody or antibody fragment comprising a heavy chain that comprises SEQ ID NO: 136, and a light chain that comprises SEQ ID NO: 140;
(32) an antibody or antibody fragment comprising a heavy chain that comprises SEQ ID NO: 141, and a light chain that comprises SEQ ID NO: 145;
(33) an antibody comprising a heavy chain that that comprises SEQ ID NO: 12, and a light chain that comprises SEQ ID NO: 25;
(34) an antibody comprising a heavy chain that that comprises SEQ ID NO: 38, and a light chain that comprises SEQ ID NO: 49;
(35) an antibody comprising a heavy chain that that comprises SEQ ID NO: 71, and a light chain that comprises SEQ ID NO: 84; or
(36) an antibody comprising a heavy chain that that comprises SEQ ID NO: 97, and a light chain that comprises SEQ ID NO: 110.

38. A nucleic acid encoding an antibody or antibody fragment of claim 37.

39. A vector comprising the nucleic acid of claim 38.

40. A host cell comprising the vector of claim 39.

41. A process for producing an antibody or antibody fragment, the process comprising cultivating the host cell of claim 40, and recovering the antibody or antibody fragment from the culture.
